SEQUENCE LISTING (57 sequences)

SEQ ID NO: 1 - DNA, 747 bp, synthetic construct
  source 1..747: mol_type = other DNA
atgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 2 - DNA, 2898 bp, synthetic construct
  source 1..2898: mol_type = other DNA
atcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattcc

SEQ ID NO: 3 - DNA, 36 bp, synthetic construct
  source 1..36: mol_type = other DNA
caagtaaacccctaccaactggtcggggtttgaaac

SEQ ID NO: 4 - DNA, 2181 bp, synthetic construct
  source 1..2181: mol_type = other DNA
atgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaacactagtggcggctctggcggcggctccggaggctctagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggacccagcgcccgcagtgactcccgaggccagtcacctgttggaggaccccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 5 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 6 - DNA, 2789 bp, synthetic construct
  source 1..2789: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggacttagcctttagtgaaccgtcagaattaattcagatcgatctaccagaaccgtcagatccgctagagattacgccaaccgccaccatgggcagcatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaacactagtggcggctctggcggcggctccggaggctctagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggacccagcgcccgcagtgactcccgaggccagtcacctgttggaggaccccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttga

SEQ ID NO: 7 - DNA, 379 bp, synthetic construct
  source 1..379: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 8 - DNA, 549 bp, synthetic construct
  source 1..549: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagaga

SEQ ID NO: 9 - DNA, 4096 bp, synthetic construct
  source 1..4096: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcaacatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtcaggctaa

SEQ ID NO: 10 - DNA, 4649 bp, synthetic construct
  source 1..4649: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaaggacttagcctttagtgaaccgtcagaattaattcagatcgatctaccagaaccgtcagatccgctagagattacgccaaccgccaccatgggcagcatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcaacatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtcaggctaaccggactctagaacatccctacaggtgatatcctcgtgctgcaagtaaacccctaccaactggtcggggtttgaaac

SEQ ID NO: 11 - DNA, 1594 bp, synthetic construct
  source 1..1594: mol_type = other DNA
atgggcagcgccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgattttt

SEQ ID NO: 12 - DNA, 891 bp, synthetic construct
  source 1..891: mol_type = other DNA
atgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggaaaccactgccatgttgtattcctgcaggacggagcggcggcggcagcagtttattcgcgcgctgctgcagcaccaccgccctatcctgatgcacgattatgactctacccccatgtcaggctaa

SEQ ID NO: 13 - DNA, 3731 bp, synthetic construct
  source 1..3731: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaaggacttagcctttagtgaaccgtcagaattaattcagatcgatctaccagaaccgtcagatccgctagagattacgccaaccgccaccatgggcagcatgggcagcgccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttcaggctaataacagcttccggactctagaacatccctacaggtgatatcctcgatcccgctcgataagcttgatatcgaattccgcccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgcgtccatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggaaaccactgccatgttgtattcctgcaggacggagcggcggcggcagcagtttattcgcgcgctgctgcagcaccaccgccctatcctgatgcacgattatgactctacccccatgtcaggctaataacagcttccggactctagaacatccctacaggtgatatcctcgtgctgcaagtaaacccctaccaactggtcggggtttgaaac

SEQ ID NO: 14 - DNA, 3620 bp, synthetic construct
  source 1..3620: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgaggacttagcctttagtgaaccgtcagaattaattcagatcgatctaccagaaccgtcagatccgctagagattacgccaaccgccaccatgggcagcatgagccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtg

SEQ ID NO: 15 - DNA, 1575 bp, synthetic construct
  source 1..1575: mol_type = other DNA
atggacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctaggcctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggccccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagaaacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttaa

SEQ ID NO: 16 - DNA, 4140 bp, synthetic construct
  source 1..4140: mol_type = other DNA
atggacaagaaatactccatcggccttgccatcgggaccaacagcgtgggttgggccgtgatcactgacgaatacaaagtgccatcgaaaaagtttaaggtgctgggcaatactgacagacatagcatcaagaaaaacctgattggagccctcctgttcgactccggggaaaccgccgaagccactcgcctgaaacggaccgcacgccggaggtacactcgccggaagaatagaatctgctaccttcaagagatctttagcaacgagatggccaaagtggacgattcgttcttccaccggctggaggagtcattcctggtcgaagaggacaaaaagcatgagagacatccgatcttcggtaacattgtggatgaggtcgcctaccacgagaagtaccccactatctaccatttgcgcaagaagttggtcgactcgactgataaggccgacctcagactcatctacctcgctttggcacacatgatcaagtttaggggtcacttcctgattgaaggggatctgaacccagacaactcggatgtggataaactgtttatccagctggtccagacttacaatcagctctttgaggagaacccgatcaatgcctccggcgtggatgctaaggcaatcctgtccgcacgcttgtcaaagagccggcggctcgaaaatctgatcgcacagcttccgggagaaaagaaaaacggactgtttggaaaccttatcgccctctcgctgggactcactcctaacttcaaatccaattttgatttggccgaggatgccaagctgcagctgtcgaaggacacctacgacgatgacctcgacaatcttctggcccagatcggggaccagtacgccgatctgttcctcgcggccaaaaacctgtcagacgcaatcttgctgagcgatatcttgagggtcaataccgaaatcactaaggctccattgtcagcatcgatgatcaagaggtacgatgaacaccatcaggacctgaccctgctcaaggcactggtgcggcagcaactgccggaaaagtacaaggagatcttcttcgatcaaagcaagaatgggtacgcagggtacatcgatggtggagcatcacaagaagagttctacaaattcatcaagcccatccttgaaaagatggacgggacggaagaactgctggtgaagctgaatcgggaagatctgctgcggaagcagcgcaccttcgacaatggttccatcccacaccaaatccatctcggcgaactgcacgcgatccttcgccggcaggaagatttctacccgttcttgaaggataacagagaaaagatcgagaaaatcctgacctttagaatcccgtactacgtgggcccgttggctcgcggaaactcaagattcgcctggatgactagaaaatccgaagaaacgattaccccgtggaactttgaagaggtcgtcgacaagggagcctcggcacagtcgttcatcgaacggatgaccaatttcgacaagaacctccccaacgaaaaggtgctcccgaaacactcgctgctgtatgagtacttcacggtctacaacgagctgaccaaggtgaagtacgtgaccgaaggaatgcggaagcccgcttttctgtccggtgaacagaaaaaggccatcgtggacctcctcttcaagactaacagaaaggtgaccgtgaaacagcttaaagaggactacttcaagaagatcgagtgttttgactccgtggaaatctcgggagtggaggataggtttaacgcttcgctggggacctaccatgaccttctcaagatcatcaaggataaggatttcctggacaacgaagaaaacgaggacatccttgaggacatcgtgttgaccttgaccctgtttgaggatcgggaaatgatcgaggagagactgaaaacttacgcgcatcttttcgacgacaaagtgatgaagcagctgaaaagaagaagatacactggctggggacggctgtcgaggaaactgattaacggaattagggataaacaaagcggaaagactatcctcgattttctcaagtcggacggcttcgccaatcggaactttatgcagctcatccacgacgatagcctgaccttcaaggaagatatccaaaaagcccaagtgtccggccagggagatagcttgcacgagcacattgctaacctggccggttcaccagccattaagaagggaatcctccaaaccgtgaaggtggtcgacgaactcgtcaaggtgatgggccgccataagccagagaacatcgtgatcgagatggctcgcgaaaatcagactactcagaagggtcaaaagaactcccgcgagcgcatgaagcgcatcgaagaaggaattaaggagctgggctcacaaatcctcaaagaacatcctgtcgagaacacccaacttcagaacgagaaactgtacctctactatctccaaaatggccgggacatgtacgtcgatcaggaattggatatcaaccgcctctcagactacgacgtggatgctatcgtgccgcagtcgtttctcaaagatgatagcatcgacaacaaggtcctgacgaggtcggataagaaccgcgggaaatccgacaatgtgccttcggaagaggtggtgaaaaagatgaagaactactggaggcaattgcttaatgccaaactgatcacccagcgcaaattcgacaaccttactaaggccgagcgcggaggactgtccgaactcgataaggcggggttcatcaaaagacaattggtcgaaactcggcaaattaccaagcatgtcgctcagatcctcgactcgcgcatgaacactaagtatgatgagaacgacaagctcattcgcgaagtgaaagtgattaccctcaaatcaaagctggtgtcggacttcagaaaagactttcaattctacaaggtgcgggagattaacaactaccaccacgcgcacgacgcctacctcaatgcagtggtgggaaccgccctcatcaagaagtatccaaagctcgaaagcgagttcgtgtacggggactataaagtgtacgatgtgcggaaaatgatcgcaaagagcgagcaagagatcggcaaagcaacggccaaatacttcttctacagcaacattatgaactttttcaagaccgagattaccctggcgaacggagaaatccggaagcggcctttgatcgaaacgaatggggaaactggagaaatcgtgtgggacaagggcagagactttgcgaccgtgagaaaggtgctgtccatgccacaagtcaacatcgtgaagaaaactgaagtgcagactggaggattttccaaggagtcaatccttccgaagcgcaacagcgacaagctgatcgccaggaagaaggattgggaccccaagaagtacggtggttttgattcacctactgtcgcttacagcgtgctggtggtggccaaggtggaaaaggggaagtcaaagaaattgaagtcggtgaaggaattgctggggattactatcatggagaggagcagcttcgaaaagaatcccatcgacttcttggaggccaagggatacaaagaggtgaagaaagacctgatcattaagctgccgaagtactccttgttcgaactggaaaacggcagaaagcggatgctggcctccgccggagagctgcagaagggtaacgagctggctctgcccagcaaatacgtgaatttcctctacctggcctcccattacgagaagctcaagggaagcccggaggataatgagcaaaaacaactgttcgtcgagcagcacaagcactacctcgacgagatcatcgaacaaatctccgagttctcgaagcgggtgattctggccgacgcaaaccttgataaagtcctcagcgcctacaacaagcaccgcgacaaaccgatcagagaacaagctgagaacatcatccacctgttcacgctgactaacttgggagcgcctgccgccttcaagtacttcgacaccactatcgatcggaaacggtacacttccactaaggaagtgctggacgcaaccctgatccatcagtccatcaccggactttatgagactcggatcgatctcagccagcttggcggtgattcaagagctgacccgaagaagaagcgcaaggtctaa

SEQ ID NO: 17 - DNA, 5718 bp, synthetic construct
  source 1..5718: mol_type = other DNA
atggacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctaggcctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggccccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagaaacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaagcctatgacaagaaatactccatcggccttgccatcgggaccaacagcgtgggttgggccgtgatcactgacgaatacaaagtgccatcgaaaaagtttaaggtgctgggcaatactgacagacatagcatcaagaaaaacctgattggagccctcctgttcgactccggggaaaccgccgaagccactcgcctgaaacggaccgcacgccggaggtacactcgccggaagaatagaatctgctaccttcaagagatctttagcaacgagatggccaaagtggacgattcgttcttccaccggctggaggagtcattcctggtcgaagaggacaaaaagcatgagagacatccgatcttcggtaacattgtggatgaggtcgcctaccacgagaagtaccccactatctaccatttgcgcaagaagttggtcgactcgactgataaggccgacctcagactcatctacctcgctttggcacacatgatcaagtttaggggtcacttcctgattgaaggggatctgaacccagacaactcggatgtggataaactgtttatccagctggtccagacttacaatcagctctttgaggagaacccgatcaatgcctccggcgtggatgctaaggcaatcctgtccgcacgcttgtcaaagagccggcggctcgaaaatctgatcgcacagcttccgggagaaaagaaaaacggactgtttggaaaccttatcgccctctcgctgggactcactcctaacttcaaatccaattttgatttggccgaggatgccaagctgcagctgtcgaaggacacctacgacgatgacctcgacaatcttctggcccagatcggggaccagtacgccgatctgttcctcgcggccaaaaacctgtcagacgcaatcttgctgagcgatatcttgagggtcaataccgaaatcactaaggctccattgtcagcatcgatgatcaagaggtacgatgaacaccatcaggacctgaccctgctcaaggcactggtgcggcagcaactgccggaaaagtacaaggagatcttcttcgatcaaagcaagaatgggtacgcagggtacatcgatggtggagcatcacaagaagagttctacaaattcatcaagcccatccttgaaaagatggacgggacggaagaactgctggtgaagctgaatcgggaagatctgctgcggaagcagcgcaccttcgacaatggttccatcccacaccaaatccatctcggcgaactgcacgcgatccttcgccggcaggaagatttctacccgttcttgaaggataacagagaaaagatcgagaaaatcctgacctttagaatcccgtactacgtgggcccgttggctcgcggaaactcaagattcgcctggatgactagaaaatccgaagaaacgattaccccgtggaactttgaagaggtcgtcgacaagggagcctcggcacagtcgttcatcgaacggatgaccaatttcgacaagaacctccccaacgaaaaggtgctcccgaaacactcgctgctgtatgagtacttcacggtctacaacgagctgaccaaggtgaagtacgtgaccgaaggaatgcggaagcccgcttttctgtccggtgaacagaaaaaggccatcgtggacctcctcttcaagactaacagaaaggtgaccgtgaaacagcttaaagaggactacttcaagaagatcgagtgttttgactccgtggaaatctcgggagtggaggataggtttaacgcttcgctggggacctaccatgaccttctcaagatcatcaaggataaggatttcctggacaacgaagaaaacgaggacatccttgaggacatcgtgttgaccttgaccctgtttgaggatcgggaaatgatcgaggagagactgaaaacttacgcgcatcttttcgacgacaaagtgatgaagcagctgaaaagaagaagatacactggctggggacggctgtcgaggaaactgattaacggaattagggataaacaaagcggaaagactatcctcgattttctcaagtcggacggcttcgccaatcggaactttatgcagctcatccacgacgatagcctgaccttcaaggaagatatccaaaaagcccaagtgtccggccagggagatagcttgcacgagcacattgctaacctggccggttcaccagccattaagaagggaatcctccaaaccgtgaaggtggtcgacgaactcgtcaaggtgatgggccgccataagccagagaacatcgtgatcgagatggctcgcgaaaatcagactactcagaagggtcaaaagaactcccgcgagcgcatgaagcgcatcgaagaaggaattaaggagctgggctcacaaatcctcaaagaacatcctgtcgagaacacccaacttcagaacgagaaactgtacctctactatctccaaaatggccgggacatgtacgtcgatcaggaattggatatcaaccgcctctcagactacgacgtggatgctatcgtgccgcagtcgtttctcaaagatgatagcatcgacaacaaggtcctgacgaggtcggataagaaccgcgggaaatccgacaatgtgccttcggaagaggtggtgaaaaagatgaagaactactggaggcaattgcttaatgccaaactgatcacccagcgcaaattcgacaaccttactaaggccgagcgcggaggactgtccgaactcgataaggcggggttcatcaaaagacaattggtcgaaactcggcaaattaccaagcatgtcgctcagatcctcgactcgcgcatgaacactaagtatgatgagaacgacaagctcattcgcgaagtgaaagtgattaccctcaaatcaaagctggtgtcggacttcagaaaagactttcaattctacaaggtgcgggagattaacaactaccaccacgcgcacgacgcctacctcaatgcagtggtgggaaccgccctcatcaagaagtatccaaagctcgaaagcgagttcgtgtacggggactataaagtgtacgatgtgcggaaaatgatcgcaaagagcgagcaagagatcggcaaagcaacggccaaatacttcttctacagcaacattatgaactttttcaagaccgagattaccctggcgaacggagaaatccggaagcggcctttgatcgaaacgaatggggaaactggagaaatcgtgtgggacaagggcagagactttgcgaccgtgagaaaggtgctgtccatgccacaagtcaacatcgtgaagaaaactgaagtgcagactggaggattttccaaggagtcaatccttccgaagcgcaacagcgacaagctgatcgccaggaagaaggattgggaccccaagaagtacggtggttttgattcacctactgtcgcttacagcgtgctggtggtggccaaggtggaaaaggggaagtcaaagaaattgaagtcggtgaaggaattgctggggattactatcatggagaggagcagcttcgaaaagaatcccatcgacttcttggaggccaagggatacaaagaggtgaagaaagacctgatcattaagctgccgaagtactccttgttcgaactggaaaacggcagaaagcggatgctggcctccgccggagagctgcagaagggtaacgagctggctctgcccagcaaatacgtgaatttcctctacctggcctcccattacgagaagctcaagggaagcccggaggataatgagcaaaaacaactgttcgtcgagcagcacaagcactacctcgacgagatcatcgaacaaatctccgagttctcgaagcgggtgattctggccgacgcaaaccttgataaagtcctcagcgcctacaacaagcaccgcgacaaaccgatcagagaacaagctgagaacatcatccacctgttcacgctgactaacttgggagcgcctgccgccttcaagtacttcgacaccactatcgatcggaaacggtacacttccactaaggaagtgctggacgcaaccctgatccatcagtccatcaccggactttatgagactcggatcgatctcagccagcttggcggtgattcaagagctgacccgaagaagaagcgcaaggtctaa

SEQ ID NO: 18 - DNA, 264 bp, synthetic construct
  source 1..264: mol_type = other DNA
atgaacatcaacgatctgatccgggagatcaagaacaaggactacaccgtgaagctgagcggcaccgatagcaacagcatcacccagctgattatccgcgtgaacaacgacggcaacgagtacgtgatctccgagagcgagaacgagagcatcgtggagaagttcatcagcgccttcaagaacggttggaaccaggagtacgaggacgaggaggagttctacaacgacatgcagaccatcaccctgaagagcgagctgaactaa

SEQ ID NO: 19 - skipped

SEQ ID NO: 20 - DNA, 4436 bp, synthetic construct
  source 1..4436: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgaggacttagcctttagtgaaccgtcagaattaattcagatcgatctaccagaaccgtcagatccgctagagattacgccaaccgccaccatgggcagcatgagccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgctagcggctcaggcgagggcagaggaagtctgctaacatgcggtgacgtcgaggagaatcctggcccagcgtccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 21 - DNA, 99 bp, synthetic construct
  source 1..99: mol_type = other DNA
atgggggaaattgctgctctggaagccaaaaatgcggcgttgaaagccgagattgcggccttggaagctaagatcgctgctttaaaggccggatactaa

SEQ ID NO: 22 - DNA, 84 bp, synthetic construct
  source 1..84: mol_type = other DNA
atgttaaaggcggagaacgcagctctggaagccaagattgcagccttaaaagcggagattgctgcgttagaggcaggctactaa

SEQ ID NO: 23 - DNA, 420 bp, synthetic construct
  source 1..420: mol_type = other DNA
atgaacatcaacgatctgatccgggagatcaagaacaaggactacaccgtgaagctgagcggcaccgatagcaacagcatcacccagctgattatccgcgtgaacaacgacggcaacgagtacgtgatctccgagagcgagaacgagagcatcgtggagaagttcatcagcgccttcaagaacggttggaaccaggagtacgaggacgaggaggagttctacaacgacatgcagaccatcaccctgaagagcgagctgaactcaggctctggactcagatctggcagcggaggaggcagcgcatctggcggaagcggagcgtccggggaaattgctgctctggaagccaaaaatgcggcgttgaaagccgagattgcggccttggaagctaagatcgctgctttaaaggccggatactaa

SEQ ID NO: 24 - DNA, 5859 bp, synthetic construct
  source 1..5859: mol_type = other DNA
atggacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctaggcctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggccccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagaaacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaagcctatgacaagaaatactccatcggccttgccatcgggaccaacagcgtgggttgggccgtgatcactgacgaatacaaagtgccatcgaaaaagtttaaggtgctgggcaatactgacagacatagcatcaagaaaaacctgattggagccctcctgttcgactccggggaaaccgccgaagccactcgcctgaaacggaccgcacgccggaggtacactcgccggaagaatagaatctgctaccttcaagagatctttagcaacgagatggccaaagtggacgattcgttcttccaccggctggaggagtcattcctggtcgaagaggacaaaaagcatgagagacatccgatcttcggtaacattgtggatgaggtcgcctaccacgagaagtaccccactatctaccatttgcgcaagaagttggtcgactcgactgataaggccgacctcagactcatctacctcgctttggcacacatgatcaagtttaggggtcacttcctgattgaaggggatctgaacccagacaactcggatgtggataaactgtttatccagctggtccagacttacaatcagctctttgaggagaacccgatcaatgcctccggcgtggatgctaaggcaatcctgtccgcacgcttgtcaaagagccggcggctcgaaaatctgatcgcacagcttccgggagaaaagaaaaacggactgtttggaaaccttatcgccctctcgctgggactcactcctaacttcaaatccaattttgatttggccgaggatgccaagctgcagctgtcgaaggacacctacgacgatgacctcgacaatcttctggcccagatcggggaccagtacgccgatctgttcctcgcggccaaaaacctgtcagacgcaatcttgctgagcgatatcttgagggtcaataccgaaatcactaaggctccattgtcagcatcgatgatcaagaggtacgatgaacaccatcaggacctgaccctgctcaaggcactggtgcggcagcaactgccggaaaagtacaaggagatcttcttcgatcaaagcaagaatgggtacgcagggtacatcgatggtggagcatcacaagaagagttctacaaattcatcaagcccatccttgaaaagatggacgggacggaagaactgctggtgaagctgaatcgggaagatctgctgcggaagcagcgcaccttcgacaatggttccatcccacaccaaatccatctcggcgaactgcacgcgatccttcgccggcaggaagatttctacccgttcttgaaggataacagagaaaagatcgagaaaatcctgacctttagaatcccgtactacgtgggcccgttggctcgcggaaactcaagattcgcctggatgactagaaaatccgaagaaacgattaccccgtggaactttgaagaggtcgtcgacaagggagcctcggcacagtcgttcatcgaacggatgaccaatttcgacaagaacctccccaacgaaaaggtgctcccgaaacactcgctgctgtatgagtacttcacggtctacaacgagctgaccaaggtgaagtacgtgaccgaaggaatgcggaagcccgcttttctgtccggtgaacagaaaaaggccatcgtggacctcctcttcaagactaacagaaaggtgaccgtgaaacagcttaaagaggactacttcaagaagatcgagtgttttgactccgtggaaatctcgggagtggaggataggtttaacgcttcgctggggacctaccatgaccttctcaagatcatcaaggataaggatttcctggacaacgaagaaaacgaggacatccttgaggacatcgtgttgaccttgaccctgtttgaggatcgggaaatgatcgaggagagactgaaaacttacgcgcatcttttcgacgacaaagtgatgaagcagctgaaaagaagaagatacactggctggggacggctgtcgaggaaactgattaacggaattagggataaacaaagcggaaagactatcctcgattttctcaagtcggacggcttcgccaatcggaactttatgcagctcatccacgacgatagcctgaccttcaaggaagatatccaaaaagcccaagtgtccggccagggagatagcttgcacgagcacattgctaacctggccggttcaccagccattaagaagggaatcctccaaaccgtgaaggtggtcgacgaactcgtcaaggtgatgggccgccataagccagagaacatcgtgatcgagatggctcgcgaaaatcagactactcagaagggtcaaaagaactcccgcgagcgcatgaagcgcatcgaagaaggaattaaggagctgggctcacaaatcctcaaagaacatcctgtcgagaacacccaacttcagaacgagaaactgtacctctactatctccaaaatggccgggacatgtacgtcgatcaggaattggatatcaaccgcctctcagactacgacgtggatgctatcgtgccgcagtcgtttctcaaagatgatagcatcgacaacaaggtcctgacgaggtcggataagaaccgcgggaaatccgacaatgtgccttcggaagaggtggtgaaaaagatgaagaactactggaggcaattgcttaatgccaaactgatcacccagcgcaaattcgacaaccttactaaggccgagcgcggaggactgtccgaactcgataaggcggggttcatcaaaagacaattggtcgaaactcggcaaattaccaagcatgtcgctcagatcctcgactcgcgcatgaacactaagtatgatgagaacgacaagctcattcgcgaagtgaaagtgattaccctcaaatcaaagctggtgtcggacttcagaaaagactttcaattctacaaggtgcgggagattaacaactaccaccacgcgcacgacgcctacctcaatgcagtggtgggaaccgccctcatcaagaagtatccaaagctcgaaagcgagttcgtgtacggggactataaagtgtacgatgtgcggaaaatgatcgcaaagagcgagcaagagatcggcaaagcaacggccaaatacttcttctacagcaacattatgaactttttcaagaccgagattaccctggcgaacggagaaatccggaagcggcctttgatcgaaacgaatggggaaactggagaaatcgtgtgggacaagggcagagactttgcgaccgtgagaaaggtgctgtccatgccacaagtcaacatcgtgaagaaaactgaagtgcagactggaggattttccaaggagtcaatccttccgaagcgcaacagcgacaagctgatcgccaggaagaaggattgggaccccaagaagtacggtggttttgattcacctactgtcgcttacagcgtgctggtggtggccaaggtggaaaaggggaagtcaaagaaattgaagtcggtgaaggaattgctggggattactatcatggagaggagcagcttcgaaaagaatcccatcgacttcttggaggccaagggatacaaagaggtgaagaaagacctgatcattaagctgccgaagtactccttgttcgaactggaaaacggcagaaagcggatgctggcctccgccggagagctgcagaagggtaacgagctggctctgcccagcaaatacgtgaatttcctctacctggcctcccattacgagaagctcaagggaagcccggaggataatgagcaaaaacaactgttcgtcgagcagcacaagcactacctcgacgagatcatcgaacaaatctccgagttctcgaagcgggtgattctggccgacgcaaaccttgataaagtcctcagcgcctacaacaagcaccgcgacaaaccgatcagagaacaagctgagaacatcatccacctgttcacgctgactaacttgggagcgcctgccgccttcaagtacttcgacaccactatcgatcggaaacggtacacttccactaaggaagtgctggacgcaaccctgatccatcagtccatcaccggactttatgagactcggatcgatctcagccagcttggcggtgattcaagagctgacccgaagaagaagcgcaaggtctcaggctctggactcagatctggcagcggaggaggcagcgcatctggcggaagcggagcgtccttaaaggcggagaacgcagctctggaagccaagattgcagccttaaaagcggagattgctgcgttagaggcaggctactaa

SEQ ID NO: 25 - DNA, 1044 bp, synthetic construct
  source 1..1044: mol_type = other DNA
atgggagtgcaagtggaaaccatctccccaggagatgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggcagcgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtcaggctaa

SEQ ID NO: 26 - DNA, 1075 bp, synthetic construct
  source 1..1075: mol_type = other DNA
atgggagtgcaagtggaaaccatctccccaggagatgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggcagctctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggttaa

SEQ ID NO: 27 - DNA, 1373 bp, synthetic construct
  source 1..1373: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagtcgtcgacgagggacttagcctttagtgaaccgtcagaattaattcagatcgatctaccagaaccgtcagatccgctagagattacgccaaccgccaccatgggcagcgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 28 - DNA, 1250 bp, synthetic construct
  source 1..1250: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggacttagcctttagtgaaccgtcagaattaattcagatcgatctaccagaaccgtcagatccgctagagattacgccaaccgccaccatgggcagcggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgacaggctaataacagcttccggactctagaacatccctacaggtgatatcctcgtgctgcaagtaaacccctaccaactggtcggggtttgaaac

SEQ ID NO: 29 - DNA, 1121 bp, synthetic construct
  source 1..1121: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaaggacttagcctttagtgaaccgtcagaattaattcagatcgatctaccagaaccgtcagatccgctagagattacgccaaccgccaccatgggcagcggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgacaggctaataacagcttccggactctagaacatccctacaggtgatatcctcgtgctgcaagtaaacccctaccaactggtcggggtttgaaac

SEQ ID NO: 30 - DNA, 1085 bp, synthetic construct
  source 1..1085: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaaggacttagcctttagtgaaccgtcagaattaattcagatcgatctaccagaaccgtcagatccgctagagattacgccaaccgccaccatgggcagcggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgacaggctaataacagcttccggactctagaacatccctacaggtgatatcctcgggtaa

SEQ ID NO: 31 - DNA, 2480 bp, synthetic construct
  source 1..2480: mol_type = other DNA
ccctgcgggtcacgtgacgggtcacgtgacgggtcacgtgacgggtcacgtgaaaagtgaaagtcgagtttaccactcacgtgaaaagtgaaagtcgagtttaccactcacgtgaaaagtgaaagtcgagtttaccactcacgtgaccctgcgggaagcttagacactagagggtatataatggaagctcgacttccagggacttagcctttagtgaaccgtcagaattaattcagatcgatctaccagaaccgtcagatccgctagagattacgccaaccgccaccatgggcagcatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaacactagtggcggctctggcggcggctccggaggctctagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggacccagcgcccgcagtgactcccgaggccagtcacctgttggaggaccccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttga

SEQ ID NO: 32 - DNA, 57 bp, synthetic construct
  source 1..57: mol_type = other DNA
gctactaacttcagcctgctgaagcaggctggcgacgtggaggagaaccctggacct

SEQ ID NO: 33 - DNA, 678 bp, synthetic construct
  source 1..678: mol_type = other DNA
caggctaataacagcttccggactctagaacatccctacaggtgatatcctcgatcccgctcgataagcttgatatcgaattccgcccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 34 - DNA, 3643 bp, synthetic construct
  source 1..3643: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggacttagcctttagtgaaccgtcagaattaattcagatcgatctaccagaaccgtcagatccgctagagattacgccaaccgccaccatgggcagcgccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttcaggcgctactaacttcagcctgctgaagcaggctggcgacgtggaggagaaccctggacctgcgtccatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaatgctgaggctaactgaaacacggaaggagacaatacaggaaggaacggtaacttgtttattgcagcttataatggttacaaataaagcaatagcatcacaaatttcacaaataaagcatttttttcactgcattctagttgtggtttgtccaaactcatcaatgtatcttatcatgtctgtcgtctatgatgaggatgttggtggagagcatgtggaggaagtggatagggaaggttgtagagtagatccggttgaagtgatgaggataggaggaggtcacaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagctttttt

SEQ ID NO: 35 - DNA, 3642 bp, synthetic construct
  source 1..3642: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggacttagcctttagtgaaccgtcagaattaattcagatcgatctaccagaaccgtcagatccgctagagattacgccaaccgccaccatgggcagcactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtcaggcgctactaacttcagcctgctgaagcaggctggcgacgtggaggagaaccctggacctgcgtccatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgattttaatgctgaggctaactgaaacacggaaggagacaatacaggaaggaacggtaacttgtttattgcagcttataatggttacaaataaagcaatagcatcacaaatttcacaaataaagcatttttttcactgcattctagttgtggtttgtccaaactcatcaatgtatcttatcatgtctgtcgtctatgatgaggatgttggtggagagcatgtggaggaagtggatagggaaggttgtagagtagatccggttgaagtgatgaggataggaggaggtcacaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagctttttt

SEQ ID NO: 36 - DNA, 4259 bp, synthetic construct
  source 1..4259: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggacttagcctttagtgaaccgtcagaattaattcagatcgatctaccagaaccgtcagatccgctagagattacgccaaccgccaccatgggcagcgccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttcaggctaataacagcttccggactctagaacatccctacaggtgatatcctcgatcccgctcgataagcttgatatcgaattccgcccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgcgtccatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaatgctgaggctaactgaaacacggaaggagacaatacaggaaggaacggtaacttgtttattgcagcttataatggttacaaataaagcaatagcatcacaaatttcacaaataaagcatttttttcactgcattctagttgtggtttgtccaaactcatcaatgtatcttatcatgtctgtcgtctatgatgaggatgttggtggagagcatgtggaggaagtggatagggaaggttgtagagtagatccggttgaagtgatgaggataggaggaggtcacaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagctttttt

SEQ ID NO: 37 - DNA, 4257 bp, synthetic construct
  source 1..4257: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggacttagcctttagtgaaccgtcagaattaattcagatcgatctaccagaaccgtcagatccgctagagattacgccaaccgccaccatgggcagcactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgcaggctaataacagcttccggactctagaacatccctacaggtgatatcctcgatcccgctcgataagcttgatatcgaattccgcccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgcgtccatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgattttaatgctgaggctaactgaaacacggaaggagacaatacaggaaggaacggtaacttgtttattgcagcttataatggttacaaataaagcaatagcatcacaaatttcacaaataaagcatttttttcactgcattctagttgtggtttgtccaaactcatcaatgtatcttatcatgtctgtcgtctatgatgaggatgttggtggagagcatgtggaggaagtggatagggaaggttgtagagtagatccggttgaagtgatgaggataggaggaggtcacaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagctttttt

SEQ ID NO: 38 - DNA, 3314 bp, synthetic construct
  source 1..3314: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggacttagcctttagtgaaccgtcagaattaattcagatcgatctaccagaaccgtcagatccgctagagattacgccaaccgccaccatgggcagcgccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttcaggcgctactaacttcagcctgctgaagcaggctggcgacgtggaggagaaccctggacctgcgtccatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaatgctgaggctaactgaaacacggaaggagacaatacaggaaggaacggtaacttgtttattgcagcttataatggttacaaataaagcaatagcatcacaaatttcacaaataaagcatttttttcactgcattctagttgtggtttgtccaaactcatcaatgtatctta

SEQ ID NO: 39 - DNA, 3313 bp, synthetic construct
  source 1..3313: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggacttagcctttagtgaaccgtcagaattaattcagatcgatctaccagaaccgtcagatccgctagagattacgccaaccgccaccatgggcagcactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtcaggcgctactaacttcagcctgctgaagcaggctggcgacgtggaggagaaccctggacctgcgtccatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgattttaatgctgaggctaactgaaacacggaaggagacaatacaggaaggaacggtaacttgtttattgcagcttataatggttacaaataaagcaatagcatcacaaatttcacaaataaagcatttttttcactgcattctagttgtggtttgtccaaactcatcaatgtatctta

SEQ ID NO: 40 - DNA, 3930 bp, synthetic construct
  source 1..3930: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggacttagcctttagtgaaccgtcagaattaattcagatcgatctaccagaaccgtcagatccgctagagattacgccaaccgccaccatgggcagcgccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttcaggctaataacagcttccggactctagaacatccctacaggtgatatcctcgatcccgctcgataagcttgatatcgaattccgcccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgcgtccatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaatgctgaggctaactgaaacacggaaggagacaatacaggaaggaacggtaacttgtttattgcagcttataatggttacaaataaagcaatagcatcacaaatttcacaaataaagcatttttttcactgcattctagttgtggtttgtccaaactcatcaatgtatctta

SEQ ID NO: 41 - DNA, 3928 bp, synthetic construct
  source 1..3928: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggacttagcctttagtgaaccgtcagaattaattcagatcgatctaccagaaccgtcagatccgctagagattacgccaaccgccaccatgggcagcactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgcaggctaataacagcttccggactctagaacatccctacaggtgatatcctcgatcccgctcgataagcttgatatcgaattccgcccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgcgtccatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgattttaatgctgaggctaactgaaacacggaaggagacaatacaggaaggaacggtaacttgtttattgcagcttataatggttacaaataaagcaatagcatcacaaatttcacaaataaagcatttttttcactgcattctagttgtggtttgtccaaactcatcaatgtatctta

SEQ ID NO: 42 - DNA, 199 bp, synthetic construct
  source 1..199: mol_type = other DNA
ccctgcgggtcacgtgacgggtcacgtgacgggtcacgtgacgggtcacgtgaaaagtgaaagtcgagtttaccactcacgtgaaaagtgaaagtcgagtttaccactcacgtgaaaagtgaaagtcgagtttaccactcacgtgaccctgcgggaagcttagacactagagggtatataatggaagctcgacttccag

SEQ ID NO: 43 - DNA, 2137 bp, synthetic construct
  source 1..2137: mol_type = other DNA
ccctgcgggtcacgtgacgggtcacgtgacgggtcacgtgacgggtcacgtgaaaagtgaaagtcgagtttaccactcacgtgaaaagtgaaagtcgagtttaccactcacgtgaaaagtgaaagtcgagtttaccactcacgtgaccctgcgggaagcttagacactagagggtatataatggaagctcgacttccagtttagtgaaccgtcagaattaattcagccgtcagaattaattcagatcgatctaccagaaccgtcagatccgctagagattacgccaaccgccaccatgggcagcgaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattctgcttgcaagaactggttcagtagcttaagccactttgtgatccaccttaacagccacggcttccctcccgaggtggaggagcaggccgccggcaccctgcccatgagctgcgcccaggagagcggcatggatagacaccctgctgcttgcgccagcgccaggatcaacgtctcaggctaa

SEQ ID NO: 44 - DNA, 703 bp, synthetic construct
  source 1..703: mol_type = other DNA
ccctgcgggtcacgtgacgggtcacgtgacgggtcacgtgacgggtcacgtgaaaagtgaaagtcgagtttaccactcacgtgaaaagtgaaagtcgagtttaccactcacgtgaaaagtgaaagtcgagtttaccactcacgtgaccctgcgggaagcttagacactagagggtatataatggaagctcgacttccagtttagtgaaccgtcagaattaattcagatcgatctaccagaaccgtcagatccgctagagattacgccaaccgccaccatgggcagcaacatcaacgatctgatccgggagatcaagaacaaggactacaccgtgaagctgagcggcaccgatagcaacagcatcacccagctgattatccgcgtgaacaacgacggcaacgagtacgtgatctccgagagcgagaacgagagcatcgtggagaagttcatcagcgccttcaagaacggttggaaccaggagtacgaggacgaggaggagttctacaacgacatgcagaccatcaccctgaagagcgagctgaactcaggctctggactcagatctggcagcggaggaggcagcgcatctggcggaagcggagcgtccggggaaattgctgctctggaagccaaaaatgcggcgttgaaagccgagattgcggccttggaagctaagatcgctgctttaaaggccggatactaa

SEQ ID NO: 45 - DNA, 115 bp, synthetic construct
  source 1..115: mol_type = other DNA
tgcactccgcccgagctctgcactccgcccgagctctgcactccgcccgagctctgcactccgcccgtcgaccttgggcatactgcaggtcgactctagagggtatataatggat

SEQ ID NO: 46 - DNA, 432 bp, synthetic construct
  source 1..432: mol_type = other DNA
ccgtcttccgtttccacatcttcacggataccaaggtggcaccggtttgattgcacagcccgtcttccgtttccacatcttcacggtgctggcagccggggtaccgcaggcaagtcgcccgtcttccgtttccacatcttcacggatggaatcacggcggccgcccgtatctcatccccgtcttccgtttccacatcttcacggaatgcgtcggcgtgcggccgctgccagttgcaccgtcttccgtttccacatcttcacggttggctgtcctagggtacctggacgctccttgccgtcttccgtttccacatcttcacggtggctggtgccgcaccggtaagcagacggccgccgtcttccgtttccacatcttcacggggatcctgtacgggccagatagagggtatataatggaagctcgacttccag

SEQ ID NO: 47 - DNA, 1665 bp, synthetic construct
  source 1..1665: mol_type = other DNA
atgggcagcgaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtcaggctaa

SEQ ID NO: 48 - DNA, 595 bp, synthetic construct
  source 1..595: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgccgtcttccgtttccacatcttcacggataccaaggtggcaccggtttgattgcacagcccgtcttccgtttccacatcttcacgg

SEQ ID NO: 49 - DNA, 1742 bp, synthetic construct
  source 1..1742: mol_type = other DNA
atgggcagcgaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtcaggctaaccggactctagaacatccctacaggtgatatcctcgtgctgcaagtaaacccctaccaactggtcggggtttgaaac

SEQ ID NO: 50 - DNA, 848 bp, synthetic construct
  source 1..848: mol_type = other DNA
atgggcagcatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtcaggctaataacagcttccggactctagaacatccctacaggtgatatcctcgtgctgcaagtaaacccctaccaactggtcggggtttgaaac

SEQ ID NO: 51 - DNA, 1049 bp, synthetic construct
  source 1..1049: mol_type = other DNA
ccctgcgggtcacgtgacgggtcacgtgacgggtcacgtgacgggtcacgtgaaaagtgaaagtcgagtttaccactcacgtgaaaagtgaaagtcgagtttaccactcacgtgaaaagtgaaagtcgagtttaccactcacgtgaccctgcgggaagcttagacactagagggtatataatggaagctcgacttccagggacttagcctttagtgaaccgtcagaattaattcagatcgatctaccagaaccgtcagatccgctagagattacgccaaccgccaccatgggcagcatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtcaggctaa

SEQ ID NO: 52 - DNA, 978 bp, synthetic construct
  source 1..978: mol_type = other DNA
cttgagctcgaggagctctgcactccgcccgagctctgcactccgcccgagctctgcactccgcccgagctctgcactccgcccgtcgaccttgggcatactgcaggtcgactctagagggtatataaggacttagcctttagtgaaccgtcagaattaattcagatcgatctaccagaaccgtcagatccgctagagattacgccaaccgccaccatgggcagcatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtcaggctaa

SEQ ID NO: 53 - DNA, 1137 bp, synthetic construct
  source 1..1137: mol_type = other DNA
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactcaggctaa

SEQ ID NO: 54 - DNA, 1712 bp, synthetic construct
  source 1..1712: mol_type = other DNA
gtacacgccacctcgacatactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagacttagcctttagtgaaccgtcagaattaattcagatcgatctaccagaaccgtcagatccgctagagattacgccaaccgccaccatgggcagcatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactcaggctaaccggactctagaacatccctacaggtgatatcctcgtgctgcaagtaaacccctaccaactggtcggggtttgaaac

SEQ ID NO: 55 - DNA, 3700 bp, synthetic construct
  source 1..3700: mol_type = other DNA
gtacacgccacctcgacatactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaaggacttagcctttagtgaaccgtcagaattaattcagatcgatctaccagaaccgtcagatccgctagagattacgccaaccgccaccatgggcagcgccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttgactctagaacatccctacaggtgatatcctcgatcccgctcgataagcttgatatcgaattccgcccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgcgtccatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaatgctgaggctaactgaaacacggaagtgctgcaagtaaacccctaccaactggtcggggtttgaaac

SEQ ID NO: 56 - DNA, 4168 bp, synthetic construct
  source 1..4168: mol_type = other DNA
tgggtatttaagcccgagtgagcacgcaggatctccattttgaagcgggaggtttgaacgcgcagccgccttaattaaatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcaacatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 57 - DNA, 6376 bp, synthetic construct
  source 1..6376: mol_type = other DNA
gtacacgccacctcgacatactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaaggacttagcctttagtgaaccgtcagaattaattcagatcgatctaccagaaccggatctaccagaaccgtcagatccgctagagattacgccaaccgccaccatgggcagccgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctaggcctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggccccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagaaacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaagcctatgacaagaaatactccatcggccttgccatcgggaccaacagcgtgggttgggccgtgatcactgacgaatacaaagtgccatcgaaaaagtttaaggtgctgggcaatactgacagacatagcatcaagaaaaacctgattggagccctcctgttcgactccggggaaaccgccgaagccactcgcctgaaacggaccgcacgccggaggtacactcgccggaagaatagaatctgctaccttcaagagatctttagcaacgagatggccaaagtggacgattcgttcttccaccggctggaggagtcattcctggtcgaagaggacaaaaagcatgagagacatccgatcttcggtaacattgtggatgaggtcgcctaccacgagaagtaccccactatctaccatttgcgcaagaagttggtcgactcgactgataaggccgacctcagactcatctacctcgctttggcacacatgatcaagtttaggggtcacttcctgattgaaggggatctgaacccagacaactcggatgtggataaactgtttatccagctggtccagacttacaatcagctctttgaggagaacccgatcaatgcctccggcgtggatgctaaggcaatcctgtccgcacgcttgtcaaagagccggcggctcgaaaatctgatcgcacagcttccgggagaaaagaaaaacggactgtttggaaaccttatcgccctctcgctgggactcactcctaacttcaaatccaattttgatttggccgaggatgccaagctgcagctgtcgaaggacacctacgacgatgacctcgacaatcttctggcccagatcggggaccagtacgccgatctgttcctcgcggccaaaaacctgtcagacgcaatcttgctgagcgatatcttgagggtcaataccgaaatcactaaggctccattgtcagcatcgatgatcaagaggtacgatgaacaccatcaggacctgaccctgctcaaggcactggtgcggcagcaactgccggaaaagtacaaggagatcttcttcgatcaaagcaagaatgggtacgcagggtacatcgatggtggagcatcacaagaagagttctacaaattcatcaagcccatccttgaaaagatggacgggacggaagaactgctggtgaagctgaatcgggaagatctgctgcggaagcagcgcaccttcgacaatggttccatcccacaccaaatccatctcggcgaactgcacgcgatccttcgccggcaggaagatttctacccgttcttgaaggataacagagaaaagatcgagaaaatcctgacctttagaatcccgtactacgtgggcccgttggctcgcggaaactcaagattcgcctggatgactagaaaatccgaagaaacgattaccccgtggaactttgaagaggtcgtcgacaagggagcctcggcacagtcgttcatcgaacggatgaccaatttcgacaagaacctccccaacgaaaaggtgctcccgaaacactcgctgctgtatgagtacttcacggtctacaacgagctgaccaaggtgaagtacgtgaccgaaggaatgcggaagcccgcttttctgtccggtgaacagaaaaaggccatcgtggacctcctcttcaagactaacagaaaggtgaccgtgaaacagcttaaagaggactacttcaagaagatcgagtgttttgactccgtggaaatctcgggagtggaggataggtttaacgcttcgctggggacctaccatgaccttctcaagatcatcaaggataaggatttcctggacaacgaagaaaacgaggacatccttgaggacatcgtgttgaccttgaccctgtttgaggatcgggaaatgatcgaggagagactgaaaacttacgcgcatcttttcgacgacaaagtgatgaagcagctgaaaagaagaagatacactggctggggacggctgtcgaggaaactgattaacggaattagggataaacaaagcggaaagactatcctcgattttctcaagtcggacggcttcgccaatcggaactttatgcagctcatccacgacgatagcctgaccttcaaggaagatatccaaaaagcccaagtgtccggccagggagatagcttgcacgagcacattgctaacctggccggttcaccagccattaagaagggaatcctccaaaccgtgaaggtggtcgacgaactcgtcaaggtgatgggccgccataagccagagaacatcgtgatcgagatggctcgcgaaaatcagactactcagaagggtcaaaagaactcccgcgagcgcatgaagcgcatcgaagaaggaattaaggagctgggctcacaaatcctcaaagaacatcctgtcgagaacacccaacttcagaacgagaaactgtacctctactatctccaaaatggccgggacatgtacgtcgatcaggaattggatatcaaccgcctctcagactacgacgtggatgctatcgtgccgcagtcgtttctcaaagatgatagcatcgacaacaaggtcctgacgaggtcggataagaaccgcgggaaatccgacaatgtgccttcggaagaggtggtgaaaaagatgaagaactactggaggcaattgcttaatgccaaactgatcacccagcgcaaattcgacaaccttactaaggccgagcgcggaggactgtccgaactcgataaggcggggttcatcaaaagacaattggtcgaaactcggcaaattaccaagcatgtcgctcagatcctcgactcgcgcatgaacactaagtatgatgagaacgacaagctcattcgcgaagtgaaagtgattaccctcaaatcaaagctggtgtcggacttcagaaaagactttcaattctacaaggtgcgggagattaacaactaccaccacgcgcacgacgcctacctcaatgcagtggtgggaaccgccctcatcaagaagtatccaaagctcgaaagcgagttcgtgtacggggactataaagtgtacgatgtgcggaaaatgatcgcaaagagcgagcaagagatcggcaaagcaacggccaaatacttcttctacagcaacattatgaactttttcaagaccgagattaccctggcgaacggagaaatccggaagcggcctttgatcgaaacgaatggggaaactggagaaatcgtgtgggacaagggcagagactttgcgaccgtgagaaaggtgctgtccatgccacaagtcaacatcgtgaagaaaactgaagtgcagactggaggattttccaaggagtcaatccttccgaagcgcaacagcgacaagctgatcgccaggaagaaggattgggaccccaagaagtacggtggttttgattcacctactgtcgcttacagcgtgctggtggtggccaaggtggaaaaggggaagtcaaagaaattgaagtcggtgaaggaattgctggggattactatcatggagaggagcagcttcgaaaagaatcccatcgacttcttggaggccaagggatacaaagaggtgaagaaagacctgatcattaagctgccgaagtactccttgttcgaactggaaaacggcagaaagcggatgctggcctccgccggagagctgcagaagggtaacgagctggctctgcccagcaaatacgtgaatttcctctacctggcctcccattacgagaagctcaagggaagcccggaggataatgagcaaaaacaactgttcgtcgagcagcacaagcactacctcgacgagatcatcgaacaaatctccgagttctcgaagcgggtgattctggccgacgcaaaccttgataaagtcctcagcgcctacaacaagcaccgcgacaaaccgatcagagaacaagctgagaacatcatccacctgttcacgctgactaacttgggagcgcctgccgccttcaagtacttcgacaccactatcgatcggaaacggtacacttccactaaggaagtgctggacgcaaccctgatccatcagtccatcaccggactttatgagactcggatcgatctcagccagcttggcggtgattcaagagctgacccgaagaagaagcgcaaggtctcaggctctggactcagatctggcagcggaggaggcagcgcatctggcggaagcggagcgtccttaaaggcggagaacgcagctctggaagccaagattgcagccttaaaagcggagattgctgcgttagaggcaggctactaa

(19) United States
(12) Patent Application Publication
(10) Pub. No.: US 2026/0022396 A1
(43) Pub. Date: Jan. 22, 2026

(54) GENETIC CIRCUITS

(71) Applicant: FONDAZIONE TELETHON ETS, Roma (IT)

(72) Inventors: Diego Di Bernardo, Pozzuoli (NA) (IT); Giuliano De Carluccio, Pozzuoli (NA) (IT); Alessio Mallozzi, Pozzuoli (NA) (IT); Virginia Fusco, Pozzuoli (NA) (IT); Francesco Ragazzini, Pozzuoli (NA) (IT)

(73) Assignee: FONDAZIONE TELETHON ETS, ROMA (IT)

(21) Appl. No.: 18/996,298

(22) PCT Filed: Jul. 28, 2023
(86) PCT No.: PCT/IB2023/057674

§ 371 Date: Jan. 17, 2025

(30) Foreign Application Priority Data
Jul. 28, 2022 (IT) 102022000015981

(51) Int. Cl. C12N 15/86; C12N 9/22; C12N 15/11

(52) U.S. Cl. CPC C12N 15/86; C12N 9/226; C12N 15/11; C12N 2310/20; C12N 2750/14122; C12N 2750/14143; C12N 2830/002

(57) ABSTRACT

The present invention is directed to engineered genetic circuits for controlling gene expression in cells, cells comprising said genetic circuits and uses of the cells and genetic circuits.

### FIELD OF THE INVENTION

The present invention is directed to engineered genetic circuits for controlling expression of genes in a cell, to cells comprising said genetic circuits and to uses of said cells and genetic circuits.

### BACKGROUND

Expression of genes in a cell is regulated by circuits of biomolecules genetically encoded (genetic circuits). Engineering of genetic circuits is an application of synthetic biology to provide cells with novel abilities to modulate expression of genes of interest, in response to a bioactive molecule, drug, chemical, or even pathogen (analytes), that is present within the cell or in the surrounding microenvironment. Engineered genetic circuits can be advantageously used in many fields, such as toxicology and drug discovery, biomanufacturing and biosecurity. Transcription-based circuits are typically obtained by engineering a promoter regulated by a transcription factor whose activity is preferably modulated by an inducer, such as a drug or an analyte: binding of the inducer to the transcription factor can result in a conformational change of the transcription factor with consequent binding of the same to the engineered promoter and then to transcription of the mRNA of a reporter gene driven by the engineered promoter. Transcription-based circuits can then be used to sense and report on the cell internal state or on the surrounding microenvironment; they can also be coupled to effector proteins to control a biological process of interest by regulating expression of said effector proteins.

The doxycycline-inducible Tet-Off/Tet-On gene expression system is the most widely used “on demand” regulator of expression of a gene of interest by a cell by adding, or removing, doxycycline (a tetracycline derivative) in the cell microenvironment. It consists of a tetracycline-responsive promoter (pTRE), driving expression of a gene of interest (GOI), whose activation depends on binding of a recombinant tetracycline-controlled transcriptional activator, tTA or rtTA. In the Tet-Off system, tetracycline prevents the tTA from binding the pTRE promoter, thus silencing gene expression; conversely in the Tet-On system, tetracycline allows rtTA to bind the pTRE promoter, thus inducing gene expression (see FIG. 1).

Design of efficient genetic circuits is very challenging, as compared with other areas of genetic engineering since circuits require the precise balancing of their components to generate the proper response. Biological processes regulated by engineered genetic circuits can be schematized as dynamic input-output systems. The input can be any molecular species (e.g., small molecule, metabolite, or protein, hereafter referred to as the ‘analyte’, or the ‘inducer molecule’) whose changes have a measurable effect on the output of the biological process.

For instance, in the case of transcription-based biosensors, the input is the analyte and the output is a reporter protein that needs to be regulated to a reference value proportional to the analyte's concentration. Alternatively, the input can be the activation of the promoter itself, driving expression of a gene of interest (GOI), said activation being optionally constitutive.

However, current state-of-the-art in the gene-expression systems follow an open-loop paradigm, where the analyte directly controls the level of the reporter, thus making the reporter vulnerable to “perturbations” of the cell state (i.e., stress, burden, noise), even when the analyte concentration is constant.

In general, the currently available systems for modulating gene expression in a cell have three main drawbacks: (1) basal leakiness when the gene is expressed by the promoter even in the absence of the inducer molecule, (2) lack of tunability when a specific level of gene expression is needed but it cannot be achieved by titrating the inducer molecule (e.g., doxycycline in tet-on or tet-off systems) and (3) lack of robustness when the expression level of the gene is not precisely maintained over time because of fluctuations in the intra- or extra-cellular environment.

These limitations prevent the use of such gene expression systems in several applications with a huge impact, such as: (a) generation of stable “producer cell lines” of biomolecules where said biomolecules induce cytotoxicity (e.g. biomanufacturing of AAV viral vectors, or of toxic recombinant proteins); (b) construction of whole cell biosensors able to sense and report, without perturbations, on pathway activation in response to bioactive molecules (analytes) relevant to human health that are present within the cell or in the surrounding microenvironment; (c) engineering of cells for ex-vivo therapy such as CAR-T cells, requiring precise control of the expression of the gene of interest; (d) engineering of human ES/iPS cells with inducible differentiation system to express lineage-determining transcription factors and differentiate cells in specific cell types.

In the field of biosensors, present-day technology struggles with the task of achieving expansive operational ranges, a critical requirement for seamless and accurate input detection. In parallel, a wide dynamic range is essential to improve the biosensor's signal-to-noise ratio and ultimately strengthen its ability to sense analytes reliably. Similarly, an optimal sensitivity response is required as well, as it ensures a pronounced alteration in output that is clearly distinguishable from previous measurements. Linearity plays a crucial role in enabling the generation of an analog response, enhancing the informativeness and ease of analysis of the responses when compared to commonly studied nonlinear biosensors. Achieving identification and quantification of diverse analytes requires careful optimization of these key parameters, which collectively shape the biosensor's input-output response, forming the foundation for its overall functionality. Moreover, mammalian whole cell biosensors (mWCBs) of the art are not robust to changes in intra- and extra-cellular environments, increasing the likelihood of false positives or negatives during analyte detection. Another significant challenge in deploying this technology is the need for a cumbersome trial-and-error process to optimize WCB performance. There is therefore the need to provide improved gene expression systems that overcome the above limitations, also in view of the important potential applications of said gene expression systems.

### BRIEF DESCRIPTION OF THE INVENTION

The limitations of the prior art's systems for modulating gene expression are overcome by the present invention, providing novel engineered genetic circuits for controlling gene expression in cells.

The present invention is then directed to the genetic circuits as defined in the enclosed claims.

Moreover, the present invention is directed to cells that incorporate said circuits, to methods for incorporating said genetic circuits in cells and to the use of said circuits in the claimed applications.

### BRIEF DESCRIPTION OF FIGURES

FIG. 1 exhibits a schematic diagram of an expression system of the prior art including an activator (X) that induces expression of an output species (Z) and an exemplary implementation based on the tet-on system, wherein X is the inducible transcriptional factor rtTA, that, in the presence of doxycycline (not shown), can bind to the cognate tetracycline responsive promoter pTRE3G inducing high expression of the downstream Gene of Interest, Gol, which embodies the output species Z.

FIG. 2 exhibits: A) a schematic diagram of a Coherent Feed Forward Loop (CFFL) genetic circuit according to the invention, including an activator (X) that induces expression of an output species (Z) and that negatively regulates a controller (Y) that would otherwise negatively regulate Z, and its implementation according to a preferred embodiment of the invention, wherein: X is the inducible rtTA, that, in the presence of doxycycline (not shown), binds to the cognate tetracycline responsive promoter pTRE3G (pathway i), inducing high expression of the downstream Gene of Interest (Gol), which embodies the output species Z, and also represses, through a steric hindrance mechanism, the pCMV/TO promoter (pathway ii) that drives the expression of a controller endoribonuclease CasRx that would otherwise repress the output gene Z through a short specific hairpin-structured sequence called Direct Repeat (DR) placed in the Z transcript's 3′UTR. B) schematic diagrams of the genetic circuit shown in A underlying the functional mutual inhibition of controller and GOI in its implementation according to preferred embodiments, wherein the activator (X) is rtTA3G, the controller (Y) is endoribonuclease CasRx and the output species (Z) includes DR in its 3′UTR. C) schematic diagrams and implementation of a genetic circuit wherein the controller Y is constitutively expressed and of the functional mutual inhibition with the output species Z, whose expression is induced by the activator X.

FIG. 3 shows the results of Example 1 of mCherry positive cells quantification by flow cytometry analysis in the absence and presence (1000 ng/ml) of doxycycline. pCMV/TO is efficiently repressed by rtTA-VPR in the presence of doxycycline. A condition without rtTA-VPR transfection is used as a negative control to account for any doxycycline treatment bias on mCherry expression. Percentual mean values of at least three biological replicates are shown. Error bars indicate standard error.

FIG. 4 shows the results of Example 2. Black bars indicate a Gaussia Luciferase transcript with the hairpin-structured sequence Direct Repeat in its 3′UTR (+DR). Gray bars represent a Gaussia Luciferase transcript without Direct Repeat (−DR). Error bars indicate standard error.

FIG. 5 shows: a) a scheme of the genetic circuit according to preferred embodiments of the invention and of the Tet-On system of the prior art used in Example 3; in the genetic circuit the rtTA-VPR transcription factor is driven by the strong constitutive promoter pCMV; the CasRx is controlled by the pCMV/TO promoter; the Gene of Interest is a Gaussia Luciferase reporter gene driven by the tetracycline responsive pTRE3G promoter harboring in its 3′UTR the short CasRx-specific hairpin-structured sequence Direct Repeat (DR); each of the genetic circuit parts is encoded by a different plasmid, for a total of three plasmids. b) the results of a dose-response curve obtained in Example 3, as gLuc A.U., in the presence of doxycycline, with the circuit of the invention compared with the system of the prior art; c) the results of a dose-response curve obtained in Example 3, as relative gLuc A.U, relative to the maximum gLuc A.U. value obtained by the TET-ON3G system at the highest concentration of Doxycycline (500 ng/mL). d) the measured fold change activation (FCA) levels of genetic circuit and Tet-ON by increasing doxycycline concentration up to 500 ng/mL. Each point represents the ratio between the gLuc A.U. value reached by the system divided by the value obtained in the absence of doxycycline. At least, three biological replicates were analyzed for each condition. Error bars indicate standard error.

FIG. 6 shows a scheme of a preferred circuit according to the invention controlling expression of Human Adenovirus 5 Helper genes and AAV2 Rep/Cap genes construct in a cell, for use in a method of production of AAV viral vectors; the expression of the gene of interest is driven by the pTRE3G promoter and the relative transcription units harbor the Direct Repeat in their 3′UTR. Here the genes of interest are Human Adenovirus 5 Helper genes E2A(DBP) and E4(Orf6), and AAV2 Rep/Cap genes. The controller construct CasRx is under the control of the pCMV/TO promoter.

FIG. 7 shows the results of Example 4. Black bars indicate the absence of doxycycline, instead, gray bars its presence (1000 ng/mL). Vg/mL represents the concentration of viral genome for each production condition quantified by means of absolute qRT-PCR. Mean values of at least three biological replicates are shown. Error bars indicate standard error.

FIG. 8 exhibits a schematic diagram of a Negative Feedback Loop (NFL) genetic circuit according to a preferred embodiment of the invention (closed loop): the constitutive promoter CMV drives the expression of VPR-dCas9-N8 protein, which, in the presence of the guide gRNA_B, expressed by the U6 promoter, binds the 7B_pMin promoter driving the expression of the transcription factor rtTA. In the presence of doxycycline, rtTA binds the TRE3G promoter and induces expression of the AcrII4A-N7 protein, which binds to, and inhibits, VPR-dCas9-N8, thus closing the loop. The luciferase (fLuc) protein is downstream of the 7B_pMin promoter and it is used as a reporter to track rtTA activity. A comparative open loop configuration is also shown, wherein rtTA is under the control of the constitutive promoter EF1α.

FIG. 9 shows NFL genetic circuits according to preferred embodiments of the invention, suitable for different applications: a) for CAR-T therapy; b) as a biosensor to detect skin reaction to chemical substances; c) for controlling activity of an endogenous transcription factor TFEB d) for controlling activity of an endogenous transcription factor NEUROD1.

FIG. 10 shows a scheme of a genetic circuit according to preferred embodiments of the invention, combining at least two genetic circuits according to different preferred aspects of the invention and acting as an ideal biosensor.

FIG. 11 shows the results of cytofluorimetry carried out in Example 5 to measure fluorescence in HEK293T cells transfected with the VPR_dCas9 downstream of a CMV promoter, the guideRNA downstream of the U6 promoter and the mCherry reporter cloned downstream of three different synthetic promoters consisting of different numbers of gRNA binding sites upstream of a minimal promoter: a) 1 binding site for the gRNA AB (1AB), b) 10 binding sites for the gRNA AB (10 AB) and c) 7 binding sites for the gRNA B (7B). As a negative control, cells were transfected without the guideRNA but only with the mCherry protein and the VPR dCas9.

FIG. 12 shows the results of example 6. a, c: Fold repression in luminescence obtained dividing the expression values of Renilla-normalized FireFly Luciferase luminescence in the presence of either VPR_dCas9 or VPR_dCas9-N8, by the values obtained when adding either AcrII4A or AcrII4A-N7, respectively. Data are presented in HEK293T cells and Hela cells, respectively. Each value has been normalized on Renilla Luciferase, expressed under the control of the constitutive promoter phTK. Molar ratio between VPR-dCas9(-N8) and AcrII4A(-N7) has been calculated to be 1:1. b, d: Renilla-normalized Firefly Luciferase luminescence is measured for the indicated conditions: VPR-dCas9 and its custom variant VPR-dCas9-N8 in absence of Acr proteins (N.C.), in presence of AcrII4A, and its custom variant AcrII4A-N7. Data are presented in HEK293T and HeLa cells, respectively. Firefly Luciferase values have been normalized on the constitutively expressed Renilla Luciferase. Molar ratio between VPR-dCas9(-N8) and AcrII4A(-N7) has been calculated to be 1:1.

FIG. 13 shows a scheme of the genetic circuit tested in example 7 (a) and the results of a dose-response experiment in HEK293T cells (b). Points represent experimental data while solid lines represent simulated data obtained using a mathematical model of the circuits. Experimental data have been first normalized on constitutively expressed Renilla Luciferase and then on the value obtained in the absence of Doxycycline, to obtain Fold Changes, indicated as Relative fLuc in the panel. The comparative open loop configuration is also shown, wherein rtTA is under the control of the constitutive promoter EF1α.

FIG. 14 shows: a) a scheme of the genetic circuit tested in example 8; b) the dose-response curve of the Closed Loop system at increasing Doxycycline concentrations obtained in example 8; c, d) the fLuc luminescence obtained with the closed loop circuit and with the comparative open loop, at the indicated constant concentration of Doxycycline, with decreasing concentration of Shield1; e, f: Heatmaps of the Open Loop and Closed Loop circuits, respectively, reporting fLuc luminescence for the indicated concentrations of doxycycline and Shield1.

FIG. 15 shows: a) a scheme of the working mechanism of the rtTA protein fused with the FKBP-derived Destabilization Domain (DD-rtTA); b) Results of cytofluorimetry carried out in HEK293T and HeLa cells, in absence of both Doxycycline and Shield1, with each of the two drugs individually and with both the drugs together.

FIG. 16 shows: a) a scheme of the genetic circuit employed in Example 9 to sense the activation of the starvation responsive TFEB transcription factor and b) Gaussia Luciferase expression measured in cells 48 hours after transfection, activated in the presence of doxycycline (1000 ng/mL). Gaussia Luciferase expression increase in the starvation condition as TFEB activation, compared to the Growth condition where there is no TFEB activity. gLuc arbitrary units (A.U). were calculated as the ratio between the Gaussia and Red Firefly normalizer luminescence. At least, three biological replicates were analyzed for each condition. Error bars indicate standard deviation.

FIG. 17 shows (a) a scheme of the μHelper plasmids bearing the minimal gene sets for expressing adenovirus helper proteins E2A and E4, tested in Example 10 and (b) the rAAV vector production capacity of said μHelper plasmids evaluated by transient triple transfection and quantified through absolute quantitative Real Time PCR.

FIG. 18: (a) shows a scheme of an NFL circuit according to preferred embodiments of the invention for controlling an endogenous gene of interest (GOI): the constitutive VPR-dCas9-N8 in presence of gRNA complementary to the endogenous promoter of the GOI, binds the endogenous promoter itself, promoting GOI expression. GOI is an endogenous transcription factor, that binds to a responsive promoter, driving the expression of the AcrII4A-N7 controller protein, that inhibits VPR-dCas9-N8, thus closing the loop; (b) shows a scheme of a comparative system according to the prior art for overexpressing NEUROD1: the constitutively expressed VPR-dCas9-N8, in presence of the gRNA (1, 2, 3 or a mix of them) binds to the endogenous promoter of NEUROD1, thus promoting its transcription; (c-d) show the results of Example 11: (c) quantification of NEUROD1 RNA overexpressed by the comparative system of b), detected by Real Time PCR. gRNA B is a control scramble guide used to normalize the values, (d) NiClear promoter activation detected with Luciferase assay. NiClear_fLuc is transfected in HEK293T cells with or without EF1α_NEUROD1 expressing plasmid; firefly Luciferase luminescence has been normalized on the constitutively expressed Renilla Luciferase. (e) shows a scheme of NFL circuit according to preferred aspects of the invention, for NEUROD1 overexpression, in which VPR-dCas9-N8 is constitutively expressed and AcrII4A-N7 controller protein is under the control of NiClear promoter (P.CLEAR in the figure); (f) shows scheme of the a comparative circuit for NEUROD1 overexpression in which AcrII4A-N7 protein is constitutively expressed, driven by the strong CMV promoter; (g) shows the results of quantification of NEUROD1 RNA performed by Real Time-PCR in Example 11: NEUROD1 has been overexpressed with a constitutively expressed VPR-dCas9-N8, together with the mix of all the three tested gRNAs, in absence of AcrII4A-N7 protein (CRISPRa), in the presence of AcrII4A-N7 protein controlled by the NEUROD1-respondive NiClear promoter (CRISPRaTOR, in accordance with the invention), and in the presence of the AcrII4A-N7 protein controlled by the strong constitutive CMV promoter (CMV_Acr).

FIG. 19: (a) shows a scheme of a tunable system for NEUROD1 overexpression according to the prior art: in the presence of doxycycline, the constitutively expressed rtTA transcription factor binds to the TRE3G promoter, hence expressing VPR-dCas9-N8, which in turn overexpressed NEUROD1. (b) shows a scheme of an NFL circuit according to the invention for tunable NEUROD1 overexpression: the overexpressed NEUROD1 protein binds to the NiClear promoter, hence driving the transcription and expression of AcrII4A-N7 protein, that binds to VPR-dCas9-N8, thus closing the loop. (c) NEUROD1 RNA levels detected by Real Time-PCR in Example 11. All the values for both circuits have been normalized on the RNA amount measured when doxycycline is not administered. The graph shows the fold change of expression when increasing Doxycycline doses. (e) shows RNA level of AcrII4A when NEUROD1 is overexpressed (gRNA_Mix) and when it is not overexpressed (gRNA_B) as measured in Example 11.

FIG. 20: (a) shows a basic biosensor (B) according to the prior art, wherein the species Z, that works as an output, is driven by an analyte-responding promoter. (b) shows a transcription factor (TF) biosensor of the prior art, wherein transcription factor X, driven by the analyte-responding promoter, is added to express Z, thus increasing the output. (c) shows an Antithetic Integral Controller (AIC) biosensor based on the NFL circuit of the invention, wherein a controller biomolecule Y, induced by Z, is added, to annihilate with X and inhibit it, in order to confer robustness and linearity. (d) shows a Mutual Inhibition (MI) biosensor, wherein X expresses Z, which inhibits and is itself inhibited by the controller W that is constitutively expressed. This topology confers high fold change and low leakiness. (e) shows a Mutual Inhibition-Feed Forward Loop (MI-FFL) biosensor, based on the CFFL genetic circuit of the invention, wherein X drives Z and also inhibits a controller biomolecule W; W and Z mutually inhibit each other. This enhances the fold change and further reduces leakiness. (f) a New Generation Biosensor-Mutual Inhibition (NGB_{MI}), which combines biosensors AIC and MI above and wherein X, driven by the analyte-responding promoter, induces Z, that in turns mutually inhibits with controller biomolecule W, constitutively expressed. A second controller biomolecule Y, induced by Z, annihilates with X, inhibiting it. (g) shows a New Generation Biosensor-Cascade (NGB_{C}), which combines NFL and CFFL circuits according to the invention, wherein: X drives Z, that mutual inhibits with W at the post-translational level, Z repressing W at the transcriptional level. The species Y is induced by Z and inhibits X. (h) shows a New Generation Biosensor-Integrated (NGB_{I}) in which: X induces Y, that annihilates with X, inhibiting it by negative feedback; X also induces Z and represses W, that mutually inhibits with Z itself.

FIG. 21 shows implementations of the circuits of FIG. 20. The rectangular shapes denote the coding sequences in the gene network responsible for specific functions, while promoters are represented by arrow-shaped boxes. The lines with an arrowhead indicate activation, while the lines with a T-shaped arrowhead indicate repression. Additionally, a barred circle and two joining arrows signify the annihilation mechanism. (a) shows Biosensor “B” of the prior art, in which the expression of the Luciferase protein (Z) is driven by the copper-responsive MRE promoter. (b) shows Biosensor “TF”, in which the VPR-dCas9-N8 protein (X) is driven by the MRE promoter and, in presence of the gRNA_B, constitutively expressed by the U6 promoter, binds to the 7B_pMin promoter, driving fLuc (Z) expression. (c) shows biosensor “AIC”, in which the VPR-dCas9-N8 protein (X) is driven by the MRE promoter and, with the constitutively expressed gRNA_B, drives the expression of the rtTA protein (Z), that in turns activate its reporter fLuc and the AcrII4A-N7 protein (Y), both driven by TRE3G promoter. Y then inhibits X. (d) shows biosensor “MI”, in which the VPR-dCas9-N8 protein (X) is driven by the MRE promoter. In the presence of the gRNA_B, X drives the expression of fLuc-DR (Z). The presence of the Direct Repeat (DR) allows the fLuc protein to mutual inhibits with the constitutively expressed CasRx (W). (e) shows biosensor “MI-FFL”, wherein the VPR-dCas9-N8 protein (X) activates fLuc-DR (Z) that mutual inhibits with CasRx (W). In this case, the CasRx protein is driven by custom promoter CMV/2B, allowing to be repressed by VPR-dCas9-N8. (f) shows Biosensor “NGB_{MI}” with 1 DR, in which the VPR-dCas9-N8 protein (X) is driven by the MRE promoter and, in presence of the gRNA_B, constitutively expressed by the U6 promoter, binds to the 7B_pMin promoter, inducing the expression of rtTA protein (Z), that, in turns, activates fLuc-DR (Z) under the control of the TRE3G promoter. The rtTA protein (Z) also activates the AcrII4a-N7 protein (Y) under the control of the TRE3G promoter, that inhibits VPR-dCas9-N8 preventing its binding to the DNA. In this topology, fLuc-DR perform mutual inhibition with the CasRx protein (W), constitutively expressed by the strong promoter CMV. (g) shows Biosensor “NGB_{MI}”, with 2 DR, in this case the Direct Repeat (DR) recognized by the CasRx (W) is placed also on rtTA (Z) and not only on the fLuc (Z). (h) shows Biosensor “NGB_{C}”, with 1 DR, wherein MRE-driven VPR-dCas9-N8 induces the rtTA protein (Z) under the control of 7B_pMin promoter, that drives the expression of fLuc-DR (Z) and AcrII4a-N7 (Y), both under the control of the TRE3G promoter, while repressing the transcription of CasRx, under the control of the CMV/TO promoter. The AcrII4A-N7 protein (Y) inhibits VPR-dCas9-N8 (X), while fLuc-DR (Z) and CasRx (W) performs mutual inhibition. (i) shows Biosensor “NGB_{C}”, with 2 DR, wherein the Direct Repeat (DR) recognized by the CasRx (W) is placed also on rtTA (Z) and not only on the fLuc (Z). (j) shows Biosensor “NGB_{I}”, with 1DR, wherein the MRE-driven VPR-dCas9-N8 (X), drives the expression of rtTA (Z), under the control of the 7B_pMin promoter, while repressing transcription of the CasRx protein (W), under the control of custom CMV/2B promoter. The rtTA protein (Z), induces the expression of TRE3G-driven AcrII4A-N7 (Y), that performs negative feedback with VPR-dCas9-N8, and expresses the TRE3G-driven fLuc-DR (Z), that performs mutual inhibition with the CasRx protein (W). (k) shows Biosensor “NGB_{I}”, with 2 DR, wherein the Direct Repeat (DR) recognized by the CasRx (W) is placed also on rtTA (Z) and not only on the fLuc (Z).

FIG. 22 shows repression of CMV/2B promoter in the presence of VPR-dCas9-N8 protein, as tested in Example 12 (a) shows the Relative luminescence responses (RLU) of the firefly luciferase driven by the CMV/2B promoter, in presence of the VPR-dCas9-N8 protein together with the gRNA_B, directed towards the fLuc promoter, or with the scramble guide gRNA_AB. (b) shows the percentage of repression of the CMV/2B promoter, obtained by calculating the ones' complement on the values obtained when using gRNA_B against the values obtained when using gRNA_AB.

FIG. 23 shows input-output response and performance of MI, MI-FFL, NGB_{MI} 1DR, and NGB_{MI} 2DR biosensors of FIG. 21, as assessed in Example 12. (a) shows performance of the AIC biosensor compared to the performance of the Basal biosensor B; (b) shows performance of the MI biosensor compared to the performance of biosensor B; (c) shows Performance of the MI-FFL biosensor compared to the performance of the biosensor B; (d) shows Performance of the NGB_{MI} biosensor with 1 DR compared to the performance of biosensor B; € shows Performance of the NGB_{MI} biosensor with 2 DRs compared to the performance of biosensor B. The left panels show relative luminescence responses in logarithmic scale (Log RLU) as the copper concentration ranges from 0 to 150 μM, obtained performing Luc assay after transfecting the genetic constructs for each system in HEK293T cells. The central panels show their normalization to the first and uninduced point (copper 0 μM), to calculate the Fold activation. For each system, the mean and the 9 points collected over three independent biological experiments are represented. The right panels show the radar plot of the systems' performance (Leakiness, Fold change, Sensitivity to input, Operation range, and Linearity) compared against the basic biosensor. Each performance is normalized to assume a score ranging from 0 (assigned to the worst system) to 1 (assigned to the best system).

FIG. 24 shows input-output response and performance of NGB_{C} 1DR, NGB_{C} 2DR, NGB_{I} 1DR, and NGB_{I} 2DR biosensors of FIG. 21, as assessed in Example 12. Left, center and right panels are as in FIG. 23 (a) shows performance of the NGB_{C} biosensor with 1 DR compared to the performance of the Basal biosensor (B); (b) shows Performance of the NGB_{C} biosensor with 2 DRs compared to the performance of biosensor B; (c) shows Performance of the NGB_{I} biosensor with 1 DR compared to the performance of biosensor B; (d) shows Performance of the NGB_{I} biosensor with 2 DRs compared to the performance of biosensor B.

FIG. 25 shows: (a) a scheme the mutual repression between species “Y” and “Z”, and its biological implementation where species (Y) is CasRx endoribonuclease and species (Z) is Gaussia Luciferase (gLuc) transcript with e Direct Repeat (DR) in the 3′Untranslated Region (UTR) Upon binding to the DR, the CasRx cleaves the polyA tail (pA) from gLuc transcript causing its degradation mediated by the cellular exonucleases, hence, achieving Y-mediated repression of Z. After cleavage, the CasRx remains bound to the DR, forming the mRNA-Cas binary complex. Consequently, the DR-bound CasRx is no more available to cut another transcript. This implements the Z-mediated repression of Y, through a DR-sponge effect. (b) shows validation of the mutual inhibition assessed in Example 13: CasRx efficiently represses a transcript bearing a single DR. Instead, increasing the amount of DR repetition inhibits the cleavage efficacy of CasRx. Error bars correspond to the standard deviation for n=4 biological replicates. AAA: poly(A) tail; DR: Direct Repeat.

FIG. 26 shows a representation of mutual repression between species “Y” and “Z” in genetic circuits where species “X” activates the species “Z” upon stimulation, and their biological implementation according to the invention: (a) CASwitch v.1 system, wherein species “X” is implemented by the rtTA3G, while “Y” and “Z” by the CasRx and the gLuc gene downstream the pTRE3G promoter and having the DR in its 3′UTR, respectively. (b-h) show the results of Example 14: (b) Comparison of Tet-On3G and CASwitch v.1 dose response curves as gLuc A.U., in the presence of doxycycline. (n=4). (c) Comparison of maximum induced gLuc expression of Tet-On3G and CASwitch v.1 systems as relative gLuc A.U (relative to the maximum gLuc A.U. value obtained by the TET-ON3G system at the highest concentration of Doxycycline, 1000 ng/mL) (n=4). (d) Comparison of fold induction activation curves of Tet-On3 and CASwitch v.1 systems by increasing doxycycline concentration up to 1000 ng/ml (n=4). (e) CASwitch v.2 circuit, wherein species “X” is implemented by the rtTA3G, while “Y” and “Z” by the CasRx driven by the pCMV/TO and the gLuc gene downstream the pTRE3G promoter, having the DR in its 3′UTR, respectively. (f) Comparison of Tet-On3G and CASwitch v.2 dose response curves (n=4). (g) Comparison of maximum induced gLuc expression of Tet-On3G and CASwitch v.2 systems (n=4). (h) Comparison of fold induction activation curves of Tet-On3 and CASwitch v.2 systems (n=4). rtTA3G: reverse tetracycline TransActivator 3G; pTRE3G: Tetracycline Responsive Element promoter 3G; pCMV/TO: modified CMV promoter bearing two Tetracycline Operon (TO) sequences. Fold-induction values are calculated as the ratio between the value of gLuc expression of each data point and the mean of gLuc expression in the absence of doxycycline. Relative Luciferase A.U. values are calculated as the ratio between the Luciferase A.U. value of each data point and the mean of Luciferase A.U. values in the Tet-On3G system at 1000 ng/ml of doxycycline. Error bars indicate standard error.

FIG. 27 shows: (a) a schemes pMRE copper biosensor (top panel) and Tet-On3G-amplified biosensor (middle panels), and of CFFL genetic circuit amplified biosensor (CASwitch v.2, lower panel). (b-c) shows comparison of pMRE, Tet-On3G and CASwitch v.2 biosensors as assessed in Example 15: (b) shows a dose-response curves for increasing levels of Copper Chloride, as Luciferase A.U., in the presence of 1000 ng/ml of doxycycline. (c) shows dose-response curve, as relative Fold Induction relative to the minimum Luciferase A.U. value obtained in the absence of Copper Chloride (n=4, albeit for Cu2+=25 μM which shows 3 replicates).

FIG. 28 shows implementation of a lysosomal stress biosensor based on a CFFL genetic circuit, according to preferred embodiments of the invention and results of its assessment in Example 16 (A) shows a scheme of the CFFL-based biosensor on the left, and of comparative Tet-On3G amplified- and pNiClear-regulated biosensors (in the middle and on the right respectively). In the pNiClear-regulated biosensor, upon Torin-1 administration, endogenous TFEB transcription factor binds its cognate synthetic promoter pNiClear, inducing Firefly Luciferase (fLuc) expression in Torin1-dependent manner; in the Tet-On3G amplified configuration, the pNiClear drives the expression of the rtTA3G in a Torin1-dependent manner, which in turn induces the expression of the fLuc through the pTRE3G in the presence of doxycycline; in the CFFL-based biosensor (CASwitch amplified configuration), the pNiClear drives the expression of the rtTA3G in a Torin1-dependent manner, which in turn is induces pTRE3G-fLuc and inhibits the CasRx through the pCMV/TO promoter in the presence of doxycycline. Instead, in the absence of Torin-1, the CasRx represses fLuc expression by cleaving the Direct Repeat (DR) in the fLuc 3′untranslated region (3′UTR). (B) shows comparison of dose response curves of pNiClear, Tet-On3G and CFFL biosensor configurations (n=4) (C) shows comparison of fold induction activation curves of pNiClear, Tet-On3G and CFFL biosensors. (n=4); Luciferase luminescence level are normalized against Renilla luminescence in (b) or normalized by the value in the absence of Torin-1a in (c).

FIG. 29: (a) shows schemes of Tet-On3G and CASwitch v.2 control of HSV-TK expression. pCMV-HSV-TK represents a positive control condition in which the cytotoxic gene HSV-TK is downstream constitutive promoter CMV, being thus constitutively expressed with or without doxycycline. In the Tet-On3G system, representing the prior art, the rtTA3G induces the expression of the cytotoxic HSV-TK gene in the presence of doxycycline, but its leaky expression in the absence of doxycycline potentially causes unintended cell toxicity. Instead in the CASwitch v.2 system, according to a CFFL circuit of the invention, the leaky HSV-TK expression is repressed by the CasRx, potentially resulting in no cytotoxic effect in the absence of doxycycline. AAV: Adeno-Associated Virus; HSV-TK: Herpes Simplex Virus Thymidine Kinase (b) shows cell viability comparison between Tet-On3G and CASwitch v.2 systems, as assessed in Example 17, in the absence or in the presence of 1000 ng/ml of doxycycline in WT Hek293 cells (Mock), and in Hek293 cells transfected with each one of the three genetic circuits. The values represent the mean and standard deviation of replicates within two independent experiments (n=9). Statistical analysis was conducted on the pooled data from all experiments by the ANOVA, after determining equal or unequal variances by D'Agostino & Pearson test; *****=p<0.0001. (c) shows representative images of crystal violet staining of cell culture plates showing cell viability of Tet-On3G and CASwitch v.2 control of HSV-TK expression.

FIG. 30 shows: (a) scheme of triple transfection for AAV production according to the prior art (top panel), with a Tet-On3G inducible system (lower panel, left) and with a CFFL circuit (CASwitch v.2) controlling expression of helper genes. E2A: Early 2 A gene; E4: Early 4 gene; VaRNA: Viral associated RNA; DBP: DNA binding protein; Orf6: Open reading frame 6. (b) Representative histograms of flow cytometry analyses of HEK293T cells transduced with AAV production yielded using the Tet-On3G and CASwitch v.2 systems in the presence and absence of doxycycline. (c) Comparison of the percentage of infected cell transduced with AAV produced by the Triple Transfection (w or w/o Helper; no Helper/+Helper), Tet-On3G and CASwitch v.2 configurations. Error bars correspond to the standard deviation for at least n=6 biological replicates. Percentage of transduced cell calculated as the percentage of GFP+ cells, setting the cell autofluorescence threshold using non transduced cells. At least 10000 cells were analyzed for each point. Black bars indicate the presence of doxycycline presence (1000 ng/mL), instead, gray bars its absence in the growth medium. The “Helper” refers to the triple transfection methos, and the “No Helper” to a negative control where the triple transfection gene circuit is used but without the pHelper plasmid.

FIG. 31 shows Comparison of the percentage of infected cell transduced with AAV produced by the Triple Transfection (w or w/o Helper; no Helper/+Helper), Tet-On3G and CASwitch v.2 configurations as CasRx amounts increase. Black bars indicate the presence of doxycycline presence (1000 ng/mL), instead, gray bars its absence in the growth medium.

### DETAILED DESCRIPTION OF THE INVENTION

The present invention is then directed to genetic circuits wherein proteins and/or nucleic acid components are functionally linked, so that the expression, concentration and/or activity of at least one component of the circuit is directly and/or indirectly influenced by at least another component of the circuit, closing the loop.

For example, the circuit comprises at least one protein whose expression or activity is activated or repressed by another component of the circuit.

The invention is directed to a novel genetic circuit for regulating expression of genes in a cell, comprising transcriptional units for expressing the components of the circuit in the cell, and in particular:
a) at least one transcription unit for expressing a transcriptional activator in the cell, comprising a promoter operably linked to a polynucleotide encoding the transcriptional activator, preferably an inducible transcriptional activator, said transcriptional activator positively regulating the expression of at least one gene of interest in the cell;
b) at least one transcription unit for expressing at least one controller biomolecule in the cell, comprising a promoter operably linked to a polynucleotide encoding a controller biomolecule, said promoter being such that i) the transcriptional activator negatively regulates the expression of the controller biomolecule, or such that ii) the transcriptional activator positively regulates the expression of the controller biomolecule;
   and optionally comprising:
c) at least one transcription unit for expressing at least one gene of interest in the cell, comprising a promoter operably linked to at least one gene of interest, preferably wherein said promoter comprises a binding site for the transcriptional activator, such that binding of the transcriptional activator to said binding site positively regulates the expression of the gene of interest;
   and wherein the at least one controller biomolecule negatively regulates at least one component of the genetic circuit.

The genetic circuit of the invention is a genetic circuit having a closed loop configuration; the controller biomolecule with its regulatory activity, closes the loop in the genetic circuit of the invention.

The genetic circuit of the invention is engineered as at least one component of the circuit is recombinant (i.e., non-natural), such as a recombinant gene or protein.

Preferably all the transcription units of the engineered genetic circuit of the invention are recombinant transcription unit.

A gene of interest is a polynucleotide encoding a transcript and/or protein of interest.

A transcriptional activator is a protein comprising DNA-binding domains (DBDs) and activation domains (ADs), capable of promoting transcription of a gene or set of genes upon binding to a binding site in the promoter of the gene or set of genes. Transcriptional activators are natural transcription factors, as well as recombinant transcription activators.

The genetic circuit comprises a) at least one transcriptional unit for expressing a transcriptional activator, preferably an inducible transcriptional activator whose activity is induced by an inducer molecule. Particularly preferred are the tetracycline-inducible transcriptional activators, most preferably the inducible transcriptional activator is rtTA or variants thereof.

Therefore, the genetic circuit preferably comprises a transcriptional unit for expressing rtTA, comprising a promoter, operably linked to a polynucleotide encoding rtTA having sequence comprising or consisting of SEQ ID NO: 1 or variants thereof, or a polynucleotide encoding a modified rtTA, where the VP16 transactivation domain of the rtTA was replaced with a VPR transactivation domain, having sequence comprising or consisting of SEQ ID NO: 4 or variants thereof. A rtTA with the VPR transactivation domain substituting the VP16 domain can also be called rTetR-VPR, since it comprises the rTetR portion of the rtTA fused to the VPR domain.

The transcriptional activator of the invention positively regulates expression of at least one gene of interest.

In accordance with the present invention, the gene of interest can be any gene encoding any transcript or protein, either natural or artificial, e.g., recombinant, which is positively regulated by the transcriptional activator.

The term “positively regulated” means here that the regulating component (e.g., the transcriptional activator) positively affects the expression, concentration and/or the biological activity of the regulated component (e.g., a gene, a transcript, or a protein, of interest, or any other biomolecule in the cell) of the genetic circuit in a cell. Such positive regulation provided by a transcriptional activator may occur by direct or indirect activation of transcription (expression) of a gene encoding the regulated component or by direct or indirect activation of translation of the regulated component from mRNA.

As an example, the gene of interest can be a gene encoding a reporter molecule (such as a luminescent or fluorescent reporter molecule), whose expression is induced by the transcriptional activator (e.g., by direct binding of the transcriptional activator to a binding site in the promoter driving expression of the gene encoding the reporter molecule), said transcriptional activator being activated by an analyte of interest (e.g., activating expression of the transcriptional activator), so that the protein encoded by the gene of interest can be used as a biosensor.

Preferably, the genetic circuit comprises c) at least one recombinant transcription unit for expressing the at least one gene of interest in the cell, comprising a promoter operably linked to at least one gene of interest, wherein said promoter preferably comprises a binding site for a transcriptional activator, such that binding of the transcriptional activator to said binding site positively regulates the expression of the gene of interest.

As typically used in the art, the term the term “expression of a gene of interest” can be also used in place of “expression of a transcript of interest” or “expression of a protein of interest” to mean that the gene is transcribed and/or translated. Equally, unless otherwise indicated, “expression of a protein”, such as expression of a protein of interest, is a short term for indicating transcription and translation of a gene of interest encoding the protein.

The gene of interest can be a gene encoding a natural protein, endogenous to a cell comprising the genetic circuit, or a recombinant protein artificially expressed in the cell.

According to preferred aspects of the present invention, the transcriptional activator can regulate the expression of the gene of interest directly, by binding to the promoter operably linked to the gene of interest, or indirectly by positively regulating one or more further transcriptional activators which in turns positively regulate the gene of interest. Said further transcriptional activators can be for instance natural transcription factors expressed by the cell, such as mammalian cell, such as TFEB, GADD34, Xbp1s, Tp53, Nf-Kβ, NEUROD1, or any other transcriptional activator. For instance, Xbp1s can regulate the expression of genes that are capable of ameliorating proteins' folding in a cell, ultimately controlling the ER Stress; GADD34 is involved in Integrated Stress Response (ISR) processes and in EF2a dephosphorylation, maintaining it in an active state thus increasing the amounts of proteins produced by the cell.

In preferred embodiments, the transcription unit comprising the polynucleotide encoding the transcriptional activator can comprise a polynucleotide encoding a protein of interest linked to the polynucleotide encoding the transcriptional activator, so that the transcriptional activator is expressed together with, optionally fused to, the protein of interest.

According to the present invention, the controller biomolecule expressed by a transcriptional unit of the genetic circuit of the invention negatively regulates at least one component of the genetic circuit.

The term “negatively regulates” means here that the controller biomolecule directly or indirectly negatively affects the concentration and/or the biological activity of the regulated component of the genetic circuit in the cell. Such regulation may occur by direct or indirect reduction or repression of transcription of a gene encoding the regulated component or of translation of the regulated component from mRNA, or by direct or indirect degradation of the regulated component, or by direct or indirect inhibition of the biological activity of the regulated component. For example, the controller biomolecule can have means to physically interact with the regulated component and to cut, hydrolyse, degrade, or sequestrate the regulated component, so that the biological activity of the latter is inhibited.

Preferably, the controller biomolecule expressed by b) the transcriptional unit of the genetic circuit of the invention is a protein, whose expression is regulated by the transcriptional activator encoded by the at least one transcription unit a) of the genetic circuit.

In a first preferred aspect of the present invention, the promoter of the transcription unit b) for expressing the controller biomolecule is such that i) the transcriptional activator negatively regulates the expression of the controller biomolecule; in such preferred aspects, the at least one component of the genetic circuit that is negatively regulated by the controller biomolecule is the product of expression of the gene of interest, such as a protein encoded by the gene of interest, or a transcript of the gene of interest.

In a second preferred aspect of the present invention, the promoter of the transcription unit b) for expressing the controller biomolecule is such that ii) the transcriptional activator positively regulates the expression of the controller biomolecule; in such second preferred aspect, the at least one component of the genetic circuit that is negatively regulated by the controller biomolecule is the transcriptional activator, preferably said controller biomolecule being a protein capable of sequestering the transcriptional activator.

Positive or negative regulation of the expression of the controller biomolecule can be achieved by providing transcription units for expression of the controller biomolecule comprising a promoter that comprises a binding site for a transcriptional activator, said binding site being such that ii) binding of a transcriptional activator activates expression of the controller biomolecule or such that i) binding of the transcriptional activator inhibits expression of the controller biomolecule.

Inhibition of the expression can be obtained for example in case of a promoter whose binding site for the transcriptional activator is at a position such that binding of the transcriptional activator poses a steric hindrance to the RNA polymerase, resulting in transcription inhibition.

A preferred promoter such that i) binding of the transcriptional activator inhibits expression of the controller biomolecule is a pCMV/TO promoter: said promoter comprises one or more Tetracycline Operon (TO) sequences flanking the TATA-binding box of the promoter such that binding of a tTA transcriptional activator poses a steric hindrance to the RNA polymerase, resulting in inhibition of the controller's transcription; more preferably said promoter has sequence comprising or consisting of SEQ ID NO: 8, or variants thereof. A further preferred promoter, such that i) binding of the transcriptional activator inhibits expression of the controller biomolecule, is a pCMV/2B promoter comprising or consisting of sequence SEQ ID NO: 48, which can be repressed by VPR-Cas9-N8 protein (see Example 12).

In preferred embodiments, the transcriptional activator that regulates expression of the controller is the transcriptional activator that positively regulates the gene of interest, encoded by the at least one transcription unit of the genetic circuit.

The transcriptional activator that regulates expression of the controller can also be a further transcriptional activator that is positively regulated by the upstream transcriptional activator encoded by the transcription unit of the genetic circuit, so that the transcriptional activator encoded by the transcription unit of the genetic circuit indirectly regulates the expression of the controller biomolecule. Said further transcriptional activator positively regulated by the upstream transcriptional activator encoded by the transcription unit of the genetic circuit can be a recombinant transcriptional activator, expressed from a further transcription unit of the genetic circuit, or a natural transcriptional activator, endogenous in the cell.

In some other embodiments, the transcriptional activator that regulates expression of the controller is a natural transcriptional activator, endogenous in the cell, that is positively regulated by an upstream transcriptional activator.

Preferably, where the at least one component of the genetic circuit that is negatively regulated by the controller biomolecule is the product of the at least one gene of interest, the controller biomolecule is a nuclease, more preferably an endonuclease, most preferably an endoribonuclease; particularly preferred is a CRISPR-associated endoribonuclease.

Examples of CRISPR-associated endoribonuclease (CRISPR-Cas RNases) in accordance with the invention include, but are not limited to, Cas9 nucleases, Cas6 nucleases, Cas12a nucleases, Cas13 nucleases, Csy4 nuclease, CasE nuclease, Cse3 nuclease and variants thereof.

In accordance with the present invention, a variant of any biomolecule is a biomolecule that has a nucleic acid or aminoacidic sequence that is at least 50%, 60%, 70%, 80%, 90%, 95%, or 99% identical to the wild-type nucleic acid or aminoacidic sequence and that retains the biological activity of the wild-type biomolecule.

For example, a variant of an endoribonuclease comprises or consists of a nucleic acid sequence that is at least 50%, 60%, 70%, 80%, 90%, 95%, or 99% identical to a wild-type endoribonuclease nucleic acid sequence and that retains the endoribonuclease activity of the wild-type protein.

In accordance with degeneration of genetic code, variants include sequences where at least one base of the base sequence of a gene is replaced with a different type of base, without changing the amino acid sequence of the polypeptide expressed from the gene. Variants also include codon-optimized sequences and sequences comprising mutated or added nucleotides, e.g., for cloning needs.

In accordance with the present invention, variants also include sequences encoding fragments of any biomolecule, i.e., a shorter form of the biomolecule, such as a truncated form, that retains the biological activity of the wild-type biomolecule.

Preferred CRISPR-Cas RNases expressed as controller according to the invention are Type VI CRISPR-Cas RNases.

Particularly preferred are CRISPR-Cas RNases derived from Ruminococcus flavefaciens, such as CRISPR-Cas13d (RfxCas13d or CasRx) or variants thereof.

CasRx belongs to the Cas13 family of CRISPR-specific endonucleases which can cleave their own pre-crRNAs to produce shorter gRNA sequences used for RNA-targeting. CasRx can strongly inhibit the expression of a gene of interest at the post-transcriptional level if a DR sequence is placed in the UTR of the gene of interest, preferably in the 3′UTR. Indeed, the endoribonuclease cleaves the poly A from the gene transcript causing its degradation.

CasRx shows robust activity in human cells and CasRx-mediated knockdown has exhibited higher efficiency and specificity compared to RNA interference across diverse transcripts.

Moreover, being among the most compact single effector Cas enzymes, CasRx can also be flexibly packaged into viral vectors, for instance adeno-associated virus vectors.

Preferably, the controller is CasRx having sequence comprising or consisting of SEQ ID NO: 2 or variants thereof.

In preferred aspects of the present invention, where the controller biomolecule is a CRISPR-Cas RNase, the component of the circuit negatively regulated by the controller is the at least one gene of interest, whose sequence preferably comprises one or more Direct Repeat (DR) sequences in its UTR, preferably in its 3′UTR. A “Direct Repeat” or “DR” sequence refers to a polynucleotide that is between 10 and 40 nucleotides in length, and typically comprises an 8-10 nucleotide stem with an A/U-rich loop. In some embodiments, a DR sequence (e.g., a Casl3d DR sequence) is 30, 31, 32, 33, 34, 35, or 36 nucleotides in length.

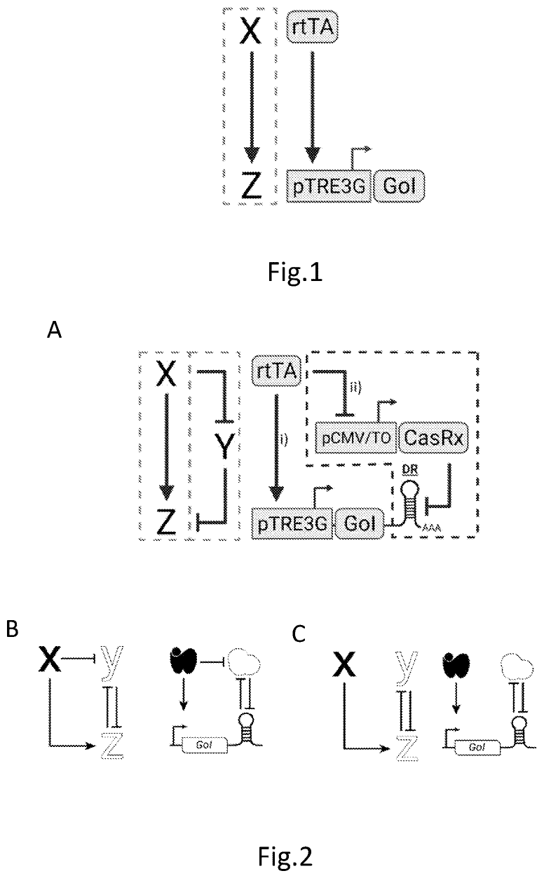

Advantageously, the controller endoribonuclease CasRx functionally implements a mutual inhibition topology between a controller biomolecule (repressor species “Y”) that negatively regulates a gene of interest and the gene of interest (an output species “Z) as shown in FIG. 2B. Without being bound to theory, in fact, CasRx endoribonuclease interacts in a 1:1 stochiometric ratio with its target molecule through the Direct Repeats forming a binary complex. Thus when CasRx acts as the controller biomolecule Y it can effectively inhibit the gene of interest Z, but at the same time, if Z increases it can “sponge” the CasRx thus effectively implementing a mutual inhibition.

Preferably, the controller biomolecule is CasRx and the sequence of the gene of interest negatively regulated by the controller biomolecule comprises Direct Repeats of sequence SEQ ID NO: 3, or variants thereof.

Therefore the present invention is preferably directed to an engineered genetic circuit for regulating expression of genes in a cell, comprising:
at least one transcription unit for expressing a transcriptional activator in the cell, comprising a promoter operably linked to a polynucleotide encoding the transcriptional activator, preferably an inducible transcriptional activator, said transcriptional activator positively regulating the expression of at least one gene of interest in the cell;
at least one transcription unit for expressing at least one controller biomolecule in the cell, comprising a promoter operably linked to a polynucleotide encoding a controller biomolecule; and
at least one transcription unit for expressing at least one gene of interest in the cell, comprising a promoter operably linked to at least one gene of interest, said promoter comprising a binding site for the transcriptional activator, such that binding of the transcriptional activator to said binding site positively regulates the expression of the gene of interest;
   wherein the at least one controller biomolecule and the at least one gene of interest negatively regulate each other, through mutual inhibition, more preferably wherein the controller biomolecule is CasRx (SEQ ID NO: 2 or variants thereof) and the sequence of the gene of interest negatively regulated by the controller biomolecule comprises Direct Repeats (SEQ ID NO: 3, or variants thereof).

According to alternative aspects of the invention, the promoter of the controller biomolecule is a constitutive promoter.

A constitutively expressed CasRx, combined with its cognate direct repeat, can serve as a plug-and-play platform to enhance a transcriptional inducible gene system, such as Tet-On3G, by reducing leakiness, retaining high maximum expression, and amplifying its fold-induction levels.

The present invention is therefore directed, in alternative aspects, also to a genetic circuit for regulating expression of genes in a cell, comprising at least one transcription unit for expressing a transcriptional activator in the cell, comprising a promoter operably linked to a polynucleotide encoding the transcriptional activator, preferably an inducible transcriptional activator, said transcriptional activator positively regulating the expression of at least one gene of interest in the cell;
at least one transcription unit for expressing at least one controller biomolecule in the cell, comprising a constitutive promoter operably linked to a polynucleotide encoding a controller biomolecule;
and at least one transcription unit for expressing at least one gene of interest in the cell, comprising a promoter operably linked to at least one gene of interest, said promoter comprising a binding site for the transcriptional activator, such that binding of the transcriptional activator to said binding site positively regulates the expression of the gene of interest;
wherein the at least one controller biomolecule and the at least one gene of interest negatively regulate each other, through mutual inhibition; more preferably wherein the controller biomolecule is CasRx (SEQ ID NO: 2 or variants thereof) and the sequence of the gene of interest negatively regulated by the controller biomolecule comprises Direct Repeats (SEQ ID NO: 3, or variants thereof).

Further preferred controller biomolecules that can be employed in the engineered genetic circuits of the invention include: LwaCas13a, PspCas13b, RanCas13b, PguCas13b Csy4, CasE, Cse3, Cas6 RNases, more preferably LwaCas13a from Leptotrichia wadei (Abudayyeh O O et al. RNA targeting with CRISPR-Cas13. Nature. 2017 Oct. 12; 550 (7675): 280-284), and variants thereof. Promoters of the transcription units of the genetic circuit of the invention can be constitutive or inducible promoters. Examples of constitutive promoters include, without limitation, the retroviral Rous sarcoma virus (RSV) LTR promoter (optionally with the RSV enhancer), the cytomegalovirus (CMV) promoter, the SV40 promoter, the dihydrofolate reductase promoter, the b-actin promoter, the phosphoglycerol kinase (PGK) promoter, and the EF1 a promoter [Invitrogen]. In some embodiments, a promoter is an RNA pol III promoter, such as U6 or H1. In some embodiments, a promoter is an RNA pol II promoter.

Preferably, the polynucleotide encoding the transcriptional activator is under control of a constitutive promoter, such as a CMV promoter of sequence SEQ ID NO: 5 or variants thereof.

According to a first preferred aspect of the present invention, the engineered genetic circuit comprises:
a) at least one transcription unit for expressing a transcriptional activator in the cell, comprising a promoter, preferably a constitutive promoter, operably linked to a polynucleotide encoding for the transcriptional activator, preferably for an inducible transcriptional activator, that positively regulates the expression of at least one gene of interest;
b) at least one transcription unit for expressing at least one controller biomolecule in the cell, comprising a promoter operably linked to at least one polynucleotide encoding for a controller biomolecule, said promoter being such that i) the transcriptional activator negatively regulates the expression of the controller biomolecule; and
wherein the at least one controller biomolecule negatively regulates the expression of the at least one gene of interest.

Such a genetic circuit has a Coherent Feed Forward Loop (CFFL) configuration: CFFL is an ideal biomolecular circuit that can achieve high fold-change and increased dynamic range, by quenching leakiness. A feedforward loop is ‘coherent’ when an input signal is split into two downstream pathways, both of which positively regulate the output. A low level of input species X does not activate expression of Z output and does not repress Y, therefore Y is highly active and strongly inhibits the output Z. At high level of species X, Z is activated and Y is repressed, resulting in maximum output level of Z.

For the first time, the present inventors have provided implementations of a CFFL genetic circuit for controlling gene expression in a cell.

According to the first preferred aspect of the present invention, the CFFL engineered genetic circuit preferably comprises:
a) at least one transcription unit for expressing a transcriptional activator in the cell, comprising a promoter, preferably a constitutive promoter, operably linked to a polynucleotide encoding for the transcriptional activator, preferably for an inducible transcriptional activator;
b) at least one transcription unit for expressing at least one controller biomolecule in the cell, comprising a promoter operably linked to at least one polynucleotide encoding for a controller biomolecule, said promoter comprising a binding site for the transcriptional activator, such that i) binding of the transcriptional activator to said binding site negatively regulates the expression of the controller biomolecule; and
c) at least one transcription unit for expressing at least one gene of interest in the cell, comprising a promoter operably linked to at least one gene of interest, wherein said promoter comprises a binding site for the transcriptional activator, such that binding of the transcriptional activator to said binding site positively regulates the expression of the gene of interest;
wherein the at least one controller biomolecule negatively regulates the expression of the at least one gene of interest.

Preferably, the genetic circuit having a CFFL configuration employs the doxycycline-inducible gene expression system (Tet-ON or Tet-ON 3G): the Tet-ON system represents one of the pathways, where the rtTA is the input transcription factor (X) that activates the output (Z/Gene of Interest, GOI), binding to the upstream pTRE promoter in the presence of the inducer molecule doxycycline (See FIG. 1) and a second pathway is plugged on the Tet-ON system, positively regulating the gene of interest by repressing a controller biomolecule that negatively regulates the gene of interest (see FIG. 2).

More preferably, the genetic circuit then comprises:
a1) at least one transcription unit for expressing a rtTA inducible transcriptional activator, comprising a promoter, preferably a constitutive promoter, such as a CMV constitutive promoter of sequence SEQ ID NO: 5 or variants thereof, operably linked to a polynucleotide encoding for the rtTA inducible transcriptional activator, preferably said polynucleotide having sequence SEQ ID NO: 1 or 4 or variants thereof; most preferably said at least one transcription unit for expressing a rtTA inducible transcriptional activator has sequence SEQ ID NO: 6 or variants thereof;
b1) at least one transcription unit for expressing at least one controller biomolecule in the cell, comprising a promoter operably linked to at least one polynucleotide encoding for a controller biomolecule, wherein said promoter comprises a binding site for the rtTA inducible transcriptional activator such that i) binding of the rtTA activator to said binding site negatively regulates the expression of the controller biomolecule, preferably said promoter comprising one or more Tetracycline Operon (TO) sequences flanking the TATA-binding box of the promoter such that binding of the transcriptional activator poses a steric hindrance to the RNA polymerase, resulting in inhibition of the controller's transcription, more preferably said promoter being pCMV/TO having sequence SEQ ID NO: 8 or variants thereof;
c1) at least one transcription unit for expressing at least one gene of interest in the cell, comprising a promoter responsive to the rtTA inducible transcriptional activator, such as a the pTRE promoter, the pTRE-Tight promoter, and the pTRE3G promoter (all available from Clontech Laboratories, Mountain View, CA) or variants thereof, more preferably comprising a pTRE3G promoter having sequence SEQ ID NO: 7 or variants thereof, said promoter being operably linked to the at least one gene of interest, wherein binding of the transcriptional activator to the promoter positively regulates the expression of the gene of interest;
   wherein the controller biomolecule negatively regulates the expression of the at least one gene of interest of the genetic circuit, preferably wherein the controller biomolecule is a CRISPR-Cas RNase, and wherein the sequence of the gene of interest comprises one or more Direct Repeat (DR) sequences.

Advantageously, a preferred genetic circuit having a CFFL configuration, as described above, can be used to control expression of toxic genes in a cell.

In a preferred embodiment, the invention is directed to a genetic circuit, having a Coherent Feed Forward Loop (CFFL) configuration, for controlling expression of toxic viral genes in a cell, and to its uses in a method of producing viral vectors, preferably for gene therapy.

Particularly preferred is a genetic circuit that can be used in a method of producing AAV viral vectors in mammalian cells. So far, the most promising gene therapy vehicles are in fact vectors based on Adeno Associated Virus (AAV vectors). However, their manufacturing yields are low and do not match the ongoing increasing demand for clinical trials and commercialization of approved gene therapies. The development of an AAV vector producer cell line stably harboring the viral genes is strongly needed. An expression system wherein viral proteins are under the control of an inducible activator-responsive promoter, such as a tetracycline responsive promoter inducing the expression of viral toxic genes after administration of the inducer molecule, would be desirable to manufacture AAV vectors on demand. However, previous attempts to engineer an AAV producer cell line failed so far because of viral toxic's leaky expression stemming from the tetracycline responsive promoter. Moreover, the wild-type Rep gene required for AAV production harbors in its coding sequence the p 19 promoter, which cannot be deleted/substituted without disrupting the coding sequence of the Rep gene.

According to further preferred embodiments, genetic circuits of the present invention can be used in a method of producing lentiviral vectors in mammalian cells, preferably lentiviral vectors for gene therapy.

Advantageously, the preferred genetic circuit of the present invention, having a Coherent Feed Forward Loop (CFFL) configuration and being capable of quenching leakiness can overcome the above restrictions, for instance by degrading leaky transcripts by means of a CRISPR-Cas RNAase in the absence of doxycycline, while in the presence of doxycycline, the repression of the genes of interests would be inhibited and the expression of the same would be activated.

Therefore, preferably the genetic circuit of the invention comprises:
a2) at least one transcription unit for expressing a rtTA inducible transcriptional activator, comprising a promoter, preferably a constitutive promoter, such as a CMV constitutive promoter of sequence SEQ ID NO: 5 or variants thereof, operably linked to a polynucleotide encoding for the rtTA inducible transcriptional activator, preferably said polynucleotide having sequence SEQ ID NO: 1 or 4 or variants thereof; most preferably said at least one transcription unit for expressing a rtTA inducible transcriptional activator having sequence SEQ ID NO: 6, or variants thereof;
b2) at least one transcription unit for expressing at least one controller biomolecule in the cell, comprising a promoter operably linked to at least one polynucleotide encoding for a controller biomolecule, said promoter comprising a binding site for the rtTA inducible transcriptional activator such that i) binding of the rtTA activator to said binding site negatively regulates the expression of the controller biomolecule, preferably said promoter having sequence SEQ ID NO: 8 or variants thereof, said polynucleotide preferably encoding a CRISPR-Cas RNase, more preferably encoding for CasRx and having sequence comprising or consisting of SEQ ID NO: 2 or variants thereof; preferably said transcription unit for expressing at least one controller biomolecule comprising or consisting of sequence SEQ ID NO: 14 or variants thereof;
and
c2) at least one transcription unit for expressing an AAV Rep protein, AAV Cap protein and one or more adenoviral helper proteins in a cell, wherein expression of AAV Rep and Cap proteins together with the one or more adenoviral helper proteins in the cell produce recombinant AAV (rAAV) virions, said at least one transcription unit c2) comprising a promoter responsive to the rtTA inducible transcriptional activator, preferably a pTRE3G promoter having sequence SEQ ID NO: 7 or variants thereof, driving the expression of at least one polynucleotide encoding the AAV Rep and Cap proteins and of one or more adenoviral helper proteins, upon binding of the transcriptional activator; preferably said at least one transcription unit comprising one or more Direct Repeat (DR) sequences in the 3′UTRs of the encoding polynucleotides, more preferably of sequence SEQ ID NO: 3, or variants thereof.

Preferably said AAV Rep and Cap proteins are AAV-2 Rep and Cap proteins and said adenoviral helper proteins are human adenovirus 5 (Had5) helper proteins.

Preferably, the polynucleotide encoding AAV Rep and Cap proteins has sequence comprising or consisting of SEQ ID NO: 9, SEQ ID NO: 56, or variants thereof.

Preferably, the polynucleotide encoding engineered human adenovirus helper proteins comprises a minimal gene set encoding E2A DNA binding protein and E4(Orf6) binding protein (μHelper gene set), more preferably the E2A DNA binding protein having sequence comprising or consisting of SEQ ID NO: 11 or variants thereof, and the E4(Orf6) binding protein having sequence comprising or consisting of SEQ ID NO: 12 or variants thereof, wherein E2A(DBP) and E4(Orf6) are cloned into a single transcriptional unit by means of bicistronic sequences, separated by a skipping ribosome sequence, such as P2A (SEQ ID NO: 32) or the ECMV IRES (SEQ ID NO: 33), said minimal gene set being preferably of sequence comprising or consisting of SEQ ID NO: 55, or variants thereof.

According to particularly preferred embodiments, the at least one transcription unit c2) comprises a first transcription unit for expressing AAV Rep and Cap proteins, more preferably said first transcription unit having sequence comprising or consisting of SEQ ID NO:10, SEQ ID NO: 56, or variants thereof, and a second transcription unit for expressing the engineered human adenovirus 5 (Had5) helper proteins, more preferably comprising a minimal gene set encoding E2A DNA binding protein and E4(Orf6) binding proteins, preferably separated by one or more spacer sequences, said second transcription unit preferably having sequence comprising or consisting of SEQ ID NO: 55 or variants thereof.

Surprisingly, a small plasmid comprising said minimal gene set is capable of producing rAAV viral particles together, in the presence of AAV Rep/cap proteins.

Therefore, the present invention is also directed to a transcription unit for expressing E2A(DBP) and E4(Orf6), as described above and to plasmids bearing said transcription unit. Preferably, the invention is also directed to a polycistronic plasmid comprising the transcription unit for expressing E2A(DBP) and E4(Orf6), and the transcription unit for expressing Rep/Cap, as described above.

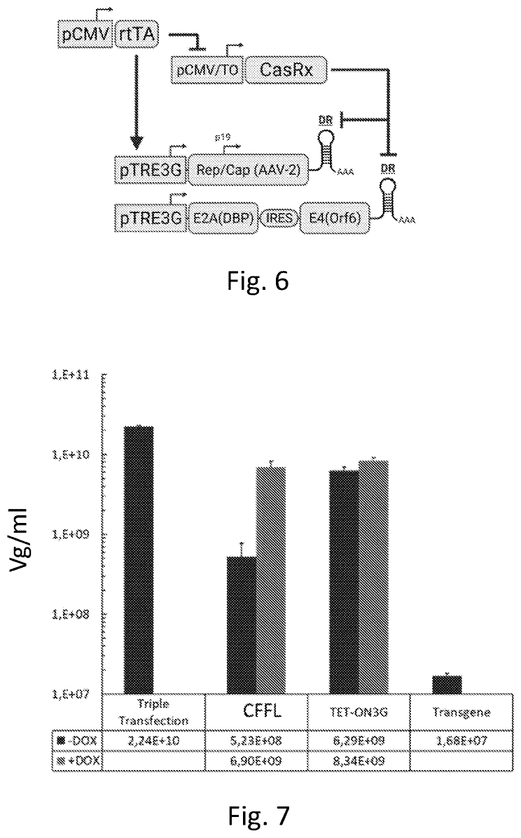

A schematic diagram of preferred embodiments of the genetic circuit comprising a2)-c2), as described above, is shown in FIG. 6.

The present invention is then directed also to a cell for stable production of viral particles, comprising the genetic circuit comprising a2)-c2), described above; preferably said cell is a human-derived cell, such as a HEK293 cell, comprising the genetic circuit. The production of viral particles can be carried out by methods known in the art.

An exemplary production method, according to preferred embodiments of the invention, is described in the Examples that follow.

It is noted that advantageously, a rAAV production platform based on cells comprising the genetic circuit of the invention overcomes the drawbacks of inducible expression systems of the prior art, wherein an rAAV producer cell line equipped with the genes required for rAAV vector production could undesirably express toxic proteins due to leakage of the inducible expression system. In cells according to the present invention, the expression of AAV genes, thus the production of rAAV particles, is instead tightly regulated, being repressed in the absence of an inducer molecule and being robustly activated in the presence of an inducer molecule. As an example, in an AAV manufacturing process, a suspension of HEK293 inducible rAAV producer cells would grow in large bioreactors until their optimal maximum expansion in the absence of the inducer molecule, thus without expression of AAV genes (not even leaky expression). Once the maximum number of producer cells is reached, production of rAAV vectors would then be switched on by the administration of the inducer molecule.

According to a second preferred aspect of the invention, the genetic circuit comprises:
a) at least one transcription unit for expressing a transcriptional activator in the cell, comprising a promoter operably linked to a polynucleotide encoding for the transcriptional activator, preferably an inducible transcriptional activator, said transcriptional activator positively regulating the expression of at least one gene of interest;
b) at least one transcription unit for expressing at least one controller biomolecule in the cell, comprising a promoter operably linked to a polynucleotide encoding for a controller biomolecule, said promoter being such that ii) the transcriptional activator positively regulates the expression of the controller biomolecule; according to said preferred aspect, the at least one component of the genetic circuit that is negatively regulated by the controller biomolecule is the transcriptional activator, preferably said transcriptional activator being sequestered by the controller biomolecule through protein-protein interaction.

Protein-protein pairs, as pairs of controller-controlled molecules, are more advantageous for genetic circuits to be expressed in eukaryotic cells, compared to DNA or RNA pairs, such as sense-antisense mRNAs, since protein pairs are not recognized as pathogenic molecules (e.g., viral RNA).

A genetic circuit according to said preferred embodiment has a negative feedback loop (NFL) configuration and provides robustness to perturbations and uncertainties, which are inherent to any biological system. This capacity to maintain the desired output stable, despite perturbations, is also called Robust Perfect Adaptation (RPA). RPA is here provided by employing proteins as controller biomolecules, that interact with other proteins or with polynucleotides, regulating the expression of a gene of interest (GOI).

Preferably, said engineered genetic circuit having an NFL configuration comprises:
a′) a first transcription unit for expressing a first transcriptional activator in the cell, comprising a promoter operably linked to a polynucleotide encoding for the first transcriptional activator, and
a″) a second transcription unit for expressing a second transcriptional activator in the cell, comprising a promoter operably linked to a polynucleotide encoding for the second transcriptional activator, said second transcriptional activator positively regulating the at least one gene of interest and wherein:
   the promoter operably linked to the polynucleotide encoding the controller biomolecule comprises a binding site for the second transcriptional activator, preferably such that ii) binding of the second transcriptional activator to said binding site positively regulates the expression of the controller biomolecule,
   the promoter operably linked to the polynucleotide encoding the second transcriptional activator comprises a binding site for the first transcriptional activator, such that binding of the first transcriptional activator to said binding site positively regulates the expression of the second transcriptional activator,
   and
   the at least one component of the genetic circuit that is negatively regulated by the controller biomolecule is the first transcriptional activator, preferably wherein said controller biomolecule is a protein capable of sequestering the first transcriptional activator.

More preferably, the transcriptional activator negatively regulated by the controller biomolecule is a modified CRISPR-associated endoribonuclease which is nuclease-deficient and which is fused to a transactivation domain and more preferably the controller biomolecule negatively regulating such transcriptional activator is an inhibitor of a CRISPR-associated endoribonuclease (Anti-CRISPR).

Said modified CRISPR-associated endoribonuclease is preferably the nuclease-deficient Cas9 (for instance dCas9, comprising or consisting of sequence SEQ ID NO: 16 or a variant thereof), fused to at least one transactivation domain, preferably a VPR transactivation domain of sequence SEQ ID NO: 15, comprising VP64, p65, and Rta linked in tandem, or variants thereof.

The VPR-dCas9 transcriptional activator sequence preferably comprises or consists of sequence SEQ ID NO: 17 or variants thereof.

The expression of the modified CRISPR-associated endoribonuclease is preferably under control of a strong constitutive promoter, such as CMV promoter.

The modified CRISPR-associated endoribonuclease, in the presence of a guide RNA (gRNA), preferably expressed from a Pol III promoter such as U6 or H1 promoter, binds a promoter comprising gRNA binding sites, activating the expression of a protein downstream said promoter.

Preferably, the genetic circuit thus further comprises a transcription unit for expressing a gRNA to direct the modified CRISPR-associated endoribonuclease to a promoter of interest comprising gRNA binding sites.

Preferably, the Anti-CRISPR is the AcrII4A protein comprising sequence SEQ ID NO: 18 or variants thereof, which has been shown to efficiently inhibit VPR-dCas9 driven transcriptional activation in mammalian cells.

In preferred embodiments, the controller and the transcription factor negatively regulated by said controller by protein-protein interaction comprise complementary coiled-coiled domains that cause dimerization of said proteins. Without being bound to theory, the presence of coiled-coiled domains, favours inhibition of VPR-dCas9 activity by AcrIIA4, according to a 1:1 stoichiometry, satisfying the property of an ideal sequestration reaction.

Preferably, said coiled-coiled domain is a N7 domain having sequence SEQ ID NO: 21 or a N8 domain having sequence SEQ ID NO: 22, or variants thereof.

Preferably, the controller is an Anti-CRISPR is AcrII4A protein fused to a N7 domain encoded by a polynucleotide having sequence SEQ ID NO: 23 or variants thereof.

Preferably, the transcription activator is a VPR-dCas9 protein fused to a N8 domain encoded by a polynucleotide having sequence SEQ ID NO: 24 or variants thereof.

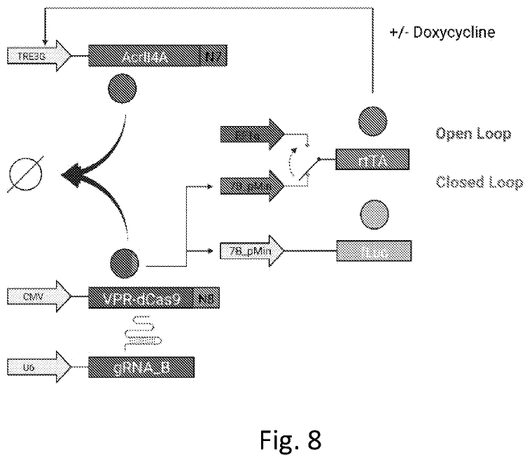

A genetic circuit according to said second preferred aspect of the invention is shown in FIG. 8; said genetic circuit comprises:
a′) a first transcription unit for constitutively expressing a first transcriptional activator in a cell, comprising a CMV promoter operably linked to a polynucleotide encoding for VPR-dCas9, and
a″) a second transcription unit for expressing a second transcriptional activator in the cell, comprising a promoter operably linked to a polynucleotide encoding for the inducible rtTA,
said first transcriptional activator positively regulating a reporter protein and wherein:
   the promoter operably linked to the polynucleotide encoding for the controller biomolecule comprises a binding site for the second transcriptional activator (pTRE3G) promoter,
   the circuit comprises a transcriptional unit for expressing a gRNA and
   the promoter operably linked to the polynucleotide encoding for the second transcriptional activator comprises RNA binding sites and, such that the VPR-dCas9-N8 protein, in the presence of the gRNA, binds said promoter driving the expression of the transcription factor rtTA; and
   the controller biomolecule is the Anti-CRISPR AcrII4A-N7 protein, which binds to, and inhibits, VPR-dCas9-N8, thus closing the loop.

Thanks to the VPR-dCas9 protein, said preferred genetic circuit of the invention can control also endogenous transcription factors, in place of the rtTA by selecting the gRNA to bind the transcription factor's endogenous promoter. Transcription factors are key regulators of biological processes inside the cells and precise control would impact biotechnologies, for example by making producer cells more resilient to apoptosis, as well as biomedicine as therapeutic target.

As a non-limiting example, transcription factor EB (TFEB) is a master regulator of autophagy and lysosomal biogenesis, and it stimulates the intracellular clearance of pathogenic factors involved in Lysosomal Storage Disorders and several neurodegenerative diseases. However, its unregulated overexpression is also causative of rare forms of renal cancer. For these reasons, TFEB gained attention as a potential therapeutic target, however its expression must be carefully regulated to avoid dangerous side effects.

As a further example, the expression level of the neuronal-differentiation transcription factor NEUROD1 (Pataskar, A. et al. EMBO J. 35, 24-45, 2016) has been shown to be important to perform neuronal differentiation (Matsuda-Ito, K., et al. Sci. Rep. 12, 1-11, 2022).

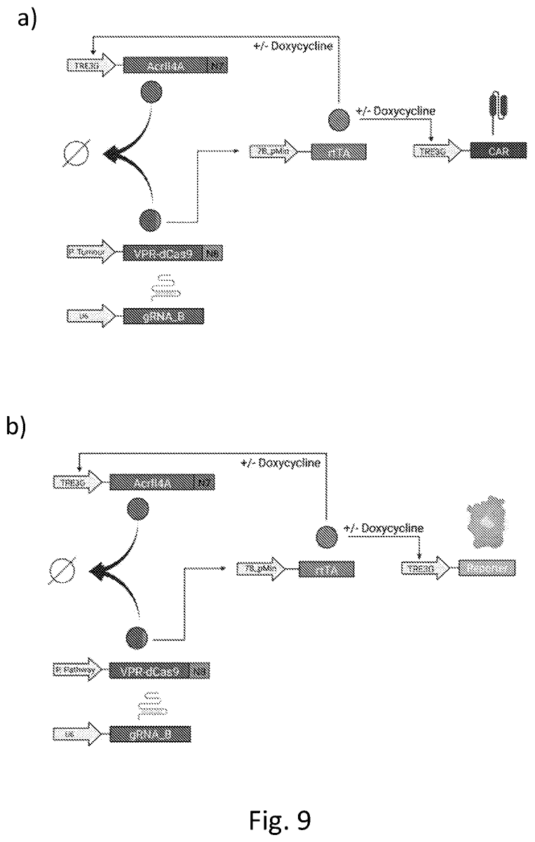

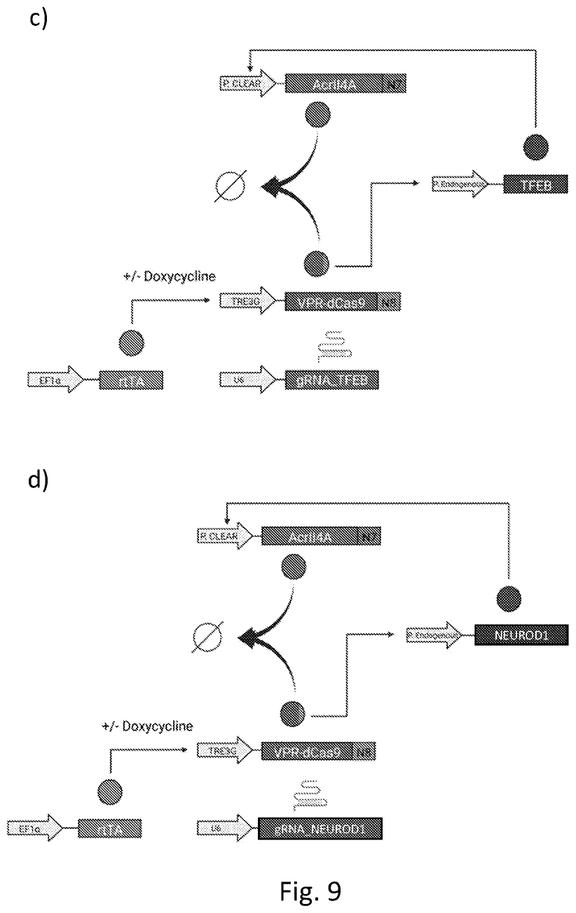

FIGS. 9 and 19 illustrate possible applications of an NFL genetic circuit system, according to preferred embodiments of the invention.

According to further preferred embodiments of the invention, at least two genetic circuits according to different preferred embodiments of the invention can also be combined to obtain biomolecular circuits with the benefits of reduced leakiness, increased tunability and robust perfect adaptation. For example, a genetic circuit with CFFL configuration as described herein can be combined with a genetic circuit having NFL configuration as described herein.

In a third preferred aspect of the invention, the genetic circuit then comprises:
a′) a first transcription unit for constitutively expressing a first transcriptional activator in a cell, comprising a promoter operably linked to a polynucleotide encoding for the first transcriptional activator, and
a″) a second transcription unit for expressing a second transcriptional activator in the cell, comprising a promoter operably linked to a polynucleotide encoding for a second transcriptional activator, preferably an inducible transcriptional activator, more preferably rtTA,
said second transcriptional activator positively regulating a reporter protein,
b′) a first transcription unit for expressing a first controller biomolecule in the cell, comprising a promoter operably linked to a polynucleotide encoding for a first controller biomolecule, said promoter being such that i) the second transcriptional activator negatively regulates the expression of the controller biomolecule;
b″) a second transcription unit for expressing a second controller biomolecule in the cell, comprising a promoter operably linked to a polynucleotide encoding a second controller biomolecule, said promoter being such that ii) the second transcriptional activator positively regulates the expression of the controller biomolecule,
wherein the second controller biomolecule negatively regulates the first transcriptional activator, preferably said first transcriptional activator being sequestered by the controller biomolecule through protein-protein interaction,
preferably wherein the first transcriptional activator is a modified CRISPR-Cas transcriptional activator, more preferably VPR-dCas9 protein or variants thereof, the second controller is an Anti-CRISPR protein, more preferably Anti-CRISPR AcrII4A protein or variants thereof, and the circuit further comprises a transcriptional unit for expressing a gRNA for guiding the modified CRISPR-Cas transcriptional activator to the promoter of the second transcriptional activator.

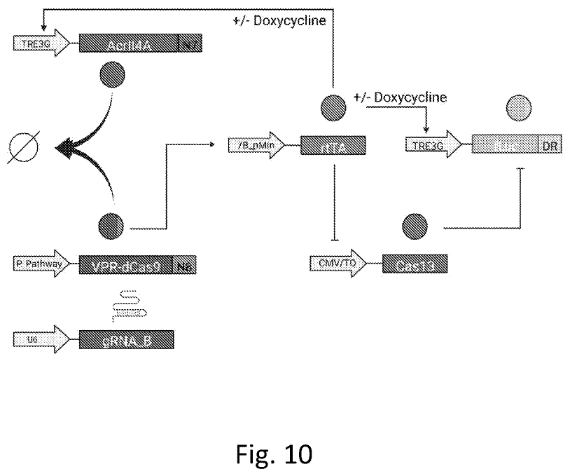

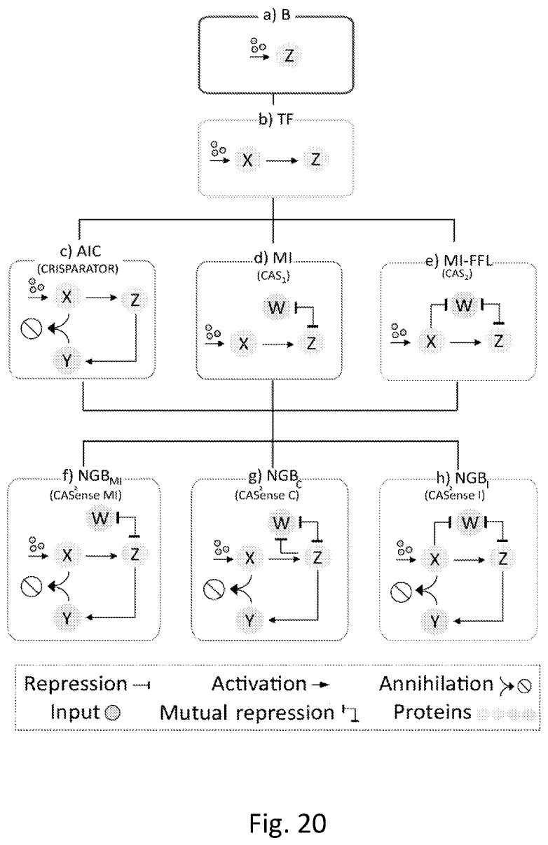

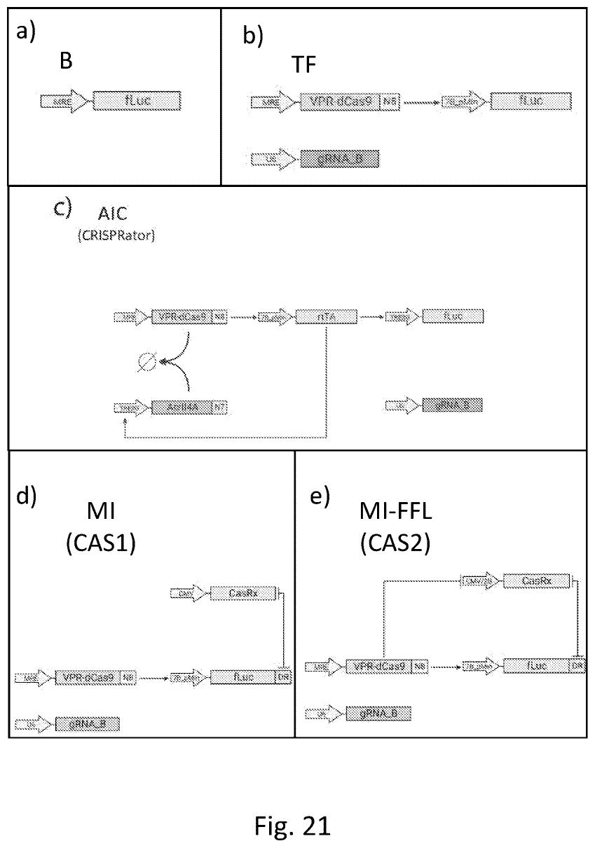

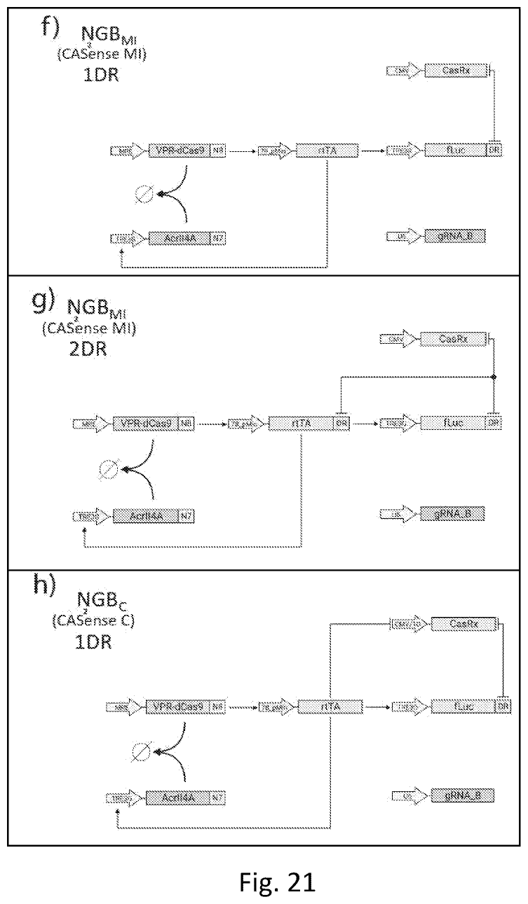

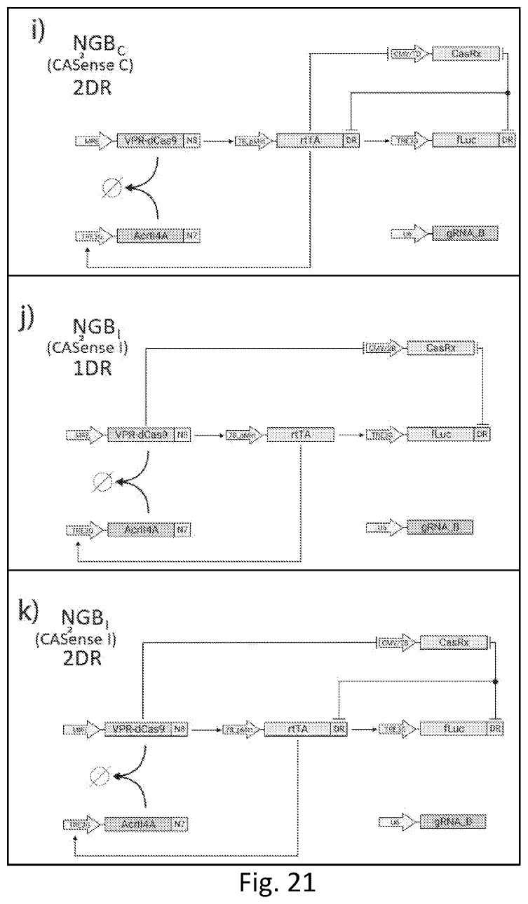

An exemplary circuit according to said third preferred aspect of the invention is shown in FIG. 10, wherein: the activator VPR-dCas9 is driven by a promoter (P. Pathway) sensitive to activation of a specific pathway (i.e., hypoxia, copper accumulatio, nutrient, ER-stress); in the absence of pathway activation the reporter protein (fLuc) expression is only weakly expressed and Cas13 can induce fLuc mRNA degradation by cleaving the DR repeat. Once the pathway is activated, rtTA expression is activated, closely tracking the pathway activity by expression of fLuc. Further preferred circuits according to said third aspect of the invention and their implementations as biosensors are shown in FIGS. 20 and 21.

Preferably the genetic circuit of the invention consists of the transcription units described herein, also in accordance to the invention and to its preferred embodiments, in the sense that it does not comprise further transcription units or components; still, such a genetic circuit can interact with other biomolecules or transcription units in a cell that comprises the genetic circuit, such as natural biomolecules or transcription units of the cell.

Also, further designs of the genetic circuit of the invention can be foreseen employing different biomolecules as components.

The present invention is also directed to a cell comprising the genetic circuit of the invention.

The cell may be a prokaryotic cell (particularly bacterial) or a eukaryotic cell (particularly fungus, plant or animal, more particularly mammalian) cell. Any suitable expression system known in the art may be used for a cell of interest. For example, the expression system may comprise one or several DNA vectors, such as plasmids, viruses or artificial chromosomes, known in the art of molecular biology.

Preferably the cell is a eukaryotic cell, particularly a mammalian cell.

The transcription units of the genetic circuit of the invention are preferably introduced in the cell by means of one or more plasmids bearing one or more of said transcription units.

For example, one or more of a first plasmid bearing a transcription unit for expressing a transcriptional activator, a second plasmid bearing a transcription unit for expressing a gene of interest and a third plasmid bearing a transcription unit for expressing a controller biomolecule, according to the invention, can be introduced in a cell, thus providing the cell with a genetic circuit according to the invention.

Therefore, the present invention is also directed to a method of controlling expression of genes in a cell, comprising: introducing in the cell one or more plasmids bearing the transcription units of the genetic circuit of the invention and expressing said transcription units in the cell.

Such a method can be carried out by any means known in the art.

The present invention is further directed to the uses of the genetic circuits or of cells of the invention, preferably in methods of production of recombinant proteins, or of viral vectors or CAR-T cells, or in methods of biosensing analytes or of drug discovery.

Examples of such uses and advantages thereof are provided hereafter in more details.

### (i) Biosensing.

Transcription-based genetic circuits according to the present invention can be employed to implement Whole Cell Biosensors (WCB) for in vitro screening of the toxicity of industrial chemicals and functional ingredients (Belkin, S. & Roda, A. Handbook of Cell Biosensors. Handbook of Cell Biosensors (2020). doi: 10.1007/978-3-319-47405-2). This is achieved by detecting the activation of specific cellular pathways in cell lines (e.g., the Keratinosense™ test for skin sensitization, measuring the activation of the Keap1-Nrf2-ARE pathway). WCBs can also be used also in preclinical drug discovery to identify small molecules that modulate a specific pathway as a therapeutic avenue, by means of High Throughput Screening (HTS) of compound libraries. The innovative genetic circuits of the invention can strongly impact these sectors by providing easy-to-implement, robust and precise in vitro biosensors thus greatly expanding their applicability.

### (ii) Bioproduction of Recombinant Proteins.

Mammalian cell lines such as Chinese Hamster Ovary Cells (CHO) and are the workhorse for recombinant protein production. Stably transfected CHO cells are routinely used for production of monoclonal antibodies as antiviral and anticancer agents, or to produce glycosylated enzymes for Enzyme Replacement Therapy (ERT) in patients with inborn error of metabolism.

Cells engineered to express the recombinant protein show high variability in production, hence the current state-of-the-art approach requires screening hundreds or thousands of cellular clones to identify the best one. The factors limiting protein yield stem both from internal and external stresses, which cause the cell to reduce or stop protein production, and eventually go into apoptosis. One of the main sources of stress is accumulation of unfolded recombinant protein activating an ER Stress Response that leads to reduction of protein's neo-synthesis until the stress is resolved, or committing the cell to apoptosis if not. There have been several attempts over the past decades to improve the performance of producer CHO cell lines in bioreactors by stably overexpressing genes for cell proliferation, stress-response or anti-apoptotic genes, but all have been met with limited success, as in many cases engineered cell lines show a decline in viability during long term cultivation. A radically innovative approach would be to dynamically adjust protein production in response to the cell status by means of genetic circuits according to the invention, e.g., by robustly controlling the expression of a transcription factor.

These “stress-aware” cells should be much more resilient to stress and thus simplify and speed up selection of high producer clones.

(iii) Bioproduction of Adeno Associated Viruses (AAV).

As previously discussed, efforts to generate stable clonal cells for inducible expression of Helper and AAV genes have failed so far because of viral genes' toxicity caused by the inherent “leakiness” of inducible promoters even in the absence of the inducer molecule. The circuits of the invention can be employed to sense the presence of an inducer molecule in the growth medium, to induce expression of Helper and AAV toxic genes, while at the same time greatly reducing their basal expression in the absence of the inducer molecule, thus enabling the generation of a stable producer cell line.

As an example, a cell enabled to express the genetic circuit of the invention can be produced by means of selectable markers, such as fluorescent proteins or eukaryotic antibiotic resistance genes. Briefly, the cell clones would be transfected with a plasmid bearing a pTRE3G-reporter (e.g., Gaussia Luciferase) transcription unit and the clone with the lower leakiness value and the highest response, resulting in the best fold change activation level would be selected. After the generation of the enabled cell line, pTRE3G-Rep/Cap (AAV-2) and pTRE3G-E2A(DBP)-IRES-E4(Orf6) plasmids bearing two different eukaryotic antibiotic resistance genes or split-resistance genes (Jillette, Nathaniel, et al. “Split selectable markers.” Nature communications 10.1 (2019): 1-8.) could be stably integrated, in order to engineer an inducible AAV packaging cell line, preferably a HEK-293 cell line (Pack-HEK293). Pack-HEK293 clones would then be screened by means of transient triple transfection, transfecting those cell clone with a pTransgene only (i.e., a plasmid bearing a transcription unit flanked by two ITR for expressing the biomolecule to be encapsidated in the recombinant AAV), in the absence and in the presence of Doxycycline. The clone with the lower production in the absence of Doxycycline and the highest production in its presence would be selected, generating an HEK293 inducible AAV packaging master cell line. This cell line could be integrated with any pTransgene encoding any therapeutic gene giving rise to a specific HEK293 inducible AAV producer cell line. As proof of principle, in the HEK293 AAV packaging master cell line a pTransgene encoding for a GFP fluorescent protein driven by the strong constitutive promoter pCMV could be integrated. Then, HEK293 AAV producer cell clone would be selected as was the HEK 293 packaging cell line, checking for the cell clone with the lowest production in the absence of doxycycline and the highest production in its presence.

### (iv) Engineering of CAR-T Cell

Chimeric Antigen Receptor (CAR)-T cells represent a breakthrough technology in cancer therapy, but they are current object of research to fight also autoimmune and metabolic diseases. They consist in lymphocytes that are engineered to be redirected against specific antigen-expressing cells, in order to recognize and eliminate them, with anti-CD19 CAR-T cell therapy against B cell malignancies approved by the FDA in 2017. Despite proven efficacy and potential, one of the main drawbacks of CAR-T cells is CAR overexpression, that leads to life-threatening toxicities and Cytokine Release Syndrome (CRS). Conditional and tunable CAR-T are indeed current object of research, trying to resolve these problems, but since traditional gene expression systems are not precise neither stable, controlling CAR expression is not an easy task. With circuits of the invention having a NFL configuration, it would be possible to express the Chimeric Antigen Receptor, both in a conditional and tunable way.

FIGS. 6 and 9 show schemes of genetic circuits for the applications above.

For example, FIG. 9a shows a genetic circuit according to preferred embodiment of the invention for CAR-T therapy, comprising the activator VPR-dCas9-N8 driven by a tumor-specific promoter, in order to be active only in the tumor microenvironment and to avoid off-target; the gRNA_B, under the control of U6 promoter; the expression of the transcription factor rtTA driven by the 7B_pMin promoter upon binding of the VPR-dCas9-N8 activator driven by the gRNA; when administering doxycycline, rtTA binds to the TRE3G promoters, allowing the expression of both the CAR and the AcrII4A-N7 protein, thus closing the loop.

FIG. 9b shows the use of a genetic circuit according to preferred embodiment of the invention as a biosensor to detect skin reaction to chemical substances, wherein the expression of the activator is driven by a promoter responding to the inflammatory Keap1-Nrf2-ARE pathway; when bound to the gRNA_B, the activator drives the expression of the transcription factor rtTA, which, in turns, activates the AcrII4A-N7 protein, that closes the loop. The reporter proteins are both under the control of the TRE3G promoter; when the doxycycline dose is fixed, the expression of the reporter only depends on the amount of the activator, hence correlating with promoter's strength and pathway activation.

FIGS. 9c and 9d show the use of a genetic circuit according to preferred embodiment of the invention to control the endogenous transcription factor TFEB (c) or NEUROD1 (d): the strong constitutive promoter EF1a drives the expression of the transcription factor rtTA, which binds to the TRE3G promoter in the presence of Doxycycline, allowing the tunable expression of the activator VPR-dCas9-N8; the U6 promoter controls the expression of a gRNA complementary to the endogenous promoter of the transcription factor, allowing to overexpress the latter when bound to the VPR-dCas9-N8; the expression of the AcrII4A protein that closes the loop is under the control of a synthetic promoter bearing the CLEAR sequences (NiClear promoter) recognized by TFEB or NEUROD1. The same elements can be used to control expression of other endogenous transcription factors.

FIG. 21c-k shows further genetic circuits for application as Whole Cell Biosensors (WCB), according to preferred embodiment of the invention.

In particular, in a first preferred embodiment, the biosensing genetic circuit (biosensor “AIC”) is an NFL circuit comprising: a′) a first transcription unit, comprising a metal responsive element (MRE) promoter, preferably having sequence SEQ ID NO: 45, operably linked to a polynucleotide encoding a first transcriptional activator, more preferably VPR-dCas9 or VPR-dCas9-N8, most preferably having sequence SEQ ID NO: 17 or SEQ ID NO: 24, which activator, in the presence of a constitutively expressed gRNA_B guide under the U6 promoter, binds the 7B_pMin promoter, preferably having sequence SEQ ID NO: 46, driving the expression of a″) a second transcription unit for expressing a second transcriptional activator, more preferably an inducible activator, most preferably rtTA, that in turns activate expression of a reporter gene, such as a fLuciferase (fLuc) reporter, preferably having sequence SEQ ID NO: 49, more preferably under the control of a TRE3G promoter of sequence SEQ ID NO: 7; the biosensing circuit further comprising: b) a transcription unit for expressing a controller biomolecule, preferably AcrII4A, or AcrII4A-N7 protein, more preferably having sequence SEQ ID NO: 18 or 23, most preferably driven by TRE3G promoter of sequence SEQ ID NO: 7, said controller biomolecule negatively regulating the first transcriptional activator. (see FIGS. 20c and 21c)

In a second preferred embodiment, the biosensing genetic circuit (biosensor “MI-FFL”) a) a transcription unit, comprising a MRE promoter, preferably having sequence SEQ ID NO: 45, operably linked to a polynucleotide encoding a transcriptional activator, more preferably VPR-dCas9 or VPR-dCas9-N8, most preferably having sequence SEQ ID NO: 17 or SEQ ID NO: 24, which transcriptional activator induces expression of a reporter gene comprising DR, preferably of fLuc-DR having sequence SEQ ID NO:49, that mutually inhibits with a CasRx controller biomolecule, preferably having sequence SEQ ID NO: 2, more preferably said controller biomolecule being under the control of a promoter which is negatively regulated by the transcriptional activator, more preferably said promoter being CMV/2B having sequence SEQ ID NO: 48 (see FIGS. 20e and 21e).

In a third preferred embodiment, the biosensing genetic circuit (Biosensor “NGB_{C}”, with 1 DR), is a combination of biosensor AIC and MI-FFL described above, more preferably as depicted in FIGS. 20g and 21g.

In a fourth preferred embodiment, the biosensing genetic circuit (Biosensor “NGB_{C}”, with 2 DR) is a circuit that differs from Biosensor NGB_{C} with 1 DR above in that also the second transcriptional activator comprises Direct Repeat (DR) recognized by the controller CasRx, preferably said second transcriptional activator having sequence SEQ ID NO: 50 (see FIGS. 20g and 21i). FIG. 10 shows an example of this NGB_{C} biosensing circuit.

In a fifth preferred embodiment, the biosensing genetic circuit (Biosensor “NGB_{I}”, with 1 DR), is a combination of biosensor AIC and MI-FFL, as depicted in FIGS. 20h and 21j.

In a sixth preferred embodiment, the biosensing genetic circuit (Biosensor “NGB_{I}”, with 2 DR), is a circuit that differs from Biosensor “NGB_{I}”, with 1 DR above, in that also the second transcriptional activator comprises Direct Repeat (DR) recognized by the controller CasRx, preferably said second transcriptional activator having sequence SEQ ID NO: 50 (See FIGS. 20h and 21k).

According to further aspects, the invention is directed to a biosensing genetic circuit (biosensor “MI”) comprising a) a transcription unit comprising a MRE promoter, preferably having sequence SEQ ID NO: 45, operably linked to a polynucleotide encoding a first transcriptional activator, more preferably VPR-dCas9 or VPR-dCas9-N8, most preferably having sequence SEQ ID NO: 17 or SEQ ID NO: 24, which activator which activator, in the presence of a constitutively expressed gRNA_B guide under the U6 promoter, binds the 7B_pMin promoter, preferably having sequence SEQ ID NO: 46, driving the expression of a reporter, preferably of fLuc-DR having sequence SEQ ID NO: 49, whose expression is inhibited by a constitutively expressed controller biomolecule, preferably CasRx (see FIGS. 20d and 21d).

According to further aspects, the invention is directed to a biosensing genetic circuit (Biosensor “NGB_{MI}”, with 1 DR), is a combination of biosensor AIC and MI above, preferably as depicted in FIGS. 20f and 21f).

According to further aspects, the invention is directed to a biosensing genetic circuit (Biosensor “NGB_{MI}”, with 2 DR, which differs from Biosensor “NGB_{MI}”, with 1 DR in that also the second transcriptional activator comprises Direct Repeat (DR) recognized by the controller CasRx, preferably said second transcriptional activator having sequence SEQ ID NO: 50 (see FIGS. 20g and 21g). The above described circuits represent preferred embodiment for the indicated uses.

Further preferred uses of the genetic circuits of the present invention include their use for controlling expression of genes for gene therapy in a cell, such a gene therapy being preferably delivered to a cell by viral vectors, most preferably by AAV vectors, or by lentiviral vectors, being delivered by non-viral vectors, more preferably by lipid- or non-lipid-nanoparticles.

Further preferred uses of the genetic circuits of the present invention include their use for controlling expression of genes in a method of gene editing.

Further preferred uses of the genetic circuits of the present invention include their use for controlling expression of genes for cancer therapy.

It should be understood that the invention is directed to any of the genetic circuits depicted in the figures accompanying the present description, as preferred embodiments of the invention.

In particular, according to preferred embodiments, the CFFL genetic circuit of the invention comprises:

It should be understood that all the possible combinations of the preferred aspects of the present invention are also described, and therefore similarly preferred.

Examples of preferred embodiments of the present invention and analyses of their efficacy are provided below for illustrative and non-limiting purposes.

### EXAMPLES

### Materials and Methods

Plasmids construction. Plasmids used in the following examples 1-10 were constructed using the Golden-Gate based EMMA assembly kit, following authors' protocol (Jones, S., Martella, A. & Cai, Y. EMMA assembly explained: A step-by-step guide to assemble synthetic mammalian vectors. Methods in Enzymology vol. 617 (Elsevier Inc., 2019), or using the Gibson assembly method (Gibson, D. G. et al. Enzymatic assembly of DNA molecules up to several hundred kilobases. Nat. Methods 6, 343-345 (2009)). The sequences of N7 and N8 coiled coils (Plaper, T. et al. Coiled-coil heterodimers with increased stability for cellular regulation and sensing SARS-COV-2 spike protein-mediated cell fusion. Sci. Rep. 11, 1-16 (2021)), AcrII4A and FKBP-derived DD (Nakamura, M. et al. Anti-CRISPR-mediated control of gene editing and synthetic circuits in eukaryotic cells. Nat. Commun. 10, 1-11 (2019)), gRNA inducible constructs with 7 binding sites for gRNA B (Praznik, A. et al. Regulation of protein secretion through chemical regulation of endoplasmic reticulum retention signal cleavage. Nat. Commun. 13, 1-14 (2022)) and 1 or 10 binding sites for gRNA AB (Lebar, T., Lainšček, D., Merljak, E., Aupič, J. & Jerala, R. A tunable orthogonal coiled-coil interaction toolbox for engineering mammalian cells. Nat. Chem. Biol. 16, 513-519 (2020)), and dCas9-VPR (Xiong, K. et al. Reduced apoptosis in Chinese hamster ovary cells via optimized CRISPR interference. Biotechnol. Bioeng. 116, 1813-1819 (2019)) have been taken from the referenced papers. All the sequences regarding CasRx and the direct repeat, used to implement the CFFL in mammalian cells have all been taken from Konermann, S. et al. (Transcriptome Engineering with RNA-Targeting Type VI-D CRISPR Effectors. Cell 173, 665-676.e14 (2018)). Gaussia Luciferase and RedFire Fly sequences were taken from pTK-Gaussia Luc (Thermofisher #16148) and PCMV-Red Firefly Luc (Thermofisher #16156), respectively. The Tet-On® 3G system, comprising the TRE3G promoter and the rtTA protein, has been acquired from Takara Bio.

Most plasmids used in Example 12 were constructed using the Golden-Gate based EMMA assembly kit (Martella, A., et al. ACS Synth. Biol. 6, 1380-1392 (2017), NEBridge Golden Gate Assembly BsaI-HFv2 and BsmBI-v2 kits (NEB) employing custom fusion sites, while in other cases the plasmids have been constructed using the Gibson assembly method (Gibson, D. G. et al. Nat. Methods 6, 343-345, 2009). All the sequences of circuit elements are as indicated; the Tet-On® 3G system, comprising the TRE3G promoter and the rtTA protein, has been acquired from Takara Bio.

Cell culture and transfection. The HEK293T cell line (ATCC) was cultured in DMEM Glutamax (Gibco) supplemented with 10% Tet-Free Fetal Bovine Serum (Euroclone) and 1% Penicillin-Streptomycin (Euroclone), while the HeLa cell line (ATCC) was cultured in DMEM (Gibco) supplemented with 10% Tet-Free Fetal Bovine Serum (Euroclone), 1% L-Glutamine (Euroclone) and 1% Penicillin-Streptomycin (Euroclone). Both the cell lines have been kept at 37° C. in a 5% CO2 environment. For luciferase experiment, 1.5×10\hat{ }4 (HeLa) and 2×10\hat{ }4 (HEK293T) cells per well were seeded in CoStar White 96-well plates (Corning), 4.5×10\hat{ }4 (HEK293T) were seeded when performing reverse transfection, while for Flow Cytometry assay the same numbers of cells have been seeded in 96-well cell culture plates (Corning). After 18 hours of seedling, or immediately after seedling, for reverse transfection, cells have been transfected using a home-made solution of PEI (MW 25000, Polysciences, stock concentration 0,324 mg/ml, pH 7.5) using 250 ng of DNA per well. For HEK293T cells, 5 μl of PEI solution for each μg of DNA were used, while for HeLa 4 μl of PEI solution for each μg of DNA were used. To normalize reporter values to transfection efficiency, 10 ng of phRL-TK (encoding for Renilla Luciferase) have been used for luciferase experiment.

Luciferase assay. The cells were collected 48 hours after transfection and lysed with 25 μL of 5×Passive Lysis Buffer (Biotin) diluted in water. Firefly Luciferase and Renilla Luciferase expression were measured using the Dual Luciferase Assay (Promega) on a Glomax Explorer plate reader (Promega). Firefly Luciferase Arbitrary Units (fLuc [A.U.]) were calculated by normalizing each sample's Firefly Luciferase activity to the constitutive Renilla activity detected in the same sample. Relative fLuc [A.U.] values have been calculated by dividing each sample's Firefly Luciferase Arbitrary Units by the Firefly Luciferase Arbitrary Units of the sample with no Doxycycline induction, in the case of Doxycycline dose-response curves. In the case of Shield1 treatment, Relative fLuc [A.U.] values have been obtained by dividing each sample's Firefly Luciferase Arbitrary Units by the Firefly Luciferase Arbitrary Units of the sample treated with the maximum dose of Shield1 used, 1000 nM. Regarding experiments comprising Gaussia Luciferase and RedFireFly Luciferase, cells expressing those luciferases were harvested 48 h after transfection. Cells were disrupted with 100 μL/well of 1×Cell Lysis buffer and shaken for 15 min. Luciferase activity was measured using Pierce™ Gaussia-Firefly Luciferase Dual Assay Kit (ThermoFisher Scientific) according to the manufacturer's instructions.

### Small-Scale MW6 AAV Vector Production

800,000 HEK293T cells were seeded in DMEM GlutaMax+10% FBS (Euroclone) per well of a 6-well plate and immediately reverse transfected with 2 μg of DNA and 10 μL of PEI solution (MW 25000, Polysciences, stock concentration 0,324 mg/ml, pH 7.5) each well. For the Triple Transient Transfection cells were transfected with an equimolar amount of pAAV2.1-CMV-EGFP plasmid (pTransgene, see Auricchio, A., Hildinger, M., O'Connor, E., Gao, G. P. & Wilson, J. M. Isolation of highly infectious and pure adeno-associated virus type 2 vectors with a single-step gravity-flow column. Hum. Gene Ther. 12, 71-76 (2001)), the pAd helper plasmid that contains the adenovirus E2A, E4, and VA RNA helper genes (pHelper, see Zhang, Y., Chirmule, N., Gao, G. & Wilson, J. CD40 Ligand-Dependent Activation of Cytotoxic T Lymphocytes by Adeno-Associated Virus Vectors In Vivo: Role of Immature Dendritic Cells. J. Virol. 74, 8003-8010 (2000)), and the pAAV2/2 packaging plasmid (Gao, G. P. et al. Novel adeno-associated viruses from rhesus monkeys as vectors for human gene therapy. Proc. Natl. Acad. Sci. U.S.A 99, 11854-11859 (2002) with AAV rep and cap genes (pPackaging). For production of AAV-producing cells of Example 4 and the Tet-ON3G systems cells were transfected with pAAV2.1, pTRE3G-E2A(DBP)-IRES-E4(Orf6)-DR (Seq ID NO: 55), pTRE3G-Rep2/Cap2-DR (Seq ID No: 10), pCMV-rtTA-VPR (Seq ID NO: 6); and only regarding the genetic circuit, cells were transfected also with pCMV/TO-CasRx-NLS-T2A-mCherry (Seq ID NO: 20). 72 hr after transfection cells were recovered, pelleted at 1000×g for 5′ and resuspended into 500 μL of PBS-MK (1×PBS, 1 mM MgCl2, and 2.5 mM KCl), then lysed by 4 cycles of freeze and thaw, and finally the crude lysate was clarified through centrifugation at 18.000×g for 10 minutes.

qRT-PCR AAV Vector Titration in Crude Lysate

Crude lysates containing rAAV particles were used to incubate with 50 units (U) of DNase I (Roche) for 15 hr at 37° C., and then inactivated at 75° C. for 30 min. Samples were then treated with 10 μL of proteinase K (>600 mAU/mL) at 56° C. for 2 hr, followed by incubation at 95° C. for 30 min to inactivate the enzyme. The digested samples were diluted 10-fold in nuclease-free water. Linearized pAAV2.1 transgene plasmid (XhoI-NheI) were serially diluted in either nuclease-free water or negative control crude lysate from 2.5×108 to 25 copies/μL in 10-fold serial dilution to generate standard curves for qPCR quantification. All qPCR reactions were composed of 5 μl of diluted sample or plasmid standard, 0.5 μM forward primer, 0.5 μM reverse primer, and 12.5 μL of LightCycler® 480 SYBR Green I Master (Roche) in a total reaction volume of 25 μL each. Analyses were performed using the LightCycler® 96 Instrument (Roche), and cycling conditions were as follows: 95° C. for 10 min, followed by 40 cycles of 95° C. for 15 s and 60° C. for 1 min. The number of genome copies per cell was calculated using the formula: Vg/mL=[(A/B)×10×1000], where Vg/mL is the Viral genomes copies per mL, A is resulting amount in the PCR reaction, B is the volume of the rAAV samples in PCR reaction, 10 and 1000 are dilution factors.

Flow Cytometry. Cells were collected 48 hours after transfection, washed and resuspended with PBS (Euroclone). Flow Cytometry analysis were carried out using an Accuri C6 (BD Biosciences), analyzing 10000 cells for each sample. A 488-nm laser with a 670 nm LP filter were used to excite and detect mCherry fluorescence.

Drug treatments. When treated with Doxycycline (Clontech) and/or Shield1 (MedChemExpress) and/or Copper dichloride (Sigma-Aldrich), cells were treated immediately before transfection. Drugs' concentrations are referred to the medium volume before adding the transfection mix. Doxycycline and Copper dichloride were dissolved in H2O while Shield1 was dissolved in DMSO.

### Real Time-PCR for NEUROD1 Expression

1.25×10⁵ HEK293T cells have been seeded in a 24-well cell culture plates (Corning) and, after 18 hours, transfected using a home-made solution of PEI (MW 25000, Polysciences, stock concentration 0,324 mg/ml, pH 7.5) using 700 ng of DNA per well. After 48 hours, mRNA has been extracted with RNeasy Mini Kit (Quiagen) and retrotranscribed to cDNA with QuantiTec Reverse Transcription Kit (Quiagen). Real-Time PCR reaction has been performed with 25 ng of template DNA, using the LightCycler® 480 SYBR Green I Master (Roche) and quantified through LightCycler® 96 System (Roche). Primers to detect NEUROD1 RNA were those published in Chavez, A. et al. Nat. Methods 13, 563-567 (2016); primers to detect AcrII4A have been designed and are as follows: AcrII4A_Fw: CAACGATCTGATCCGGGAGA AcrII4A_Rv: TACTCGTTGCCGTCGTTGTT.

### Cell Viability Assays for Cytotoxicity Assay

Doxycycline (0, 1000 ng/mL) and Ganciclovir sodium (GCV; 100 μg/mL) (MedChem Express; #HY-13637A) were added to each well of μCLEAR® Black 96 well plate (Greiner) just before cell seeding and transfection with the DNA/PEI mixture of plasmids of interest. 72 hr after transection, cell viability we assessed via CellTiter Glo kit (Promega). 100 μL of Cell Titer-Glo Reagent, reconstituted following manufacturer's instructions, were added in each well containing already 100 μL of growth medium. The entire 96-well plate was mixed for 2 minutes on luminometer built-in orbital shaker (200 rpm) and the samples incubated for 10 minutes at room temperature; then, luminescence on a Glomax Discovery plate reader (Promega) was measured. For crystal violet (CV) staining, six-well plates were coated with poly-D-lysine (Gibco) following manufacturer's recommendations before seeding 4×105 HEK293T cells each well. 24 hr after cell seeding, doxycycline (0, 1000 ng/ml) and Ganciclovir sodium (GCV; 100 μg/mL) (MedChem Express; #HY-13637A) were added and cells immediately transfected with the DNA/PEI mixture of plasmids of interest. 72 hr after transfection, cells were washed three times with PBS and incubated with 1 ml of pure EtOH each well at −80C° for 24 hr to fix them. After fixation, 1 ml of CV solution (500 mg Cristal Violet, Sigma; 25% v/v Et-OH/H2O) was added to each well and incubated for 15 min; then cells were washed three times with PBS.

### Crude Lysate Transduction Assays

20 μL of crude lysate of cells transfected to produced AAV vectors were used to transduce 2.5×10⁴ HEK293T cells in each well of 96 well plate (Corning). percentage of GFP positive cells was analyzed by flow cytometry. 48 hr after transduction, cells were harvested using trypsin-EDTA and washed three times with phosphate saline buffer (PBS). cells were kept in ice until analysis with Accuri C6 flow cytometer (BD). cell autofluorescence threshold was determined using samples transduced with crude lysate from cells transfected solely with pTransgene (p8-pAAV2.1 CMV eGFP3). 1×10⁴ cells for each sample were acquired. the data using the Accuri were analyzed C6 software (BD).

### Example 1

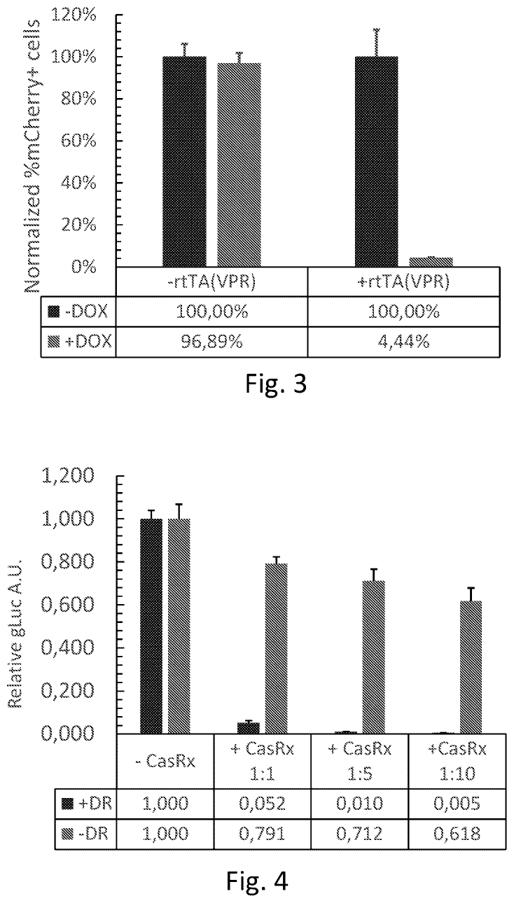

HEK293T cells were transfected with a plasmid encoding the pCMV/TO promoter driving the expression of the fluorescent protein mCherry, and with a plasmid encoding the rtTA-VPR transcription factor driven by the strong constitutive pCMV promoter. A condition treated in the same way but without rtTA-VPR transfection was used as a negative control to account for any doxycycline treatment bias on mCherry expression. The percentage of mCherry positive cells was quantified through flow cytometry analysis in the absence and presence (1000 ng/ml) of doxycycline. The results of this experiment are shown in FIG. 3, where it can be appreciated that pCMV/TO efficiently represses the expression of the mCherry almost 25 times at the transcriptional level in the presence of doxycycline. pCMV/TO is efficiently repressed by the rtTA-VPR in the presence of doxycycline. Percentual mean values of at least three biological replicates are shown. Error bars indicate standard error.

### Example 2

The findings of Example 1 were further confirmed by transfecting HEK293T cells with an increasing molar ratio of a plasmid encoding a Gaussia luciferase reporter gene, with or without Direct Repeats in its 3′UTR and a plasmid encoding the CasRx endoribonuclease. All cells were transfected also with a Red Firefly luciferase reporter gene to normalize towards transfection efficiency bias between each assessed experimental condition. gLuc arbitrary units (A.U). were calculated as the ratio between the Gaussia and Red Firefly luminescence. At least, three biological replicates were analyzed for each condition. Relative gLuc A.U. is calculated as the ratio between the gLuc A.U. values obtained transfecting the CasRx against the one which wasn't; this last corresponds to 1 gLuc A.U.

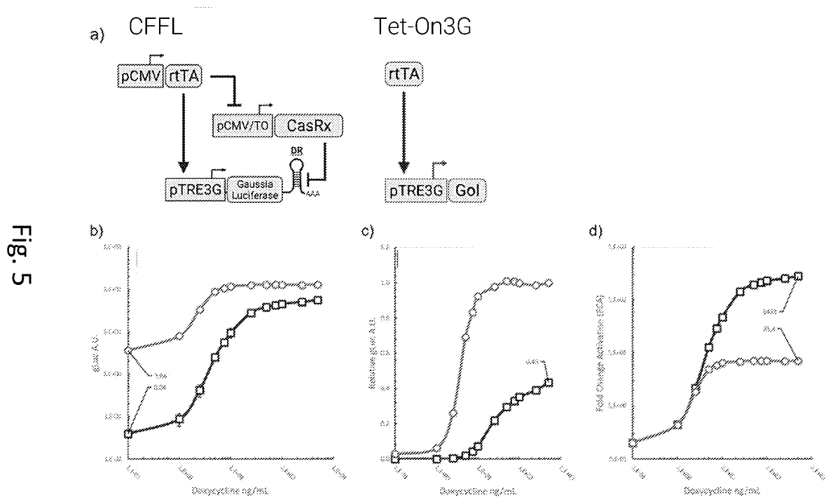

As shown in FIG. 4, only the expression of Gaussia luciferase harboring the DR from a transcript harboring a Direct Repeat in its 3′UTR plunges to 5%, in the presence of an equimolar amount of plasmid encoding the CasRx protein. The CasRx-mediate target gene expression inhibition can be appreciated very well in the condition in which a 10 times molar excess of CasRx was transfected, where the endoribonuclease can post-transcriptionally inhibit the luciferase expression by nearly 200 folds.

### Example 3

A genetic circuit having CFFL configuration according to preferred embodiments of the invention has been compared with the Tet-On3G system of the prior art (FIG. 5a), using the Gaussia Luciferase reporter as output. The three plasmids comprising the three transcription units of the genetic circuit were transfected in HEK293T cells, and the response was evaluated at increasing concentrations of the inducer molecule doxycycline.

FIG. 5b-d shows the genetic circuit according to preferred embodiments of the invention and the TET-ON3G circuits' performances in terms of normalized Gaussia Luciferase luminescence.

Dose-response curves were obtained by increasing doxycycline concentration up to 1000 ng/mL. All cells were also transfected with a Red Firefly Luciferase reporter gene to normalize towards transfection efficiency bias between each assessed experimental condition. gLuc arbitrary units (A.U). were calculated as the ratio between the Gaussia and Red Firefly luminescence. In the absence of the inducer molecule doxycycline, the genetic circuit of the invention shows a much lower basal expression value than the one obtained by the state-of-the-art TET-ON3G system, achieving an overall reduction of nearly 100-fold (FIG. 5b). Nevertheless, at a high level of doxycycline, the genetic circuit of the invention is characterized by an average maximum output value that is about half of the original TET-ON3G (FIG. 5c). However, already at a doxycycline concentration of 500 ng/ml, the genetic circuit of the invention achieves an average Fold-Change value of more than 1400 times compared to the 35 times obtained by the TET-ON3G system (FIG. 5d). The genetic circuit of the invention greatly outperforms the state-of-the-art TetON3G system in terms of leakiness and dynamic range.

### Example 4

AAV vector production in cells comprising a genetic circuit according to preferred embodiments of the invention having a CFFL configuration, or comprising the TET-ON3G systems of the prior art, is tested. In “triple transfection” samples, HEK293T cells were transfected with the standard three plasmids required for AAV vector production: pHelper, pPackaging, and pTransgene. in “Transgene” samples, HEK293T cells were transfected with only pTransgene to account for qPCR background bias in the crude lysate. The genetic circuit according to preferred embodiments of the invention modulates the expression of Human Adenovirus 5 (HAd5) Helper and AAV2 Rep/Cap proteins. The transcription unit for expressing Adenoviral helper genes comprises the ECMV IRES sequence, the E2A DNA Binding Protein (E2A-DBP) and E4(Orf6) coding sequences downstream of the pTRE3G promoter, in order to express both required helper proteins from one single transcript from the pTRE3G. In the transcription unit for expressing Rep/Cap proteins, the wild-type AAV p5 promoter is substituted with the pTRE3G promoter. Both expression cassettes just described harbor in their 3′UTR a single Direct Repeat. (see FIG. 6). The genetic circuit of the invention, through the pTRE3G promoter, drives E2A(DBP)-IRES-E4(Orf6) and Rep/Cap genes expression after doxycycline administration, instead, the Direct Repeats enables quenching of basal expression of these toxic genes by means of CasRx in the absence of doxycycline.

AAV production was quantified by means of qPCR titration of the DNase-resistant particle (DRP) from the crude lysate.

AAV production yields obtained with the genetic circuit according to preferred embodiments of the invention are compared to the ones obtained by using the state-of-the-art TET-ON3G system that does not include the transcription unit expressing CasRx under the pCMV/TO promoter. The production obtained by the two system were also compared against the current standard method of production, i.e., transient triple transfection, and as negative control to transfecting HEK293T cells with only the transgene plasmid to account for qPCR background bias in the crude lysate.

In the presence of doxycycline, the genetic circuit according to preferred embodiments of the invention induces the production of AAV vectors reaching almost the same level obtained by the Tet-ON3G. Instead, in the absence of doxycycline, the difference between the two systems can be appreciated. Whereas the Tet-ON3G achieves almost the same magnitude of production yields, because of leaky basal expression of the viral genes, the genetic circuit according to preferred embodiments of the invention shows a log decrease in manufacturing yields, proving its ability to quench the viral toxic gene leakiness (FIG. 7)

The results demonstrate that the genetic circuit of the invention can finely regulate and tune genetic expression in cells.

### Example 5

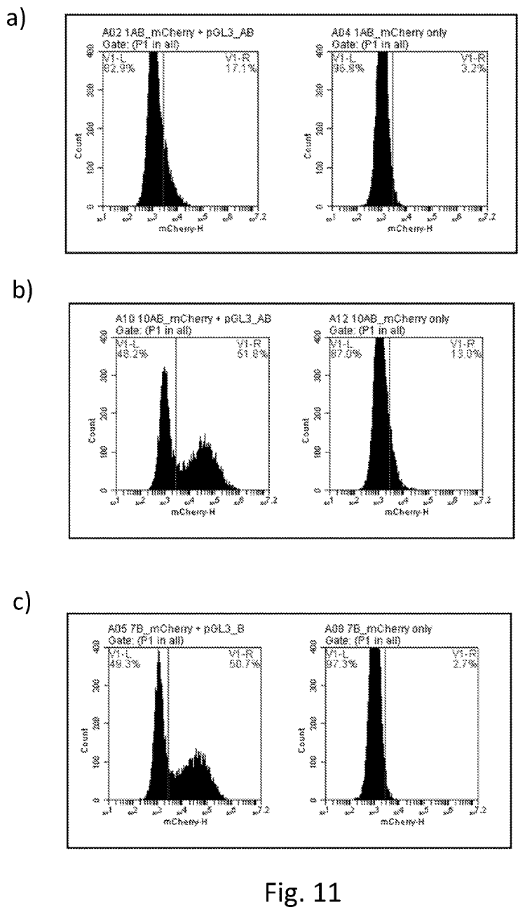

The fluorescent mCherry protein was cloned downstream of three synthetic gRNA inducible promoters (Lebar, T., Lainšček, D., Merljak, E., Aupič, J. & Jerala, R. A tunable orthogonal coiled-coil interaction toolbox for engineering mammalian cells. Nat. Chem. Biol. 16, 513-519 (2020); Praznik, A. et al. Regulation of protein secretion through chemical regulation of endoplasmic reticulum retention signal cleavage. Nat. Commun. 13, 1-14 (2022)), where a different number of gRNA binding sites are cloned upstream of a minimal CMV promoter. a single guide RNA (sgRNA) was then expressed from the RNA polymerase III promoter U6, together with the VPR_dCAs9 and the mCherry fluorescence was measured. The results are as shown in FIG. 11a-c. The promoter with 7 binding sites for the gRNA (7B_pMin), showed an optimal ratio between VPR-dCas9 driven induction and leakiness in absence of the protein and the gRNA.

### Example 6

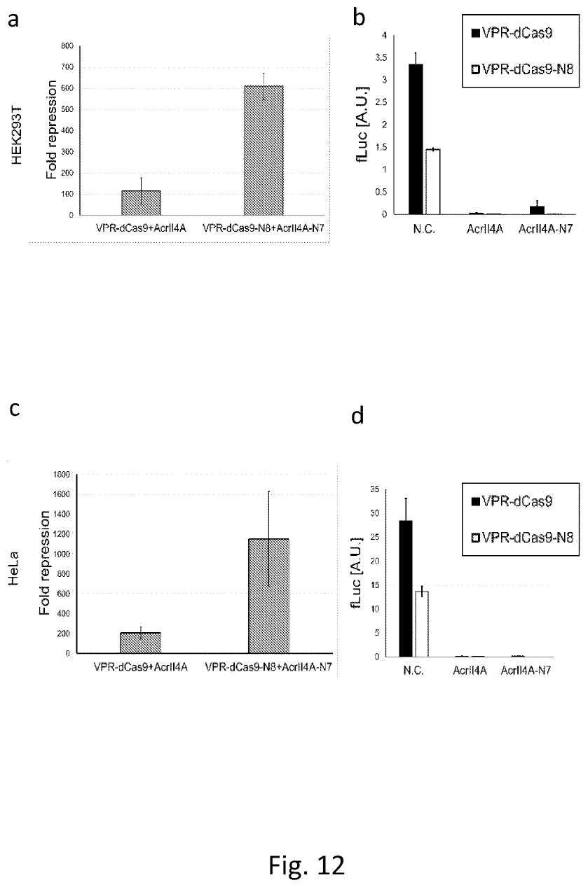

In order to confirm that AcrII4A controller protein is able to prevent VPR-dCas9 driven gene activation in human cell lines such as HeLa and HEK293T, a luciferase assay was carried out where the luciferase gene (fLuc) is expressed from the 7B_pMin synthetic promoter of example 5, whereas both the VPR-dCas9 and the AcrII4A proteins are constitutively expressed from the promoter CMV; to increase the affinity of AcrII4A towards the VPR-dCas9, Coiled Coils (CCs) protein domains, consisting of two 33 amino acid protein domains that act as “molecular magnets” and can reciprocally bind to each other and causing dimerization of proteins fused to them, were fused to the two proteins of interest: one CC (N8) was fused to VPR-dCas9 protein and the other CC (N7) to the AcrII4A protein (Seq ID NO: 24 and 23 respectively). All CRISPR and Anti-CRISPR proteins are under the control of the constitutive CMV promoter. The luciferase assay was repeated, as shown in FIG. 12a-d and demonstrated higher fold repression when pairing AcrII4A-N7 with VPR-dCas9-N8, than when using the original versions without CCs. These results demonstrate that the binding affinity between these two proteins is enhanced by the presence of the CCs that increase AcrII4A-mediated repression of the VPR_dCAs9 activator.

### Example 7

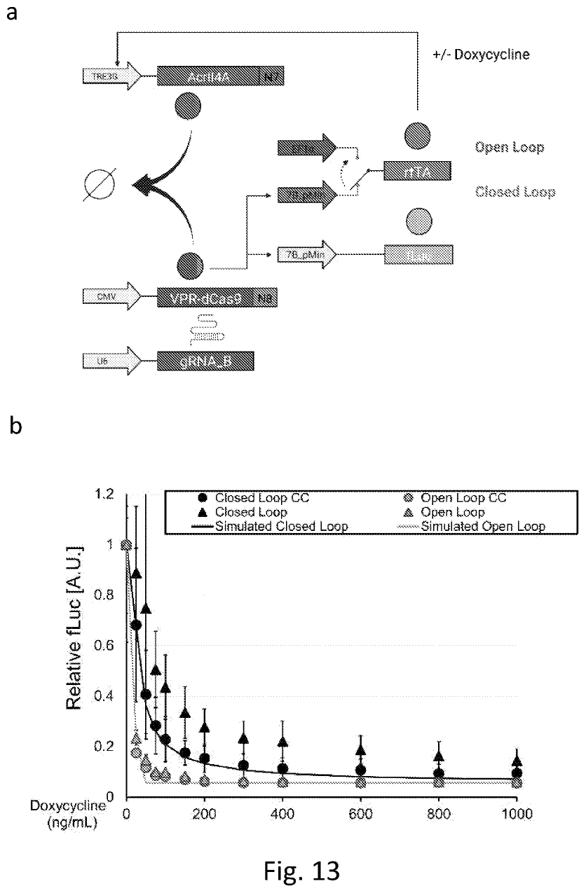

A genetic circuit according to preferred embodiment of the invention, having an NFL configuration, is provided (FIG. 13a), consisting of the strong constitutive promoter CMV driving the expression of the engineered transcriptional activator VPR-dCas9-N8 that, in the presence of a guide RNA, constitutively expressed under U6 promoter, binds the 7B_pMin promoter upstream of the rtTA gene, driving the expression of the transcription factor rtTA. The rtTA protein, in the presence of Doxycycline, binds the TRE3G promoter and drives expression of the modified anti-CRISPR protein AcrII4A-N7. This then binds and inhibits the VPR-dCas9-N8 protein, thus closing the negative feedback loop. The circuit comprises a luciferase reporter (fLuc) protein also expressed by the gRNA inducible 7B_pMin promoter. The fLuc is used in the present example to indirectly track rtTA expression, measuring its fLuc luminescence at different concentrations of Doxycycline, as reported in FIG. 13b.

As a negative control, as shown in FIG. 13a, an open loop circuit is also provided where the rtTA is under the control of the constitutive promoter EF1α, thus disabling the feedback loop. HEK293T cells were transfected with the genetic circuit, in both open and closed loop configurations, using both the original and the custom (i.e., with CCs) CRISPR and anti-CRISPR proteins.

The genetic circuit works as expected with increasing doxycycline concentration, decreasing the rtTA level, as evidenced by the very good fit between experimental data and simulated data from a mathematical model of the genetic circuit. The mathematical model is ideal in that it assumes that AcrII4A-N7 and VRP_dCAs9-N8 are stable proteins and are never degraded. To demonstrate the effect of CCs on the circuit's performance, an addition version of the same genetic circuit is built, this time using the original variants of VPR-dCas9 and AcrII4a (Seq ID NO: 17 and 18, respectively) without CCs, and the same doxycycline dose-response experiment was performed. As shown in FIG. 13b, this circuit exhibits higher luminescence values than the genetic circuit with CCs, indicative of a weaker, but still present, inhibitory action of the AcrII4a on the VRP_dCas9.

### Example 8

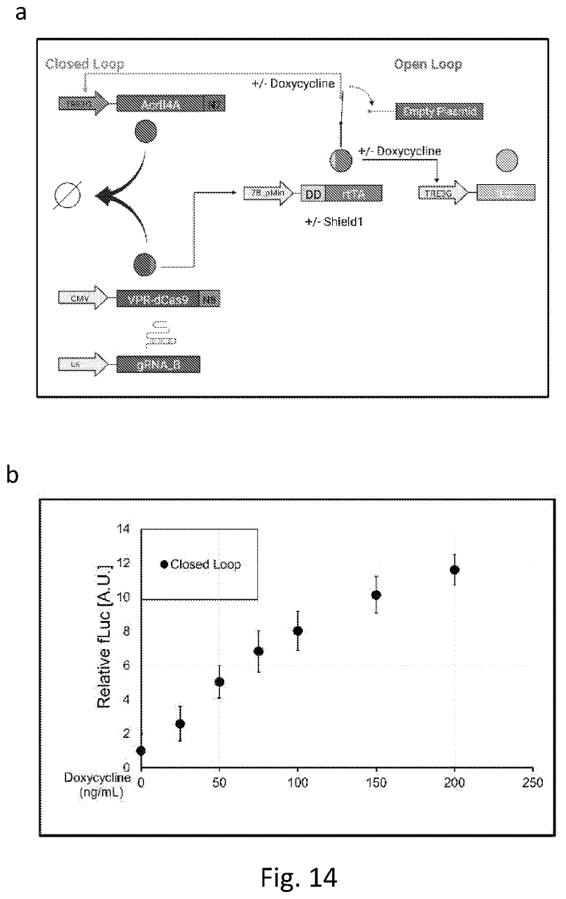

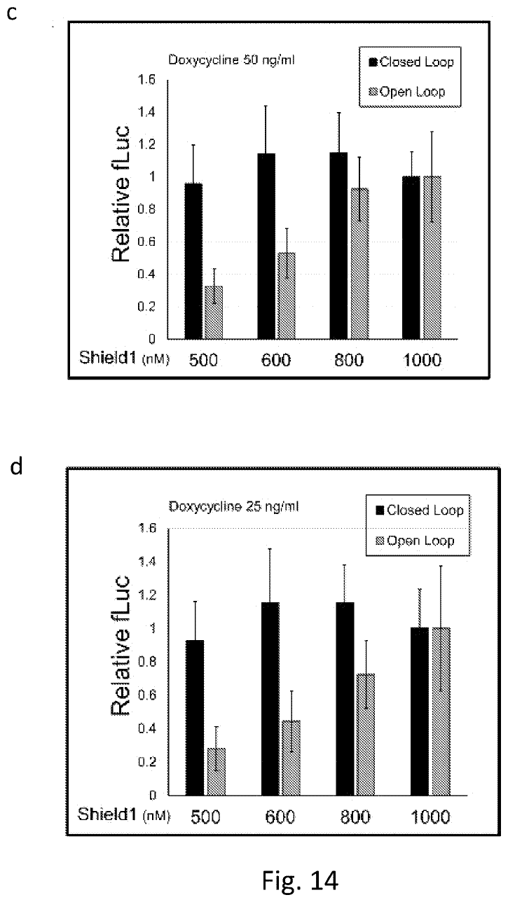

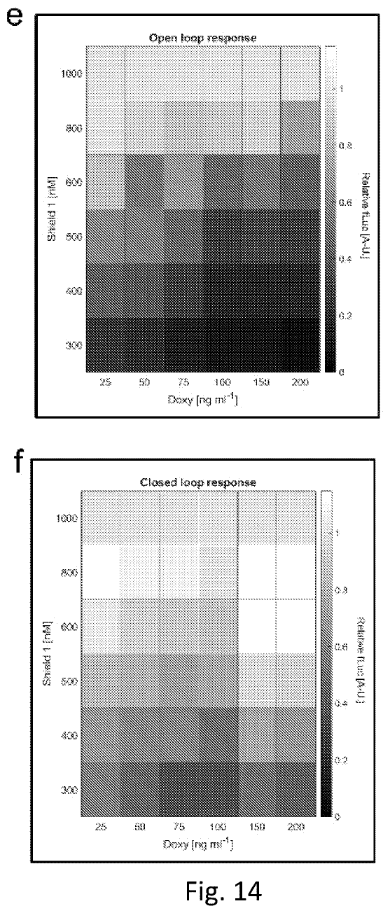

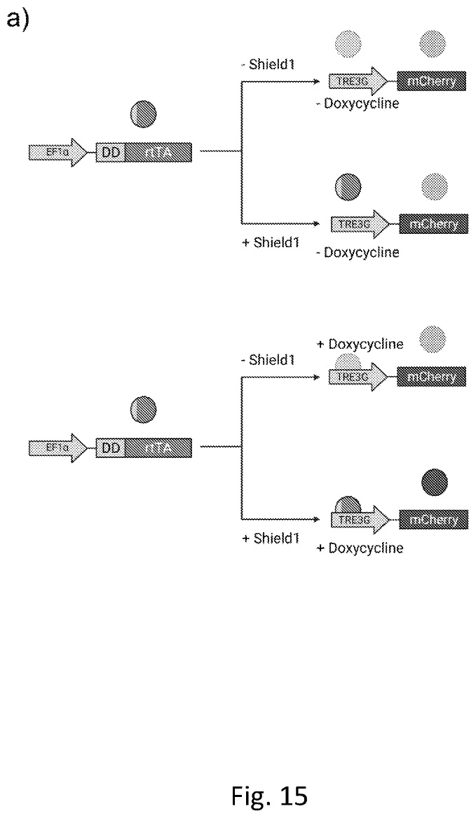

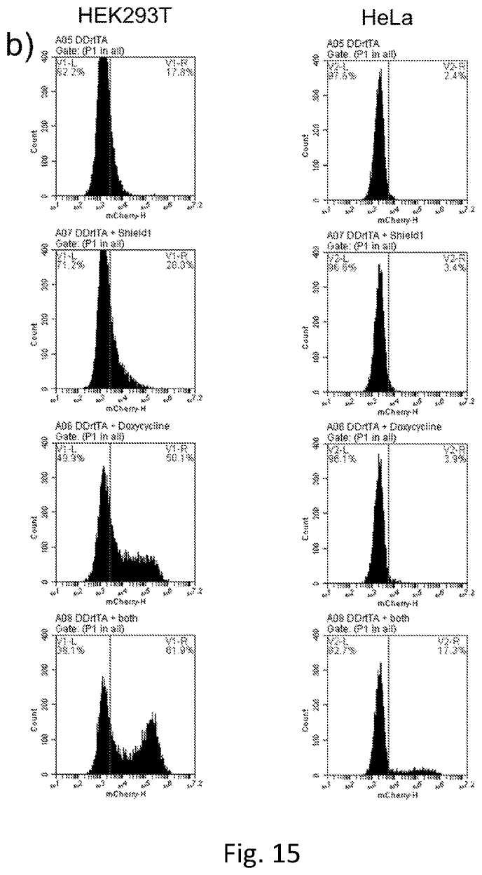

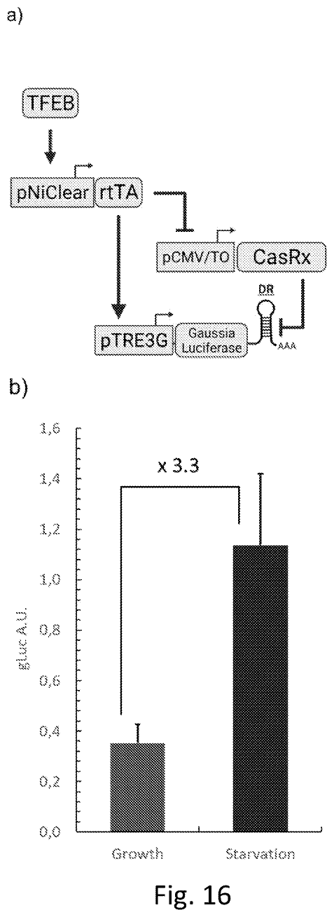

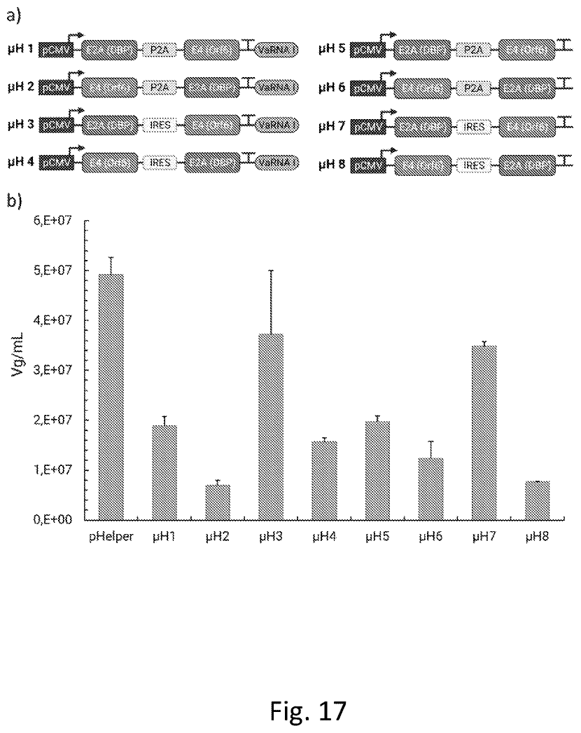

To experimentally test the ability of genetic circuits according to the invention in maintaining homeostasis in the face of perturbations, i.e., Robust Perfect Adaptation, the FKBP derived Destabilization Domain (DD) (Haugwitz, M., Garachtchenko, T., Nourzaie, O., Gandlur, S. & Sagawa, H. Rapid, on-demand protein stabilization and destabilization using the ProteoTuner™ systems. Nat. Methods 5, iii-iv (2008)) was fused to the transcription factor rtTA to obtain DD-rtTA (Seq ID No: 26), as shown in FIG. 14a. Following administration of the small molecule Shield1, which stabilizes the DD domain, the fusion protein DD-rtTA becomes stable and can accumulate in the cells, while reducing Shield1, decreases the protein stability in a dose dependent manner. To test the function of the DD domain, as shown in FIG. 15a, DD-rtTA we co-transfected with a plasmid carrying the fluorescent mCherry gene downstream of the doxycycline-responsive TRE3G promoter. As shown in FIG. 15b, it was confirmed that in both HEK293T and HeLa cells, in absence of Shield1, DD-rtTA is degraded and it does not accumulate in the cells, preventing full mCherry expression, even in the presence of a saturating dose of Doxycycline. On the contrary, when administering cells with Shield1 (1000 nM), then DD-rtTA protein accumulates and drives full express of the mCherry protein.

A circuit comprising the original rtTA with its destabilized version (DD-rtTA) and the luciferase reporter protein under the control of the TRE3G promoter was then provided, as shown in FIG. 14a. As negative control, an open loop circuit where AcrII4A-N7 is absent and substituted by an empty plasmid is also provided. As shown in FIG. 14b, fLuc expression, and hence luminescence, can be modulated in a Doxycycline-dependent manner, hence allowing to select a specific reference level by changing Doxycycline. To test the ability of the genetic circuit in maintaining the luminescence level besides perturbations, the Doxycycline concentration was fixed, while varying Shield1 concentration, in order to change the rtTA-DD stability. Luminescence values of fLuc were calculated by first normalizing against Renilla luminescence and then by dividing the normalized values by the luminescence obtained at the maximum dose of Shield1 (1000 nM), when DD-rtTA is fully stabilized. As reported in FIG. 14c, when Doxycycline is fixed at 50 ng/ml, the fLuc luminescence in the Open Loop circuit decreases proportionally with the Shield1 concentration as expected. Indeed, since DD-rtTA protein stability decreases while its mRNA level is unchanged, this causes an overall decrease in DD-rtTA protein level, and hence of the expression of fLuc downstream of the DD-rtTA regulated TRE3G promoter. On the contrary, the luminescence of Closed Loop circuit (FIG. 14c) remains constant thus demonstrating Robust Perfect Adaptation; this can be explained by the fact that as DD-rtTA protein degradation increases, this transiently decreases the DD-rtTA protein level, which in turn decreases the anti-CRISPR amount, thus more VPR_dCas9_N8 will be available to increase DD-rtTA mRNA expression thus re-establishing the correct level of DD-rtTA protein and of fLuc.

To further explore the experimental conditions in which Robust Perfect Adaptation is maintained, the same experiments was repeated for a range of Doxycycline concentrations from 0 to 200 ng/ml, gradually perturbing the system with decreasing doses of Shield 1. FIG. 14d show the results for a Doxycycline concentration of 25 ng/ml, while the heatmaps in FIG. 14e, f shows results for all the concentrations tested. fLuc luminescence values were first normalized by dividing for the constitutively expressed Renilla luminescence and then the obtained values were divided by the luminescence value obtained at a Shield1 concentration (1000 nM) and a Doxycycline concentration of 50 ng/ml.

The results demonstrate that the genetic circuit according to preferred embodiment of the invention does exhibit Robust Perfect Adaptation and that doxycycline can be used to tune expression of the reporter protein to a predetermined level.

### Example 9

A genetic circuit having CFFL configuration, according to preferred embodiments of the invention, was employed to sense the activation of the starvation responsive TFEB transcription factor. HeLa cells were transfected with a plasmid encoding the synthetic rtTA-VPR transcription factor driven by the TFEB-responsive NiClear promoter (SEQ ID NO: 31), representing the main input able to sense the cell starvation; a plasmid encoding the CasRx controlled by the pCMV/TO promoter is transfected as the repressor; finally, a plasmid encoding for the Gaussia Luciferase reporter gene driven by the tetracycline responsive pTRE3G promoter, harboring in its 3′UTR the short CasRx-specific hairpin-structured sequence Direct Repeat (DR), is transfected as final output.

Gaussia Luciferase expression is expressed in HeLa cells 48 hours after transfection. In growth condition HeLa cells were grown in DMEM medium supplied with 10% FBS for 48 hours, instead, in the Starvation condition DMEM medium was changed with HBSS (Hanks' Balanced Salt solution) 6 hours before cell lysis to quantify luciferase expression. HBSS simulate the deprivation of nutrients, i.e., starvation. HeLa cells, in both Growth and Starvation conditions, were supplied with 1000 ng/ml of doxycycline in order to make rtTA-VPR constitutively active: TFEB transcriptional factor is activated during starvation and binds to CLEAR SITES present in the NiClear promoter inducing rtTA-VPR expression. In the presence of doxycycline rtTA-VPR induces Gaussia Luc expression and represses CasRx one's. Since both conditions were supplied with 1000 ng/ml of doxycycline and rtTA-VPR is constitutively active, activation of the genetic circuit of the invention is no more related to doxycycline but depends on the amount of rtTA-VPR expression itself, that, in turn, rely on TFEB activation; in this way the activation of the genetic circuit of the invention is linked with starvation sensing. As expected, the Gaussia Luciferase expression increases in the starvation condition due to TFEB activation, compared to the Growth condition where there is no TFEB activity. HeLa cells were also transfected with a Red Firefly Luciferase reporter gene to normalize towards transfection efficiency bias between each assessed experimental condition. gLuc arbitrary units (A.U). were calculated as the ratio between the Gaussia and Red Firefly luminescence. At least, three biological replicates were analyzed for each condition.

### Example 10

E2A(DBP) and E4(Orf6) were cloned into single transcription units by means of bicistronic sequences, separated by the skipping ribosome sequence P2A (SEQ ID NO: 32) or the ECMV IRES (SEQ ID NO: 33) (Martella, A. et al. (2017) ‘EMMA: An Extensible Mammalian Modular Assembly Toolkit for the Rapid Design and Production of Diverse Expression Vectors’, ACS Synthetic Biology, 6 (7), pp. 1380-1392), generating a series of μHelper plasmids. In all plasmids the transcription unit is driven by the strong constitutive pCMV promoter. Since both bicistronic sequences P2A and IRES, cause the expression of the first coding sequence to be higher than the one of the coding sequence in second position, position of E2A(DBP) and E4(Orf6) was swapped to evaluate the impact of this expression bias on the rAAV production. The first four μHelper plasmids (μH 1-4) were cloned also including the non-coding RNA VaRNAI just after the transcriptional unit encoding for E2A(DBP) and E4(Orf6) (SEQ ID: 34, 35, 36, 37). To test the effect of VaRNAI on rAAV production, other version of μHelper 1-4 were cloned without the VaRNAI sequence, giving rise to plasmids μHelper 5-8 (SEQ ID: 38, 39, 40, 41).

μHelper plasmids rAAV vector production capacity was evaluated by means of transient triple transfection and quantified through absolute quantitative Real Time PCR. Their production yields were compared to the ones obtained by the standard pHelper plasmid. All the μHelper plasmids manage to sustain rAAV production demonstrating that E2A(DBP) and E4(Orf6) are necessary and sufficient for rAAV particles formation. Moreover, this data shows that the type of bicistronic sequence and the position expression bias impacts the rAAV production yields, where μH3 and μH7 produce the most between the μHelper plasmids. μH3 and μH7 share the same transcriptional unit composed by the E2A(DBP) coding sequence, followed by an IRES, and then the E4(Orf6) coding sequence. This data also suggests that VaRNAI doesn't give any enhancement of rAAV. A transcription unit comprising the μH7 cassette [E2A(DBP)-IRES-E4(Orf6)] was used in Example 4, driven by pTRE3G promoter and followed by DR sequences, to test the genetic circuit according to preferred embodiments of the invention to produce rAAV vectors on-demand.

### Example 11

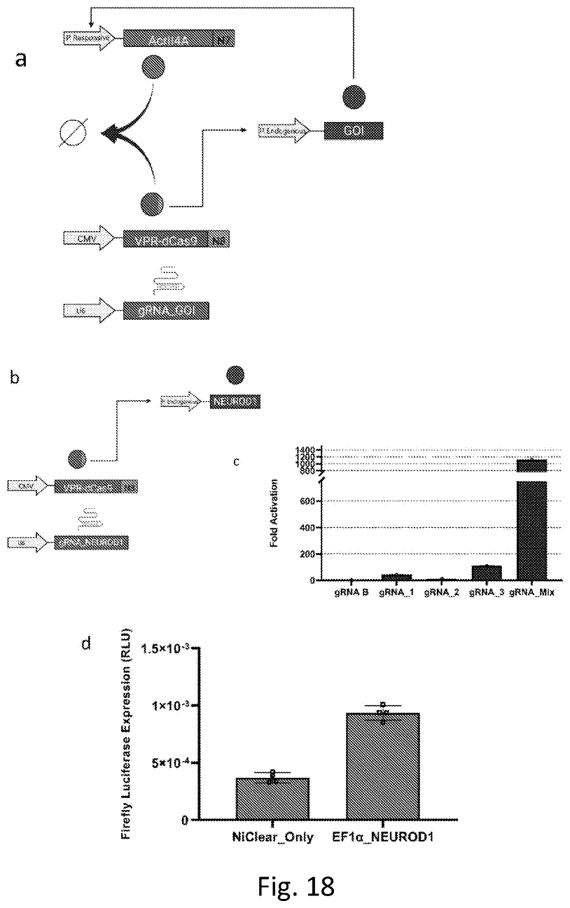

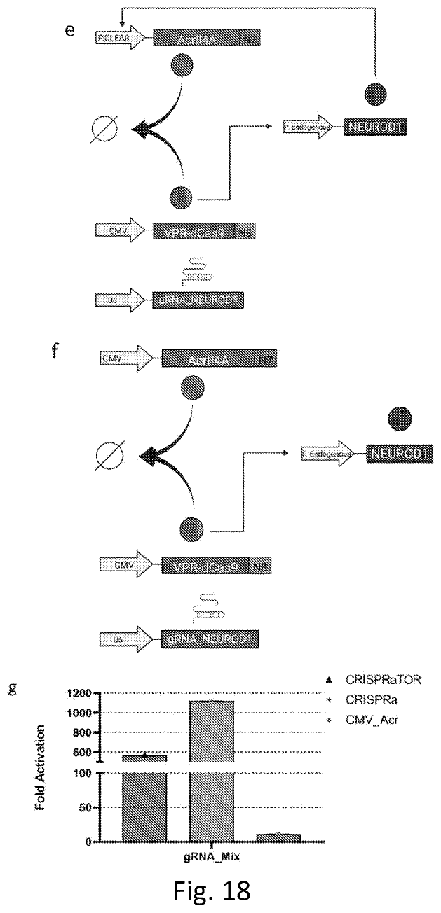

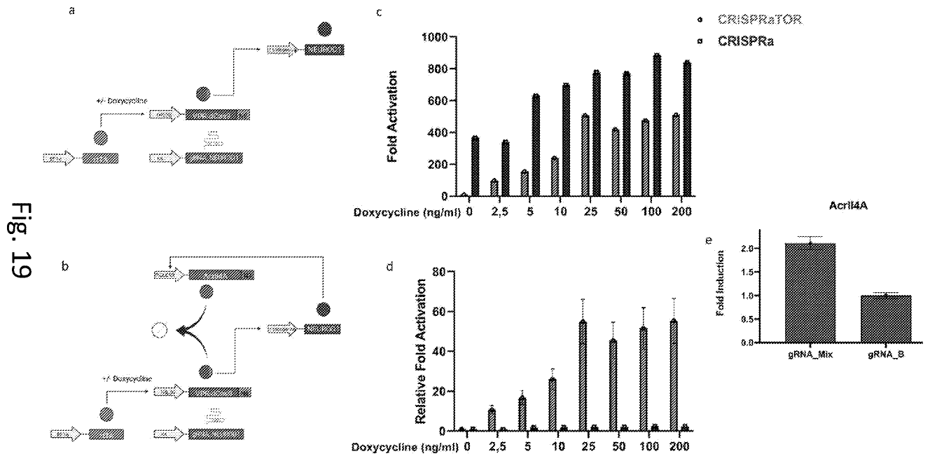

A genetic circuit having NFL configuration, according to preferred embodiments of the invention (FIG. 18a, e), was employed to regulate expression of endogenous transcription factor NEUROD1.

First, 3 gRNAs published by Chavez, A. et al. Nat. Methods 13, 563-567 (2016) were tested, transfected together with the dCas9-VPR-N8 controlled by the CMV promoter (FIG. 18b), according to the prior art. All the three gRNAs showed NEUROD1 overexpression (FIG. 18c); also, it was confirmed that mixing all the gRNAs (gRNA_Mix) led to higher level of overexpression (FIG. 18c). To properly control the transcription factor, a promoter containing 6 repetitions of the E-Box motif (to which NEUROD1 is known to bind) before a minimal promoter (NiClear promoter, SEQ ID NO: 42), was synthetized. This promoter was cloned downstream to the fLuc protein (SEQ_ID_NO: 43) and transfected together with a plasmid constitutively expressing the NEUROD1 transcription factor (Yang, X. et al. Nat. Methods 8, 659-661, 2011) under the control of the constitutive promoter EF1α, showing a weak but significant induction of the luciferase gene when NEUROD1 is overexpressed (FIG. 18d).

Further, a polynucleotide was cloned, encoding for the AcrII4A-N7 protein in front of NiClear promoter (SEQ_ID_NO: 44).

Overexpression experiments were performed with the dCas9-VPR-N8, the gRNA mix alone, and with the AcrIIa4-N7 controlled by our NEUROD1-responsive promoter NiClear (FIG. 18e) or with the same protein controlled by the strong constitutive promoter CMV, as control (FIG. 18f). NEUROD1 expression was measured through Real-Time PCR: while the constitutive expression of the AcrII4a-N7 protein completely abolishes dCas9-mediated overexpression, the NEUROD1-controlled AcrII4A-N7 protein lowers the amount of NEUROD1, still keeping it at a high level (FIG. 18g).

To demonstrate linearity, one of the characteristics of the NFL genetic circuit according to the invention, NEUROD1 overexpression was tuned by putting dCas9-VPR-N8 under the control of the doxycycline-responsive TRE3G promoter (SEQ_ID_NO: 57), using the transcription factor rtTA to induce dCas9 expression. The closed loop system according to the invention (FIG. 19b) was transfected; an open loop system without the AcrII4A-N7 protein was transfected in other samples as a comparative system (FIG. 19a).

NEUROD1 expression was measured after administration of increasing doses of doxycycline. Cells have been transfected with the tunable NFL circuit of FIG. 19b, according to the invention or with the system of FIG. 19a of the prior art. Increasing doses of doxycycline have been administered to tune VPR-dCas9-N8 overexpression. gRNA_B has been used for both circuits as a scramble guide to normalize the values. The NFL circuit according to the invention can control the endogenous transcription factor with precision and linearity, while the comparative system is unable to correlate the level of NEUROD1 with the administered dose of Doxycycline (FIG. 19c, 19d). The effect depends on AcrII4A-N7 controller's activation upon NEUROD1 overexpression. In fact, when inducing NEUROD1 with 200 ng/ml of Doxycycline, mRNA level of AcrII4A-N7 increased (FIG. 19e).

### Example 12

Biosensing genetic circuits according to the invention were tested them by transiently transfecting mammalian HEK293T cells and using a Luciferase reporter protein as output.

The input-output responses for a specific analyte (copper) were analyzed in all circuits tested, in terms of leakiness, fold change, sensitivity to input, linearity, and operation range by comparing them against each other. A score ranging from 0, representing the worst performance, to 1, representing the best one, was assigned to each system

A basal transcription-based copper biosensor (“B”, FIG. 21a) of the prior art, consisting of a firefly luciferase placed downstream of a promoter, containing four metal-responsive elements from the mouse metallothionein 1A promoter (MRE), was first tested. MREs serve as binding sites for the metal-responsive transcription factor 1 (MTF-1). Upon exposure to copper dichloride, zinc ions are displaced by intracellular metallothioneins and subsequently incorporated into MTF-1. Consequently, MTF-1 is activated, leading to the transcriptional activation of the firefly luciferase gene (FIG. 21a). Dose-response experiments were performed by transfecting the Basal Biosensor in HEK293T cells, administering increasing doses of copper dichloride, and quantifying the expression of the Luciferase protein through Luc assay. These results were used to evaluate the performance of Biosensors according to the invention. Accordingly, the proposed copper-responsive promoter MRE (SEQ_ID NO: 45) was placed upstream and the firefly luciferase downstream of the engineered networks to ensure equal conditions for testing.

A VPR-dCas9-N8 protein (encoded by polynucleotide of sequence SEQ_ID_NO: 24), driven by the copper-responsive promoter MRE (SEQ_ID NO: 45) was used as species X while, the species Z consists of a firefly luciferase (fLuc) (SEQ_ID NO: 47) cloned downstream of a VPR-dCas9-N8 responding promoter (7B_pMin) (SEQ_ID NO: 46) containing 7 binding sites for a specific single guide RNA, namely gRNA_B (Lebar, T et. al.. Nat. Chem. Biol. 16, 513-519-2020), in order to direct the VPR-dCas9-N8 protein to the promoter and to drive transcription. Thanks to the copper-responsive promoter upstream of the VPR-dCas9-N8 protein, responsiveness of a TF biosensor to changes in copper concentration was evaluated. This system revealed several limitations across all investigated parameters, including a significant increase in leakiness, inadequate fold change, and comparable linearity to that of the basal biosensor.

A first biosensing circuit (AIC biosensor, FIG. 21c) according to preferred embodiments of the invention was realized, wherein the species X consists of a VPR-dCas9-N8 transcriptional activator (SEQ_ID_NO: 24), driven by the copper-responsive MRE promoter (Seq ID NO: 45); the species Y (controller biomolecule) is AcrII4a-N7 (SEQ_ID_NO: 23) protein driven by the doxycycline-responsive TRE3G promoter (SEQ_ID_NO: 7) and the species Z (gene of interest) is a rtTA (SEQ_ID_NO: 1), driven by 7B_pMin promoter (SEQ_ID NO: 46), in order to be expressed by the VPR-dCas9-N8 protein. When VPR-dCas9-N8 is expressed, it binds to the 7B_pMin promoter, driving the expression of the species Z rtTA, that, in the presence of doxycycline, binds to the TRE3G promoter, thus promoting the transcription of the species Y, AcrII4a-N7, that binds to VPR-dCas9-N8, inhibiting it and closing the loop. As a reporter of the system, fLuc protein driven by the rtTA-responsive TRE3G promoters (SEQ ID NO: 7) was used, in order to monitor rtTA expression. For the sake of simplicity, both rtTA and fLuc can be identified as species Z. This system is an NFL system.

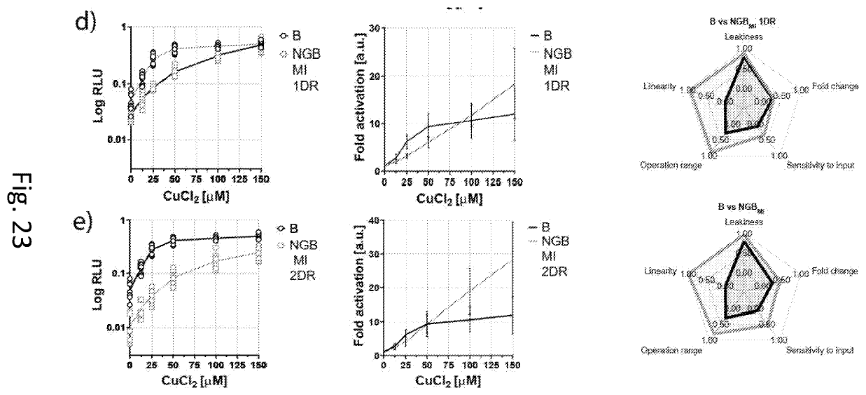

The performance of the AIC biosensor was evaluated as the copper concentration changed. Saturating concentration of doxycycline (1000 ng/ml) were used. The results revealed an increase in the signal output intensity of the AIC biosensor compared to the basal biosensor “B”, along with an elevated level of leakiness and deteriorated fold change signal when compared to the basal biosensor “B” (FIG. 23a). Conversely, significant improvements were demonstrated in the linearity of response and operation range of the AIC biosensor compared to the basal biosensor “B”. Indeed, the radar plot in FIG. 23a, summarizes the performance of AIC and B systems, indicating the AIC system dramatically improves the linearity and operation range.

A second biosensing circuit (MI biosensor, FIG. 21d) was realized incorporating a Rfx-CASRx (CasRx) (SEQ_ID_NO: 2) endoribonuclease as controller biomolecule, constitutively expressed under the control of the strong CMV promoter (SEQ_ID_NO: 5), corresponding to W in FIG. 20d; the output Z, that is the firefly luciferase includes a Direct Repeat (DR) (SEQ_ID_NO: 3) in the 3′UTR (fLuc_DR, SEQ ID NO: 49). MI biosensor was compared to MI-FFL biosensor (FIG. 21e). MI-FFL topology, according to a preferred embodiment of the invention, enhances the MI system by implementing a feedforward loop action through the inclusion of a promoter, namely CMV/2B (SEQ_ID NO: 48), that drives the expression of the CasRx (SEQ_ID_NO: 2) protein and that is repressed by the VPR-dCas9-N8 protein in presence of the gRNA_B.

The engineering CMV/2B promoter was developed in order to be repressed by the VPR-dCas9-N8, that usually works as a transcriptional activator: the DNA sequence that is complementary to the gRNA_B was cloned to be recognized and bound by the VPR-dCas9_N8 protein 9 base pairs downstream of the TATA box. Through this insertion, the VPR-dCas9-N8 protein, in complex with the gRNA_B, binds downstream of the TATA box, thus creating a steric hindrance and inhibiting the transcription, instead of promoting it, as already presented for the couple rtTA protein-CMV/TO (SEQ_ID_NO: 8) promoter.

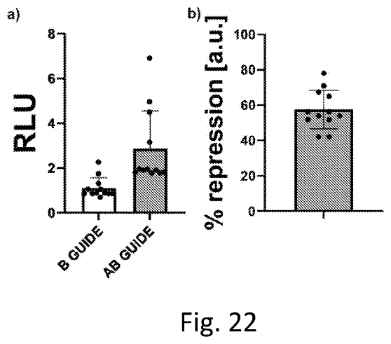

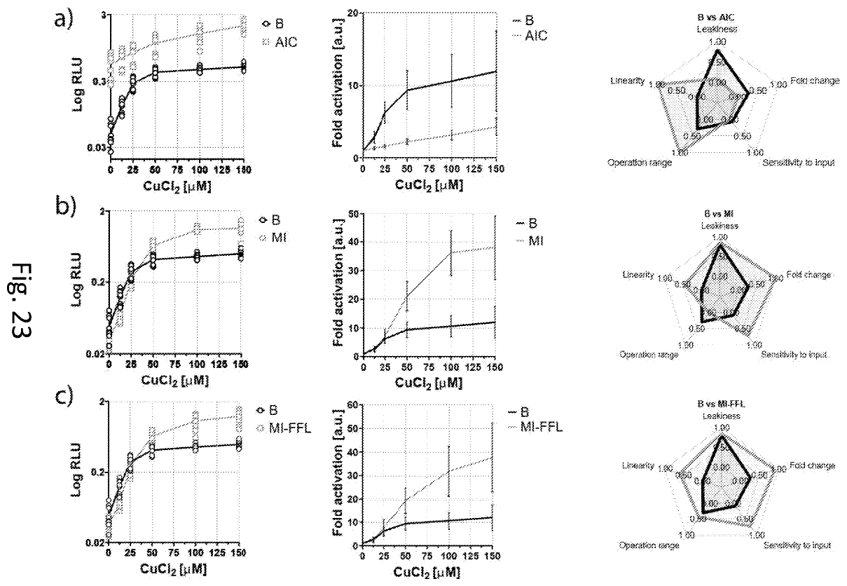

To test this newly synthetized promoter, Firefly Luciferase (SEQ_ID NO: 47) was cloned downstream of the CMV/2B (SEQ_ID NO: 48) promoter and Luc assay experiments were performed after transfecting HEK293T cells with the construct CMV/2B-fLuc, constitutively expressing VPR-dCas9-N8 and the gRNA_B. As a negative control, a non-cognate gRNA, named gRNA AB was transfected instead of the cognate gRNA_B, in order not to drive the VPR-dCas9-N8 protein on the CMV/2B promoter. As shown in FIG. 22a, a reduction in luciferase expression was observed when gRNA_B is added to the system compared to the expression levels achieved with the scrambled guide gRNA_AB. Notably, the percentage of repression of the CMV/2B when the B guide is added is around 60% of repression (FIG. 22b).

Once realized the CMV/2B promoter, both systems, MI and MI-FFL, were tested, using the VPR-dCas9-N8 protein under the control of MRE promoter, to detect copper concentrations.

All the necessary components were transfected in HEK293T cells, administering increasing doses of copper dichloride and performing Luc assay. The Basal biosensor “B” was used as a comparison to derive the metrological features. Ultimately, both the MI and MI-FFL (FIG. 21d, 21e) were confirmed to be efficient in decreasing leakiness while effectively enhancing fold-change and sensitivity (FIG. 23b, 23c), achieving the two best scores in fold-change properties. However, they exhibit an improved level of linearity performance respect with to the basic biosensor, but the scores were lower than the one of the AIC systems (FIG. 23b, 23c).

A NGB-MI biosensing circuit, that combines the AIC biosensor (FIG. 21c) with the MI one (FIG. 21d) was also tested. Notably, the NGB-MI system only requires the addition to an existing AIC biosensor of a CasRx protein (SEQ_ID_NO: 2) under the control of a constitutive promoter as CMV (SEQ_ID_NO: 5), while fusing a direct repeat (SEQ_ID_NO: 3) on the output protein Z. The CasRx, recognizing the DR on the output Z, will degrade the mRNA, thus increasing fold change. DR was fused to both the transcription factor rtTA and its driven reporter fLuc as species Z, obtaining rtTA-DR (SEQ_ID NO: 50) and fLuc-DR (SEQ_ID NO: 49) (FIG. 21f, 21g). Moreover, the NGB-MI can be further optimized by combining it with a repression action performed at the transcriptional level on the CasRx. In detail, the rtTA can repress the CasRx expression if driven by the CMV/TO promoter (SEQ_ID_NO: 14) to derive a possible realization of the NGB-C biosensing circuit (FIG. 21h, i). Similarly, the VPR-dCas9-N8 can repress the CasRx expression when the last is driven by the CMV/2B promoter (SEQ_ID NO: 48), thus generating an NGB-I biosensing circuit (FIG. 21j, 21k).

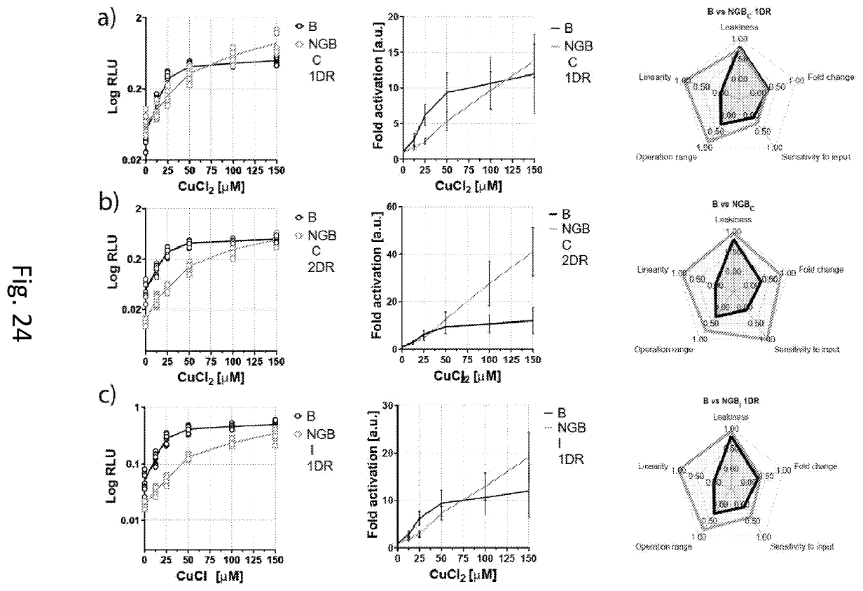

Performing transfection with different concentration of copper dichloride, followed by Luc assay, NGBs biosensors' response to changes in copper concentration was tested both by placing the direct repeat on both firefly luciferase and rtTA (NGBs 2DR, FIG. 21g, k, i) or solely on firefly luciferase (NGBs 1DR, FIG. 21f, h, j). As shown in FIG. 23e and in FIG. 24b, d, the experiments reveal that all the 2DR systems significantly reduce the biosensor's leakiness, increase linearity and operation range, and achieve high values of fold change and input sensitivity to input, with the NGB-I and NGB-C producing the best results in sensitivity to input. Moreover, the NGB-C not only enhances all the mentioned parameters but also recovers the output intensity of the basal signal. The systems with only one direct repeat on the firefly luciferase do not perform as well as the 2DR systems, as shown in FIG. 23d and in FIG. 24a, c, still exhibit good performances in terms of leakiness, linearity, and operation range. Also, the sensitivity to input increases over the basic biosensor “B”, although not to the same extent as in the 2DR systems. However, these systems evidently produce a higher output intensity.

In summary, AIC biosensors can linearize the input-output response and enhance robustness against constant disturbances; MI and MI-FFL topologies demonstrated high fold-change and sensitivity to input.

Furthermore, the combined circuit comprising the AIC biosensor with the MI or MI-FFL topologies, leverage their respective advantages. Consequently, NGB biosensors were developed that surpassed the performance of the basic biosensor across all assessed domains. By introducing mutual inhibition or both mutual inhibition and feedforward loop actions to the AIC biosensor, leakiness was successfully reduced and fold-change and sensitivity to input increased, while maintaining high levels of linearity and operation range.

### Example 13

In order to confirm the mechanism of inhibition mediated by the output species “Z” on the repressor species “Y” in genetic circuits according to preferred embodiments of the invention, HEK293 cells were co-transfected with CasRx endoribonuclease (species Y) and Gaussia Luciferase (gLuc) transcripts, the latter bearing in the 3′UTR either no Direct Repeat (DR), one DR, or four tandem DR motifs (4×DRs). The hypothesis tested was that upon CasRx binding, only one DR motifs would effectively contribute to transcript cleavage and subsequent degradation. Therefore, in transcripts bearing multiple DR motifs, the remaining DR motifs, not contributing to transcript cleavage, would act as decoys, sequestering active CasRx from the unbound pool. As a result, the CasRx repression efficacy on gLuc expression should decrease.

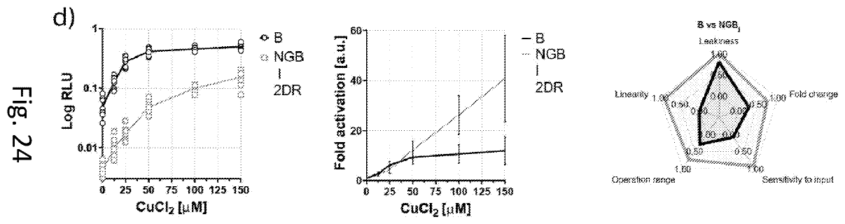

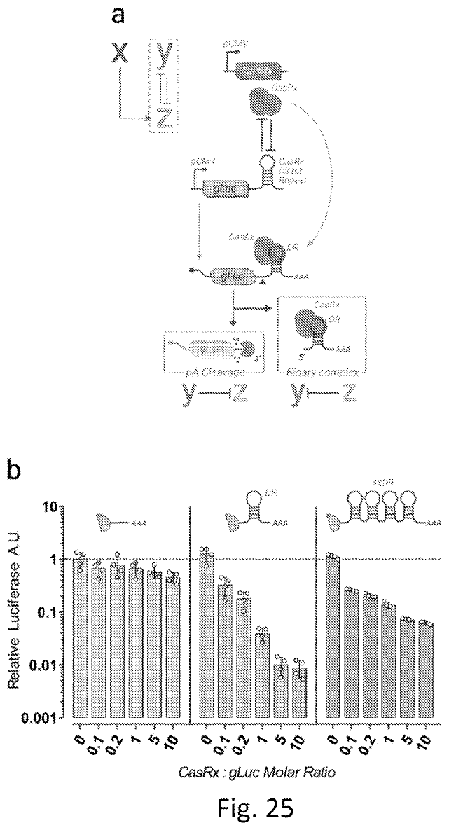

The luciferase emission of each sample was measured to quantify the expression of the gLuc at increasing amounts of co-transfected plasmid encoding the CasRx. All gLuc transcripts yielded the same luciferase expression, in the absence of the CasRx, suggesting no destabilization effect due to the addition of a DR in the 3′UTR of a transcript. As expected, for the transcript having one DR, increasing the amount of co-transfected CasRx resulted in nearly 2-log lower gLuc expression. However, the presence of 4×DRs led to higher luciferase values in the presence of CasRx compared to a single DR, confirming a decrease in cleaving function of CasRx. (FIG. 25). Increasing the amount of DR copies in the 3′UTR thus enhances the “sponge” effect, inhibiting the cleavage efficacy of CasRx.

### Example 14

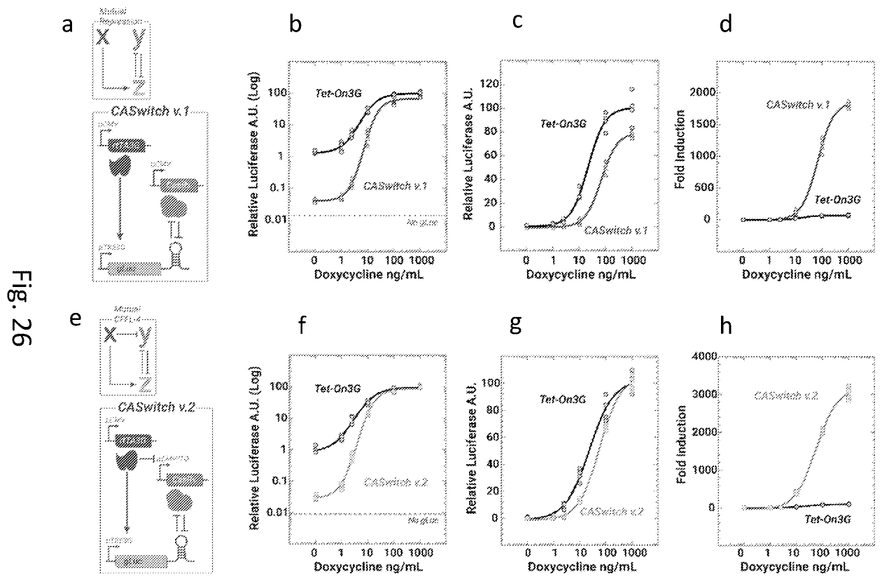

Two systems for improving control of gene expression in a cell were tested. The systems implement a mutual repression circuit wherein species Y and Z mutually repress each other, while a species X induces species Z (see FIG. 2C). When species X is active, it relieves the repression exerted by species Y on species Z by increasing the levels of Z, which in turn inhibits Y (FIG. 26a). To implement this circuit in mammalian cells, the prior art Tet-On3 system for expressing gLuc (Z) was modified by CasRx (Y) plug-in and insertion of DR in gLuc transcript. The gLuc transcript containing the (DR) sequence (Z) mutually repress each other. Additionally, the rtTA3G induces the expression of gLuc (Z) through the pTRE3G promoter. The performances of the systems according to the invention and of the prior art Tet-On3G were measured by quantifying the expression of gLuc at varying concentrations of doxycycline. For the Tet-On3G system, a gLuc transcript without the DR was used, allowing to co-transfect CasRx, thus accounting for resource burden bias between the systems.

A first system comprises CasRx constitutively expressed (CASwitch v.1, FIG. 26a).

A second system comprises CasRx gene driven by the pCMV/TO promoter (CASwitch v.2, FIG. 26e). Said second system corresponds to the CFFL, also shown in FIG. 2A, B.

Each element of the systems is encoded by a different plasmid.

As shown in FIG. 26b-d, the CASwitch v.1 system exhibited a minor decrease in induced maximum gLuc expression compared to the Tet-On3G system, demonstrating a good retention of the induced maximum response. Notably, the lower leakiness and the retention of high maximum induced expression resulted in more than 1-log higher fold-induction values for the CASwitch v.1 system compared to the Tet-On3G system (FIG. 26d). These findings demonstrate that the addition of a constitutively expressed CasRx, combined with its cognate direct repeat, can serve as a plug-and-play platform to enhance a transcriptional inducible gene system, such as Tet-On3G, by reducing leakiness, retaining high maximum expression, and amplifying its fold-induction levels.

The CASwitch v.2 system showed a reduction in leakiness comparable to CASwitch v.1, resulting in background values lower by more than 1-log compared to the Tet-On3G system (FIG. 26f). However, the CASwitch v.2 exhibited the same maximum induced gLuc expression as the Tet-On3G system, demonstrating its full retention (FIG. 26g). Taken together, these data showed that the CASwitch v.2 platform achieves more than 1-log lower leakiness retaining full maximum induced expression, compared to the Tet-On3G. This further increase in the induced maximum response respect to the CASwitch v.1 led to an amplification of fold induction levels up to 3000-fold for the CASwitch v.2 system (FIG. 26h). Overall, these data confirmed that the addition of a pCMV/TO-controlled CasRx, combined with its cognate direct repeat, can be employed as a plug-and-play platform to endow a transcriptional inducible gene system, such as Tet-On3G, with very low leakiness levels, same maximum induced expression, hence resulting in huge dynamic range amplification (on/off signal).

### Example 15

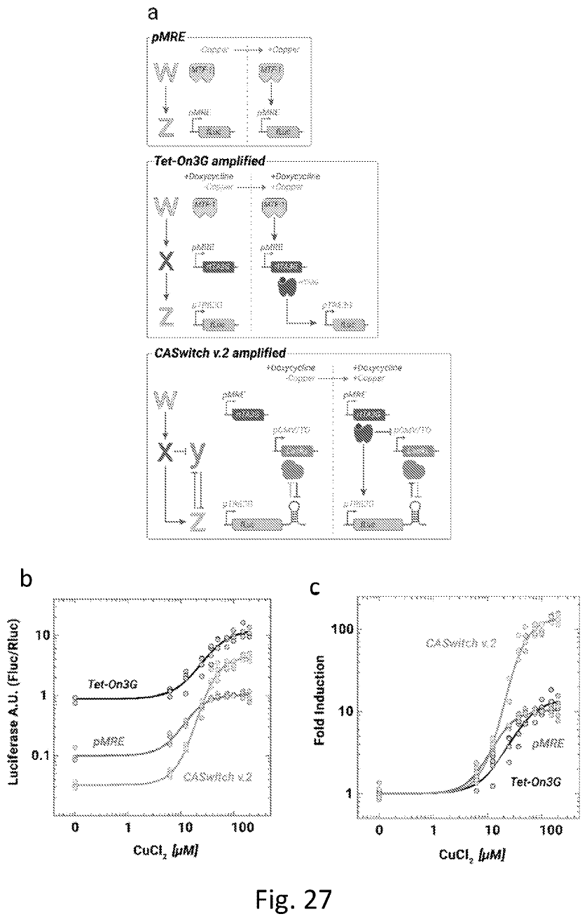

A copper biosensor of the prior art was implemented in mammalian cells with a genetic circuit according to the invention, based on a CFFL topology. The copper biosensor of the prior art, using luciferase (fLuc) as a read-out of copper concentration in the growth medium, relies on the synthetic metal-responsive promoter pMRE, which is bound by the metal response element binding transcription factor 1 (MTF-1) in the presence of zinc (Zn), copper (Cu), or cadmium (Cd), thereby inducing transcription of downstream reporter gene (FIG. 27a, top panel). This copper biosensor has several limitations, including a low absolute signal and a narrow dynamic range (on/off signal). To address these limitations, a CFFL genetic circuit according to the invention was developed with the goal of enhancing the copper biosensor's absolute signal and amplifying its dynamic range, simultaneously. In the CFFL genetic circuit (“CASwitch v.2 amplified” in FIG. 27a lower panel) expression of rtTA-VP16 transcriptional activator (X) is driven by pMRE, positively regulated by copper (W); CasRx (Y) driven by the pCMV/TO promoter mutually represses with fLuc reporter gene bearing direct repeat (DR) sequence located in the 3′ UTR. The expression of fLuc was evaluated, at increasing concentrations of copper and in the presence of doxycycline, in cells transfected with the CFFL genetic circuit, or with the basal biosensor of the prior art (FIG. 27a, top panel), or with prior art's Tet-On3G amplified biosensor, using the pMRE-rtTA-VP16 transcription unit to increase the luciferase level in the presence of copper (FIG. 27a, middle panel), as comparative samples.

The state-of-the-art copper biosensor exhibited overall low levels of reporter gene expression, achieving a maximum induction of only 10-fold (FIG. 27b,c-pMRE). Introducing the rtTA3G into the copper biosensor (Tet-On3G) resulted in a significant increase in luciferase expression, thereby amplifying the absolute signal of the biosensor (FIG. 27b-Tet-On3G). However, this enhancement did not lead to dynamic range amplification, as it also increased the background expression levels in the absence of copper (FIG. 27c-Tet-On3G). Conversely, the addition of the pCMV/TO-CasRx in the CASwitch v.2 configuration effectively reduced leakiness in the absence of copper compared to the Tet-On3G configuration, while achieving higher luciferase expression compared to the state-of-the-art copper biosensor (FIG. 27b-CASwitch v.2). Consequently, this resulted in an enhancement of the biosensor's signal and a great amplification of biosensor's dynamic range by more than 1-log, overcoming both the state-of-the-art copper biosensor and the Tet-On3G configuration (FIG. 27c-CASwitch v.2). Of note, the CASwitch v.2 yielded higher fold-induction levels at four times lower cooper concentration compared to both biosensor configurations, endowing the established copper biosensor with enhanced copper detection capacity.

### Example 16

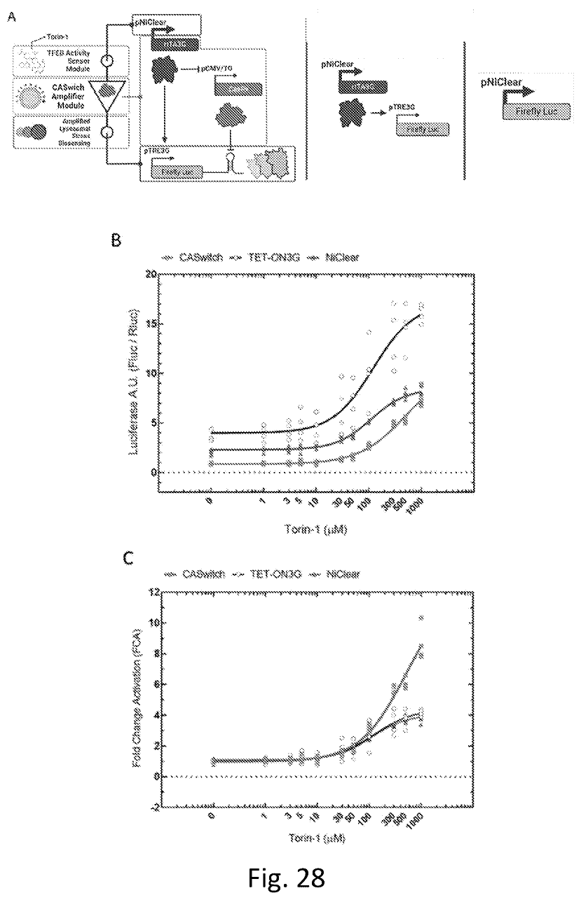

The same configurations of Example 15 were employed to enhance the performances of a Lysosomal stress biosensor based on the pNiClear promoter (FIG. 28A, pMRE-rtTA-VP16 transcription unit of SEQ ID NO: 52). Lysosomal stress response in mammalian cells is regulated by the master regulator Transcription Factor EB (TFEB). pNiClear promoter is a TFEB-responsive synthetic promoter composed of seven repeats of the CLEAR site “GTCACGTGAC” upstream of a CMV minimal promoter. This promoter can be used to drive the expression of a firefly luciferase (fLuc) reporter gene, establishing a lysosomal stress biosensor responsive to TFEB activation. Unfortunately, this biosensor is unable to sense the activation of endogenous TFEB, but responds only to exogenous TFEB overexpression, regardless of the cell lysosome stress status. The implementation of the biosensor with a CFFL circuit according to the invention, enabled a better detection of endogenous TFEB activation (FIG. 28B-C). the luciferase reporter gene expression of biosensor configurations was evaluated upon Torin-1 increases. Torin-1 is a small molecule that inhibits TFEB phosphorylation causing its translocation into the nucleus, hence its transcriptional activation. Compared to Tet-On3G and pNiClear configuration, the platform implemented with the CFFL circuit platform has the lowest leakiness values and results in the highest fold induction values (FIG. 28B-C, detailed description). Taken together this data, demonstrate that deploy a CFFL circuit according to the invention is a fast-to-implement platform to enhance the performance of established transcriptional-based biosensors.

### Example 17

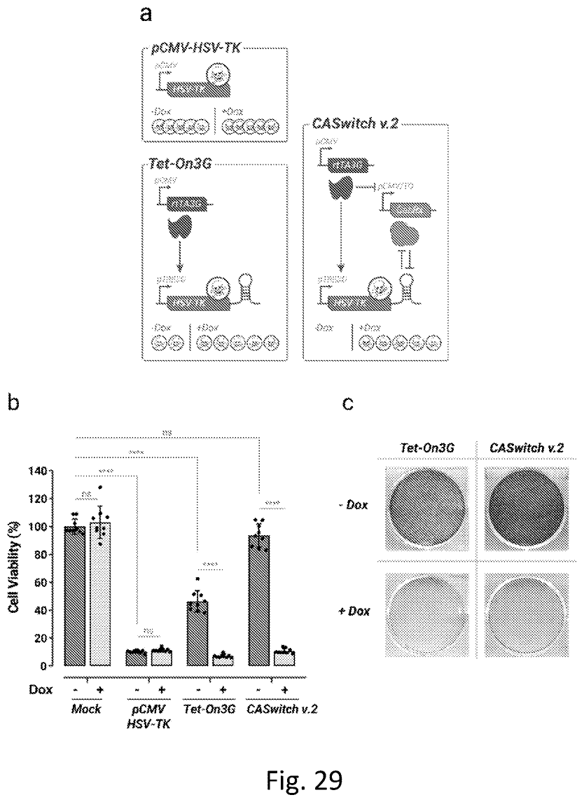

To evaluate the tightness of the CFFL circuit, its capability to tightly control the expression of a toxic gene, Herpes Simplex Virus Thymidine Kinase-1 (HSV-TK, SEQ ID NO: 53) was tested. HSV-TK1 exerts cytotoxic effects in the presence of nucleotide analogs such as ganciclovir (GCV) 12. HSV-TK gene was cloned downstream the pTRE3G promoter, incorporating the Direct Repeat in the 3′UTR. Under the influence of GCV, HSV-TK expression was controlled using either the Tet-On3G or CASwitch v.2 systems (pTRE3G-HSV_TK1-DR, SEQ ID NO: 54) based on the CFFL circuit of the invention (FIG. 29a). Cell cytotoxicity was evaluated in the absence or presence of doxycycline (FIG. 29b). To account for cytotoxic effects associated with transfection, we co-transfected HEK293T cells with a non-coding plasmid in the “Mock” condition, against which all other cell viability measurements were normalized. Furthermore, constitutive expression of HSV-TK (pCMV-HSV-TK-DR) provided a reference for the maximum achievable toxicity (FIG. 29a). Cell viability assessments demonstrated the absence of cytotoxic effects for the CASwitch v.2 system compared to the Mock condition in the absence of doxycycline; in contrast, the Tet-On3G system exhibited higher cell toxicity, resulting in approximately 50% cell death compared to the Mock condition (FIG. N29,cb). These observations were confirmed assessing cell viability also via Crystal Violet staining, which clearly showed lower amount of cell for Tet-On3G compared to the CASwitch v.2 system in the absence of doxycycline (FIG. N29c). These findings validated the tight control exerted by the CASwitch v.2 system on the expression of toxic genes, highlighting its reliability in controlling toxic viral genes expression compared to the Tet-On3G system.

### Example 18

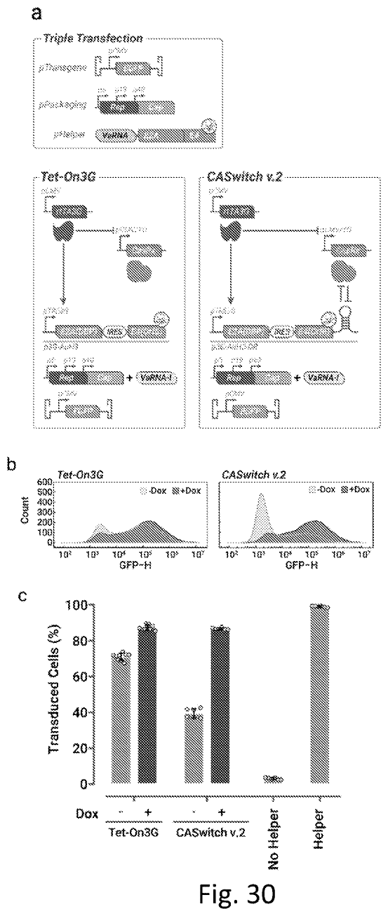

Four adenovirus μHelper plasmids were generated expressing E2A(DBP) and E4(Orf6) from a single transcript, using EMCV-IRES and P2A-skipping ribosome sequences and by interchanging the positions of E2A(DBP) and E4(Orf6) in the bicistronic transcriptional units as in Example 10. To assess the capacity of said μHelper plasmids to produce AAV vectors in comparison to the established Helper plasmid used in the prior art, the production yield through quantitative PCR (qPCR) was assessed. All μHelper plasmids were found to produce AAV vectors. Among them, the one expressing E2A [DBP]-IRES-E4 [Orf6] under CMV promoter, exhibited the highest yields (FIG. 30a, triple transfection).

A pTRE3G promoter was then cloned upstream the E2A(DBP)-IRES-E4(Orf6) transcriptional unit and the direct repeat (DR) element introduced into its 3′ untranslated region (UTR), resulting in the construction of the p3G μH-DR plasmid (SEQ ID NO: 55). Then, the effectiveness in regulating AAV vector production of a CFFL circuit wherein p3G μH-DR plasmid is a gene of interest whose expression is driven by a transcriptional activator and negatively regulated by a controller biomolecule (CASwitch v.2 system), was verified by assessing the AAV production yields in the presence or absence of doxycycline, using a “Tet-On 3G” system for comparison (FIG. 30a). Additionally, a p3G μH variant without DR was designed to account for the same expression burden imposed by the CasRx in the Tet-On3G experimental condition.

In the Triple transfection 3 plasmid are used to produce AAV vectors: pTransgene encoding for a pCMV-EGFP transcriptional unit that will be packed into AAV vectors; pPackaging containing AAV Rep and Cap genes transcribed from 3 different promoters: p5, p19 and p40; pHelper comprising the E2A, E4 and VARNA genes of Human Adenovirus 5.

In the Tet-On3G configuration, in the presence of doxycycline the rtTA3G drives the expression the E2A(DBP)-IRES-E4(Orf6) minimal helper genes transcriptional unit from the p3G-AuH3 plasmid. This condition comprises the CasRx to account for burden bias, and the other essential plasmids for AAV vector production: pPackaging, a VaRNA-I encoding plasmid, pTransgene.

In the CASwitch v.2 configuration, upon doxycycline administration, the rtTA3G drives the expression of the E2A(DBP)-IRES-E4(Orf6) minimal helper genes transcriptional unit from the p3G-AuH3-DR plasmid and, in addition, it inhibits the expression of CasRx through the pCMV/TO promoter. The CasRx can repress leaky expression of toxic helper genes in the absence of doxycycline by cleaving the E2A(DBP)-IRES-E4(Orf6) bearing the DR in its 3′UTR.HEK293T cells were co-transfected with and without the Helper plasmid to determine the upper and lower limits of production achievable by the system (FIG. 30a).

To evaluate the amount of produced AAV vectors, the percentage of GFP-positive cells was measured after transducing HEK293T cells with the same volume of crude lysate containing AAV vectors.

Crude lysates of the Hek293 cells transfected with the three gene circuits either in the presence of doxycycline presence (1000 ng/ml), or in its absence, were used to transduce non-transfected cells. Cytofluorimetry was used to measure the percentage of transduced cells; Infection results showed that the Tet-On3G system exhibited poor regulation of AAV production, displaying high infection levels even in the absence of doxycycline, thereby confirming its propensity for leaky expression of toxic viral helper genes. Conversely, the CASwitch v.2 system showed a significative reduction in the percentage of infected cells by approximately the double in the absence of doxycycline, while maintaining high production yields in its presence (FIG. 30b-c). The CASwitch v.2 infects in fact almost half less cells compared to the Tet-On3G in the absence of doxycycline, suggesting a greater repression of Helper gene leakiness respect to Tet-On3G system. This allows for an enhanced inducible control over AAV vector production using the CASwitch v.2 compared to Tet-On3G.

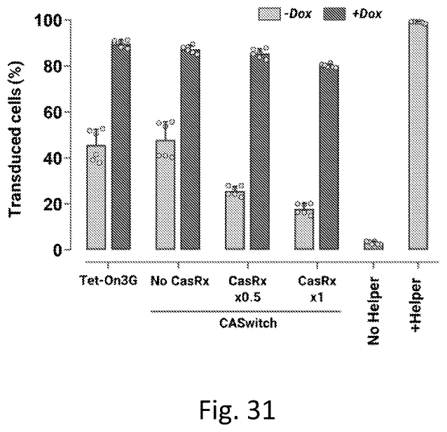

Co-transfecting half plasmid molar amount of CasRx respect to p3G-AuH3-DR (×0.5) results in almost half lower AAV vector production in the absence of doxycycline compared to Tet-On3G. Increase CasRx amount to the same co-transfected plasmid molar amount of p3G-AuH3-DR results in the even further decrease of the unintended production of AAV vectors in the absence of doxycycline to half the amount yielded by the Tet-On3G (FIG. 31). Overall, these results highlight the CASwitch v.2 as an easily implementable and promising platform to prevent unintended toxic viral gene expression in inducible AAV producer cell lines.

Sequences disclosed in conjunction with the present invention are enclosed and displayed hereafter.

Sequences
Seq ID NO: 1 rtTA-VP16
atgtctagactggacaagagcaaagtcataaactctgctctggaattactcaatggagtcggtatcgaagg
cctgacgacaaggaaactcgctcaaaagctgggagttgagcagcctaccctgtactggcacgtgaagaaca
agcgggccctgctcgatgccctgccaatcgagatgctggacaggcatcatacccactcctgccccctggaa
ggcgagtcatggcaagactttctgcggaacaacgccaagtcataccgctgtgctctcctctcacatcgcga
cggggctaaagtgcatctcggcacccgcccaacagagaaacagtacgaaaccctggaaaatcagctcgcgt
tcctgtgtcagcaaggcttctccctggagaacgcactgtacgctctgtccgccgtgggccactttacactg
ggctgcgtattggaggaacaggagcatcaagtagcaaaagaggaaagagagacacctaccaccgattctat
gcccccacttctgaaacaagcaattgagctgttcgaccggcagggagccgaacctgccttccttttcggcc
tggaactaatcatatgtggcctggagaaacagctaaagtgcgaaagcggcgggccgaccgacgcccttgac
gattttgacttagacatgctcccagccgatgcccttgacgactttgaccttgatatgctgcctgctgacgc
tcttgacgattttgaccttgacatgctccccgggtaa
Seq ID NO: 2 CasRx
atcgaaaaaaaaaagtccttcgccaagggcatgggcgtgaagtccacactcgtgtccggctccaaagtgta
catgacaaccttcgccgaaggcagcgacgccaggctggaaaagatcgtggagggcgacagcatcaggagcg
tgaatgagggcgaggccttcagcgctgaaatggccgataaaaacgccggctataagatcggcaacgccaaa
ttcagccatcctaagggctacgccgtggtggctaacaaccctctgtatacaggacccgtccagcaggatat
gctcggcctgaaggaaactctggaaaagaggtacttcggcgagagcgctgatggcaatgacaatatttgta
tccaggtgatccataacatcctggacattgaaaaaatcctcgccgaatacattaccaacgccgcctacgcc
gtcaacaatatctccggcctggataaggacattattggattcggcaagttctccacagtgtatacctacga
cgaattcaaagaccccgagcaccatagggccgctttcaacaataacgataagctcatcaacgccatcaagg
cccagtatgacgagttcgacaacttcctcgataaccccagactcggctatttcggccaggcctttttcagc
aaggagggcagaaattacatcatcaattacggcaacgaatgctatgacattctggccctcctgagcggact
gaggcactgggtggtccataacaacgaagaagagtccaggatctccaggacctggctctacaacctcgata
agaacctcgacaacgaatacatctccaccctcaactacctctacgacaggatcaccaatgagctgaccaac
tccttctccaagaactccgccgccaacgtgaactatattgccgaaactctgggaatcaaccctgccgaatt
cgccgaacaatatttcagattcagcattatgaaagagcagaaaaacctcggattcaatatcaccaagctca
gggaagtgatgctggacaggaaggatatgtccgagatcaggaaaaatcataaggtgttcgactccatcagg
accaaggtctacaccatgatggactttgtgatttataggtattacatcgaagaggatgccaaggtggctgc
cgccaataagtccctccccgataatgagaagtccctgagcgagaaggatatctttgtgattaacctgaggg
gctccttcaacgacgaccagaaggatgccctctactacgatgaagctaatagaatttggagaaagctcgaa
aatatcatgcacaacatcaaggaatttaggggaaacaagacaagagagtataagaagaaggacgcccctag
actgcccagaatcctgcccgctggccgtgatgtttccgccttcagcaaactcatgtatgccctgaccatgt
tcctggatggcaaggagatcaacgacctcctgaccaccctgattaataaattcgataacatccagagcttc
ctgaaggtgatgcctctcatcggagtcaacgctaagttcgtggaggaatacgcctttttcaaagactccgc
caagatcgccgatgagctgaggctgatcaagtccttcgctagaatgggagaacctattgccgatgccagga
gggccatgtatatcgacgccatccgtattttaggaaccaacctgtcctatgatgagctcaaggccctcgcc
gacaccttttccctggacgagaacggaaacaagctcaagaaaggcaagcacggcatgagaaatttcattat
taataacgtgatcagcaataaaaggttccactacctgatcagatacggtgatcctgcccacctccatgaga
tcgccaaaaacgaggccgtggtgaagttcgtgctcggcaggatcgctgacatccagaaaaaacagggccag
aacggcaagaaccagatcgacaggtactacgaaacttgtatcggaaaggataagggcaagagcgtgagcga
aaaggtggacgctctcacaaagatcatcaccggaatgaactacgaccaattcgacaagaaaaggagcgtca
ttgaggacaccggcagggaaaacgccgagagggagaagtttaaaaagatcatcagcctgtacctcaccgtg
atctaccacatcctcaagaatattgtcaatatcaacgccaggtacgtcatcggattccattgcgtcgagcg
tgatgctcaactgtacaaggagaaaggctacgacatcaatctcaagaaactggaagagaagggattcagct
ccgtcaccaagctctgcgctggcattgatgaaactgcccccgataagagaaaggacgtggaaaaggagatg
gctgaaagagccaaggagagcattgacagcctcgagagcgccaaccccaagctgtatgccaattacatcaa
atacagcgacgagaagaaagccgaggagttcaccaggcagattaacagggagaaggccaaaaccgccctga
acgcctacctgaggaacaccaagtggaatgtgatcatcagggaggacctcctgagaattgacaacaagaca
tgtaccctgttcagaaacaaggccgtccacctggaagtggccaggtatgtccacgcctatatcaacgacat
tgccgaggtcaattcctacttccaactgtaccattacatcatgcagagaattatcatgaatgagaggtacg
agaaaagcagcggaaaggtgtccgagtacttcgacgctgtgaatgacgagaagaagtacaacgataggctc
ctgaaactgctgtgtgtgcctttcggctactgtatccccaggtttaagaacctgagcatcgaggccctgtt
cgataggaacgaggccgccaagttcgacaaggagaaaaagaaggtgtccggcaattcc
Seq ID NO: 3-Direct Repeat
caagtaaacccctaccaactggtcggggtttgaaac
Seq ID NO: 4 rtTA-VPR
atgtctagactggacaagagcaaagtcataaactctgctctggaattactcaatggagtcggtatcgaagg
cctgacgacaaggaaactcgctcaaaagctgggagttgagcagcctaccctgtactggcacgtgaagaaca
agcgggccctgctcgatgccctgccaatcgagatgctggacaggcatcatacccactcctgccccctggaa
ggcgagtcatggcaagactttctgcggaacaacgccaagtcataccgctgtgctctcctctcacatcgcga
cggggctaaagtgcatctcggcacccgcccaacagagaaacagtacgaaaccctggaaaatcagctcgcgt
tcctgtgtcagcaaggcttctccctggagaacgcactgtacgctctgtccgccgtgggccactttacactg
ggctgcgtattggaggaacaggagcatcaagtagcaaaagaggaaagagagacacctaccaccgattctat
gcccccacttctgaaacaagcaattgagctgttcgaccggcagggagccgaacctgccttccttttcggcc
tggaactaatcatatgtggcctggagaaacagctaaagtgcgaaagcggcgacgcattggacgattttgat
ctggatatgctgggaagtgacgccctcgatgattttgaccttgacatgcttggttcggatgcccttgatga
ctttgacctcgacatgctcggcagtgacgcccttgatgatttcgacctggacatgctgattaacactagtg
gcggctctggcggcggctccggaggctctagccagtacctgcccgacaccgacgaccggcaccggatcgag
gaaaagcggaagcggacctacgagacattcaagagcatcatgaagaagtcccccttcagcggccccaccga
ccctagacctccacctagaagaatcgccgtgcccagcagatccagcgccagcgtgccaaaacctgcccccc
agccttaccccttcaccagcagcctgagcaccatcaactacgacgagttccctaccatggtgttccccagc
ggccagatctctcaggcctctgctctggctccagcccctcctcaggtgctgcctcaggctcctgctcctgc
accagctccagccatggtgtctgcactggctcaggcaccagcacccgtgcctgtgctggctcctggacctc
cacaggctgtggctccaccagcccctaaacctacacaggccggcgagggcacactgtctgaagctctgctg
cagctgcagttcgacgacgaggatctgggagccctgctgggaaacagcaccgatcctgccgtgttcaccga
cctggccagcgtggacaacagcgagttccagcagctgctgaaccagggcatccctgtggcccctcacacca
ccgagcccatgctgatggaataccccgaggccatcacccggctcgtgacaggcgctcagaggcctcctgat
ccagctcctgcccctctgggagcaccaggcctgcctaatggactgctgtctggcgacgaggacttcagctc
tatcgccgatatggatttctcagccttgctgggctctggcagcggcagccgggattccagggaagggatgt
ttttgccgaagcctgaggccggctccgctattagtgacgtgtttgagggccgcgaggtgtgccagccaaaa
cgaatccggccatttcatcctccaggaagtccatgggccaaccgcccactccccgccagcctcgcaccaac
accaaccggtccagtacatgagccagtcgggtcactgaccccggcaccagtccctcagccactggacccag
cgcccgcagtgactcccgaggccagtcacctgttggaggaccccgatgaagagacgagccaggctgtcaaa
gcccttcgggagatggccgatactgtgattccccagaaggaagaggctgcaatctgtggccaaatggacct
ttcccatccgcccccaaggggccatctggatgagctgacaaccacacttgagtccatgaccgaggatctga
acctggactcacccctgaccccggaattgaacgagattctggataccttcctgaacgacgagtgcctcttg
catgccatgcatatcagcacaggactgtccatcttcgacacatctctgttt
Seq ID NO: 5 pCMV promoter
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagct
Seq ID NO: 6 pCMV-rtTA-VPR
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctggacttagcctttagtgaaccgtcagaattaattcagatcgatctaccagaaccgtcaga
tccgctagagattacgccaaccgccaccatgggcagcatgtctagactggacaagagcaaagtcataaact
ctgctctggaattactcaatggagtcggtatcgaaggcctgacgacaaggaaactcgctcaaaagctggga
gttgagcagcctaccctgtactggcacgtgaagaacaagcgggccctgctcgatgccctgccaatcgagat
gctggacaggcatcatacccactcctgccccctggaaggcgagtcatggcaagactttctgcggaacaacg
ccaagtcataccgctgtgctctcctctcacatcgcgacggggctaaagtgcatctcggcacccgcccaaca
gagaaacagtacgaaaccctggaaaatcagctcgcgttcctgtgtcagcaaggcttctccctggagaacgc
actgtacgctctgtccgccgtgggccactttacactgggctgcgtattggaggaacaggagcatcaagtag
caaaagaggaaagagagacacctaccaccgattctatgcccccacttctgaaacaagcaattgagctgttc
gaccggcagggagccgaacctgccttccttttcggcctggaactaatcatatgtggcctggagaaacagct
aaagtgcgaaagcggcgacgcattggacgattttgatctggatatgctgggaagtgacgccctcgatgatt
ttgaccttgacatgcttggttcggatgcccttgatgactttgacctcgacatgctcggcagtgacgccctt
gatgatttcgacctggacatgctgattaacactagtggcggctctggcggcggctccggaggctctagcca
gtacctgcccgacaccgacgaccggcaccggatcgaggaaaagcggaagcggacctacgagacattcaaga
gcatcatgaagaagtcccccttcagcggccccaccgaccctagacctccacctagaagaatcgccgtgccc
agcagatccagcgccagcgtgccaaaacctgccccccagccttaccccttcaccagcagcctgagcaccat
caactacgacgagttccctaccatggtgttccccagcggccagatctctcaggcctctgctctggctccag
cccctcctcaggtgctgcctcaggctcctgctcctgcaccagctccagccatggtgtctgcactggctcag
gcaccagcacccgtgcctgtgctggctcctggacctccacaggctgtggctccaccagcccctaaacctac
acaggccggcgagggcacactgtctgaagctctgctgcagctgcagttcgacgacgaggatctgggagccc
tgctgggaaacagcaccgatcctgccgtgttcaccgacctggccagcgtggacaacagcgagttccagcag
ctgctgaaccagggcatccctgtggcccctcacaccaccgagcccatgctgatggaataccccgaggccat
cacccggctcgtgacaggcgctcagaggcctcctgatccagctcctgcccctctgggagcaccaggcctgc
ctaatggactgctgtctggcgacgaggacttcagctctatcgccgatatggatttctcagccttgctgggc
tctggcagcggcagccgggattccagggaagggatgtttttgccgaagcctgaggccggctccgctattag
tgacgtgtttgagggccgcgaggtgtgccagccaaaacgaatccggccatttcatcctccaggaagtccat
gggccaaccgcccactccccgccagcctcgcaccaacaccaaccggtccagtacatgagccagtcgggtca
ctgaccccggcaccagtccctcagccactggacccagcgcccgcagtgactcccgaggccagtcacctgtt
ggaggaccccgatgaagagacgagccaggctgtcaaagcccttcgggagatggccgatactgtgattcccc
agaaggaagaggctgcaatctgtggccaaatggacctttcccatccgcccccaaggggccatctggatgag
ctgacaaccacacttgagtccatgaccgaggatctgaacctggactcacccctgaccccggaattgaacga
gattctggataccttcctgaacgacgagtgcctcttgcatgccatgcatatcagcacaggactgtccatct
tcgacacatctctgttttga
Seq ID NO: 7-pTRE3G
gagtttactccctatcagtgatagagaacgtatgaagagtttactccctatcagtgatagagaacgtatgc
agactttactccctatcagtgatagagaacgtataaggagtttactccctatcagtgatagagaacgtatg
accagtttactccctatcagtgatagagaacgtatctacagtttactccctatcagtgatagagaacgtat
atccagtttactccctatcagtgatagagaacgtataagctttaggcgtgtacggtgggcgcctataaaag
cagagctcgtttagtgaaccgtcagatcgcctggagcaattccacaacacttttgtcttataccaactttc
cgtaccacttcctaccctcgtaaa
Seq ID NO: 8 pCMV/TO
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctctccctatcagtgatagagatctccctatcagtgatagaga
Seq ID NO: 9 Rep/Cap AAV2
atgccggggttttacgagattgtgattaaggtccccagcgaccttgacgagcatctgcccggcatttctga
cagctttgtgaactgggtggccgagaaggaatgggagttgccgccagattctgacatggatctgaatctga
ttgagcaggcacccctgaccgtggccgagaagctgcagcgcgactttctgacggaatggcgccgtgtgagt
aaggccccggaggctcttttctttgtgcaatttgagaagggagagagctacttccacatgcacgtgctcgt
ggaaaccaccggggtgaaatccatggttttgggacgtttcctgagtcagattcgcgaaaaactgattcaga
gaatttaccgcgggatcgagccgactttgccaaactggttcgcggtcacaaagaccagaaatggcgccgga
ggcgggaacaaggtggtggatgagtgctacatccccaattacttgctccccaaaacccagcctgagctcca
gtgggcgtggactaatatggaacagtatttaagcgcctgtttgaatctcacggagcgtaaacggttggtgg
cgcaacatctgacgcacgtgtcgcagacgcaggagcagaacaaagagaatcagaatcccaattctgatgcg
ccggtgatcagatcaaaaacttcagccaggtacatggagctggtcgggtggctcgtggacaaggggattac
ctcggagaagcagtggatccaggaggaccaggcctcatacatctccttcaatgcggcctccaactcgcggt
cccaaatcaaggctgccttggacaatgcgggaaagattatgagcctgactaaaaccgcccccgactacctg
gtgggccagcagcccgtggaggacatttccagcaatcggatttataaaattttggaactaaacgggtacga
tccccaatatgcggcttccgtctttctgggatgggccacgaaaaagttcggcaagaggaacaccatctggc
tgtttgggcctgcaactaccgggaagaccaacatcgcggaggccatagcccacactgtgcccttctacggg
tgcgtaaactggaccaatgagaactttcccttcaacgactgtgtcgacaagatggtgatctggtgggagga
ggggaagatgaccgccaaggtcgtggagtcggccaaagccattctcggaggaagcaaggtgcgcgtggacc
agaaatgcaagtcctcggcccagatagacccgactcccgtgatcgtcacctccaacaccaacatgtgcgcc
gtgattgacgggaactcaacgaccttcgaacaccagcagccgttgcaagaccggatgttcaaatttgaact
cacccgccgtctggatcatgactttgggaaggtcaccaagcaggaagtcaaagactttttccggtgggcaa
aggatcacgtggttgaggtggagcatgaattctacgtcaaaaagggtggagccaagaaaagacccgccccc
agtgacgcagatataagtgagcccaaacgggtgcgcgagtcagttgcgcagccatcgacgtcagacgcgga
agcttcgatcaactacgcagacaggtaccaaaacaaatgttctcgtcacgtgggcatgaatctgatgctgt
ttccctgcagacaatgcgagagaatgaatcagaattcaaatatctgcttcactcacggacagaaagactgt
ttagagtgctttcccgtgtcagaatctcaacccgtttctgtcgtcaaaaaggcgtatcagaaactgtgcta
cattcatcatatcatgggaaaggtgccagacgcttgcactgcctgcgatctggtcaatgtggatttggatg
actgcatctttgaacaataaatgatttaaatcaggtatggctgccgatggttatcttccagattggctcga
ggacactctctctgaaggaataagacagtggtggaagctcaaacctggcccaccaccaccaaagcccgcag
agcggcataaggacgacagcaggggtcttgtgcttcctgggtacaagtacctcggacccttcaacggactc
gacaagggagagccggtcaacgaggcagacgccgcggccctcgagcacgacaaagcctacgaccggcagct
cgacagcggagacaacccgtacctcaagtacaaccacgccgacgcggagtttcaggagcgccttaaagaag
atacgtcttttgggggcaacctcggacgagcagtcttccaggcgaaaaagagggttcttgaacctctgggc
ctggttgaggaacctgttaagacggctccgggaaaaaagaggccggtagagcactctcctgtggagccaga
ctcctcctcgggaaccggaaaggcgggccagcagcctgcaagaaaaagattgaattttggtcagactggag
acgcagactcagtacctgacccccagcctctcggacagccaccagcagccccctctggtctgggaactaat
acgatggctacaggcagtggcgcaccaatggcagacaataacgagggcgccgacggagtgggtaattcctc
gggaaattggcattgcgattccacatggatgggcgacagagtcatcaccaccagcacccgaacctgggccc
tgcccacctacaacaaccacctctacaaacaaatttccagccaatcaggagcctcgaacgacaatcactac
tttggctacagcaccccttgggggtattttgacttcaacagattccactgccacttttcaccacgtgactg
gcaaagactcatcaacaacaactggggattccgacccaagagactcaacttcaagctctttaacattcaag
tcaaagaggtcacgcagaatgacggtacgacgacgattgccaataaccttaccagcacggttcaggtgttt
actgactcggagtaccagctcccgtacgtcctcggctcggcgcatcaaggatgcctcccgccgttcccagc
agacgtcttcatggtgccacagtatggatacctcaccctgaacaacgggagtcaggcagtaggacgctctt
cattttactgcctggagtactttccttctcagatgctgcgtaccggaaacaactttaccttcagctacact
tttgaggacgttcctttccacagcagctacgctcacagccagagtctggaccgtctcatgaatcctctcat
cgaccagtacctgtattacttgagcagaacaaacactccaagtggaaccaccacgcagtcaaggcttcagt
tttctcaggccggagcgagtgacattcgggaccagtctaggaactggcttcctggaccctgttaccgccag
cagcgagtatcaaagacatctgcggataacaacaacagtgaatactcgtggactggagctaccaagtacca
cctcaatggcagagactctctggtgaatccgggcccggccatggcaagccacaaggacgatgaagaaaagt
tttttcctcagagcggggttctcatctttgggaagcaaggctcagagaaaacaaatgtggacattgaaaag
gtcatgattacagacgaagaggaaatcaggacaaccaatcccgtggctacggagcagtatggttctgtatc
taccaacctccagagaggcaacagacaagcagctaccgcagatgtcaacacacaaggcgttcttccaggca
tggtctggcaggacagagatgtgtaccttcaggggcccatctgggcaaagattccacacacggacggacat
tttcacccctctcccctcatgggtggattcggacttaaacaccctcctccacagattctcatcaagaacac
cccggtacctgcgaatccttcgaccaccttcagtgcggcaaagtttgcttccttcatcacacagtactcca
cgggacaggtcagcgtggagatcgagtgggagctgcagaaggaaaacagcaaacgctggaatcccgaaatt
cagtacacttccaactacaacaagtctgttaatgtggactttactgtggacactaatggcgtgtattcaga
gcctcgccccattggcaccagatacctgactcgtaatctgtcaggctaa
Seq ID NO: 10 pTRE3G-Rep/Cap AAV2-DR
gagtttactccctatcagtgatagagaacgtatgaagagtttactccctatcagtgatagagaacgtatgc
agactttactccctatcagtgatagagaacgtataaggagtttactccctatcagtgatagagaacgtatg
accagtttactccctatcagtgatagagaacgtatctacagtttactccctatcagtgatagagaacgtat
atccagtttactccctatcagtgatagagaacgtataagctttaggcgtgtacggtgggcgcctataaaag
cagagctcgtttagtgaaccgtcagatcgcctggagcaattccacaacacttttgtcttataccaactttc
cgtaccacttcctaccctcgtaaaggacttagcctttagtgaaccgtcagaattaattcagatcgatctac
cagaaccgtcagatccgctagagattacgccaaccgccaccatgggcagcatgccggggttttacgagatt
gtgattaaggtccccagcgaccttgacgagcatctgcccggcatttctgacagctttgtgaactgggtggc
cgagaaggaatgggagttgccgccagattctgacatggatctgaatctgattgagcaggcacccctgaccg
tggccgagaagctgcagcgcgactttctgacggaatggcgccgtgtgagtaaggccccggaggctcttttc
tttgtgcaatttgagaagggagagagctacttccacatgcacgtgctcgtggaaaccaccggggtgaaatc
catggttttgggacgtttcctgagtcagattcgcgaaaaactgattcagagaatttaccgcgggatcgagc
cgactttgccaaactggttcgcggtcacaaagaccagaaatggcgccggaggcgggaacaaggtggtggat
gagtgctacatccccaattacttgctccccaaaacccagcctgagctccagtgggcgtggactaatatgga
acagtatttaagcgcctgtttgaatctcacggagcgtaaacggttggtggcgcaacatctgacgcacgtgt
cgcagacgcaggagcagaacaaagagaatcagaatcccaattctgatgcgccggtgatcagatcaaaaact
tcagccaggtacatggagctggtcgggtggctcgtggacaaggggattacctcggagaagcagtggatcca
ggaggaccaggcctcatacatctccttcaatgcggcctccaactcgcggtcccaaatcaaggctgccttgg
acaatgcgggaaagattatgagcctgactaaaaccgcccccgactacctggtgggccagcagcccgtggag
gacatttccagcaatcggatttataaaattttggaactaaacgggtacgatccccaatatgcggcttccgt
ctttctgggatgggccacgaaaaagttcggcaagaggaacaccatctggctgtttgggcctgcaactaccg
ggaagaccaacatcgcggaggccatagcccacactgtgcccttctacgggtgcgtaaactggaccaatgag
aactttcccttcaacgactgtgtcgacaagatggtgatctggtgggaggaggggaagatgaccgccaaggt
cgtggagtcggccaaagccattctcggaggaagcaaggtgcgcgtggaccagaaatgcaagtcctcggccc
agatagacccgactcccgtgatcgtcacctccaacaccaacatgtgcgccgtgattgacgggaactcaacg
accttcgaacaccagcagccgttgcaagaccggatgttcaaatttgaactcacccgccgtctggatcatga
ctttgggaaggtcaccaagcaggaagtcaaagactttttccggtgggcaaaggatcacgtggttgaggtgg
agcatgaattctacgtcaaaaagggtggagccaagaaaagacccgcccccagtgacgcagatataagtgag
cccaaacgggtgcgcgagtcagttgcgcagccatcgacgtcagacgcggaagcttcgatcaactacgcaga
caggtaccaaaacaaatgttctcgtcacgtgggcatgaatctgatgctgtttccctgcagacaatgcgaga
gaatgaatcagaattcaaatatctgcttcactcacggacagaaagactgtttagagtgctttcccgtgtca
gaatctcaacccgtttctgtcgtcaaaaaggcgtatcagaaactgtgctacattcatcatatcatgggaaa
ggtgccagacgcttgcactgcctgcgatctggtcaatgtggatttggatgactgcatctttgaacaataaa
tgatttaaatcaggtatggctgccgatggttatcttccagattggctcgaggacactctctctgaaggaat
aagacagtggtggaagctcaaacctggcccaccaccaccaaagcccgcagagcggcataaggacgacagca
ggggtcttgtgcttcctgggtacaagtacctcggacccttcaacggactcgacaagggagagccggtcaac
gaggcagacgccgcggccctcgagcacgacaaagcctacgaccggcagctcgacagcggagacaacccgta
cctcaagtacaaccacgccgacgcggagtttcaggagcgccttaaagaagatacgtcttttgggggcaacc
tcggacgagcagtcttccaggcgaaaaagagggttcttgaacctctgggcctggttgaggaacctgttaag
acggctccgggaaaaaagaggccggtagagcactctcctgtggagccagactcctcctcgggaaccggaaa
ggcgggccagcagcctgcaagaaaaagattgaattttggtcagactggagacgcagactcagtacctgacc
cccagcctctcggacagccaccagcagccccctctggtctgggaactaatacgatggctacaggcagtggc
gcaccaatggcagacaataacgagggcgccgacggagtgggtaattcctcgggaaattggcattgcgattc
cacatggatgggcgacagagtcatcaccaccagcacccgaacctgggccctgcccacctacaacaaccacc
tctacaaacaaatttccagccaatcaggagcctcgaacgacaatcactactttggctacagcaccccttgg
gggtattttgacttcaacagattccactgccacttttcaccacgtgactggcaaagactcatcaacaacaa
ctggggattccgacccaagagactcaacttcaagctctttaacattcaagtcaaagaggtcacgcagaatg
acggtacgacgacgattgccaataaccttaccagcacggttcaggtgtttactgactcggagtaccagctc
ccgtacgtcctcggctcggcgcatcaaggatgcctcccgccgttcccagcagacgtcttcatggtgccaca
gtatggatacctcaccctgaacaacgggagtcaggcagtaggacgctcttcattttactgcctggagtact
ttccttctcagatgctgcgtaccggaaacaactttaccttcagctacacttttgaggacgttcctttccac
agcagctacgctcacagccagagtctggaccgtctcatgaatcctctcatcgaccagtacctgtattactt
gagcagaacaaacactccaagtggaaccaccacgcagtcaaggcttcagttttctcaggccggagcgagtg
acattcgggaccagtctaggaactggcttcctggaccctgttaccgccagcagcgagtatcaaagacatct
gcggataacaacaacagtgaatactcgtggactggagctaccaagtaccacctcaatggcagagactctct
ggtgaatccgggcccggccatggcaagccacaaggacgatgaagaaaagttttttcctcagagcggggttc
tcatctttgggaagcaaggctcagagaaaacaaatgtggacattgaaaaggtcatgattacagacgaagag
gaaatcaggacaaccaatcccgtggctacggagcagtatggttctgtatctaccaacctccagagaggcaa
cagacaagcagctaccgcagatgtcaacacacaaggcgttcttccaggcatggtctggcaggacagagatg
tgtaccttcaggggcccatctgggcaaagattccacacacggacggacattttcacccctctcccctcatg
ggtggattcggacttaaacaccctcctccacagattctcatcaagaacaccccggtacctgcgaatccttc
gaccaccttcagtgcggcaaagtttgcttccttcatcacacagtactccacgggacaggtcagcgtggaga
tcgagtgggagctgcagaaggaaaacagcaaacgctggaatcccgaaattcagtacacttccaactacaac
aagtctgttaatgtggactttactgtggacactaatggcgtgtattcagagcctcgccccattggcaccag
atacctgactcgtaatctgtcaggctaaccggactctagaacatccctacaggtgatatcctcgtgctgca
agtaaacccctaccaactggtcggggtttgaaac
Seq ID NO: 11-E2A(DBP)
atgggcagcgccagtcgggaagaggagcagcgcgaaaccacccccgagcgcggacgcggtgcggcgcgacg
tcccccaaccatggaggacgtgtcgtccccgtccccgtcgccgccgcctccccgggcgcccccaaaaaagc
ggatgaggcggcgtatcgagtccgaggacgaggaagactcatcacaagacgcgctggtgccgcgcacaccc
agcccgcggccatcgacctcggcggcggatttggccattgcgcccaagaagaaaaagaagcgcccttctcc
caagcccgagcgcccgccatcaccagaggtaatcgtggacagcgaggaagaaagagaagatgtggcgctac
aaatggtgggtttcagcaacccaccggtgctaatcaagcatggcaaaggaggtaagcgcacagtgcggcgg
ctgaatgaagacgacccagtggcgcgtggtatgcggacgcaagaggaagaggaagagcccagcgaagcgga
aagtgaaattacggtgatgaacccgctgagtgtgccgatcgtgtctgcgtgggagaagggcatggaggctg
cgcgcgcgctgatggacaagtaccacgtggataacgatctaaaggcgaacttcaaactactgcctgaccaa
gtggaagctctggcggccgtatgcaagacctggctgaacgaggagcaccgcgggttgcagctgaccttcac
cagcaacaagacctttgtgacgatgatggggcgattcctgcaggcgtacctgcagtcgtttgcagaggtga
cctacaagcatcacgagcccacgggctgcgcgttgtggctgcaccgctgcgctgagatcgaaggcgagctt
aagtgtctacacggaagcattatgataaataaggagcacgtgattgaaatggatgtgacgagcgaaaacgg
gcagcgcgcgctgaaggagcagtctagcaaggccaagatcgtgaagaaccggtggggccgaaatgtggtgc
agatctccaacaccgacgcaaggtgctgcgtgcacgacgcggcctgtccggccaatcagttttccggcaag
tcttgcggcatgttcttctctgaaggcgcaaaggctcaggtggcttttaagcagatcaaggcttttatgca
ggcgctgtatcctaacgcccagaccgggcacggtcaccttttgatgccactacggtgcgagtgcaactcaa
agcctgggcacgcgccctttttgggaaggcagctaccaaagttgactccgttcgccctgagcaacgcggag
gacctggacgcggatctgatctccgacaagagcgtgctggccagcgtgcaccacccggcgctgatagtgtt
ccagtgctgcaaccctgtgtatcgcaactcgcgcgcgcagggcggaggccccaactgcgacttcaagatat
cggcgcccgacctgctaaacgcgttggtgatggtgcgcagcctgtggagtgaaaacttcaccgagctgccg
cggatggttgtgcctgagtttaagtggagcactaaacaccagtatcgcaacgtgtccctgccagtggcgca
tagcgatgcgcggcagaacccctttgattttt
Seq ID NO: 12-E4(Orf6)
atgactacgtccggcgttccatttggcatgacactacgaccaacacgatctcggttgtctcggcgcactcc
gtacagtagggatcgtctacctccttttgagacagaaacccgcgctaccatactggaggatcatccgctgc
tgcccgaatgtaacactttgacaatgcacaacgtgagttacgtgcgaggtcttccctgcagtgtgggattt
acgctgattcaggaatgggttgttccctgggatatggttctaacgcgggaggagcttgtaatcctgaggaa
gtgtatgcacgtgtgcctgtgttgtgccaacattgatatcatgacgagcatgatgatccatggttacgagt
cctgggctctccactgtcattgttccagtcccggttccctgcagtgtatagccggcgggcaggttttggcc
agctggtttaggatggtggtggatggcgccatgtttaatcagaggtttatatggtaccgggaggtggtgaa
ttacaacatgccaaaagaggtaatgtttatgtccagcgtgtttatgaggggtcgccacttaatctacctgc
gcttgtggtatgatggccacgtgggttctgtggtccccgccatgagctttggatacagcgccttgcactgt
gggattttgaacaatattgtggtgctgtgctgcagttactgtgctgatttaagtgagatcagggtgcgctg
ctgtgcccggaggacaaggcgccttatgctgcgggcggtgcgaatcatcgctgaggaaaccactgccatgt
tgtattcctgcaggacggagcggcggcggcagcagtttattcgcgcgctgctgcagcaccaccgccctatc
ctgatgcacgattatgactctacccccatgtcaggctaa
Seq ID NO: 13-pTRE3G-E2A-IRES-E4(Orf6)-DR
gagtttactccctatcagtgatagagaacgtatgaagagtttactccctatcagtgatagagaacgtatgc
agactttactccctatcagtgatagagaacgtataaggagtttactccctatcagtgatagagaacgtatg
accagtttactccctatcagtgatagagaacgtatctacagtttactccctatcagtgatagagaacgtat
atccagtttactccctatcagtgatagagaacgtataagctttaggcgtgtacggtgggcgcctataaaag
cagagctcgtttagtgaaccgtcagatcgcctggagcaattccacaacacttttgtcttataccaactttc
cgtaccacttcctaccctcgtaaaggacttagcctttagtgaaccgtcagaattaattcagatcgatctac
cagaaccgtcagatccgctagagattacgccaaccgccaccatgggcagcatgggcagcgccagtcgggaa
gaggagcagcgcgaaaccacccccgagcgcggacgcggtgcggcgcgacgtcccccaaccatggaggacgt
gtcgtccccgtccccgtcgccgccgcctccccgggcgcccccaaaaaagcggatgaggcggcgtatcgagt
ccgaggacgaggaagactcatcacaagacgcgctggtgccgcgcacacccagcccgcggccatcgacctcg
gcggcggatttggccattgcgcccaagaagaaaaagaagcgcccttctcccaagcccgagcgcccgccatc
accagaggtaatcgtggacagcgaggaagaaagagaagatgtggcgctacaaatggtgggtttcagcaacc
caccggtgctaatcaagcatggcaaaggaggtaagcgcacagtgcggcggctgaatgaagacgacccagtg
gcgcgtggtatgcggacgcaagaggaagaggaagagcccagcgaagcggaaagtgaaattacggtgatgaa
cccgctgagtgtgccgatcgtgtctgcgtgggagaagggcatggaggctgcgcgcgcgctgatggacaagt
accacgtggataacgatctaaaggcgaacttcaaactactgcctgaccaagtggaagctctggcggccgta
tgcaagacctggctgaacgaggagcaccgcgggttgcagctgaccttcaccagcaacaagacctttgtgac
gatgatggggcgattcctgcaggcgtacctgcagtcgtttgcagaggtgacctacaagcatcacgagccca
cgggctgcgcgttgtggctgcaccgctgcgctgagatcgaaggcgagcttaagtgtctacacggaagcatt
atgataaataaggagcacgtgattgaaatggatgtgacgagcgaaaacgggcagcgcgcgctgaaggagca
gtctagcaaggccaagatcgtgaagaaccggtggggccgaaatgtggtgcagatctccaacaccgacgcaa
ggtgctgcgtgcacgacgcggcctgtccggccaatcagttttccggcaagtcttgcggcatgttcttctct
gaaggcgcaaaggctcaggtggcttttaagcagatcaaggcttttatgcaggcgctgtatcctaacgccca
gaccgggcacggtcaccttttgatgccactacggtgcgagtgcaactcaaagcctgggcacgcgccctttt
tgggaaggcagctaccaaagttgactccgttcgccctgagcaacgcggaggacctggacgcggatctgatc
tccgacaagagcgtgctggccagcgtgcaccacccggcgctgatagtgttccagtgctgcaaccctgtgta
tcgcaactcgcgcgcgcagggcggaggccccaactgcgacttcaagatatcggcgcccgacctgctaaacg
cgttggtgatggtgcgcagcctgtggagtgaaaacttcaccgagctgccgcggatggttgtgcctgagttt
aagtggagcactaaacaccagtatcgcaacgtgtccctgccagtggcgcatagcgatgcgcggcagaaccc
ctttgatttttcaggctaataacagcttccggactctagaacatccctacaggtgatatcctcgatcccgc
tcgataagcttgatatcgaattccgcccccccccctctccctcccccccccctaacgttactggccgaagc
cgcttggaataaggccggtgtgcgtttgtctatatgttattttccaccatattgccgtcttttggcaatgt
gagggcccggaaacctggccctgtcttcttgacgagcattcctaggggtctttcccctctcgccaaaggaa
tgcaaggtctgttgaatgtcgtgaaggaagcagttcctctggaagcttcttgaagacaaacaacgtctgta
gcgaccctttgcaggcagcggaaccccccacctggcgacaggtgcctctgcggccaaaagccacgtgtata
agatacacctgcaaaggcggcacaaccccagtgccacgttgtgagttggatagttgtggaaagagtcaaat
ggctctcctcaagcgtattcaacaaggggctgaaggatgcccagaaggtaccccattgtatgggatctgat
ctggggcctcggtgcacatgctttacatgtgtttagtcgaggttaaaaaaacgtctaggccccccgaacca
cggggacgtggttttcctttgaaaaacacgatgataatatggccacaaccgcgtccatgactacgtccggc
gttccatttggcatgacactacgaccaacacgatctcggttgtctcggcgcactccgtacagtagggatcg
tctacctccttttgagacagaaacccgcgctaccatactggaggatcatccgctgctgcccgaatgtaaca
ctttgacaatgcacaacgtgagttacgtgcgaggtcttccctgcagtgtgggatttacgctgattcaggaa
tgggttgttccctgggatatggttctaacgcgggaggagcttgtaatcctgaggaagtgtatgcacgtgtg
cctgtgttgtgccaacattgatatcatgacgagcatgatgatccatggttacgagtcctgggctctccact
gtcattgttccagtcccggttccctgcagtgtatagccggcgggcaggttttggccagctggtttaggatg
gtggtggatggcgccatgtttaatcagaggtttatatggtaccgggaggtggtgaattacaacatgccaaa
agaggtaatgtttatgtccagcgtgtttatgaggggtcgccacttaatctacctgcgcttgtggtatgatg
gccacgtgggttctgtggtccccgccatgagctttggatacagcgccttgcactgtgggattttgaacaat
attgtggtgctgtgctgcagttactgtgctgatttaagtgagatcagggtgcgctgctgtgcccggaggac
aaggcgccttatgctgcgggcggtgcgaatcatcgctgaggaaaccactgccatgttgtattcctgcagga
cggagcggcggcggcagcagtttattcgcgcgctgctgcagcaccaccgccctatcctgatgcacgattat
gactctacccccatgtcaggctaataacagcttccggactctagaacatccctacaggtgatatcctcgtg
ctgcaagtaaacccctaccaactggtcggggtttgaaac
Seq ID NO: 14-pCMV/TO-CasRx with NLS
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctctccctatcagtgatagagatctccctatcagtgatagagatcgtcgacgaggacttagc
ctttagtgaaccgtcagaattaattcagatcgatctaccagaaccgtcagatccgctagagattacgccaa
ccgccaccatgggcagcatgagccccaagaagaagagaaaggtggaggccagcatcgaaaaaaaaaagtcc
ttcgccaagggcatgggcgtgaagtccacactcgtgtccggctccaaagtgtacatgacaaccttcgccga
aggcagcgacgccaggctggaaaagatcgtggagggcgacagcatcaggagcgtgaatgagggcgaggcct
tcagcgctgaaatggccgataaaaacgccggctataagatcggcaacgccaaattcagccatcctaagggc
tacgccgtggtggctaacaaccctctgtatacaggacccgtccagcaggatatgctcggcctgaaggaaac
tctggaaaagaggtacttcggcgagagcgctgatggcaatgacaatatttgtatccaggtgatccataaca
tcctggacattgaaaaaatcctcgccgaatacattaccaacgccgcctacgccgtcaacaatatctccggc
ctggataaggacattattggattcggcaagttctccacagtgtatacctacgacgaattcaaagaccccga
gcaccatagggccgctttcaacaataacgataagctcatcaacgccatcaaggcccagtatgacgagttcg
acaacttcctcgataaccccagactcggctatttcggccaggcctttttcagcaaggagggcagaaattac
atcatcaattacggcaacgaatgctatgacattctggccctcctgagcggactgaggcactgggtggtcca
taacaacgaagaagagtccaggatctccaggacctggctctacaacctcgataagaacctcgacaacgaat
acatctccaccctcaactacctctacgacaggatcaccaatgagctgaccaactccttctccaagaactcc
gccgccaacgtgaactatattgccgaaactctgggaatcaaccctgccgaattcgccgaacaatatttcag
attcagcattatgaaagagcagaaaaacctcggattcaatatcaccaagctcagggaagtgatgctggaca
ggaaggatatgtccgagatcaggaaaaatcataaggtgttcgactccatcaggaccaaggtctacaccatg
atggactttgtgatttataggtattacatcgaagaggatgccaaggtggctgccgccaataagtccctccc
cgataatgagaagtccctgagcgagaaggatatctttgtgattaacctgaggggctccttcaacgacgacc
agaaggatgccctctactacgatgaagctaatagaatttggagaaagctcgaaaatatcatgcacaacatc
aaggaatttaggggaaacaagacaagagagtataagaagaaggacgcccctagactgcccagaatcctgcc
cgctggccgtgatgtttccgccttcagcaaactcatgtatgccctgaccatgttcctggatggcaaggaga
tcaacgacctcctgaccaccctgattaataaattcgataacatccagagcttcctgaaggtgatgcctctc
atcggagtcaacgctaagttcgtggaggaatacgcctttttcaaagactccgccaagatcgccgatgagct
gaggctgatcaagtccttcgctagaatgggagaacctattgccgatgccaggagggccatgtatatcgacg
ccatccgtattttaggaaccaacctgtcctatgatgagctcaaggccctcgccgacaccttttccctggac
gagaacggaaacaagctcaagaaaggcaagcacggcatgagaaatttcattattaataacgtgatcagcaa
taaaaggttccactacctgatcagatacggtgatcctgcccacctccatgagatcgccaaaaacgaggccg
tggtgaagttcgtgctcggcaggatcgctgacatccagaaaaaacagggccagaacggcaagaaccagatc
gacaggtactacgaaacttgtatcggaaaggataagggcaagagcgtgagcgaaaaggtggacgctctcac
aaagatcatcaccggaatgaactacgaccaattcgacaagaaaaggagcgtcattgaggacaccggcaggg
aaaacgccgagagggagaagtttaaaaagatcatcagcctgtacctcaccgtgatctaccacatcctcaag
aatattgtcaatatcaacgccaggtacgtcatcggattccattgcgtcgagcgtgatgctcaactgtacaa
ggagaaaggctacgacatcaatctcaagaaactggaagagaagggattcagctccgtcaccaagctctgcg
ctggcattgatgaaactgcccccgataagagaaaggacgtggaaaaggagatggctgaaagagccaaggag
agcattgacagcctcgagagcgccaaccccaagctgtatgccaattacatcaaatacagcgacgagaagaa
agccgaggagttcaccaggcagattaacagggagaaggccaaaaccgccctgaacgcctacctgaggaaca
ccaagtggaatgtgatcatcagggaggacctcctgagaattgacaacaagacatgtaccctgttcagaaac
aaggccgtccacctggaagtggccaggtatgtccacgcctatatcaacgacattgccgaggtcaattccta
cttccaactgtaccattacatcatgcagagaattatcatgaatgagaggtacgagaaaagcagcggaaagg
tgtccgagtacttcgacgctgtgaatgacgagaagaagtacaacgataggctcctgaaactgctgtgtgtg
cctttcggctactgtatccccaggtttaagaacctgagcatcgaggccctgttcgataggaacgaggccgc
caagttcgacaaggagaaaaagaaggtgtccggcaattccggatccggacctaagaaaaagaggaaggtg
Seq ID NO: 15-VPR with Sv40 NLS
atggacgcattggacgattttgatctggatatgctgggaagtgacgccctcgatgattttgaccttgacat
gcttggttcggatgcccttgatgactttgacctcgacatgctcggcagtgacgcccttgatgatttcgacc
tggacatgctgattaactctagaagttccggatctccgaaaaagaaacgcaaagttggtagccagtacctg
cccgacaccgacgaccggcaccggatcgaggaaaagcggaagcggacctacgagacattcaagagcatcat
gaagaagtcccccttcagcggccccaccgaccctaggcctccacctagaagaatcgccgtgcccagcagat
ccagcgccagcgtgccaaaacctgccccccagccttaccccttcaccagcagcctgagcaccatcaactac
gacgagttccctaccatggtgttccccagcggccagatctctcaggcctctgctctggccccagcccctcc
tcaggtgctgcctcaggctcctgctcctgcaccagctccagccatggtgtctgcactggctcaggcaccag
cacccgtgcctgtgctggctcctggacctccacaggctgtggctccaccagcccctaaacctacacaggcc
ggcgagggcacactgtctgaagctctgctgcagctgcagttcgacgacgaggatctgggagccctgctggg
aaacagcaccgatcctgccgtgttcaccgacctggccagcgtggacaacagcgagttccagcagctgctga
accagggcatccctgtggcccctcacaccaccgagcccatgctgatggaataccccgaggccatcacccgg
ctcgtgacaggcgctcagaggcctcctgatccagctcctgcccctctgggagcaccaggcctgcctaatgg
actgctgtctggcgacgaggacttcagctctatcgccgatatggatttctcagccttgctgggctctggca
gcggcagccgggattccagggaagggatgtttttgccgaagcctgaggccggctccgctattagtgacgtg
tttgagggccgcgaggtgtgccagccaaaacgaatccggccatttcatcctccaggaagtccatgggccaa
ccgcccactccccgccagcctcgcaccaacaccaaccggtccagtacatgagccagtcgggtcactgaccc
cggcaccagtccctcagccactggatccagcgcccgcagtgactcccgaggccagtcacctgttggaggat
cccgatgaagaaacgagccaggctgtcaaagcccttcgggagatggccgatactgtgattccccagaagga
agaggctgcaatctgtggccaaatggacctttcccatccgcccccaaggggccatctggatgagctgacaa
ccacacttgagtccatgaccgaggatctgaacctggactcacccctgaccccggaattgaacgagattctg
gataccttcctgaacgacgagtgcctcttgcatgccatgcatatcagcacaggactgtccatcttcgacac
atctctgttttaa
Seq ID NO: 16-dCas9 with SV40 NLS
atggacaagaaatactccatcggccttgccatcgggaccaacagcgtgggttgggccgtgatcactgacga
atacaaagtgccatcgaaaaagtttaaggtgctgggcaatactgacagacatagcatcaagaaaaacctga
ttggagccctcctgttcgactccggggaaaccgccgaagccactcgcctgaaacggaccgcacgccggagg
tacactcgccggaagaatagaatctgctaccttcaagagatctttagcaacgagatggccaaagtggacga
ttcgttcttccaccggctggaggagtcattcctggtcgaagaggacaaaaagcatgagagacatccgatct
tcggtaacattgtggatgaggtcgcctaccacgagaagtaccccactatctaccatttgcgcaagaagttg
gtcgactcgactgataaggccgacctcagactcatctacctcgctttggcacacatgatcaagtttagggg
tcacttcctgattgaaggggatctgaacccagacaactcggatgtggataaactgtttatccagctggtcc
agacttacaatcagctctttgaggagaacccgatcaatgcctccggcgtggatgctaaggcaatcctgtcc
gcacgcttgtcaaagagccggcggctcgaaaatctgatcgcacagcttccgggagaaaagaaaaacggact
gtttggaaaccttatcgccctctcgctgggactcactcctaacttcaaatccaattttgatttggccgagg
atgccaagctgcagctgtcgaaggacacctacgacgatgacctcgacaatcttctggcccagatcggggac
cagtacgccgatctgttcctcgcggccaaaaacctgtcagacgcaatcttgctgagcgatatcttgagggt
caataccgaaatcactaaggctccattgtcagcatcgatgatcaagaggtacgatgaacaccatcaggacc
tgaccctgctcaaggcactggtgcggcagcaactgccggaaaagtacaaggagatcttcttcgatcaaagc
aagaatgggtacgcagggtacatcgatggtggagcatcacaagaagagttctacaaattcatcaagcccat
ccttgaaaagatggacgggacggaagaactgctggtgaagctgaatcgggaagatctgctgcggaagcagc
gcaccttcgacaatggttccatcccacaccaaatccatctcggcgaactgcacgcgatccttcgccggcag
gaagatttctacccgttcttgaaggataacagagaaaagatcgagaaaatcctgacctttagaatcccgta
ctacgtgggcccgttggctcgcggaaactcaagattcgcctggatgactagaaaatccgaagaaacgatta
ccccgtggaactttgaagaggtcgtcgacaagggagcctcggcacagtcgttcatcgaacggatgaccaat
ttcgacaagaacctccccaacgaaaaggtgctcccgaaacactcgctgctgtatgagtacttcacggtcta
caacgagctgaccaaggtgaagtacgtgaccgaaggaatgcggaagcccgcttttctgtccggtgaacaga
aaaaggccatcgtggacctcctcttcaagactaacagaaaggtgaccgtgaaacagcttaaagaggactac
ttcaagaagatcgagtgttttgactccgtggaaatctcgggagtggaggataggtttaacgcttcgctggg
gacctaccatgaccttctcaagatcatcaaggataaggatttcctggacaacgaagaaaacgaggacatcc
ttgaggacatcgtgttgaccttgaccctgtttgaggatcgggaaatgatcgaggagagactgaaaacttac
gcgcatcttttcgacgacaaagtgatgaagcagctgaaaagaagaagatacactggctggggacggctgtc
gaggaaactgattaacggaattagggataaacaaagcggaaagactatcctcgattttctcaagtcggacg
gcttcgccaatcggaactttatgcagctcatccacgacgatagcctgaccttcaaggaagatatccaaaaa
gcccaagtgtccggccagggagatagcttgcacgagcacattgctaacctggccggttcaccagccattaa
gaagggaatcctccaaaccgtgaaggtggtcgacgaactcgtcaaggtgatgggccgccataagccagaga
acatcgtgatcgagatggctcgcgaaaatcagactactcagaagggtcaaaagaactcccgcgagcgcatg
aagcgcatcgaagaaggaattaaggagctgggctcacaaatcctcaaagaacatcctgtcgagaacaccca
acttcagaacgagaaactgtacctctactatctccaaaatggccgggacatgtacgtcgatcaggaattgg
atatcaaccgcctctcagactacgacgtggatgctatcgtgccgcagtcgtttctcaaagatgatagcatc
gacaacaaggtcctgacgaggtcggataagaaccgcgggaaatccgacaatgtgccttcggaagaggtggt
gaaaaagatgaagaactactggaggcaattgcttaatgccaaactgatcacccagcgcaaattcgacaacc
ttactaaggccgagcgcggaggactgtccgaactcgataaggcggggttcatcaaaagacaattggtcgaa
actcggcaaattaccaagcatgtcgctcagatcctcgactcgcgcatgaacactaagtatgatgagaacga
caagctcattcgcgaagtgaaagtgattaccctcaaatcaaagctggtgtcggacttcagaaaagactttc
aattctacaaggtgcgggagattaacaactaccaccacgcgcacgacgcctacctcaatgcagtggtggga
accgccctcatcaagaagtatccaaagctcgaaagcgagttcgtgtacggggactataaagtgtacgatgt
gcggaaaatgatcgcaaagagcgagcaagagatcggcaaagcaacggccaaatacttcttctacagcaaca
ttatgaactttttcaagaccgagattaccctggcgaacggagaaatccggaagcggcctttgatcgaaacg
aatggggaaactggagaaatcgtgtgggacaagggcagagactttgcgaccgtgagaaaggtgctgtccat
gccacaagtcaacatcgtgaagaaaactgaagtgcagactggaggattttccaaggagtcaatccttccga
agcgcaacagcgacaagctgatcgccaggaagaaggattgggaccccaagaagtacggtggttttgattca
cctactgtcgcttacagcgtgctggtggtggccaaggtggaaaaggggaagtcaaagaaattgaagtcggt
gaaggaattgctggggattactatcatggagaggagcagcttcgaaaagaatcccatcgacttcttggagg
ccaagggatacaaagaggtgaagaaagacctgatcattaagctgccgaagtactccttgttcgaactggaa
aacggcagaaagcggatgctggcctccgccggagagctgcagaagggtaacgagctggctctgcccagcaa
atacgtgaatttcctctacctggcctcccattacgagaagctcaagggaagcccggaggataatgagcaaa
aacaactgttcgtcgagcagcacaagcactacctcgacgagatcatcgaacaaatctccgagttctcgaag
cgggtgattctggccgacgcaaaccttgataaagtcctcagcgcctacaacaagcaccgcgacaaaccgat
cagagaacaagctgagaacatcatccacctgttcacgctgactaacttgggagcgcctgccgccttcaagt
acttcgacaccactatcgatcggaaacggtacacttccactaaggaagtgctggacgcaaccctgatccat
cagtccatcaccggactttatgagactcggatcgatctcagccagcttggcggtgattcaagagctgaccc
gaagaagaagcgcaaggtctaa
Seq ID NO: 17 VPR-dCas9 with NLS
atggacgcattggacgattttgatctggatatgctgggaagtgacgccctcgatgattttgaccttgacat
gcttggttcggatgcccttgatgactttgacctcgacatgctcggcagtgacgcccttgatgatttcgacc
tggacatgctgattaactctagaagttccggatctccgaaaaagaaacgcaaagttggtagccagtacctg
cccgacaccgacgaccggcaccggatcgaggaaaagcggaagcggacctacgagacattcaagagcatcat
gaagaagtcccccttcagcggccccaccgaccctaggcctccacctagaagaatcgccgtgcccagcagat
ccagcgccagcgtgccaaaacctgccccccagccttaccccttcaccagcagcctgagcaccatcaactac
gacgagttccctaccatggtgttccccagcggccagatctctcaggcctctgctctggccccagcccctcc
tcaggtgctgcctcaggctcctgctcctgcaccagctccagccatggtgtctgcactggctcaggcaccag
cacccgtgcctgtgctggctcctggacctccacaggctgtggctccaccagcccctaaacctacacaggcc
ggcgagggcacactgtctgaagctctgctgcagctgcagttcgacgacgaggatctgggagccctgctggg
aaacagcaccgatcctgccgtgttcaccgacctggccagcgtggacaacagcgagttccagcagctgctga
accagggcatccctgtggcccctcacaccaccgagcccatgctgatggaataccccgaggccatcacccgg
ctcgtgacaggcgctcagaggcctcctgatccagctcctgcccctctgggagcaccaggcctgcctaatgg
actgctgtctggcgacgaggacttcagctctatcgccgatatggatttctcagccttgctgggctctggca
gcggcagccgggattccagggaagggatgtttttgccgaagcctgaggccggctccgctattagtgacgtg
tttgagggccgcgaggtgtgccagccaaaacgaatccggccatttcatcctccaggaagtccatgggccaa
ccgcccactccccgccagcctcgcaccaacaccaaccggtccagtacatgagccagtcgggtcactgaccc
cggcaccagtccctcagccactggatccagcgcccgcagtgactcccgaggccagtcacctgttggaggat
cccgatgaagaaacgagccaggctgtcaaagcccttcgggagatggccgatactgtgattccccagaagga
agaggctgcaatctgtggccaaatggacctttcccatccgcccccaaggggccatctggatgagctgacaa
ccacacttgagtccatgaccgaggatctgaacctggactcacccctgaccccggaattgaacgagattctg
gataccttcctgaacgacgagtgcctcttgcatgccatgcatatcagcacaggactgtccatcttcgacac
atctctgtttaaagcctatgacaagaaatactccatcggccttgccatcgggaccaacagcgtgggttggg
ccgtgatcactgacgaatacaaagtgccatcgaaaaagtttaaggtgctgggcaatactgacagacatagc
atcaagaaaaacctgattggagccctcctgttcgactccggggaaaccgccgaagccactcgcctgaaacg
gaccgcacgccggaggtacactcgccggaagaatagaatctgctaccttcaagagatctttagcaacgaga
tggccaaagtggacgattcgttcttccaccggctggaggagtcattcctggtcgaagaggacaaaaagcat
gagagacatccgatcttcggtaacattgtggatgaggtcgcctaccacgagaagtaccccactatctacca
tttgcgcaagaagttggtcgactcgactgataaggccgacctcagactcatctacctcgctttggcacaca
tgatcaagtttaggggtcacttcctgattgaaggggatctgaacccagacaactcggatgtggataaactg
tttatccagctggtccagacttacaatcagctctttgaggagaacccgatcaatgcctccggcgtggatgc
taaggcaatcctgtccgcacgcttgtcaaagagccggcggctcgaaaatctgatcgcacagcttccgggag
aaaagaaaaacggactgtttggaaaccttatcgccctctcgctgggactcactcctaacttcaaatccaat
tttgatttggccgaggatgccaagctgcagctgtcgaaggacacctacgacgatgacctcgacaatcttct
ggcccagatcggggaccagtacgccgatctgttcctcgcggccaaaaacctgtcagacgcaatcttgctga
gcgatatcttgagggtcaataccgaaatcactaaggctccattgtcagcatcgatgatcaagaggtacgat
gaacaccatcaggacctgaccctgctcaaggcactggtgcggcagcaactgccggaaaagtacaaggagat
cttcttcgatcaaagcaagaatgggtacgcagggtacatcgatggtggagcatcacaagaagagttctaca
aattcatcaagcccatccttgaaaagatggacgggacggaagaactgctggtgaagctgaatcgggaagat
ctgctgcggaagcagcgcaccttcgacaatggttccatcccacaccaaatccatctcggcgaactgcacgc
gatccttcgccggcaggaagatttctacccgttcttgaaggataacagagaaaagatcgagaaaatcctga
cctttagaatcccgtactacgtgggcccgttggctcgcggaaactcaagattcgcctggatgactagaaaa
tccgaagaaacgattaccccgtggaactttgaagaggtcgtcgacaagggagcctcggcacagtcgttcat
cgaacggatgaccaatttcgacaagaacctccccaacgaaaaggtgctcccgaaacactcgctgctgtatg
agtacttcacggtctacaacgagctgaccaaggtgaagtacgtgaccgaaggaatgcggaagcccgctttt
ctgtccggtgaacagaaaaaggccatcgtggacctcctcttcaagactaacagaaaggtgaccgtgaaaca
gcttaaagaggactacttcaagaagatcgagtgttttgactccgtggaaatctcgggagtggaggataggt
ttaacgcttcgctggggacctaccatgaccttctcaagatcatcaaggataaggatttcctggacaacgaa
gaaaacgaggacatccttgaggacatcgtgttgaccttgaccctgtttgaggatcgggaaatgatcgagga
gagactgaaaacttacgcgcatcttttcgacgacaaagtgatgaagcagctgaaaagaagaagatacactg
gctggggacggctgtcgaggaaactgattaacggaattagggataaacaaagcggaaagactatcctcgat
tttctcaagtcggacggcttcgccaatcggaactttatgcagctcatccacgacgatagcctgaccttcaa
ggaagatatccaaaaagcccaagtgtccggccagggagatagcttgcacgagcacattgctaacctggccg
gttcaccagccattaagaagggaatcctccaaaccgtgaaggtggtcgacgaactcgtcaaggtgatgggc
cgccataagccagagaacatcgtgatcgagatggctcgcgaaaatcagactactcagaagggtcaaaagaa
ctcccgcgagcgcatgaagcgcatcgaagaaggaattaaggagctgggctcacaaatcctcaaagaacatc
ctgtcgagaacacccaacttcagaacgagaaactgtacctctactatctccaaaatggccgggacatgtac
gtcgatcaggaattggatatcaaccgcctctcagactacgacgtggatgctatcgtgccgcagtcgtttct
caaagatgatagcatcgacaacaaggtcctgacgaggtcggataagaaccgcgggaaatccgacaatgtgc
cttcggaagaggtggtgaaaaagatgaagaactactggaggcaattgcttaatgccaaactgatcacccag
cgcaaattcgacaaccttactaaggccgagcgcggaggactgtccgaactcgataaggcggggttcatcaa
aagacaattggtcgaaactcggcaaattaccaagcatgtcgctcagatcctcgactcgcgcatgaacacta
agtatgatgagaacgacaagctcattcgcgaagtgaaagtgattaccctcaaatcaaagctggtgtcggac
ttcagaaaagactttcaattctacaaggtgcgggagattaacaactaccaccacgcgcacgacgcctacct
caatgcagtggtgggaaccgccctcatcaagaagtatccaaagctcgaaagcgagttcgtgtacggggact
ataaagtgtacgatgtgcggaaaatgatcgcaaagagcgagcaagagatcggcaaagcaacggccaaatac
ttcttctacagcaacattatgaactttttcaagaccgagattaccctggcgaacggagaaatccggaagcg
gcctttgatcgaaacgaatggggaaactggagaaatcgtgtgggacaagggcagagactttgcgaccgtga
gaaaggtgctgtccatgccacaagtcaacatcgtgaagaaaactgaagtgcagactggaggattttccaag
gagtcaatccttccgaagcgcaacagcgacaagctgatcgccaggaagaaggattgggaccccaagaagta
cggtggttttgattcacctactgtcgcttacagcgtgctggtggtggccaaggtggaaaaggggaagtcaa
agaaattgaagtcggtgaaggaattgctggggattactatcatggagaggagcagcttcgaaaagaatccc
atcgacttcttggaggccaagggatacaaagaggtgaagaaagacctgatcattaagctgccgaagtactc
cttgttcgaactggaaaacggcagaaagcggatgctggcctccgccggagagctgcagaagggtaacgagc
tggctctgcccagcaaatacgtgaatttcctctacctggcctcccattacgagaagctcaagggaagcccg
gaggataatgagcaaaaacaactgttcgtcgagcagcacaagcactacctcgacgagatcatcgaacaaat
ctccgagttctcgaagcgggtgattctggccgacgcaaaccttgataaagtcctcagcgcctacaacaagc
accgcgacaaaccgatcagagaacaagctgagaacatcatccacctgttcacgctgactaacttgggagcg
cctgccgccttcaagtacttcgacaccactatcgatcggaaacggtacacttccactaaggaagtgctgga
cgcaaccctgatccatcagtccatcaccggactttatgagactcggatcgatctcagccagcttggcggtg
attcaagagctgacccgaagaagaagcgcaaggtctaa
Seq ID NO: 18-AcrII4A
atgaacatcaacgatctgatccgggagatcaagaacaaggactacaccgtgaagctgagcggcaccgatag
caacagcatcacccagctgattatccgcgtgaacaacgacggcaacgagtacgtgatctccgagagcgaga
acgagagcatcgtggagaagttcatcagcgccttcaagaacggttggaaccaggagtacgaggacgaggag
gagttctacaacgacatgcagaccatcaccctgaagagcgagctgaactaa
Seq ID NO: 19-skipped
Seq ID NO: 20-pCMV/TO-CasRx-NLS-T2A-mCherry
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctctccctatcagtgatagagatctccctatcagtgatagagatcgtcgacgaggacttagc
ctttagtgaaccgtcagaattaattcagatcgatctaccagaaccgtcagatccgctagagattacgccaa
ccgccaccatgggcagcatgagccccaagaagaagagaaaggtggaggccagcatcgaaaaaaaaaagtcc
ttcgccaagggcatgggcgtgaagtccacactcgtgtccggctccaaagtgtacatgacaaccttcgccga
aggcagcgacgccaggctggaaaagatcgtggagggcgacagcatcaggagcgtgaatgagggcgaggcct
tcagcgctgaaatggccgataaaaacgccggctataagatcggcaacgccaaattcagccatcctaagggc
tacgccgtggtggctaacaaccctctgtatacaggacccgtccagcaggatatgctcggcctgaaggaaac
tctggaaaagaggtacttcggcgagagcgctgatggcaatgacaatatttgtatccaggtgatccataaca
tcctggacattgaaaaaatcctcgccgaatacattaccaacgccgcctacgccgtcaacaatatctccggc
ctggataaggacattattggattcggcaagttctccacagtgtatacctacgacgaattcaaagaccccga
gcaccatagggccgctttcaacaataacgataagctcatcaacgccatcaaggcccagtatgacgagttcg
acaacttcctcgataaccccagactcggctatttcggccaggcctttttcagcaaggagggcagaaattac
atcatcaattacggcaacgaatgctatgacattctggccctcctgagcggactgaggcactgggtggtcca
taacaacgaagaagagtccaggatctccaggacctggctctacaacctcgataagaacctcgacaacgaat
acatctccaccctcaactacctctacgacaggatcaccaatgagctgaccaactccttctccaagaactcc
gccgccaacgtgaactatattgccgaaactctgggaatcaaccctgccgaattcgccgaacaatatttcag
attcagcattatgaaagagcagaaaaacctcggattcaatatcaccaagctcagggaagtgatgctggaca
ggaaggatatgtccgagatcaggaaaaatcataaggtgttcgactccatcaggaccaaggtctacaccatg
atggactttgtgatttataggtattacatcgaagaggatgccaaggtggctgccgccaataagtccctccc
cgataatgagaagtccctgagcgagaaggatatctttgtgattaacctgaggggctccttcaacgacgacc
agaaggatgccctctactacgatgaagctaatagaatttggagaaagctcgaaaatatcatgcacaacatc
aaggaatttaggggaaacaagacaagagagtataagaagaaggacgcccctagactgcccagaatcctgcc
cgctggccgtgatgtttccgccttcagcaaactcatgtatgccctgaccatgttcctggatggcaaggaga
tcaacgacctcctgaccaccctgattaataaattcgataacatccagagcttcctgaaggtgatgcctctc
atcggagtcaacgctaagttcgtggaggaatacgcctttttcaaagactccgccaagatcgccgatgagct
gaggctgatcaagtccttcgctagaatgggagaacctattgccgatgccaggagggccatgtatatcgacg
ccatccgtattttaggaaccaacctgtcctatgatgagctcaaggccctcgccgacaccttttccctggac
gagaacggaaacaagctcaagaaaggcaagcacggcatgagaaatttcattattaataacgtgatcagcaa
taaaaggttccactacctgatcagatacggtgatcctgcccacctccatgagatcgccaaaaacgaggccg
tggtgaagttcgtgctcggcaggatcgctgacatccagaaaaaacagggccagaacggcaagaaccagatc
gacaggtactacgaaacttgtatcggaaaggataagggcaagagcgtgagcgaaaaggtggacgctctcac
aaagatcatcaccggaatgaactacgaccaattcgacaagaaaaggagcgtcattgaggacaccggcaggg
aaaacgccgagagggagaagtttaaaaagatcatcagcctgtacctcaccgtgatctaccacatcctcaag
aatattgtcaatatcaacgccaggtacgtcatcggattccattgcgtcgagcgtgatgctcaactgtacaa
ggagaaaggctacgacatcaatctcaagaaactggaagagaagggattcagctccgtcaccaagctctgcg
ctggcattgatgaaactgcccccgataagagaaaggacgtggaaaaggagatggctgaaagagccaaggag
agcattgacagcctcgagagcgccaaccccaagctgtatgccaattacatcaaatacagcgacgagaagaa
agccgaggagttcaccaggcagattaacagggagaaggccaaaaccgccctgaacgcctacctgaggaaca
ccaagtggaatgtgatcatcagggaggacctcctgagaattgacaacaagacatgtaccctgttcagaaac
aaggccgtccacctggaagtggccaggtatgtccacgcctatatcaacgacattgccgaggtcaattccta
cttccaactgtaccattacatcatgcagagaattatcatgaatgagaggtacgagaaaagcagcggaaagg
tgtccgagtacttcgacgctgtgaatgacgagaagaagtacaacgataggctcctgaaactgctgtgtgtg
cctttcggctactgtatccccaggtttaagaacctgagcatcgaggccctgttcgataggaacgaggccgc
caagttcgacaaggagaaaaagaaggtgtccggcaattccggatccggacctaagaaaaagaggaaggtgg
cggccgcttacccatacgatgttccagattacgctagcggctcaggcgagggcagaggaagtctgctaaca
tgcggtgacgtcgaggagaatcctggcccagcgtccgtgagcaagggcgaggaggataacatggccatcat
caaggagttcatgcgcttcaaggtgcacatggagggctccgtgaacggccacgagttcgagatcgagggcg
agggcgagggccgcccctacgagggcacccagaccgccaagctgaaggtgaccaagggtggccccctgccc
ttcgcctgggacatcctgtcccctcagttcatgtacggctccaaggcctacgtgaagcaccccgccgacat
ccccgactacttgaagctgtccttccccgagggcttcaagtgggagcgcgtgatgaacttcgaggacggcg
gcgtggtgaccgtgacccaggactcctccctgcaggacggcgagttcatctacaaggtgaagctgcgcggc
accaacttcccctccgacggccccgtaatgcagaagaagaccatgggctgggaggcctcctccgagcggat
gtaccccgaggacggcgccctgaagggcgagatcaagcagaggctgaagctgaaggacggcggccactacg
acgctgaggtcaagaccacctacaaggccaagaagcccgtgcagctgcccggcgcctacaacgtcaacatc
aagttggacatcacctcccacaacgaggactacaccatcgtggaacagtacgaacgcgccgagggccgcca
ctccaccggcggcatggacgagctgtacaagtaa
Seq ID NO: 21 N7
atgggggaaattgctgctctggaagccaaaaatgcggcgttgaaagccgagattgcggccttggaagctaa
gatcgctgctttaaaggccggatactaa
Seq ID NO: 22 N8
atgttaaaggcggagaacgcagctctggaagccaagattgcagccttaaaagcggagattgctgcgttaga
ggcaggctactaa
Seq ID NO: 23 AcrII4A-N7
atgaacatcaacgatctgatccgggagatcaagaacaaggactacaccgtgaagctgagcggcaccgatag
caacagcatcacccagctgattatccgcgtgaacaacgacggcaacgagtacgtgatctccgagagcgaga
acgagagcatcgtggagaagttcatcagcgccttcaagaacggttggaaccaggagtacgaggacgaggag
gagttctacaacgacatgcagaccatcaccctgaagagcgagctgaactcaggctctggactcagatctgg
cagcggaggaggcagcgcatctggcggaagcggagcgtccggggaaattgctgctctggaagccaaaaatg
cggcgttgaaagccgagattgcggccttggaagctaagatcgctgctttaaaggccggatactaa
Seq ID NO: 24 VPR-Cas9-N8
atggacgcattggacgattttgatctggatatgctgggaagtgacgccctcgatgattttgaccttgacat
gcttggttcggatgcccttgatgactttgacctcgacatgctcggcagtgacgcccttgatgatttcgacc
tggacatgctgattaactctagaagttccggatctccgaaaaagaaacgcaaagttggtagccagtacctg
cccgacaccgacgaccggcaccggatcgaggaaaagcggaagcggacctacgagacattcaagagcatcat
gaagaagtcccccttcagcggccccaccgaccctaggcctccacctagaagaatcgccgtgcccagcagat
ccagcgccagcgtgccaaaacctgccccccagccttaccccttcaccagcagcctgagcaccatcaactac
gacgagttccctaccatggtgttccccagcggccagatctctcaggcctctgctctggccccagcccctcc
tcaggtgctgcctcaggctcctgctcctgcaccagctccagccatggtgtctgcactggctcaggcaccag
cacccgtgcctgtgctggctcctggacctccacaggctgtggctccaccagcccctaaacctacacaggcc
ggcgagggcacactgtctgaagctctgctgcagctgcagttcgacgacgaggatctgggagccctgctggg
aaacagcaccgatcctgccgtgttcaccgacctggccagcgtggacaacagcgagttccagcagctgctga
accagggcatccctgtggcccctcacaccaccgagcccatgctgatggaataccccgaggccatcacccgg
ctcgtgacaggcgctcagaggcctcctgatccagctcctgcccctctgggagcaccaggcctgcctaatgg
actgctgtctggcgacgaggacttcagctctatcgccgatatggatttctcagccttgctgggctctggca
gcggcagccgggattccagggaagggatgtttttgccgaagcctgaggccggctccgctattagtgacgtg
tttgagggccgcgaggtgtgccagccaaaacgaatccggccatttcatcctccaggaagtccatgggccaa
ccgcccactccccgccagcctcgcaccaacaccaaccggtccagtacatgagccagtcgggtcactgaccc
cggcaccagtccctcagccactggatccagcgcccgcagtgactcccgaggccagtcacctgttggaggat
cccgatgaagaaacgagccaggctgtcaaagcccttcgggagatggccgatactgtgattccccagaagga
agaggctgcaatctgtggccaaatggacctttcccatccgcccccaaggggccatctggatgagctgacaa
ccacacttgagtccatgaccgaggatctgaacctggactcacccctgaccccggaattgaacgagattctg
gataccttcctgaacgacgagtgcctcttgcatgccatgcatatcagcacaggactgtccatcttcgacac
atctctgtttaaagcctatgacaagaaatactccatcggccttgccatcgggaccaacagcgtgggttggg
ccgtgatcactgacgaatacaaagtgccatcgaaaaagtttaaggtgctgggcaatactgacagacatagc
atcaagaaaaacctgattggagccctcctgttcgactccggggaaaccgccgaagccactcgcctgaaacg
gaccgcacgccggaggtacactcgccggaagaatagaatctgctaccttcaagagatctttagcaacgaga
tggccaaagtggacgattcgttcttccaccggctggaggagtcattcctggtcgaagaggacaaaaagcat
gagagacatccgatcttcggtaacattgtggatgaggtcgcctaccacgagaagtaccccactatctacca
tttgcgcaagaagttggtcgactcgactgataaggccgacctcagactcatctacctcgctttggcacaca
tgatcaagtttaggggtcacttcctgattgaaggggatctgaacccagacaactcggatgtggataaactg
tttatccagctggtccagacttacaatcagctctttgaggagaacccgatcaatgcctccggcgtggatgc
taaggcaatcctgtccgcacgcttgtcaaagagccggcggctcgaaaatctgatcgcacagcttccgggag
aaaagaaaaacggactgtttggaaaccttatcgccctctcgctgggactcactcctaacttcaaatccaat
tttgatttggccgaggatgccaagctgcagctgtcgaaggacacctacgacgatgacctcgacaatcttct
ggcccagatcggggaccagtacgccgatctgttcctcgcggccaaaaacctgtcagacgcaatcttgctga
gcgatatcttgagggtcaataccgaaatcactaaggctccattgtcagcatcgatgatcaagaggtacgat
gaacaccatcaggacctgaccctgctcaaggcactggtgcggcagcaactgccggaaaagtacaaggagat
cttcttcgatcaaagcaagaatgggtacgcagggtacatcgatggtggagcatcacaagaagagttctaca
aattcatcaagcccatccttgaaaagatggacgggacggaagaactgctggtgaagctgaatcgggaagat
ctgctgcggaagcagcgcaccttcgacaatggttccatcccacaccaaatccatctcggcgaactgcacgc
gatccttcgccggcaggaagatttctacccgttcttgaaggataacagagaaaagatcgagaaaatcctga
cctttagaatcccgtactacgtgggcccgttggctcgcggaaactcaagattcgcctggatgactagaaaa
tccgaagaaacgattaccccgtggaactttgaagaggtcgtcgacaagggagcctcggcacagtcgttcat
cgaacggatgaccaatttcgacaagaacctccccaacgaaaaggtgctcccgaaacactcgctgctgtatg
agtacttcacggtctacaacgagctgaccaaggtgaagtacgtgaccgaaggaatgcggaagcccgctttt
ctgtccggtgaacagaaaaaggccatcgtggacctcctcttcaagactaacagaaaggtgaccgtgaaaca
gcttaaagaggactacttcaagaagatcgagtgttttgactccgtggaaatctcgggagtggaggataggt
ttaacgcttcgctggggacctaccatgaccttctcaagatcatcaaggataaggatttcctggacaacgaa
gaaaacgaggacatccttgaggacatcgtgttgaccttgaccctgtttgaggatcgggaaatgatcgagga
gagactgaaaacttacgcgcatcttttcgacgacaaagtgatgaagcagctgaaaagaagaagatacactg
gctggggacggctgtcgaggaaactgattaacggaattagggataaacaaagcggaaagactatcctcgat
tttctcaagtcggacggcttcgccaatcggaactttatgcagctcatccacgacgatagcctgaccttcaa
ggaagatatccaaaaagcccaagtgtccggccagggagatagcttgcacgagcacattgctaacctggccg
gttcaccagccattaagaagggaatcctccaaaccgtgaaggtggtcgacgaactcgtcaaggtgatgggc
cgccataagccagagaacatcgtgatcgagatggctcgcgaaaatcagactactcagaagggtcaaaagaa
ctcccgcgagcgcatgaagcgcatcgaagaaggaattaaggagctgggctcacaaatcctcaaagaacatc
ctgtcgagaacacccaacttcagaacgagaaactgtacctctactatctccaaaatggccgggacatgtac
gtcgatcaggaattggatatcaaccgcctctcagactacgacgtggatgctatcgtgccgcagtcgtttct
caaagatgatagcatcgacaacaaggtcctgacgaggtcggataagaaccgcgggaaatccgacaatgtgc
cttcggaagaggtggtgaaaaagatgaagaactactggaggcaattgcttaatgccaaactgatcacccag
cgcaaattcgacaaccttactaaggccgagcgcggaggactgtccgaactcgataaggcggggttcatcaa
aagacaattggtcgaaactcggcaaattaccaagcatgtcgctcagatcctcgactcgcgcatgaacacta
agtatgatgagaacgacaagctcattcgcgaagtgaaagtgattaccctcaaatcaaagctggtgtcggac
ttcagaaaagactttcaattctacaaggtgcgggagattaacaactaccaccacgcgcacgacgcctacct
caatgcagtggtgggaaccgccctcatcaagaagtatccaaagctcgaaagcgagttcgtgtacggggact
ataaagtgtacgatgtgcggaaaatgatcgcaaagagcgagcaagagatcggcaaagcaacggccaaatac
ttcttctacagcaacattatgaactttttcaagaccgagattaccctggcgaacggagaaatccggaagcg
gcctttgatcgaaacgaatggggaaactggagaaatcgtgtgggacaagggcagagactttgcgaccgtga
gaaaggtgctgtccatgccacaagtcaacatcgtgaagaaaactgaagtgcagactggaggattttccaag
gagtcaatccttccgaagcgcaacagcgacaagctgatcgccaggaagaaggattgggaccccaagaagta
cggtggttttgattcacctactgtcgcttacagcgtgctggtggtggccaaggtggaaaaggggaagtcaa
agaaattgaagtcggtgaaggaattgctggggattactatcatggagaggagcagcttcgaaaagaatccc
atcgacttcttggaggccaagggatacaaagaggtgaagaaagacctgatcattaagctgccgaagtactc
cttgttcgaactggaaaacggcagaaagcggatgctggcctccgccggagagctgcagaagggtaacgagc
tggctctgcccagcaaatacgtgaatttcctctacctggcctcccattacgagaagctcaagggaagcccg
gaggataatgagcaaaaacaactgttcgtcgagcagcacaagcactacctcgacgagatcatcgaacaaat
ctccgagttctcgaagcgggtgattctggccgacgcaaaccttgataaagtcctcagcgcctacaacaagc
accgcgacaaaccgatcagagaacaagctgagaacatcatccacctgttcacgctgactaacttgggagcg
cctgccgccttcaagtacttcgacaccactatcgatcggaaacggtacacttccactaaggaagtgctgga
cgcaaccctgatccatcagtccatcaccggactttatgagactcggatcgatctcagccagcttggcggtg
attcaagagctgacccgaagaagaagcgcaaggtctcaggctctggactcagatctggcagcggaggaggc
agcgcatctggcggaagcggagcgtccttaaaggcggagaacgcagctctggaagccaagattgcagcctt
aaaagcggagattgctgcgttagaggcaggctactaa
Seq ID NO: 25-DD-mCherry
atgggagtgcaagtggaaaccatctccccaggagatgggcgcaccttccccaagcgcggccagacctgtgt
ggtgcactacaccgggatgcttgaagatggaaagaaagtcgattcctcccgggacagaaacaagcccttta
agtttatgctaggcaagcaggaggtgatccgaggctgggaagaaggggttgcccagatgagtgtgggtcag
agagccaaactgactatatctccagattatgcctatggtgccactgggcacccaggcatcatcccaccaca
tgccactctcgtcttcgatgtggagcttctaaaacctgaaggcagcgtgagcaagggcgaggaggataaca
tggccatcatcaaggagttcatgcgcttcaaggtgcacatggagggctccgtgaacggccacgagttcgag
atcgagggcgagggcgagggccgcccctacgagggcacccagaccgccaagctgaaggtgaccaagggtgg
ccccctgcccttcgcctgggacatcctgtcccctcagttcatgtacggctccaaggcctacgtgaagcacc
ccgccgacatccccgactacttgaagctgtccttccccgagggcttcaagtgggagcgcgtgatgaacttc
gaggacggcggcgtggtgaccgtgacccaggactcctccctgcaggacggcgagttcatctacaaggtgaa
gctgcgcggcaccaacttcccctccgacggccccgtaatgcagaagaagaccatgggctgggaggcctcct
ccgagcggatgtaccccgaggacggcgccctgaagggcgagatcaagcagaggctgaagctgaaggacggc
ggccactacgacgctgaggtcaagaccacctacaaggccaagaagcccgtgcagctgcccggcgcctacaa
cgtcaacatcaagttggacatcacctcccacaacgaggactacaccatcgtggaacagtacgaacgcgccg
agggccgccactccaccggcggcatggacgagctgtacaagtcaggctaa
Seq ID NO: 26-DD-rtTA
atgggagtgcaagtggaaaccatctccccaggagatgggcgcaccttccccaagcgcggccagacctgtgt
ggtgcactacaccgggatgcttgaagatggaaagaaagtcgattcctcccgggacagaaacaagcccttta
agtttatgctaggcaagcaggaggtgatccgaggctgggaagaaggggttgcccagatgagtgtgggtcag
agagccaaactgactatatctccagattatgcctatggtgccactgggcacccaggcatcatcccaccaca
tgccactctcgtcttcgatgtggagcttctaaaacctgaaggcagctctagactggacaagagcaaagtca
taaactctgctctggaattactcaatggagtcggtatcgaaggcctgacgacaaggaaactcgctcaaaag
ctgggagttgagcagcctaccctgtactggcacgtgaagaacaagcgggccctgctcgatgccctgccaat
cgagatgctggacaggcatcatacccactcctgccccctggaaggcgagtcatggcaagactttctgcgga
acaacgccaagtcataccgctgtgctctcctctcacatcgcgacggggctaaagtgcatctcggcacccgc
ccaacagagaaacagtacgaaaccctggaaaatcagctcgcgttcctgtgtcagcaaggcttctccctgga
gaacgcactgtacgctctgtccgccgtgggccactttacactgggctgcgtattggaggaacaggagcatc
aagtagcaaaagaggaaagagagacacctaccaccgattctatgcccccacttctgaaacaagcaattgag
ctgttcgaccggcagggagccgaacctgccttccttttcggcctggaactaatcatatgtggcctggagaa
acagctaaagtgcgaaagcggcgggccgaccgacgcccttgacgattttgacttagacatgctcccagccg
atgcccttgacgactttgaccttgatatgctgcctgctgacgctcttgacgattttgaccttgacatgctc
cccgggttaa
Seq ID NO: 27-pCMV/TO-mCherry
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctctccctatcagtgatagagatctccctatcagtgatagagatcgtcgacgagtcgtcgac
gagggacttagcctttagtgaaccgtcagaattaattcagatcgatctaccagaaccgtcagatccgctag
agattacgccaaccgccaccatgggcagcgtgagcaagggcgaggaggataacatggccatcatcaaggag
ttcatgcgcttcaaggtgcacatggagggctccgtgaacggccacgagttcgagatcgagggcgagggcga
gggccgcccctacgagggcacccagaccgccaagctgaaggtgaccaagggtggccccctgcccttcgcct
gggacatcctgtcccctcagttcatgtacggctccaaggcctacgtgaagcaccccgccgacatccccgac
tacttgaagctgtccttccccgagggcttcaagtgggagcgcgtgatgaacttcgaggacggcggcgtggt
gaccgtgacccaggactcctccctgcaggacggcgagttcatctacaaggtgaagctgcgcggcaccaact
tcccctccgacggccccgtaatgcagaagaagaccatgggctgggaggcctcctccgagcggatgtacccc
gaggacggcgccctgaagggcgagatcaagcagaggctgaagctgaaggacggcggccactacgacgctga
ggtcaagaccacctacaaggccaagaagcccgtgcagctgcccggcgcctacaacgtcaacatcaagttgg
acatcacctcccacaacgaggactacaccatcgtggaacagtacgaacgcgccgagggccgccactccacc
ggcggcatggacgagctgtacaag
Seq ID NO: 28 pCMV-Gaussia luciferase-DR
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctggacttagcctttagtgaaccgtcagaattaattcagatcgatctaccagaaccgtcaga
tccgctagagattacgccaaccgccaccatgggcagcggagtcaaagttctgtttgccctgatctgcatcg
ctgtggccgaggccaagcccaccgagaacaacgaagacttcaacatcgtggccgtggccagcaacttcgcg
accacggatctcgatgctgaccgcgggaagttgcccggcaagaagctgccgctggaggtgctcaaagagat
ggaagccaatgcccggaaagctggctgcaccaggggctgtctgatctgcctgtcccacatcaagtgcacgc
ccaagatgaagaagttcatcccaggacgctgccacacctacgaaggcgacaaagagtccgcacagggcggc
ataggcgaggcgatcgtcgacattcctgagattcctgggttcaaggacttggagcccatggagcagttcat
cgcacaggtcgatctgtgtgtggactgcacaactggctgcctcaaagggcttgccaacgtgcagtgttctg
acctgctcaagaagtggctgccgcaacgctgtgcgacctttgccagcaagatccagggccaggtggacaag
atcaagggggccggtggtgacaggctaataacagcttccggactctagaacatccctacaggtgatatcct
cgtgctgcaagtaaacccctaccaactggtcggggtttgaaac
Seq ID NO: 29 pTRE3G-Gaussia luciferase-DR
gagtttactccctatcagtgatagagaacgtatgaagagtttactccctatcagtgatagagaacgtatgc
agactttactccctatcagtgatagagaacgtataaggagtttactccctatcagtgatagagaacgtatg
accagtttactccctatcagtgatagagaacgtatctacagtttactccctatcagtgatagagaacgtat
atccagtttactccctatcagtgatagagaacgtataagctttaggcgtgtacggtgggcgcctataaaag
cagagctcgtttagtgaaccgtcagatcgcctggagcaattccacaacacttttgtcttataccaactttc
cgtaccacttcctaccctcgtaaaggacttagcctttagtgaaccgtcagaattaattcagatcgatctac
cagaaccgtcagatccgctagagattacgccaaccgccaccatgggcagcggagtcaaagttctgtttgcc
ctgatctgcatcgctgtggccgaggccaagcccaccgagaacaacgaagacttcaacatcgtggccgtggc
cagcaacttcgcgaccacggatctcgatgctgaccgcgggaagttgcccggcaagaagctgccgctggagg
tgctcaaagagatggaagccaatgcccggaaagctggctgcaccaggggctgtctgatctgcctgtcccac
atcaagtgcacgcccaagatgaagaagttcatcccaggacgctgccacacctacgaaggcgacaaagagtc
cgcacagggcggcataggcgaggcgatcgtcgacattcctgagattcctgggttcaaggacttggagccca
tggagcagttcatcgcacaggtcgatctgtgtgtggactgcacaactggctgcctcaaagggcttgccaac
gtgcagtgttctgacctgctcaagaagtggctgccgcaacgctgtgcgacctttgccagcaagatccaggg
ccaggtggacaagatcaagggggccggtggtgacaggctaataacagcttccggactctagaacatcccta
caggtgatatcctcgtgctgcaagtaaacccctaccaactggtcggggtttgaaac
SEQ ID NO: 30 pTRE3G-Gaussia Luciferase (GLuc)
gagtttactccctatcagtgatagagaacgtatgaagagtttactccctatcagtgatagagaacgtatgc
agactttactccctatcagtgatagagaacgtataaggagtttactccctatcagtgatagagaacgtatg
accagtttactccctatcagtgatagagaacgtatctacagtttactccctatcagtgatagagaacgtat
atccagtttactccctatcagtgatagagaacgtataagctttaggcgtgtacggtgggcgcctataaaag
cagagctcgtttagtgaaccgtcagatcgcctggagcaattccacaacacttttgtcttataccaactttc
cgtaccacttcctaccctcgtaaaggacttagcctttagtgaaccgtcagaattaattcagatcgatctac
cagaaccgtcagatccgctagagattacgccaaccgccaccatgggcagcggagtcaaagttctgtttgcc
ctgatctgcatcgctgtggccgaggccaagcccaccgagaacaacgaagacttcaacatcgtggccgtggc
cagcaacttcgcgaccacggatctcgatgctgaccgcgggaagttgcccggcaagaagctgccgctggagg
tgctcaaagagatggaagccaatgcccggaaagctggctgcaccaggggctgtctgatctgcctgtcccac
atcaagtgcacgcccaagatgaagaagttcatcccaggacgctgccacacctacgaaggcgacaaagagtc
cgcacagggcggcataggcgaggcgatcgtcgacattcctgagattcctgggttcaaggacttggagccca
tggagcagttcatcgcacaggtcgatctgtgtgtggactgcacaactggctgcctcaaagggcttgccaac
gtgcagtgttctgacctgctcaagaagtggctgccgcaacgctgtgcgacctttgccagcaagatccaggg
ccaggtggacaagatcaagggggccggtggtgacaggctaataacagcttccggactctagaacatcccta
caggtgatatcctcgggtaa
SEQ ID NO: 31-NiClear-rtTA-VPR (clear binding sites in bold)
ccctgcgggtcacgtgacgggtcacgtgacgggtcacgtgacgggtcacgtgaaaagtgaaagtcgagttt
accactcacgtgaaaagtgaaagtcgagtttaccactcacgtgaaaagtgaaagtcgagtttaccactcac
gtgaccctgcgggaagcttagacactagagggtatataatggaagctcgacttccagggacttagccttta
gtgaaccgtcagaattaattcagatcgatctaccagaaccgtcagatccgctagagattacgccaaccgcc
accatgggcagcatgtctagactggacaagagcaaagtcataaactctgctctggaattactcaatggagt
cggtatcgaaggcctgacgacaaggaaactcgctcaaaagctgggagttgagcagcctaccctgtactggc
acgtgaagaacaagcgggccctgctcgatgccctgccaatcgagatgctggacaggcatcatacccactcc
tgccccctggaaggcgagtcatggcaagactttctgcggaacaacgccaagtcataccgctgtgctctcct
ctcacatcgcgacggggctaaagtgcatctcggcacccgcccaacagagaaacagtacgaaaccctggaaa
atcagctcgcgttcctgtgtcagcaaggcttctccctggagaacgcactgtacgctctgtccgccgtgggc
cactttacactgggctgcgtattggaggaacaggagcatcaagtagcaaaagaggaaagagagacacctac
caccgattctatgcccccacttctgaaacaagcaattgagctgttcgaccggcagggagccgaacctgcct
tccttttcggcctggaactaatcatatgtggcctggagaaacagctaaagtgcgaaagcggcgacgcattg
gacgattttgatctggatatgctgggaagtgacgccctcgatgattttgaccttgacatgcttggttcgga
tgcccttgatgactttgacctcgacatgctcggcagtgacgcccttgatgatttcgacctggacatgctga
ttaacactagtggcggctctggcggcggctccggaggctctagccagtacctgcccgacaccgacgaccgg
caccggatcgaggaaaagcggaagcggacctacgagacattcaagagcatcatgaagaagtcccccttcag
cggccccaccgaccctagacctccacctagaagaatcgccgtgcccagcagatccagcgccagcgtgccaa
aacctgccccccagccttaccccttcaccagcagcctgagcaccatcaactacgacgagttccctaccatg
gtgttccccagcggccagatctctcaggcctctgctctggctccagcccctcctcaggtgctgcctcaggc
tcctgctcctgcaccagctccagccatggtgtctgcactggctcaggcaccagcacccgtgcctgtgctgg
ctcctggacctccacaggctgtggctccaccagcccctaaacctacacaggccggcgagggcacactgtct
gaagctctgctgcagctgcagttcgacgacgaggatctgggagccctgctgggaaacagcaccgatcctgc
cgtgttcaccgacctggccagcgtggacaacagcgagttccagcagctgctgaaccagggcatccctgtgg
cccctcacaccaccgagcccatgctgatggaataccccgaggccatcacccggctcgtgacaggcgctcag
aggcctcctgatccagctcctgcccctctgggagcaccaggcctgcctaatggactgctgtctggcgacga
ggacttcagctctatcgccgatatggatttctcagccttgctgggctctggcagcggcagccgggattcca
gggaagggatgtttttgccgaagcctgaggccggctccgctattagtgacgtgtttgagggccgcgaggtg
tgccagccaaaacgaatccggccatttcatcctccaggaagtccatgggccaaccgcccactccccgccag
cctcgcaccaacaccaaccggtccagtacatgagccagtcgggtcactgaccccggcaccagtccctcagc
cactggacccagcgcccgcagtgactcccgaggccagtcacctgttggaggaccccgatgaagagacgagc
caggctgtcaaagcccttcgggagatggccgatactgtgattccccagaaggaagaggctgcaatctgtgg
ccaaatggacctttcccatccgcccccaaggggccatctggatgagctgacaaccacacttgagtccatga
ccgaggatctgaacctggactcacccctgaccccggaattgaacgagattctggataccttcctgaacgac
gagtgcctcttgcatgccatgcatatcagcacaggactgtccatcttcgacacatctctgttttga
SEQ ID NO: 32 P2A SEQUENCE
gctactaacttcagcctgctgaagcaggctggcgacgtggaggagaaccctggacct
SEQ ID NO: 33-IRES
caggctaataacagcttccggactctagaacatccctacaggtgatatcctcgatcccgctcgataagctt
gatatcgaattccgcccccccccctctccctcccccccccctaacgttactggccgaagccgcttggaata
aggccggtgtgcgtttgtctatatgttattttccaccatattgccgtcttttggcaatgtgagggcccgga
aacctggccctgtcttcttgacgagcattcctaggggtctttcccctctcgccaaaggaatgcaaggtctg
ttgaatgtcgtgaaggaagcagttcctctggaagcttcttgaagacaaacaacgtctgtagcgaccctttg
caggcagcggaaccccccacctggcgacaggtgcctctgcggccaaaagccacgtgtataagatacacctg
caaaggcggcacaaccccagtgccacgttgtgagttggatagttgtggaaagagtcaaatggctctcctca
agcgtattcaacaaggggctgaaggatgcccagaaggtaccccattgtatgggatctgatctggggcctcg
gtgcacatgctttacatgtgtttagtcgaggttaaaaaaacgtctaggccccccgaaccacggggacgtgg
ttttcctttgaaaaacacgatgataatatggccacaacc
Seqs 34-41 that follow are minimal gene sets for expressing E2A and E4
or Ad helper, comprising pCMV PROMOTER, E2A(DBP), P2A SEQUENCE, IRES
SEQUENCE, E4(ORF6), SV40 PA, VARNAI
SEQ ID NO: 34-μH 1
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctggacttagcctttagtgaaccgtcagaattaattcagatcgatctaccagaaccgtcaga
tccgctagagattacgccaaccgccaccatgggcagcgccagtcgggaagaggagcagcgcgaaaccaccc
ccgagcgcggacgcggtgcggcgcgacgtcccccaaccatggaggacgtgtcgtccccgtccccgtcgccg
ccgcctccccgggcgcccccaaaaaagcggatgaggcggcgtatcgagtccgaggacgaggaagactcatc
acaagacgcgctggtgccgcgcacacccagcccgcggccatcgacctcggcggcggatttggccattgcgc
ccaagaagaaaaagaagcgcccttctcccaagcccgagcgcccgccatcaccagaggtaatcgtggacagc
gaggaagaaagagaagatgtggcgctacaaatggtgggtttcagcaacccaccggtgctaatcaagcatgg
caaaggaggtaagcgcacagtgcggcggctgaatgaagacgacccagtggcgcgtggtatgcggacgcaag
aggaagaggaagagcccagcgaagcggaaagtgaaattacggtgatgaacccgctgagtgtgccgatcgtg
tctgcgtgggagaagggcatggaggctgcgcgcgcgctgatggacaagtaccacgtggataacgatctaaa
ggcgaacttcaaactactgcctgaccaagtggaagctctggcggccgtatgcaagacctggctgaacgagg
agcaccgcgggttgcagctgaccttcaccagcaacaagacctttgtgacgatgatggggcgattcctgcag
gcgtacctgcagtcgtttgcagaggtgacctacaagcatcacgagcccacgggctgcgcgttgtggctgca
ccgctgcgctgagatcgaaggcgagcttaagtgtctacacggaagcattatgataaataaggagcacgtga
ttgaaatggatgtgacgagcgaaaacgggcagcgcgcgctgaaggagcagtctagcaaggccaagatcgtg
aagaaccggtggggccgaaatgtggtgcagatctccaacaccgacgcaaggtgctgcgtgcacgacgcggc
ctgtccggccaatcagttttccggcaagtcttgcggcatgttcttctctgaaggcgcaaaggctcaggtgg
cttttaagcagatcaaggcttttatgcaggcgctgtatcctaacgcccagaccgggcacggtcaccttttg
atgccactacggtgcgagtgcaactcaaagcctgggcacgcgccctttttgggaaggcagctaccaaagtt
gactccgttcgccctgagcaacgcggaggacctggacgcggatctgatctccgacaagagcgtgctggcca
gcgtgcaccacccggcgctgatagtgttccagtgctgcaaccctgtgtatcgcaactcgcgcgcgcagggc
ggaggccccaactgcgacttcaagatatcggcgcccgacctgctaaacgcgttggtgatggtgcgcagcct
gtggagtgaaaacttcaccgagctgccgcggatggttgtgcctgagtttaagtggagcactaaacaccagt
atcgcaacgtgtccctgccagtggcgcatagcgatgcgcggcagaacccctttgatttttcaggcgctact
aacttcagcctgctgaagcaggctggcgacgtggaggagaaccctggacctgcgtccatgactacgtccgg
cgttccatttggcatgacactacgaccaacacgatctcggttgtctcggcgcactccgtacagtagggatc
gtctacctccttttgagacagaaacccgcgctaccatactggaggatcatccgctgctgcccgaatgtaac
actttgacaatgcacaacgtgagttacgtgcgaggtcttccctgcagtgtgggatttacgctgattcagga
atgggttgttccctgggatatggttctaacgcgggaggagcttgtaatcctgaggaagtgtatgcacgtgt
gcctgtgttgtgccaacattgatatcatgacgagcatgatgatccatggttacgagtcctgggctctccac
tgtcattgttccagtcccggttccctgcagtgtatagccggcgggcaggttttggccagctggtttaggat
ggtggtggatggcgccatgtttaatcagaggtttatatggtaccgggaggtggtgaattacaacatgccaa
aagaggtaatgtttatgtccagcgtgtttatgaggggtcgccacttaatctacctgcgcttgtggtatgat
ggccacgtgggttctgtggtccccgccatgagctttggatacagcgccttgcactgtgggattttgaacaa
tattgtggtgctgtgctgcagttactgtgctgatttaagtgagatcagggtgcgctgctgtgcccggagga
caaggcgccttatgctgcgggcggtgcgaatcatcgctgaggagaccactgccatgttgtattcctgcagg
acggagcggcggcggcagcagtttattcgcgcgctgctgcagcaccaccgccctatcctgatgcacgatta
tgactctacccccatgtaatgctgaggctaactgaaacacggaaggagacaatacaggaaggaacggtaac
ttgtttattgcagcttataatggttacaaataaagcaatagcatcacaaatttcacaaataaagcattttt
ttcactgcattctagttgtggtttgtccaaactcatcaatgtatcttatcatgtctgtcgtctatgatgag
gatgttggtggagagcatgtggaggaagtggatagggaaggttgtagagtagatccggttgaagtgatgag
gataggaggaggtcacaaaggagagcctgtaagcgggcactcttccgtggtctggtggataaattcgcaag
ggtatcatggcggacgaccggggttcgagccccgtatccggccgtccgccgtgatccatgcggttaccgcc
cgcgtgtcgaacccaggtgtgcgacgtcagacaacgggggagtgctccttttggcttccttccaggcgcgg
cggctgctgcgctagctttttt
SEQ ID NO: 35 μH 2
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctggacttagcctttagtgaaccgtcagaattaattcagatcgatctaccagaaccgtcaga
tccgctagagattacgccaaccgccaccatgggcagcactacgtccggcgttccatttggcatgacactac
gaccaacacgatctcggttgtctcggcgcactccgtacagtagggatcgtctacctccttttgagacagaa
acccgcgctaccatactggaggatcatccgctgctgcccgaatgtaacactttgacaatgcacaacgtgag
ttacgtgcgaggtcttccctgcagtgtgggatttacgctgattcaggaatgggttgttccctgggatatgg
ttctaacgcgggaggagcttgtaatcctgaggaagtgtatgcacgtgtgcctgtgttgtgccaacattgat
atcatgacgagcatgatgatccatggttacgagtcctgggctctccactgtcattgttccagtcccggttc
cctgcagtgtatagccggcgggcaggttttggccagctggtttaggatggtggtggatggcgccatgttta
atcagaggtttatatggtaccgggaggtggtgaattacaacatgccaaaagaggtaatgtttatgtccagc
gtgtttatgaggggtcgccacttaatctacctgcgcttgtggtatgatggccacgtgggttctgtggtccc
cgccatgagctttggatacagcgccttgcactgtgggattttgaacaatattgtggtgctgtgctgcagtt
actgtgctgatttaagtgagatcagggtgcgctgctgtgcccggaggacaaggcgccttatgctgcgggcg
gtgcgaatcatcgctgaggagaccactgccatgttgtattcctgcaggacggagcggcggcggcagcagtt
tattcgcgcgctgctgcagcaccaccgccctatcctgatgcacgattatgactctacccccatgtcaggcg
ctactaacttcagcctgctgaagcaggctggcgacgtggaggagaaccctggacctgcgtccatggccagt
cgggaagaggagcagcgcgaaaccacccccgagcgcggacgcggtgcggcgcgacgtcccccaaccatgga
ggacgtgtcgtccccgtccccgtcgccgccgcctccccgggcgcccccaaaaaagcggatgaggcggcgta
tcgagtccgaggacgaggaagactcatcacaagacgcgctggtgccgcgcacacccagcccgcggccatcg
acctcggcggcggatttggccattgcgcccaagaagaaaaagaagcgcccttctcccaagcccgagcgccc
gccatcaccagaggtaatcgtggacagcgaggaagaaagagaagatgtggcgctacaaatggtgggtttca
gcaacccaccggtgctaatcaagcatggcaaaggaggtaagcgcacagtgcggcggctgaatgaagacgac
ccagtggcgcgtggtatgcggacgcaagaggaagaggaagagcccagcgaagcggaaagtgaaattacggt
gatgaacccgctgagtgtgccgatcgtgtctgcgtgggagaagggcatggaggctgcgcgcgcgctgatgg
acaagtaccacgtggataacgatctaaaggcgaacttcaaactactgcctgaccaagtggaagctctggcg
gccgtatgcaagacctggctgaacgaggagcaccgcgggttgcagctgaccttcaccagcaacaagacctt
tgtgacgatgatggggcgattcctgcaggcgtacctgcagtcgtttgcagaggtgacctacaagcatcacg
agcccacgggctgcgcgttgtggctgcaccgctgcgctgagatcgaaggcgagcttaagtgtctacacgga
agcattatgataaataaggagcacgtgattgaaatggatgtgacgagcgaaaacgggcagcgcgcgctgaa
ggagcagtctagcaaggccaagatcgtgaagaaccggtggggccgaaatgtggtgcagatctccaacaccg
acgcaaggtgctgcgtgcacgacgcggcctgtccggccaatcagttttccggcaagtcttgcggcatgttc
ttctctgaaggcgcaaaggctcaggtggcttttaagcagatcaaggcttttatgcaggcgctgtatcctaa
cgcccagaccgggcacggtcaccttttgatgccactacggtgcgagtgcaactcaaagcctgggcacgcgc
cctttttgggaaggcagctaccaaagttgactccgttcgccctgagcaacgcggaggacctggacgcggat
ctgatctccgacaagagcgtgctggccagcgtgcaccacccggcgctgatagtgttccagtgctgcaaccc
tgtgtatcgcaactcgcgcgcgcagggcggaggccccaactgcgacttcaagatatcggcgcccgacctgc
taaacgcgttggtgatggtgcgcagcctgtggagtgaaaacttcaccgagctgccgcggatggttgtgcct
gagtttaagtggagcactaaacaccagtatcgcaacgtgtccctgccagtggcgcatagcgatgcgcggca
gaacccctttgattttaatgctgaggctaactgaaacacggaaggagacaatacaggaaggaacggtaact
tgtttattgcagcttataatggttacaaataaagcaatagcatcacaaatttcacaaataaagcatttttt
tcactgcattctagttgtggtttgtccaaactcatcaatgtatcttatcatgtctgtcgtctatgatgagg
atgttggtggagagcatgtggaggaagtggatagggaaggttgtagagtagatccggttgaagtgatgagg
ataggaggaggtcacaaaggagagcctgtaagcgggcactcttccgtggtctggtggataaattcgcaagg
gtatcatggcggacgaccggggttcgagccccgtatccggccgtccgccgtgatccatgcggttaccgccc
gcgtgtcgaacccaggtgtgcgacgtcagacaacgggggagtgctccttttggcttccttccaggcgcggc
ggctgctgcgctagctttttt
SEQ ID NO: 36 μH 3
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctggacttagcctttagtgaaccgtcagaattaattcagatcgatctaccagaaccgtcaga
tccgctagagattacgccaaccgccaccatgggcagcgccagtcgggaagaggagcagcgcgaaaccaccc
ccgagcgcggacgcggtgcggcgcgacgtcccccaaccatggaggacgtgtcgtccccgtccccgtcgccg
ccgcctccccgggcgcccccaaaaaagcggatgaggcggcgtatcgagtccgaggacgaggaagactcatc
acaagacgcgctggtgccgcgcacacccagcccgcggccatcgacctcggcggcggatttggccattgcgc
ccaagaagaaaaagaagcgcccttctcccaagcccgagcgcccgccatcaccagaggtaatcgtggacagc
gaggaagaaagagaagatgtggcgctacaaatggtgggtttcagcaacccaccggtgctaatcaagcatgg
caaaggaggtaagcgcacagtgcggcggctgaatgaagacgacccagtggcgcgtggtatgcggacgcaag
aggaagaggaagagcccagcgaagcggaaagtgaaattacggtgatgaacccgctgagtgtgccgatcgtg
tctgcgtgggagaagggcatggaggctgcgcgcgcgctgatggacaagtaccacgtggataacgatctaaa
ggcgaacttcaaactactgcctgaccaagtggaagctctggcggccgtatgcaagacctggctgaacgagg
agcaccgcgggttgcagctgaccttcaccagcaacaagacctttgtgacgatgatggggcgattcctgcag
gcgtacctgcagtcgtttgcagaggtgacctacaagcatcacgagcccacgggctgcgcgttgtggctgca
ccgctgcgctgagatcgaaggcgagcttaagtgtctacacggaagcattatgataaataaggagcacgtga
ttgaaatggatgtgacgagcgaaaacgggcagcgcgcgctgaaggagcagtctagcaaggccaagatcgtg
aagaaccggtggggccgaaatgtggtgcagatctccaacaccgacgcaaggtgctgcgtgcacgacgcggc
ctgtccggccaatcagttttccggcaagtcttgcggcatgttcttctctgaaggcgcaaaggctcaggtgg
cttttaagcagatcaaggcttttatgcaggcgctgtatcctaacgcccagaccgggcacggtcaccttttg
atgccactacggtgcgagtgcaactcaaagcctgggcacgcgccctttttgggaaggcagctaccaaagtt
gactccgttcgccctgagcaacgcggaggacctggacgcggatctgatctccgacaagagcgtgctggcca
gcgtgcaccacccggcgctgatagtgttccagtgctgcaaccctgtgtatcgcaactcgcgcgcgcagggc
ggaggccccaactgcgacttcaagatatcggcgcccgacctgctaaacgcgttggtgatggtgcgcagcct
gtggagtgaaaacttcaccgagctgccgcggatggttgtgcctgagtttaagtggagcactaaacaccagt
atcgcaacgtgtccctgccagtggcgcatagcgatgcgcggcagaacccctttgatttttcaggctaataa
cagcttccggactctagaacatccctacaggtgatatcctcgatcccgctcgataagcttgatatcgaatt
ccgcccccccccctctccctcccccccccctaacgttactggccgaagccgcttggaataaggccggtgtg
cgtttgtctatatgttattttccaccatattgccgtcttttggcaatgtgagggcccggaaacctggccct
gtcttcttgacgagcattcctaggggtctttcccctctcgccaaaggaatgcaaggtctgttgaatgtcgt
gaaggaagcagttcctctggaagcttcttgaagacaaacaacgtctgtagcgaccctttgcaggcagcgga
accccccacctggcgacaggtgcctctgcggccaaaagccacgtgtataagatacacctgcaaaggcggca
caaccccagtgccacgttgtgagttggatagttgtggaaagagtcaaatggctctcctcaagcgtattcaa
caaggggctgaaggatgcccagaaggtaccccattgtatgggatctgatctggggcctcggtgcacatgct
ttacatgtgtttagtcgaggttaaaaaaacgtctaggccccccgaaccacggggacgtggttttcctttga
aaaacacgatgataatatggccacaaccgcgtccatgactacgtccggcgttccatttggcatgacactac
gaccaacacgatctcggttgtctcggcgcactccgtacagtagggatcgtctacctccttttgagacagaa
acccgcgctaccatactggaggatcatccgctgctgcccgaatgtaacactttgacaatgcacaacgtgag
ttacgtgcgaggtcttccctgcagtgtgggatttacgctgattcaggaatgggttgttccctgggatatgg
ttctaacgcgggaggagcttgtaatcctgaggaagtgtatgcacgtgtgcctgtgttgtgccaacattgat
atcatgacgagcatgatgatccatggttacgagtcctgggctctccactgtcattgttccagtcccggttc
cctgcagtgtatagccggcgggcaggttttggccagctggtttaggatggtggtggatggcgccatgttta
atcagaggtttatatggtaccgggaggtggtgaattacaacatgccaaaagaggtaatgtttatgtccagc
gtgtttatgaggggtcgccacttaatctacctgcgcttgtggtatgatggccacgtgggttctgtggtccc
cgccatgagctttggatacagcgccttgcactgtgggattttgaacaatattgtggtgctgtgctgcagtt
actgtgctgatttaagtgagatcagggtgcgctgctgtgcccggaggacaaggcgccttatgctgcgggcg
gtgcgaatcatcgctgaggagaccactgccatgttgtattcctgcaggacggagcggcggcggcagcagtt
tattcgcgcgctgctgcagcaccaccgccctatcctgatgcacgattatgactctacccccatgtaatgct
gaggctaactgaaacacggaaggagacaatacaggaaggaacggtaacttgtttattgcagcttataatgg
ttacaaataaagcaatagcatcacaaatttcacaaataaagcatttttttcactgcattctagttgtggtt
tgtccaaactcatcaatgtatcttatcatgtctgtcgtctatgatgaggatgttggtggagagcatgtgga
ggaagtggatagggaaggttgtagagtagatccggttgaagtgatgaggataggaggaggtcacaaaggag
agcctgtaagcgggcactcttccgtggtctggtggataaattcgcaagggtatcatggcggacgaccgggg
ttcgagccccgtatccggccgtccgccgtgatccatgcggttaccgcccgcgtgtcgaacccaggtgtgcg
acgtcagacaacgggggagtgctccttttggcttccttccaggcgcggcggctgctgcgctagctttttt
SEQ ID NO: 37 μH 4
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctggacttagcctttagtgaaccgtcagaattaattcagatcgatctaccagaaccgtcaga
tccgctagagattacgccaaccgccaccatgggcagcactacgtccggcgttccatttggcatgacactac
gaccaacacgatctcggttgtctcggcgcactccgtacagtagggatcgtctacctccttttgagacagaa
acccgcgctaccatactggaggatcatccgctgctgcccgaatgtaacactttgacaatgcacaacgtgag
ttacgtgcgaggtcttccctgcagtgtgggatttacgctgattcaggaatgggttgttccctgggatatgg
ttctaacgcgggaggagcttgtaatcctgaggaagtgtatgcacgtgtgcctgtgttgtgccaacattgat
atcatgacgagcatgatgatccatggttacgagtcctgggctctccactgtcattgttccagtcccggttc
cctgcagtgtatagccggcgggcaggttttggccagctggtttaggatggtggtggatggcgccatgttta
atcagaggtttatatggtaccgggaggtggtgaattacaacatgccaaaagaggtaatgtttatgtccagc
gtgtttatgaggggtcgccacttaatctacctgcgcttgtggtatgatggccacgtgggttctgtggtccc
cgccatgagctttggatacagcgccttgcactgtgggattttgaacaatattgtggtgctgtgctgcagtt
actgtgctgatttaagtgagatcagggtgcgctgctgtgcccggaggacaaggcgccttatgctgcgggcg
gtgcgaatcatcgctgaggagaccactgccatgttgtattcctgcaggacggagcggcggcggcagcagtt
tattcgcgcgctgctgcagcaccaccgccctatcctgatgcacgattatgactctacccccatgcaggcta
ataacagcttccggactctagaacatccctacaggtgatatcctcgatcccgctcgataagcttgatatcg
aattccgcccccccccctctccctcccccccccctaacgttactggccgaagccgcttggaataaggccgg
tgtgcgtttgtctatatgttattttccaccatattgccgtcttttggcaatgtgagggcccggaaacctgg
ccctgtcttcttgacgagcattcctaggggtctttcccctctcgccaaaggaatgcaaggtctgttgaatg
tcgtgaaggaagcagttcctctggaagcttcttgaagacaaacaacgtctgtagcgaccctttgcaggcag
cggaaccccccacctggcgacaggtgcctctgcggccaaaagccacgtgtataagatacacctgcaaaggc
ggcacaaccccagtgccacgttgtgagttggatagttgtggaaagagtcaaatggctctcctcaagcgtat
tcaacaaggggctgaaggatgcccagaaggtaccccattgtatgggatctgatctggggcctcggtgcaca
tgctttacatgtgtttagtcgaggttaaaaaaacgtctaggccccccgaaccacggggacgtggttttcct
ttgaaaaacacgatgataatatggccacaaccgcgtccatggccagtcgggaagaggagcagcgcgaaacc
acccccgagcgcggacgcggtgcggcgcgacgtcccccaaccatggaggacgtgtcgtccccgtccccgtc
gccgccgcctccccgggcgcccccaaaaaagcggatgaggcggcgtatcgagtccgaggacgaggaagact
catcacaagacgcgctggtgccgcgcacacccagcccgcggccatcgacctcggcggcggatttggccatt
gcgcccaagaagaaaaagaagcgcccttctcccaagcccgagcgcccgccatcaccagaggtaatcgtgga
cagcgaggaagaaagagaagatgtggcgctacaaatggtgggtttcagcaacccaccggtgctaatcaagc
atggcaaaggaggtaagcgcacagtgcggcggctgaatgaagacgacccagtggcgcgtggtatgcggacg
caagaggaagaggaagagcccagcgaagcggaaagtgaaattacggtgatgaacccgctgagtgtgccgat
cgtgtctgcgtgggagaagggcatggaggctgcgcgcgcgctgatggacaagtaccacgtggataacgatc
taaaggcgaacttcaaactactgcctgaccaagtggaagctctggcggccgtatgcaagacctggctgaac
gaggagcaccgcgggttgcagctgaccttcaccagcaacaagacctttgtgacgatgatggggcgattcct
gcaggcgtacctgcagtcgtttgcagaggtgacctacaagcatcacgagcccacgggctgcgcgttgtggc
tgcaccgctgcgctgagatcgaaggcgagcttaagtgtctacacggaagcattatgataaataaggagcac
gtgattgaaatggatgtgacgagcgaaaacgggcagcgcgcgctgaaggagcagtctagcaaggccaagat
cgtgaagaaccggtggggccgaaatgtggtgcagatctccaacaccgacgcaaggtgctgcgtgcacgacg
cggcctgtccggccaatcagttttccggcaagtcttgcggcatgttcttctctgaaggcgcaaaggctcag
gtggcttttaagcagatcaaggcttttatgcaggcgctgtatcctaacgcccagaccgggcacggtcacct
tttgatgccactacggtgcgagtgcaactcaaagcctgggcacgcgccctttttgggaaggcagctaccaa
agttgactccgttcgccctgagcaacgcggaggacctggacgcggatctgatctccgacaagagcgtgctg
gccagcgtgcaccacccggcgctgatagtgttccagtgctgcaaccctgtgtatcgcaactcgcgcgcgca
gggcggaggccccaactgcgacttcaagatatcggcgcccgacctgctaaacgcgttggtgatggtgcgca
gcctgtggagtgaaaacttcaccgagctgccgcggatggttgtgcctgagtttaagtggagcactaaacac
cagtatcgcaacgtgtccctgccagtggcgcatagcgatgcgcggcagaacccctttgattttaatgctga
ggctaactgaaacacggaaggagacaatacaggaaggaacggtaacttgtttattgcagcttataatggtt
acaaataaagcaatagcatcacaaatttcacaaataaagcatttttttcactgcattctagttgtggtttg
tccaaactcatcaatgtatcttatcatgtctgtcgtctatgatgaggatgttggtggagagcatgtggagg
aagtggatagggaaggttgtagagtagatccggttgaagtgatgaggataggaggaggtcacaaaggagag
cctgtaagcgggcactcttccgtggtctggtggataaattcgcaagggtatcatggcggacgaccggggtt
cgagccccgtatccggccgtccgccgtgatccatgcggttaccgcccgcgtgtcgaacccaggtgtgcgac
gtcagacaacgggggagtgctccttttggcttccttccaggcgcggcggctgctgcgctagctttttt
SEQ ID NO: 38 μH 5
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctggacttagcctttagtgaaccgtcagaattaattcagatcgatctaccagaaccgtcaga
tccgctagagattacgccaaccgccaccatgggcagcgccagtcgggaagaggagcagcgcgaaaccaccc
ccgagcgcggacgcggtgcggcgcgacgtcccccaaccatggaggacgtgtcgtccccgtccccgtcgccg
ccgcctccccgggcgcccccaaaaaagcggatgaggcggcgtatcgagtccgaggacgaggaagactcatc
acaagacgcgctggtgccgcgcacacccagcccgcggccatcgacctcggcggcggatttggccattgcgc
ccaagaagaaaaagaagcgcccttctcccaagcccgagcgcccgccatcaccagaggtaatcgtggacagc
gaggaagaaagagaagatgtggcgctacaaatggtgggtttcagcaacccaccggtgctaatcaagcatgg
caaaggaggtaagcgcacagtgcggcggctgaatgaagacgacccagtggcgcgtggtatgcggacgcaag
aggaagaggaagagcccagcgaagcggaaagtgaaattacggtgatgaacccgctgagtgtgccgatcgtg
tctgcgtgggagaagggcatggaggctgcgcgcgcgctgatggacaagtaccacgtggataacgatctaaa
ggcgaacttcaaactactgcctgaccaagtggaagctctggcggccgtatgcaagacctggctgaacgagg
agcaccgcgggttgcagctgaccttcaccagcaacaagacctttgtgacgatgatggggcgattcctgcag
gcgtacctgcagtcgtttgcagaggtgacctacaagcatcacgagcccacgggctgcgcgttgtggctgca
ccgctgcgctgagatcgaaggcgagcttaagtgtctacacggaagcattatgataaataaggagcacgtga
ttgaaatggatgtgacgagcgaaaacgggcagcgcgcgctgaaggagcagtctagcaaggccaagatcgtg
aagaaccggtggggccgaaatgtggtgcagatctccaacaccgacgcaaggtgctgcgtgcacgacgcggc
ctgtccggccaatcagttttccggcaagtcttgcggcatgttcttctctgaaggcgcaaaggctcaggtgg
cttttaagcagatcaaggcttttatgcaggcgctgtatcctaacgcccagaccgggcacggtcaccttttg
atgccactacggtgcgagtgcaactcaaagcctgggcacgcgccctttttgggaaggcagctaccaaagtt
gactccgttcgccctgagcaacgcggaggacctggacgcggatctgatctccgacaagagcgtgctggcca
gcgtgcaccacccggcgctgatagtgttccagtgctgcaaccctgtgtatcgcaactcgcgcgcgcagggc
ggaggccccaactgcgacttcaagatatcggcgcccgacctgctaaacgcgttggtgatggtgcgcagcct
gtggagtgaaaacttcaccgagctgccgcggatggttgtgcctgagtttaagtggagcactaaacaccagt
atcgcaacgtgtccctgccagtggcgcatagcgatgcgcggcagaacccctttgatttttcaggcgctact
aacttcagcctgctgaagcaggctggcgacgtggaggagaaccctggacctgcgtccatgactacgtccgg
cgttccatttggcatgacactacgaccaacacgatctcggttgtctcggcgcactccgtacagtagggatc
gtctacctccttttgagacagaaacccgcgctaccatactggaggatcatccgctgctgcccgaatgtaac
actttgacaatgcacaacgtgagttacgtgcgaggtcttccctgcagtgtgggatttacgctgattcagga
atgggttgttccctgggatatggttctaacgcgggaggagcttgtaatcctgaggaagtgtatgcacgtgt
gcctgtgttgtgccaacattgatatcatgacgagcatgatgatccatggttacgagtcctgggctctccac
tgtcattgttccagtcccggttccctgcagtgtatagccggcgggcaggttttggccagctggtttaggat
ggtggtggatggcgccatgtttaatcagaggtttatatggtaccgggaggtggtgaattacaacatgccaa
aagaggtaatgtttatgtccagcgtgtttatgaggggtcgccacttaatctacctgcgcttgtggtatgat
ggccacgtgggttctgtggtccccgccatgagctttggatacagcgccttgcactgtgggattttgaacaa
tattgtggtgctgtgctgcagttactgtgctgatttaagtgagatcagggtgcgctgctgtgcccggagga
caaggcgccttatgctgcgggcggtgcgaatcatcgctgaggagaccactgccatgttgtattcctgcagg
acggagcggcggcggcagcagtttattcgcgcgctgctgcagcaccaccgccctatcctgatgcacgatta
tgactctacccccatgtaatgctgaggctaactgaaacacggaaggagacaatacaggaaggaacggtaac
ttgtttattgcagcttataatggttacaaataaagcaatagcatcacaaatttcacaaataaagcattttt
ttcactgcattctagttgtggtttgtccaaactcatcaatgtatctta
SEQ ID NO: 39 μH 6
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctggacttagcctttagtgaaccgtcagaattaattcagatcgatctaccagaaccgtcaga
tccgctagagattacgccaaccgccaccatgggcagcactacgtccggcgttccatttggcatgacactac
gaccaacacgatctcggttgtctcggcgcactccgtacagtagggatcgtctacctccttttgagacagaa
acccgcgctaccatactggaggatcatccgctgctgcccgaatgtaacactttgacaatgcacaacgtgag
ttacgtgcgaggtcttccctgcagtgtgggatttacgctgattcaggaatgggttgttccctgggatatgg
ttctaacgcgggaggagcttgtaatcctgaggaagtgtatgcacgtgtgcctgtgttgtgccaacattgat
atcatgacgagcatgatgatccatggttacgagtcctgggctctccactgtcattgttccagtcccggttc
cctgcagtgtatagccggcgggcaggttttggccagctggtttaggatggtggtggatggcgccatgttta
atcagaggtttatatggtaccgggaggtggtgaattacaacatgccaaaagaggtaatgtttatgtccagc
gtgtttatgaggggtcgccacttaatctacctgcgcttgtggtatgatggccacgtgggttctgtggtccc
cgccatgagctttggatacagcgccttgcactgtgggattttgaacaatattgtggtgctgtgctgcagtt
actgtgctgatttaagtgagatcagggtgcgctgctgtgcccggaggacaaggcgccttatgctgcgggcg
gtgcgaatcatcgctgaggagaccactgccatgttgtattcctgcaggacggagcggcggcggcagcagtt
tattcgcgcgctgctgcagcaccaccgccctatcctgatgcacgattatgactctacccccatgtcaggcg
ctactaacttcagcctgctgaagcaggctggcgacgtggaggagaaccctggacctgcgtccatggccagt
cgggaagaggagcagcgcgaaaccacccccgagctcggactcggtgcggcgcgacgtcccccaaccatgga
ggacgtgtcgtccccgtccccgtcgccgccgcctccccgggcgcccccaaaaaagcggatgattcggctta
tcgagtccgaggacgaggaagactcatcacaagactcgctggttccgctcacacccagcccgcggccatcg
acctcggcggcggatttggccattgcgcccaagaagaaaaagaagcgcccttctcccaagcccgagcgccc
gccatcaccagatttaatcgtggacagcgaggaagaaagagaagatgtggcgctacaaatggtgggtttca
gcaacccaccggtgctaatcaagcatggcaaaggaggtaagcgcacagttcttctgctgaatgaagacgac
ccagtggcgcgtggtatgcggacgcaagaggaagaggaagagcccagcgaagcggaaagtgaaattacggt
gatgaacccgctgagtgtgccgatcgtgtctgcgtgggagaagggcatggattctgctctcgcgctgatgg
acaagtaccacgtggataacgatctaaaggcgaacttcaaactactgcctgaccaagtggaagctctggcg
gccgtatgcaagacctggctgaacgaggagcaccgcgggttgcagctgaccttcaccagcaacaagacctt
tgtgacgatgatggggcgattcctgcaggcgtacctgcagtcgtttgcagaggtgacctacaagcatcacg
agcccacgggctgctcgtttttgctgcaccgcttcgctgagatcgaaggcgagcttaagtgtctacacgga
agcattatgataaataaggagcacgtgattgaaatggatttgacgagcgaaaacgggcagcgcgcgctgaa
ggagcagtctagcaaggccaagatcgtgaagaaccggtggggccgaaattttttgcagatctccaacaccg
actcaaggtgcttcttgcacgacgcggcctttccggccaatcagttttccggcaagtcttgcggcatgttc
ttctctgaaggcgcaaaggctcaggtggcttttaagcagatcaaggcttttattcaggcgctgtatcctaa
cgcccagaccgggcacggtcaccttttgatgccactacgttgcgattgcaactcaaagcctgggcacgcgc
cctttttgggaaggcagctaccaaagttgactccgttcgccctgagcaacgcggaggacctggacgcggat
ctgatctccgacaagagcttgctggccagcttgcaccacccggcgctgatagtgttccagtgctgcaaccc
tgtgtatcgcaactctctcgcgcagggcggaggccccaactgcgacttcaagatatcggcgcccgacctgc
taaacgcgttggtgatggttcgcagccttttgagtgaaaacttcaccgagctgccgcggatggttttgcct
gagtttaagtggagcactaaacaccagtatcgcaacgtgtccctgccagtggcgcatagcgatgcgcggca
gaacccctttgattttaatgctgaggctaactgaaacacggaaggagacaatacaggaaggaacggtaact
tgtttattgcagcttataatggttacaaataaagcaatagcatcacaaatttcacaaataaagcatttttt
tcactgcattctagttgtggtttgtccaaactcatcaatgtatctta
SEQ ID NO: 40 μH 7
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctggacttagcctttagtgaaccgtcagaattaattcagatcgatctaccagaaccgtcaga
tccgctagagattacgccaaccgccaccatgggcagcgccagtcgggaagaggagcagcgcgaaaccaccc
ccgagcgcggacgcggtgcggcgcgacgtcccccaaccatggaggacgtgtcgtccccgtccccgtcgccg
ccgcctccccgggcgcccccaaaaaagcggatgaggcggcgtatcgagtccgaggacgaggaagactcatc
acaagacgcgctggtgccgcgcacacccagcccgcggccatcgacctcggcggcggatttggccattgcgc
ccaagaagaaaaagaagcgcccttctcccaagcccgagcgcccgccatcaccagaggtaatcgtggacagc
gaggaagaaagagaagatgtggcgctacaaatggtgggtttcagcaacccaccggtgctaatcaagcatgg
caaaggaggtaagcgcacagtgcggcggctgaatgaagacgacccagtggcgcgtggtatgcggacgcaag
aggaagaggaagagcccagcgaagcggaaagtgaaattacggtgatgaacccgctgagtgtgccgatcgtg
tctgcgtgggagaagggcatggaggctgcgcgcgcgctgatggacaagtaccacgtggataacgatctaaa
ggcgaacttcaaactactgcctgaccaagtggaagctctggcggccgtatgcaagacctggctgaacgagg
agcaccgcgggttgcagctgaccttcaccagcaacaagacctttgtgacgatgatggggcgattcctgcag
gcgtacctgcagtcgtttgcagaggtgacctacaagcatcacgagcccacgggctgcgcgttgtggctgca
ccgctgcgctgagatcgaaggcgagcttaagtgtctacacggaagcattatgataaataaggagcacgtga
ttgaaatggatgtgacgagcgaaaacgggcagcgcgcgctgaaggagcagtctagcaaggccaagatcgtg
aagaaccggtggggccgaaatgtggtgcagatctccaacaccgacgcaaggtgctgcgtgcacgacgcggc
ctgtccggccaatcagttttccggcaagtcttgcggcatgttcttctctgaaggcgcaaaggctcaggtgg
cttttaagcagatcaaggcttttatgcaggcgctgtatcctaacgcccagaccgggcacggtcaccttttg
atgccactacggtgcgagtgcaactcaaagcctgggcacgcgccctttttgggaaggcagctaccaaagtt
gactccgttcgccctgagcaacgcggaggacctggacgcggatctgatctccgacaagagcgtgctggcca
gcgtgcaccacccggcgctgatagtgttccagtgctgcaaccctgtgtatcgcaactcgcgcgcgcagggc
ggaggccccaactgcgacttcaagatatcggcgcccgacctgctaaacgcgttggtgatggtgcgcagcct
gtggagtgaaaacttcaccgagctgccgcggatggttgtgcctgagtttaagtggagcactaaacaccagt
atcgcaacgtgtccctgccagtggcgcatagcgatgcgcggcagaacccctttgatttttcaggctaataa
cagcttccggactctagaacatccctacaggtgatatcctcgatcccgctcgataagcttgatatcgaatt
ccgcccccccccctctccctcccccccccctaacgttactggccgaagccgcttggaataaggccggtgtg
cgtttgtctatatgttattttccaccatattgccgtcttttggcaatgtgagggcccggaaacctggccct
gtcttcttgacgagcattcctaggggtctttcccctctcgccaaaggaatgcaaggtctgttgaatgtcgt
gaaggaagcagttcctctggaagcttcttgaagacaaacaacgtctgtagcgaccctttgcaggcagcgga
accccccacctggcgacaggtgcctctgcggccaaaagccacgtgtataagatacacctgcaaaggcggca
caaccccagtgccacgttgtgagttggatagttgtggaaagagtcaaatggctctcctcaagcgtattcaa
caaggggctgaaggatgcccagaaggtaccccattgtatgggatctgatctggggcctcggtgcacatgct
ttacatgtgtttagtcgaggttaaaaaaacgtctaggccccccgaaccacggggacgtggttttcctttga
aaaacacgatgataatatggccacaaccgcgtccatgactacgtccggcgttccatttggcatgacactac
gaccaacacgatctcggttgtctcggcgcactccgtacagtagggatcgtctacctccttttgagacagaa
acccgcgctaccatactggaggatcatccgctgctgcccgaatgtaacactttgacaatgcacaacgtgag
ttacgtgcgaggtcttccctgcagtgtgggatttacgctgattcaggaatgggttgttccctgggatatgg
ttctaacgcgggaggagcttgtaatcctgaggaagtgtatgcacgtgtgcctgtgttgtgccaacattgat
atcatgacgagcatgatgatccatggttacgagtcctgggctctccactgtcattgttccagtcccggttc
cctgcagtgtatagccggcgggcaggttttggccagctggtttaggatggtggtggatggcgccatgttta
atcagaggtttatatggtaccgggaggtggtgaattacaacatgccaaaagaggtaatgtttatgtccagc
gtgtttatgaggggtcgccacttaatctacctgcgcttgtggtatgatggccacgtgggttctgtggtccc
cgccatgagctttggatacagcgccttgcactgtgggattttgaacaatattgtggtgctgtgctgcagtt
actgtgctgatttaagtgagatcagggtgcgctgctgtgcccggaggacaaggcgccttatgctgcgggcg
gtgcgaatcatcgctgaggagaccactgccatgttgtattcctgcaggacggagcggcggcggcagcagtt
tattcgcgcgctgctgcagcaccaccgccctatcctgatgcacgattatgactctacccccatgtaatgct
gaggctaactgaaacacggaaggagacaatacaggaaggaacggtaacttgtttattgcagcttataatgg
ttacaaataaagcaatagcatcacaaatttcacaaataaagcatttttttcactgcattctagttgtggtt
tgtccaaactcatcaatgtatctta
SEQ ID NO: 41 μH 8
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctggacttagcctttagtgaaccgtcagaattaattcagatcgatctaccagaaccgtcaga
tccgctagagattacgccaaccgccaccatgggcagcactacgtccggcgttccatttggcatgacactac
gaccaacacgatctcggttgtctcggcgcactccgtacagtagggatcgtctacctccttttgagacagaa
acccgcgctaccatactggaggatcatccgctgctgcccgaatgtaacactttgacaatgcacaacgtgag
ttacgtgcgaggtcttccctgcagtgtgggatttacgctgattcaggaatgggttgttccctgggatatgg
ttctaacgcgggaggagcttgtaatcctgaggaagtgtatgcacgtgtgcctgtgttgtgccaacattgat
atcatgacgagcatgatgatccatggttacgagtcctgggctctccactgtcattgttccagtcccggttc
cctgcagtgtatagccggcgggcaggttttggccagctggtttaggatggtggtggatggcgccatgttta
atcagaggtttatatggtaccgggaggtggtgaattacaacatgccaaaagaggtaatgtttatgtccagc
gtgtttatgaggggtcgccacttaatctacctgcgcttgtggtatgatggccacgtgggttctgtggtccc
cgccatgagctttggatacagcgccttgcactgtgggattttgaacaatattgtggtgctgtgctgcagtt
actgtgctgatttaagtgagatcagggtgcgctgctgtgcccggaggacaaggcgccttatgctgcgggcg
gtgcgaatcatcgctgaggagaccactgccatgttgtattcctgcaggacggagcggcggcggcagcagtt
tattcgcgcgctgctgcagcaccaccgccctatcctgatgcacgattatgactctacccccatgcaggcta
ataacagcttccggactctagaacatccctacaggtgatatcctcgatcccgctcgataagcttgatatcg
aattccgcccccccccctctccctcccccccccctaacgttactggccgaagccgcttggaataaggccgg
tgtgcgtttgtctatatgttattttccaccatattgccgtcttttggcaatgtgagggcccggaaacctgg
ccctgtcttcttgacgagcattcctaggggtctttcccctctcgccaaaggaatgcaaggtctgttgaatg
tcgtgaaggaagcagttcctctggaagcttcttgaagacaaacaacgtctgtagcgaccctttgcaggcag
cggaaccccccacctggcgacaggtgcctctgcggccaaaagccacgtgtataagatacacctgcaaaggc
ggcacaaccccagtgccacgttgtgagttggatagttgtggaaagagtcaaatggctctcctcaagcgtat
tcaacaaggggctgaaggatgcccagaaggtaccccattgtatgggatctgatctggggcctcggtgcaca
tgctttacatgtgtttagtcgaggttaaaaaaacgtctaggccccccgaaccacggggacgtggttttcct
ttgaaaaacacgatgataatatggccacaaccgcgtccatggccagtcgggaagaggagcagcgcgaaacc
acccccgagcgcggacgcggtgcggcgcgacgtcccccaaccatggaggacgtgtcgtccccgtccccgtc
gccgccgcctccccgggcgcccccaaaaaagcggatgaggcggcgtatcgagtccgaggacgaggaagact
catcacaagacgcgctggtgccgcgcacacccagcccgcggccatcgacctcggcggcggatttggccatt
gcgcccaagaagaaaaagaagcgcccttctcccaagcccgagcgcccgccatcaccagaggtaatcgtgga
cagcgaggaagaaagagaagatgtggcgctacaaatggtgggtttcagcaacccaccggtgctaatcaagc
atggcaaaggaggtaaccgcacagtgcggcggctgaatgaagacgacccagtggcgcgtggtatgcggacg
caagaggaagaggaagagcccagcgaagcggaaagtgaaattacggtgatgaacccgctgagtgtgccgat
cgtgtctgcgtgggagaagggcatggaggctgcgcgcgcgctgatggacaagtaccacgtggataacgatc
taaaggcgaacttcaaactactgcctgaccaagtggaagctctggcggccgtatgcaagacctggctgaac
gaggagcaccgcgggttgcagctgaccttcaccagcaacaagacctttgtgacgatgatggggcgattcct
gcaggcgtacctgcagtcgtttgcagaggtgacctacaagcatcacgagcccacgggctgcgcgttgtggc
tgcaccgctgcgctgagatcgaaggcgagcttaagtgtctacacggaagcattatgataaataaggagcac
gtgattgaaatggatgtgacgagcgaaaacgggcagcgcgcgctgaaggagcagtctagcaaggccaagat
cgtgaagaaccggtggggccgaaatgtggtgcagatctccaacaccgacgcaaggtgctgcgtgcacgacg
cggcctgtccggccaatcagttttccggcaagtcttgcggcatgttcttctctgaaggcgcaaaggctcag
gtggcttttaagcagatcaaggcttttatgcaggcgctgtatcctaacgcccagaccgggcacggtcacct
tttgatgccactacggtgcgagtgcaactcaaagcctgggcacgcgccctttttgggaaggcagctaccaa
agttgactccgttcgccctgagcaacgcggaggacctggacgcggatctgatctccgacaagagcgtgctg
gccagcgtgcaccacccggcgctgatagtgttccagtgctgcaaccctgtgtatcgcaactcgcgcgcgca
gggcggaggccccaactgcgacttcaagatatcggcgcccgacctgctaaacgcgttggtgatggtgcgca
gcctgtggagtgaaaacttcaccgagctgccgcggatggttgtgcctgagtttaagtggagcactaaacac
cagtatcgcaacgtgtccctgccagtggcgcatagcgatgcgcggcagaacccctttgattttaatgctga
ggctaactgaaacacggaaggagacaatacaggaaggaacggtaacttgtttattgcagcttataatggtt
acaaataaagcaatagcatcacaaatttcacaaataaagcatttttttcactgcattctagttgtggtttg
tccaaactcatcaatgtatctta
SEQ ID NO: 42 NiClear Promoter (E-Box, minimal Promoter)
Ccctgcgggtcacgtgacgggtcacgtgacgggtcacgtgacgggtcacgtgaaaagtgaaagtcgagtttacca
ctcacgtgaaaagtgaaagtcgagtttaccactcacgtgaaaagtgaaagtcgagtttaccactcacgtgaccct
gcgggaagcttagacactagagggtatataatggaagctcgacttccag
SEQ ID NO: 43 NiClear_fLuc_2CP
ccctgcgggtcacgtgacgggtcacgtgacgggtcacgtgacgggtcacgtgaaaagtgaaagtcgagtttacca
ctcacgtgaaaagtgaaagtcgagtttaccactcacgtgaaaagtgaaagtcgagtttaccactcacgtgaccct
gcgggaagcttagacactagagggtatataatggaagctcgacttccagtttagtgaaccgtcagaattaattca
gccgtcagaattaattcagatcgatctaccagaaccgtcagatccgctagagattacgccaaccgccaccatggg
cagcgaagatgccaaaaacattaagaagggcccagcgccattctacccactcgaagacgggaccgccggcgagca
gctgcacaaagccatgaagcgctacgccctggtgcccggcaccatcgcctttaccgacgcacatatcgaggtgga
cattacctacgccgagtacttcgagatgagcgttcggctggcagaagctatgaagcgctatgggctgaatacaaa
ccatcggatcgtggtgtgcagcgagaatagcttgcagttcttcatgcccgtgttgggtgccctgttcatcggtgt
ggctgtggccccagctaacgacatctacaacgagcgcgagctgctgaacagcatgggcatcagccagcccaccgt
cgtattcgtgagcaagaaagggctgcaaaagatcctcaacgtgcaaaagaagctaccgatcatacaaaagatcat
catcatggatagcaagaccgactaccagggcttccaaagcatgtacaccttcgtgacttcccatttgccacccgg
cttcaacgagtacgacttcgtgcccgagagcttcgaccgggacaaaaccatcgccctgatcatgaacagtagtgg
cagtaccggattgcccaagggcgtagccctaccgcaccgcaccgcttgtgtccgattcagtcatgcccgcgaccc
catcttcggcaaccagatcatccccgacaccgctatcctcagcgtggtgccatttcaccacggcttcggcatgtt
caccacgctgggctacttgatctgcggctttcgggtcgtgctcatgtaccgcttcgaggaggagctattcttgcg
cagcttgcaagactataagattcaatctgccctgctggtgcccacactatttagcttcttcgctaagagcactct
catcgacaagtacgacctaagcaacttgcacgagatcgccagcggcggggcgccgctcagcaaggaggtaggtga
ggccgtggccaaacgcttccacctaccaggcatccgccagggctacggcctgacagaaacaaccagcgccattct
gatcacccccgaaggggacgacaagcctggcgcagtaggcaaggtggtgcccttcttcgaggctaaggtggtgga
cttggacaccggtaagacactgggtgtgaaccagcgcggcgagctgtgcgtccgtggccccatgatcatgagcgg
ctacgttaacaaccccgaggctacaaacgctctcatcgacaaggacggctggctgcacagcggcgacatcgccta
ctgggacgaggacgagcacttcttcatcgtggaccggctgaagagcctgatcaaatacaagggctaccaggtagc
cccagccgaactggagagcatcctgctgcaacaccccaacatcttcgacgccggggtcgccggcctgcccgacga
cgatgccggcgagctgcccgccgcagtcgtcgtgctggaacacggtaaaaccatgaccgagaaggagatcgtgga
ctatgtggccagccaggttacaaccgccaagaagctgcgcggtggtgttgtgttcgtggacgaggtgcctaaagg
actgaccggcaagttggacgcccgcaagatccgcgagattctcattaaggccaagaagggcggcaagatcgccgt
gaattctgcttgcaagaactggttcagtagcttaagccactttgtgatccaccttaacagccacggcttccctcc
cgaggtggaggagcaggccgccggcaccctgcccatgagctgcgcccaggagagcggcatggatagacaccctgc
tgcttgcgccagcgccaggatcaacgtcTCAGGCtaa
SEQ ID NO: 44: NiClear-AcrII4A_N7
Ccctgcgggtcacgtgacgggtcacgtgacgggtcacgtgacgggtcacgtgaaaagtgaaagtcgagtttacca
ctcacgtgaaaagtgaaagtcgagtttaccactcacgtgaaaagtgaaagtcgagtttaccactcacgtgaccct
gcgggaagcttagacactagagggtatataatggaagctcgacttccagtttagtgaaccgtcagaattaattca
gatcgatctaccagaaccgtcagatccgctagagattacgccaaccgccaccatgggcagcaacatcaacgatct
gatccgggagatcaagaacaaggactacaccgtgaagctgagcggcaccgatagcaacagcatcacccagctgat
tatccgcgtgaacaacgacggcaacgagtacgtgatctccgagagcgagaacgagagcatcgtggagaagttcat
cagcgccttcaagaacggttggaaccaggagtacgaggacgaggaggagttctacaacgacatgcagaccatcac
cctgaagagcgagctgaacTCAGGCtctggactcagatctggcagcggaggaggcagcgcatctggcggaagcgg
agcgtCCggggaaattgctgctctggaagccaaaaatgcggcgttgaaagccgagattgcggccttggaagctaa
gatcgctgctttaaaggccggatactaa
SEQ_ID_NO: 45: pMRE_Promoter (MRE4x binding sites-E1b minimal promoter)
tgcactccgcccgagctctgcactccgcccgagctctgcactccgcccgagctctgcactccgcccgtcga
ccttgggcatactgcaggtcgactctagagggtatataatggat
SEQ ID NO: 46: 7B_pMin (B binding site-pMin)
ccgtcttccgtttccacatcttcacggataccaaggtggcaccggtttgattgcacagcccgtcttccgtt
tccacatcttcacggtgctggcagccggggtaccgcaggcaagtcgcccgtcttccgtttccacatcttca
cggatggaatcacggcggccgcccgtatctcatccccgtcttccgtttccacatcttcacggaatgcgtcg
gcgtgcggccgctgccagttgcaccgtcttccgtttccacatcttcacggttggctgtcctagggtacctg
gacgctccttgccgtcttccgtttccacatcttcacggtggctggtgccgcaccggtaagcagacggccgc
cgtcttccgtttccacatcttcacggggatcctgtacgggccagatagagggtatataatggaagctcgac
ttccag
SEQ_ID NO: 47: Firefly Luciferase (fLuc)
atgggcagcgaagatgccaaaaacattaagaagggcccagcgccattctacccactcgaagacgggaccgc
cggcgagcagctgcacaaagccatgaagcgctacgccctggtgcccggcaccatcgcctttaccgacgcac
atatcgaggtggacattacctacgccgagtacttcgagatgagcgttcggctggcagaagctatgaagcgc
tatgggctgaatacaaaccatcggatcgtggtgtgcagcgagaatagcttgcagttcttcatgcccgtgtt
gggtgccctgttcatcggtgtggctgtggccccagctaacgacatctacaacgagcgcgagctgctgaaca
gcatgggcatcagccagcccaccgtcgtattcgtgagcaagaaagggctgcaaaagatcctcaacgtgcaa
aagaagctaccgatcatacaaaagatcatcatcatggatagcaagaccgactaccagggcttccaaagcat
gtacaccttcgtgacttcccatttgccacccggcttcaacgagtacgacttcgtgcccgagagcttcgacc
gggacaaaaccatcgccctgatcatgaacagtagtggcagtaccggattgcccaagggcgtagccctaccg
caccgcaccgcttgtgtccgattcagtcatgcccgcgaccccatcttcggcaaccagatcatccccgacac
cgctatcctcagcgtggtgccatttcaccacggcttcggcatgttcaccacgctgggctacttgatctgcg
gctttcgggtcgtgctcatgtaccgcttcgaggaggagctattcttgcgcagcttgcaagactataagatt
caatctgccctgctggtgcccacactatttagcttcttcgctaagagcactctcatcgacaagtacgacct
aagcaacttgcacgagatcgccagcggcggggcgccgctcagcaaggaggtaggtgaggccgtggccaaac
gcttccacctaccaggcatccgccagggctacggcctgacagaaacaaccagcgccattctgatcaccccc
gaaggggacgacaagcctggcgcagtaggcaaggtggtgcccttcttcgaggctaaggtggtggacttgga
caccggtaagacactgggtgtgaaccagcgcggcgagctgtgcgtccgtggccccatgatcatgagcggct
acgttaacaaccccgaggctacaaacgctctcatcgacaaggacggctggctgcacagcggcgacatcgcc
tactgggacgaggacgagcacttcttcatcgtggaccggctgaagagcctgatcaaatacaagggctacca
ggtagccccagccgaactggagagcatcctgctgcaacaccccaacatcttcgacgccggggtcgccggcc
tgcccgacgacgatgccggcgagctgcccgccgcagtcgtcgtgctggaacacggtaaaaccatgaccgag
aaggagatcgtggactatgtggccagccaggttacaaccgccaagaagctgcgcggtggtgttgtgttcgt
ggacgaggtgcctaaaggactgaccggcaagttggacgcccgcaagatccgcgagattctcattaaggcca
agaagggcggcaagatcgccgtgtcaggctaa
SEQ_ID NO: 48: CMV/2B promoter
cgttacataacttacggtaaatggcccgcctggctgaccgcccaacgacccccgcccattgacgtcaataa
tgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaa
atggcccgcctggcattatgcccagtacatgaccttatgggactttcctacttggcagtacatctacgtat
tagtcatcgctattaccatggtgatgcggttttggcagtacatcaatgggcgtggatagcggtttgactca
cggggatttccaagtctccaccccattgacgtcaatgggagtttgttttggcaccaaaatcaacgggactt
tccaaaatgtcgtaacaactccgccccattgacgcaaatgggcggtaggcgtgtacggtgggaggtctata
taagcagagctgccgtcttccgtttccacatcttcacggataccaaggtggcaccggtttgattgcacagc
ccgtcttccgtttccacatcttcacgg
SEQ_ID NO: 49: fLuc_DR
atgggcagcgaagatgccaaaaacattaagaagggcccagcgccattctacccactcgaagacgggaccgc
cggcgagcagctgcacaaagccatgaagcgctacgccctggtgcccggcaccatcgcctttaccgacgcac
atatcgaggtggacattacctacgccgagtacttcgagatgagcgttcggctggcagaagctatgaagcgc
tatgggctgaatacaaaccatcggatcgtggtgtgcagcgagaatagcttgcagttcttcatgcccgtgtt
gggtgccctgttcatcggtgtggctgtggccccagctaacgacatctacaacgagcgcgagctgctgaaca
gcatgggcatcagccagcccaccgtcgtattcgtgagcaagaaagggctgcaaaagatcctcaacgtgcaa
aagaagctaccgatcatacaaaagatcatcatcatggatagcaagaccgactaccagggcttccaaagcat
gtacaccttcgtgacttcccatttgccacccggcttcaacgagtacgacttcgtgcccgagagcttcgacc
gggacaaaaccatcgccctgatcatgaacagtagtggcagtaccggattgcccaagggcgtagccctaccg
caccgcaccgcttgtgtccgattcagtcatgcccgcgaccccatcttcggcaaccagatcatccccgacac
cgctatcctcagcgtggtgccatttcaccacggcttcggcatgttcaccacgctgggctacttgatctgcg
gctttcgggtcgtgctcatgtaccgcttcgaggaggagctattcttgcgcagcttgcaagactataagatt
caatctgccctgctggtgcccacactatttagcttcttcgctaagagcactctcatcgacaagtacgacct
aagcaacttgcacgagatcgccagcggcggggcgccgctcagcaaggaggtaggtgaggccgtggccaaac
gcttccacctaccaggcatccgccagggctacggcctgacagaaacaaccagcgccattctgatcaccccc
gaaggggacgacaagcctggcgcagtaggcaaggtggtgcccttcttcgaggctaaggtggtggacttgga
caccggtaagacactgggtgtgaaccagcgcggcgagctgtgcgtccgtggccccatgatcatgagcggct
acgttaacaaccccgaggctacaaacgctctcatcgacaaggacggctggctgcacagcggcgacatcgcc
tactgggacgaggacgagcacttcttcatcgtggaccggctgaagagcctgatcaaatacaagggctacca
ggtagccccagccgaactggagagcatcctgctgcaacaccccaacatcttcgacgccggggtcgccggcc
tgcccgacgacgatgccggcgagctgcccgccgcagtcgtcgtgctggaacacggtaaaaccatgaccgag
aaggagatcgtggactatgtggccagccaggttacaaccgccaagaagctgcgcggtggtgttgtgttcgt
ggacgaggtgcctaaaggactgaccggcaagttggacgcccgcaagatccgcgagattctcattaaggcca
agaagggcggcaagatcgccgtgTCAGGCTAAccggactctagaacatccctacaggtgatatcctcgtgc
tgcaagtaaacccctaccaactggtcggggtttgaaac
SEQ_ID NO: 50: rtTA_DR
atgggcagcatgtctagactggacaagagcaaagtcataaactctgctctggaattactcaatggagtcgg
tatcgaaggcctgacgacaaggaaactcgctcaaaagctgggagttgagcagcctaccctgtactggcacg
tgaagaacaagcgggccctgctcgatgccctgccaatcgagatgctggacaggcatcatacccactcctgc
cccctggaaggcgagtcatggcaagactttctgcggaacaacgccaagtcataccgctgtgctctcctctc
acatcgcgacggggctaaagtgcatctcggcacccgcccaacagagaaacagtacgaaaccctggaaaatc
agctcgcgttcctgtgtcagcaaggcttctccctggagaacgcactgtacgctctgtccgccgtgggccac
tttacactgggctgcgtattggaggaacaggagcatcaagtagcaaaagaggaaagagagacacctaccac
cgattctatgcccccacttctgaaacaagcaattgagctgttcgaccggcagggagccgaacctgccttcc
ttttcggcctggaactaatcatatgtggcctggagaaacagctaaagtgcgaaagcggcgggccgaccgac
gcccttgacgattttgacttagacatgctcccagccgatgcccttgacgactttgaccttgatatgctgcc
tgctgacgctcttgacgattttgaccttgacatgctccccgggtcaggctaataacagcttccggactcta
gaacatccctacaggtgatatcctcgtgctgcaagtaaacccctaccaactggtcggggtttgaaac
SEQ ID NO: 51 pNiClear-rtTA-VP16
ccctgcgggtcacgtgacgggtcacgtgacgggtcacgtgacgggtcacgtgaaaagtgaaagtcgagttt
accactcacgtgaaaagtgaaagtcgagtttaccactcacgtgaaaagtgaaagtcgagtttaccactcac
gtgaccctgcgggaagcttagacactagagggtatataatggaagctcgacttccagggacttagccttta
gtgaaccgtcagaattaattcagatcgatctaccagaaccgtcagatccgctagagattacgccaaccgcc
accatgggcagcatgtctagactggacaagagcaaagtcataaactctgctctggaattactcaatggagt
cggtatcgaaggcctgacgacaaggaaactcgctcaaaagctgggagttgagcagcctaccctgtactggc
acgtgaagaacaagcgggccctgctcgatgccctgccaatcgagatgctggacaggcatcatacccactcc
tgccccctggaaggcgagtcatggcaagactttctgeggaacaacgccaagtcataccgctgtgctctcct
ctcacatcgcgacggggctaaagtgcatctcggcacccgcccaacagagaaacagtacgaaaccctggaaa
atcagctcgcgttcctgtgtcagcaaggcttctccctggagaacgcactgtacgctctgtccgccgtgggc
cactttacactgggctgcgtattggaggaacaggagcatcaagtagcaaaagaggaaagagagacacctac
caccgattctatgcccccacttctgaaacaagcaattgagctgttcgaccggcagggagccgaacctgcct
tccttttcggcctggaactaatcatatgtggcctggagaaacagctaaagtgcgaaagcggcgggccgacc
gacgcccttgacgattttgacttagacatgctcccagccgatgcccttgacgactttgaccttgatatgct
gcctgctgacgctcttgacgattttgaccttgacatgctccccgggtcaggctaa
SEQ ID NO: 52 pMRE-rtTA3G
cttgagctcgaggagctctgcactccgcccgagctctgcactccgcccgagctctgcactccgcccgagct
ctgcactccgcccgtcgaccttgggcatactgcaggtcgactctagagggtatataaggacttagccttta
gtgaaccgtcagaattaattcagatcgatctaccagaaccgtcagatccgctagagattacgccaaccgcc
accatgggcagcatgtctagactggacaagagcaaagtcataaactctgctctggaattactcaatggagt
cggtatcgaaggcctgacgacaaggaaactcgctcaaaagctgggagttgagcagcctaccctgtactggc
acgtgaagaacaagcgggccctgctcgatgccctgccaatcgagatgctggacaggcatcatacccactcc
tgccccctggaaggcgagtcatggcaagactttctgcggaacaacgccaagtcataccgctgtgctctcct
ctcacatcgcgacggggctaaagtgcatctcggcacccgcccaacagagaaacagtacgaaaccctggaaa
atcagctcgcgttcctgtgtcagcaaggcttctccctggagaacgcactgtacgctctgtccgccgtgggc
cactttacactgggctgcgtattggaggaacaggagcatcaagtagcaaaagaggaaagagagacacctac
caccgattctatgcccccacttctgaaacaagcaattgagctgttcgaceggcagggagccgaacctgcct
tccttttcggcctggaactaatcatatgtggcctggagaaacagctaaagtgcgaaageggegggccgacc
gacgcccttgacgattttgacttagacatgctcccagccgatgcccttgacgactttgaccttgatatgct
gcctgctgacgctcttgacgattttgaccttgacatgctccccgggtcaggctaa
SEQ ID NO: 53 HSV_TK1
Atggcttcgtacccctgccatcaacacgcgtctgcgttcgaccaggctgcgcgttctcgcggccatagcaa
ccgacgtacggcgttgcgccctcgccggcagcaagaagccacggaagtccgcctggagcagaaaatgccca
cgctactgcgggtttatatagacggtcctcacgggatggggaaaaccaccaccacgcaactgctggtggcc
ctgggttcgcgcgacgatatcgtctacgtacccgagccgatgacttactggcaggtgctgggggcttccga
gacaatcgcgaacatctacaccacacaacaccgcctcgaccagggtgagatatcggccggggacgcggcgg
tggtaatgacaagcgcccagataacaatgggcatgccttatgccgtgaccgacgccgttctggctcctcat
atcgggggggaggctgggagctcacatgccccgcccccggccctcaccctcatcttcgaccgccatcccat
cgccgccctcctgtgctacccggccgcgcgataccttatgggcagcatgaccccccaggccgtgctggcgt
tcgtggccctcatcccgccgaccttgcccggcacaaacatcgtgttgggggcccttccggaggacagacac
atcgaccgcctggccaaacgccagcgccccggcgagcggcttgacctggctatgctggccgcgattcgccg
cgtttacgggctgcttgccaatacggtgcggtatctgcagggcggcgggtcgtggcgggaggattggggac
agctttcggggacggccgtgccgccccagggtgccgagccccagagcaacgcgggcccacgaccccatatc
ggggacacgttatttaccctgtttcgggcccccgagttgctggcccccaacggcgacctgtacaacgtgtt
tgcctgggccttggacgtcttggccaaacgcctccgtcccatgcacgtctttatcctggattacgaccaat
cgcccgccggctgccgggacgccctgctgcaacttacctccgggatggtccagacccacgtcaccaccccc
ggctccataccgacgatctgcgacctggcgcgcacgtttgcccgggagatgggggaggctaactcaggcta
a
SEQ ID NO: 54 pTRE3G-HSV_TK1-DR
gtacacgccacctcgacatactcgagtttactccctatcagtgatagagaacgtatgaagagtttactccc
tatcagtgatagagaacgtatgcagactttactccctatcagtgatagagaacgtataaggagtttactcc
ctatcagtgatagagaacgtatgaccagtttactccctatcagtgatagagaacgtatctacagtttactc
cctatcagtgatagagaacgtatatccagtttactccctatcagtgatagagaacgtataagctttaggcg
tgtacggtgggcgcctataaaagcagagctcgtttagtgaaccgtcagatcgcctggagcaattccacaac
acttttgtcttataccaactttccgtaccacttcctaccctcgtaaagacttagcctttagtgaaccgtca
gaattaattcagatcgatctaccagaaccgtcagatccgctagagattacgccaaccgccaccatgggcag
catggcttcgtacccctgccatcaacacgcgtctgcgttcgaccaggctgcgcgttctcgcggccatagca
accgacgtacggcgttgcgccctcgccggcagcaagaagccacggaagtccgcctggagcagaaaatgccc
acgctactgcgggtttatatagacggtcctcacgggatggggaaaaccaccaccacgcaactgctggtggc
cctgggttcgcgcgacgatatcgtctacgtacccgagccgatgacttactggcaggtgctgggggcttccg
agacaatcgcgaacatctacaccacacaacaccgcctcgaccagggtgagatateggccggggacgeggcg
gtggtaatgacaagcgcccagataacaatgggcatgccttatgccgtgaccgacgccgttctggctcctca
tatcgggggggaggctgggagctcacatgccccgcccccggccctcaccctcatcttcgaccgccatccca
tcgccgccctcctgtgctacccggccgcgcgataccttatgggcagcatgaccccccaggccgtgctggcg
ttcgtggccctcatcccgccgaccttgcccggcacaaacatcgtgttgggggcccttccggaggacagaca
catcgaccgcctggccaaacgccagcgccccggcgageggcttgacctggctatgctggccgcgattegcc
gcgtttacgggctgcttgccaatacggtgcggtatctgcagggcggcgggtcgtggcgggaggattgggga
cagctttcggggacggccgtgccgccccagggtgccgagccccagagcaacgegggcccacgaccccatat
cggggacacgttatttaccctgtttcgggcccccgagttgctggcccccaacggcgacctgtacaacgtgt
ttgcctgggccttggacgtcttggccaaacgcctccgtcccatgcacgtctttatcctggattacgaccaa
tcgcccgccggctgccgggacgccctgctgcaacttacctccgggatggtccagacccacgtcaccacccc
cggctccataccgacgatctgcgacctggegcgcacgtttgcccgggagatgggggaggctaactcaggct
aaccggactctagaacatccctacaggtgatatcctcgtgctgcaagtaaacccctaccaactggtcgggg
tttgaaac
SEQ ID NO: 55 p3G μH-DR (pTRE3G-E2A(DBP)-IRES-E4(Orf6)-DR)
gtacacgccacctcgacatactcgagtttactccctatcagtgatagagaacgtatgaagagtttactccc
tatcagtgatagagaacgtatgcagactttactccctatcagtgatagagaacgtataaggagtttactcc
ctatcagtgatagagaacgtatgaccagtttactccctatcagtgatagagaacgtatctacagtttactc
cctatcagtgatagagaacgtatatccagtttactccctatcagtgatagagaacgtataagctttaggcg
tgtacggtgggcgcctataaaagcagagctcgtttagtgaaccgtcagatcgcctggagcaattccacaac
acttttgtcttataccaactttccgtaccacttcctaccctcgtaaaggacttagcctttagtgaaccgtc
agaattaattcagatcgatctaccagaaccgtcagatccgctagagattacgccaaccgccaccatgggca
gcgccagtcgggaagaggagcagcgcgaaaccacccccgagcgcggacgcggtgcggcgcgacgtccccca
accatggaggacgtgtcgtccccgtccccgtcgccgccgcctccccgggcgcccccaaaaaagcggatgag
gcggcgtatcgagtccgaggacgaggaagactcatcacaagacgcgctggtgccgcgcacacccagcccgc
ggccatcgacctcggcggcggatttggccattgcgcccaagaagaaaaagaagcgcccttctcccaagccc
gagcgcccgccatcaccagaggtaatcgtggacagcgaggaagaaagagaagatgtggcgctacaaatggt
gggtttcagcaacccaccggtgctaatcaagcatggcaaaggaggtaagcgcacagtgcggcggctgaatg
aagacgacccagtggcgcgtggtatgcggacgcaagaggaagaggaagagcccagcgaagcggaaagtgaa
attacggtgatgaacccgctgagtgtgccgatcgtgtctgcgtgggagaagggcatggaggctgcgcgcgc
gctgatggacaagtaccacgtggataacgatctaaaggcgaacttcaaactactgcctgaccaagtggaag
ctctggcggccgtatgcaagacctggctgaacgaggagcaccgcgggttgcagctgaccttcaccagcaac
aagacctttgtgacgatgatggggcgattcctgcaggcgtacctgcagtcgtttgcagaggtgacctacaa
gcatcacgagcccacgggctgcgcgttgtggctgcaccgctgcgctgagatcgaaggcgagcttaagtgtc
tacacggaagcattatgataaataaggagcacgtgattgaaatggatgtgacgagcgaaaacgggcagcgc
gcgctgaaggagcagtctagcaaggccaagatcgtgaagaaccggtggggccgaaatgtggtgcagatctc
caacaccgacgcaaggtgctgcgtgcacgacgcggcctgtccggccaatcagttttccggcaagtcttgcg
gcatgttcttctctgaaggcgcaaaggctcaggtggcttttaagcagatcaaggcttttatgcaggcgctg
tatcctaacgcccagaccgggcacggtcaccttttgatgccactacggtgcgagtgcaactcaaagcctgg
gcacgcgccctttttgggaaggcagctaccaaagttgactccgttcgccctgagcaacgcggaggacctgg
acgcggatctgatctccgacaagagcgtgctggccagcgtgcaccacccggcgctgatagtgttccagtgc
tgcaaccctgtgtatcgcaactcgcgcgcgcagggcggaggccccaactgcgacttcaagatatcggcgcc
cgacctgctaaacgcgttggtgatggtgcgcagcctgtggagtgaaaacttcaccgagctgccgcggatgg
ttgtgcctgagtttaagtggagcactaaacaccagtatcgcaacgtgtccctgccagtggcgcatagcgat
gcgcggcagaacccctttgatttttgactctagaacatccctacaggtgatatcctcgatcccgctcgata
agcttgatatcgaattccgcccccccccctctccctcccccccccctaacgttactggccgaagccgcttg
gaataaggccggtgtgcgtttgtctatatgttattttccaccatattgccgtcttttggcaatgtgagggc
ccggaaacctggccctgtcttcttgacgagcattcctaggggtctttcccctctcgccaaaggaatgcaag
gtctgttgaatgtcgtgaaggaagcagttcctctggaagcttcttgaagacaaacaacgtctgtagcgacc
ctttgcaggcagcggaaccccccacctggcgacaggtgcctctgcggccaaaagccacgtgtataagatac
acctgcaaaggcggcacaaccccagtgccacgttgtgagttggatagttgtggaaagagtcaaatggctct
cctcaagcgtattcaacaaggggctgaaggatgcccagaaggtaccccattgtatgggatctgatctgggg
cctcggtgcacatgctttacatgtgtttagtcgaggttaaaaaaacgtctaggccccccgaaccacgggga
cgtggttttcctttgaaaaacacgatgataatatggccacaaccgcgtccatgactacgtccggcgttcca
tttggcatgacactacgaccaacacgatctcggttgtctcggcgcactccgtacagtagggatcgtctacc
tccttttgagacagaaacccgcgctaccatactggaggatcatccgctgctgcccgaatgtaacactttga
caatgcacaacgtgagttacgtgcgaggtcttccctgcagtgtgggatttacgctgattcaggaatgggtt
gttccctgggatatggttctaacgcgggaggagcttgtaatcctgaggaagtgtatgcacgtgtgcctgtg
ttgtgccaacattgatatcatgacgagcatgatgatccatggttacgagtcctgggctctccactgtcatt
gttccagtcccggttccctgcagtgtatagccggcgggcaggttttggccagctggtttaggatggtggtg
gatggcgccatgtttaatcagaggtttatatggtaccgggaggtggtgaattacaacatgccaaaagaggt
aatgtttatgtccagcgtgtttatgaggggtcgccacttaatctacctgcgcttgtggtatgatggccacg
tgggttctgtggtccccgccatgagctttggatacagcgccttgcactgtgggattttgaacaatattgtg
gtgctgtgctgcagttactgtgctgatttaagtgagatcagggtgcgctgctgtgcccggaggacaaggcg
ccttatgctgcgggcggtgcgaatcatcgctgaggagaccactgccatgttgtattcctgcaggacggagc
ggcggcggcagcagtttattcgcgcgctgctgcagcaccaccgccctatcctgatgcacgattatgactct
acccccatgtaatgctgaggctaactgaaacacggaagtgctgcaagtaaacccctaccaactggtcgggg
tttgaaac
SEQ ID NO: 56 p5-Rep2/Cap2
tgggtatttaagcccgagtgagcacgcaggatctccattttgaagcgggaggtttgaacgcgcagccgcct
taattaaatgccggggttttacgagattgtgattaaggtccccagcgaccttgacgagcatctgcccggca
tttctgacagctttgtgaactgggtggccgagaaggaatgggagttgccgccagattctgacatggatctg
aatctgattgagcaggcacccctgaccgtggccgagaagctgcagcgcgactttctgacggaatggcgccg
tgtgagtaaggccccggaggctcttttctttgtgcaatttgagaagggagagagctacttccacatgcacg
tgctcgtggaaaccaccggggtgaaatccatggttttgggacgtttcctgagtcagattcgcgaaaaactg
attcagagaatttaccgcgggatcgagccgactttgccaaactggttcgcggtcacaaagaccagaaatgg
cgccggaggcgggaacaaggtggtggatgagtgctacatccccaattacttgctccccaaaacccagcctg
agctccagtgggcgtggactaatatggaacagtatttaagcgcctgtttgaatctcacggagcgtaaacgg
ttggtggcgcaacatctgacgcacgtgtcgcagacgcaggagcagaacaaagagaatcagaatcccaattc
tgatgcgccggtgatcagatcaaaaacttcagccaggtacatggagctggtcgggtggctcgtggacaagg
ggattacctcggagaagcagtggatccaggaggaccaggcctcatacatctccttcaatgcggcctccaac
tcgcggtcccaaatcaaggctgccttggacaatgcgggaaagattatgagcctgactaaaaccgcccccga
ctacctggtgggccagcagcccgtggaggacatttccagcaatcggatttataaaattttggaactaaacg
ggtacgatccccaatatgcggcttccgtctttctgggatgggccacgaaaaagttcggcaagaggaacacc
atctggctgtttgggcctgcaactaccgggaagaccaacatcgcggaggccatagcccacactgtgccctt
ctacgggtgcgtaaactggaccaatgagaactttcccttcaacgactgtgtcgacaagatggtgatctggt
gggaggaggggaagatgaccgccaaggtcgtggagtcggccaaagccattctcggaggaagcaaggtgcgc
gtggaccagaaatgcaagtcctcggcccagatagacccgactcccgtgatcgtcacctccaacaccaacat
gtgcgccgtgattgacgggaactcaacgaccttcgaacaccagcagccgttgcaagaccggatgttcaaat
ttgaactcacccgccgtctggatcatgactttgggaaggtcaccaagcaggaagtcaaagactttttccgg
tgggcaaaggatcacgtggttgaggtggagcatgaattctacgtcaaaaagggtggagccaagaaaagacc
cgcccccagtgacgcagatataagtgagcccaaacgggtgcgcgagtcagttgcgcagccatcgacgtcag
acgcggaagcttcgatcaactacgcagacaggtaccaaaacaaatgttctcgtcacgtgggcatgaatctg
atgctgtttccctgcagacaatgcgagagaatgaatcagaattcaaatatctgcttcactcacggacagaa
agactgtttagagtgctttcccgtgtcagaatctcaacccgtttctgtcgtcaaaaaggcgtatcagaaac
tgtgctacattcatcatatcatgggaaaggtgccagacgcttgcactgcctgcgatctggtcaatgtggat
ttggatgactgcatctttgaacaataaatgatttaaatcaggtatggctgccgatggttatcttccagatt
ggctcgaggacactctctctgaaggaataagacagtggtggaagctcaaacctggcccaccaccaccaaag
cccgcagagcggcataaggacgacagcaggggtcttgtgcttcctgggtacaagtacctcggacccttcaa
cggactcgacaagggagagccggtcaacgaggcagacgccgcggccctcgagcacgacaaagcctacgacc
ggcagctcgacagcggagacaacccgtacctcaagtacaaccacgccgacgcggagtttcaggagcgcctt
aaagaagatacgtcttttgggggcaacctcggacgagcagtcttccaggcgaaaaagagggttcttgaacc
tctgggcctggttgaggaacctgttaagacggctccgggaaaaaagaggccggtagagcactctcctgtgg
agccagactcctcctcgggaaccggaaaggcgggccagcagcctgcaagaaaaagattgaattttggtcag
actggagacgcagactcagtacctgacccccagcctctcggacagccaccagcagccccctctggtctggg
aactaatacgatggctacaggcagtggcgcaccaatggcagacaataacgagggcgccgacggagtgggta
attcctcgggaaattggcattgcgattccacatggatgggcgacagagtcatcaccaccagcacccgaacc
tgggccctgcccacctacaacaaccacctctacaaacaaatttccagccaatcaggagcctcgaacgacaa
tcactactttggctacagcaccccttgggggtattttgacttcaacagattccactgccacttttcaccac
gtgactggcaaagactcatcaacaacaactggggattccgacccaagagactcaacttcaagctctttaac
attcaagtcaaagaggtcacgcagaatgacggtacgacgacgattgccaataaccttaccagcacggttca
ggtgtttactgactcggagtaccagctcccgtacgtcctcggctcggcgcatcaaggatgcctcccgccgt
tcccagcagacgtcttcatggtgccacagtatggatacctcaccctgaacaacgggagtcaggcagtagga
cgctcttcattttactgcctggagtactttccttctcagatgctgcgtaccggaaacaactttaccttcag
ctacacttttgaggacgttcctttccacagcagctacgctcacagccagagtctggaccgtctcatgaatc
ctctcatcgaccagtacctgtattacttgagcagaacaaacactccaagtggaaccaccacgcagtcaagg
cttcagttttctcaggccggagcgagtgacattcgggaccagtctaggaactggcttcctggaccctgtta
ccgccagcagcgagtatcaaagacatctgcggataacaacaacagtgaatactcgtggactggagctacca
agtaccacctcaatggcagagactctctggtgaatccgggcccggccatggcaagccacaaggacgatgaa
gaaaagttttttcctcagagcggggttctcatctttgggaagcaaggctcagagaaaacaaatgtggacat
tgaaaaggtcatgattacagacgaagaggaaatcaggacaaccaatcccgtggctacggagcagtatggtt
ctgtatctaccaacctccagagaggcaacagacaagcagctaccgcagatgtcaacacacaaggcgttctt
ccaggcatggtctggcaggacagagatgtgtaccttcaggggcccatctgggcaaagattccacacacgga
cggacattttcacccctctcccctcatgggtggattcggacttaaacaccctcctccacagattctcatca
agaacaccccggtacctgcgaatccttcgaccaccttcagtgcggcaaagtttgcttccttcatcacacag
tactccacgggacaggtcagcgtggagatcgagtgggagctgcagaaggaaaacagcaaacgctggaatcc
cgaaattcagtacacttccaactacaacaagtctgttaatgtggactttactgtggacactaatggcgtgt
attcagagcctcgccccattggcaccagatacctgactcgtaatctgtaa
SEQ ID NO: 57: TRE3G-VPR-dCas9-N8
GTACACGCCACCTCGACATACTCGAGTTTACTCCCTATCAGTGATAGAGAACGTATGAAGAGTTTACTCCCTATC
AGTGATAGAGAACGTATGCAGACTTTACTCCCTATCAGTGATAGAGAACGTATAAGGAGTTTACTCCCTATCAGT
GATAGAGAACGTATGACCAGTTTACTCCCTATCAGTGATAGAGAACGTATCTACAGTTTACTCCCTATCAGTGAT
AGAGAACGTATATCCAGTTTACTCCCTATCAGTGATAGAGAACGTATAAGCTTTAGGCGTGTACGGTGGGCGCCT
ATAAAAGCAGAGCTCGTTTAGTGAACCGTCAGATCGCCTGGAGCAATTCCACAACACTTTTGTCTTATACCAACT
TTCCGTACCACTTCCTACCCTCGTAAAGGACttagcctttagtgaaccgtcagaattaattcagatcgatctacc
agaaccggatctaccagaaccgtcagatccgctagagattacgccaaccgccaccatgggcagccgggctgacgc
attggacgattttgatctggatatgctgggaagtgacgccctcgatgattttgaccttgacatgcttggttcgga
tgcccttgatgactttgacctcgacatgctcggcagtgacgcccttgatgatttcgacctggacatgctgattaa
ctctagaagttccggatctccgaaaaagaaacgcaaagttggtagccagtacctgcccgacaccgacgaccggca
ccggatcgaggaaaagcggaagcggacctacgagacattcaagagcatcatgaagaagtcccccttcagcggccc
caccgaccctaggcctccacctagaagaatcgccgtgcccagcagatccagcgccagcgtgccaaaacctgcccc
ccagccttaccccttcaccagcagcctgagcaccatcaactacgacgagttccctaccatggtgttccccagcgg
ccagatctctcaggcctctgctctggccccagcccctcctcaggtgctgcctcaggctcctgctcctgcaccagc
tccagccatggtgtctgcactggctcaggcaccagcacccgtgcctgtgctggctcctggacctccacaggctgt
ggctccaccagcccctaaacctacacaggccggcgagggcacactgtctgaagctctgctgcagctgcagttcga
cgacgaggatctgggagccctgctgggaaacagcaccgatcctgccgtgttcaccgacctggccagcgtggacaa
cagcgagttccagcagctgctgaaccagggcatccctgtggcccctcacaccaccgagcccatgctgatggaata
ccccgaggccatcacccggctcgtgacaggcgctcagaggcctcctgatccagctcctgcccctctgggagcacc
aggcctgcctaatggactgctgtctggcgacgaggacttcagctctatcgccgatatggatttctcagccttgct
gggctctggcagcggcagccgggattccagggaagggatgtttttgccgaagcctgaggccggctccgctattag
tgacgtgtttgagggccgcgaggtgtgccagccaaaacgaatccggccatttcatcctccaggaagtccatgggc
caaccgcccactccccgccagcctcgcaccaacaccaaccggtccagtacatgagccagtcgggtcactgacccc
ggcaccagtccctcagccactggatccagcgcccgcagtgactcccgaggccagtcacctgttggaggatcccga
tgaagaaacgagccaggctgtcaaagcccttcgggagatggccgatactgtgattccccagaaggaagaggctgc
aatctgtggccaaatggacctttcccatccgcccccaaggggccatctggatgagctgacaaccacacttgagtc
catgaccgaggatctgaacctggactcacccctgaccccggaattgaacgagattctggataccttcctgaacga
cgagtgcctcttgcatgccatgcatatcagcacaggactgtccatcttcgacacatctctgtttaaagcctatga
caagaaatactccatcggccttgccatcgggaccaacagcgtgggttgggccgtgatcactgacgaatacaaagt
gccatcgaaaaagtttaaggtgctgggcaatactgacagacatagcatcaagaaaaacctgattggagccctcct
gttcgactccggggaaaccgccgaagccactcgcctgaaacggaccgcacgccggaggtacactcgccggaagaa
tagaatctgctaccttcaagagatctttagcaacgagatggccaaagtggacgattcgttcttccaccggctgga
ggagtcattcctggtcgaagaggacaaaaagcatgagagacatccgatcttcggtaacattgtggatgaggtcgc
ctaccacgagaagtaccccactatctaccatttgcgcaagaagttggtcgactcgactgataaggccgacctcag
actcatctacctcgctttggcacacatgatcaagtttaggggtcacttcctgattgaaggggatctgaacccaga
caactcggatgtggataaactgtttatccagctggtccagacttacaatcagctctttgaggagaacccgatcaa
tgcctccggcgtggatgctaaggcaatcctgtccgcacgcttgtcaaagagccggcggctcgaaaatctgatcgc
acagcttccgggagaaaagaaaaacggactgtttggaaaccttatcgccctctcgctgggactcactcctaactt
caaatccaattttgatttggccgaggatgccaagctgcagctgtcgaaggacacctacgacgatgacctcgacaa
tcttctggcccagatcggggaccagtacgccgatctgttcctcgcggccaaaaacctgtcagacgcaatcttgct
gagcgatatcttgagggtcaataccgaaatcactaaggctccattgtcagcatcgatgatcaagaggtacgatga
acaccatcaggacctgaccctgctcaaggcactggtgcggcagcaactgccggaaaagtacaaggagatcttctt
cgatcaaagcaagaatgggtacgcagggtacatcgatggtggagcatcacaagaagagttctacaaattcatcaa
gcccatccttgaaaagatggacgggacggaagaactgctggtgaagctgaatcgggaagatctgctgcggaagca
gcgcaccttcgacaatggttccatcccacaccaaatccatctcggcgaactgcacgcgatccttcgccggcagga
agatttctacccgttcttgaaggataacagagaaaagatcgagaaaatcctgacctttagaatcccgtactacgt
gggcccgttggctcgcggaaactcaagattcgcctggatgactagaaaatccgaagaaacgattaccccgtggaa
ctttgaagaggtcgtcgacaagggagcctcggcacagtcgttcatcgaacggatgaccaatttcgacaagaacct
ccccaacgaaaaggtgctcccgaaacactcgctgctgtatgagtacttcacggtctacaacgagctgaccaaggt
gaagtacgtgaccgaaggaatgcggaagcccgcttttctgtccggtgaacagaaaaaggccatcgtggacctcct
cttcaagactaacagaaaggtgaccgtgaaacagcttaaagaggactacttcaagaagatcgagtgttttgactc
cgtggaaatctcgggagtggaggataggtttaacgcttcgctggggacctaccatgaccttctcaagatcatcaa
ggataaggatttcctggacaacgaagaaaacgaggacatccttgaggacatcgtgttgaccttgaccctgtttga
ggatcgggaaatgatcgaggagagactgaaaacttacgcgcatcttttcgacgacaaagtgatgaagcagctgaa
aagaagaagatacactggctggggacggctgtcgaggaaactgattaacggaattagggataaacaaagcggaaa
gactatcctcgattttctcaagtcggacggcttcgccaatcggaactttatgcagctcatccacgacgatagcct
gaccttcaaggaagatatccaaaaagcccaagtgtccggccagggagatagcttgcacgagcacattgctaacct
ggccggttcaccagccattaagaagggaatcctccaaaccgtgaaggtggtcgacgaactcgtcaaggtgatggg
ccgccataagccagagaacatcgtgatcgagatggctcgcgaaaatcagactactcagaagggtcaaaagaactc
ccgcgagcgcatgaagcgcatcgaagaaggaattaaggagctgggctcacaaatcctcaaagaacatcctgtcga
gaacacccaacttcagaacgagaaactgtacctctactatctccaaaatggccgggacatgtacgtcgatcagga
attggatatcaaccgcctctcagactacgacgtggatgctatcgtgccgcagtcgtttctcaaagatgatagcat
cgacaacaaggtcctgacgaggtcggataagaaccgcgggaaatccgacaatgtgccttcggaagaggtggtgaa
aaagatgaagaactactggaggcaattgcttaatgccaaactgatcacccagcgcaaattcgacaaccttactaa
ggccgagcgcggaggactgtccgaactcgataaggcggggttcatcaaaagacaattggtcgaaactcggcaaat
taccaagcatgtcgctcagatcctcgactcgcgcatgaacactaagtatgatgagaacgacaagctcattcgcga
agtgaaagtgattaccctcaaatcaaagctggtgtcggacttcagaaaagactttcaattctacaaggtgcggga
gattaacaactaccaccacgcgcacgacgcctacctcaatgcagtggtgggaaccgccctcatcaagaagtatcc
aaagctcgaaagcgagttcgtgtacggggactataaagtgtacgatgtgcggaaaatgatcgcaaagagcgagca
agagatcggcaaagcaacggccaaatacttcttctacagcaacattatgaactttttcaagaccgagattaccct
ggcgaacggagaaatccggaagcggcctttgatcgaaacgaatggggaaactggagaaatcgtgtgggacaaggg
cagagactttgcgaccgtgagaaaggtgctgtccatgccacaagtcaacatcgtgaagaaaactgaagtgcagac
tggaggattttccaaggagtcaatccttccgaagcgcaacagcgacaagctgatcgccaggaagaaggattggga
ccccaagaagtacggtggttttgattcacctactgtcgcttacagcgtgctggtggtggccaaggtggaaaaggg
gaagtcaaagaaattgaagtcggtgaaggaattgctggggattactatcatggagaggagcagcttcgaaaagaa
tcccatcgacttcttggaggccaagggatacaaagaggtgaagaaagacctgatcattaagctgccgaagtactc
cttgttcgaactggaaaacggcagaaagcggatgctggcctccgccggagagctgcagaagggtaacgagctggc
tctgcccagcaaatacgtgaatttcctctacctggcctcccattacgagaagctcaagggaagcccggaggataa
tgagcaaaaacaactgttcgtcgagcagcacaagcactacctcgacgagatcatcgaacaaatctccgagttctc
gaagcgggtgattctggccgacgcaaaccttgataaagtcctcagcgcctacaacaagcaccgcgacaaaccgat
cagagaacaagctgagaacatcatccacctgttcacgctgactaacttgggagcgcctgccgccttcaagtactt
cgacaccactatcgatcggaaacggtacacttccactaaggaagtgctggacgcaaccctgatccatcagtccat
caccggactttatgagactcggatcgatctcagccagcttggcggtgattcaagagctgacccgaagaagaagcg
caaggtcTCAGGCtctggactcagatctggcagcggaggaggcagcgcatctggcggaagcggagcgtCCttaaa
ggcggagaacgcagctctggaagccaagattgcagccttaaaagcggagattgctgcgttagaggcaggctacTA
A

1. An engineered genetic circuit for controlled expression of genes in a cell,
said genetic circuit comprising:
a) at least one transcription unit for expressing a transcriptional activator in the cell, comprising a promoter operably linked to a polynucleotide encoding the transcriptional activator, preferably an inducible transcriptional activator, said transcriptional activator positively regulating the expression of at least one gene of interest;
b) at least one transcription unit for expressing a controller biomolecule in the cell, comprising a promoter operably linked to a polynucleotide encoding for the controller biomolecule;
and optionally comprising:
c) at least one transcription unit for expressing the at least one gene of interest in the cell, comprising a promoter operably linked to at least one gene of interest, preferably wherein said promoter comprises a binding site for the transcriptional activator, such that binding of the transcriptional activator to said binding site positively regulates the expression of the gene of interest;
wherein the at least one controller biomolecule negatively regulates at least one component of the genetic circuit; and wherein:
i) the promoter of b) the at least one transcription unit for expressing the controller biomolecule is such that the transcriptional activator negatively regulates the expression of said controller biomolecule, and wherein the at least one component of the genetic circuit that is negatively regulated by the controller biomolecule is the product of the at least one gene of interest; or
ii) the promoter of b) the at least one transcription unit for expressing the controller biomolecule is such that the transcriptional activator positively regulates the expression of the controller biomolecule and the at least one component of the genetic circuit that is negatively regulated by the controller biomolecule is a transcriptional activator.

2. The engineered genetic circuit of claim 1, wherein the promoter of b) the transcription unit for expressing the controller biomolecule is such that i) the transcriptional activator negatively regulates the expression of said controller biomolecule, and wherein the at least one component of the genetic circuit that is negatively regulated by the controller biomolecule is the product of the at least one gene of interest.

3. The engineered genetic circuit of claim 2, wherein said promoter of b) the transcription unit for expressing the controller biomolecule comprises a binding site for binding of the transcriptional activator such that binding of the transcriptional activator prevents binding of the RNA polymerase to said promoter.

4. The engineered genetic circuit of claim 1, wherein the controller biomolecule is a CRISPR-associated endoribonuclease.

5. The engineered genetic circuit of claim claim 1, wherein the controller biomolecule is CasRx endoribonuclease encoded by a polynucleotide having sequence comprising SEQ ID NO: 2; and wherein the sequence of the gene of interest negatively regulated by the controller biomolecule comprises at least one Direct Repeat having sequence SEQ ID NO: 3, or variants thereof; preferably wherein the transcription unit for expressing said controller biomolecule has sequence comprising SEQ ID NO: 14.

6. (canceled)

7. The engineered genetic circuit of claim 1, wherein c) the at least one transcription unit for expressing the at least one gene of interest in the cell comprises:
c′) at least one first transcription unit comprising a polynucleotide encoding for an AAV Rep protein and an AAV Cap protein, having sequence comprising SEQ ID NO: 10, or SEQ ID NO: 56, or variants thereof, and
c″) at least one second transcription unit for expressing the at least one gene of interest, comprising a polynucleotide encoding for one or more adenoviral helper proteins, preferably encoding for E2A(DBP) having sequence SEQ ID NO: 11 and for E4(Orf6) having sequence SEQ ID NO: 12.

8. The engineered genetic circuit of claim 7, wherein:
b) the transcription unit for expressing the controller biomolecule comprises a promoter having sequence SEQ ID NO: 8, or variants thereof operably linked to a polynucleotide encoding for a CasRx controller biomolecule having sequence SEQ ID NO: 2, or variants thereof;
and wherein c″) the at least one second transcription unit for expressing one or more adenoviral helper proteins, has sequence comprising SEQ ID NO: 55 or variants thereof.

9. The engineered genetic circuit of claim 7, comprising:
a) at least one transcription unit for expressing a rtTA inducible transcriptional activator having sequence SEQ ID NO: 6, or variants thereof;
b) a transcription unit for expressing at least one controller biomolecule comprising sequence SEQ ID NO: 14 or variants thereof;
c′) at least one transcription unit for expressing an AAV Rep protein and AAV Cap protein having sequence comprising of SEQ ID NO: 10, SEQ ID NO: 56, or variants thereof; and
c″) the at least one second transcription unit for expressing one or more adenoviral helper proteins having sequence comprising of SEQ ID NO: 55 or variants thereof.

10. The engineered genetic circuit of claim 1, wherein the gene of interest is a cytotoxic gene or a reporter gene, preferably wherein c) the at least one transcription unit for expressing the at least one gene of interest in the cell has sequence comprising SEQ ID NO: 28, 29, 30, or 47 or variants thereof.

11. The engineered genetic circuit of claim 1, wherein
a) the at least one transcription unit for expressing a transcriptional activator in the cell, comprising a promoter operably linked to the polynucleotide encoding the transcriptional activator, said promoter being responsive to endogenous transcription factor in the cell, preferably said promoter being a NiClear promoter having sequence SEQ ID NO: 42, more preferably the a) at least one transcription unit for expressing a transcriptional activator having sequence SEQ ID NO: 31, 51 or variants thereof.

12. The engineered genetic circuit of claim 1, wherein the promoter of b) the transcription unit for expressing the controller biomolecule is such that:
ii) the transcriptional activator positively regulates the expression of the controller biomolecule,
and wherein the at least one component of the genetic circuit that is negatively regulated by the controller biomolecule is a transcriptional activator of the genetic circuit.

13. The engineered genetic circuit of claim 12 comprising:
a′) a first transcription unit for expressing a first transcriptional activator in the cell, comprising a promoter operably linked to a polynucleotide encoding the first transcriptional activator, and
a″) a second transcription unit for expressing a second transcriptional activator in the cell, comprising a promoter operably linked to a polynucleotide encoding the second transcriptional activator,
said first or second transcriptional activator positively regulating the expression of at least one gene of interest,
and wherein:
the promoter operably linked to the polynucleotide encoding the controller biomolecule comprises a binding site for the second transcriptional activator, such that ii) binding of the second transcriptional activator to said binding site positively regulates the expression of the controller biomolecule,
the promoter operably linked to the polynucleotide encoding for the second transcriptional activator comprises a binding site for the first transcriptional activator, such that binding of the first transcriptional activator to said binding site positively regulates the expression of the second transcriptional activator, and
the at least one component of the genetic circuit that is negatively regulated by the controller biomolecule is the first transcriptional activator.

14. The engineered genetic circuit of claim 12, wherein said controller biomolecule is a protein capable of sequestering the transcriptional activator; and/or wherein the transcriptional activator that positively regulates the expression of at least one gene of interest is a modified CRISPR-associated endoribonuclease which is nuclease-deficient and which is fused to a transactivation domain, and the controller biomolecule that negatively regulates the transcriptional activator is an inhibitor of a CRISPR-associated endoribonuclease.

15. (canceled)

16. The engineered genetic circuit of claim 14, wherein the controller biomolecule is AcrII4A and the polynucleotide encoding said controller biomolecule in the cell has sequence comprising SEQ ID NO: 18 or 23, or variants thereof, and wherein the transcriptional activator negatively regulated by the controller biomolecule is VPR-dCas9 and the polynucleotide encoding said transcriptional activator has sequence comprising SEQ ID NO: 17 or 24, or variants thereof; and/or wherein the gene of interest is positively regulated by a″) the second transcription unit for expressing a second transcriptional activator in the cell.

17. (canceled)

18. The engineered genetic circuit of claim 14, wherein the gene of interest is positively regulated by a″) the second transcription unit for expressing a second transcriptional activator in the cell, and wherein the second transcriptional activator, expressed by a″) the second transcription unit, is rtTA and the polynucleotide encoding said transcriptional activator has sequence comprising SEQ ID NO: 1 or 4 or variants thereof.

19. The engineered genetic circuit of claim 14, wherein the gene of interest is positively regulated by a″) the second transcription unit for expressing a second transcriptional activator in the cell, and wherein:
a′) the first transcription unit for expressing a first transcriptional activator in the cell, comprises a promoter operably linked to a polynucleotide encoding the first transcriptional activator VPR-dCas9, said polynucleotide having sequence comprising SEQ ID NO: 17 or 24, or variants thereof;
the genetic circuit further comprising a gRNA complementary to the promoter of a″) the second transcription unit;
wherein said first activator, in the presence of said gRNA, positively regulates the a″) the second transcription unit;
preferably wherein the promoter of a″) the second transcription unit is 7B-pMin having sequence comprising or consisting of SEQ ID NO: 46 or variants thereof.

20. (canceled)

21. The engineered genetic circuit of claim 14, wherein the gene of interest is positively regulated by a′) the first transcription unit for expressing a second transcriptional activator in the cell, wherein:
a′) the first transcription unit for expressing a first transcriptional activator in the cell, comprises a promoter operably linked to a polynucleotide encoding the first transcriptional activator VPR-dCas9 having sequence comprising SEQ ID NO: 17 or 24, or variants thereof;
the genetic circuit further comprising a gRNA complementary to the promoter of the gene of interest and/or of the second activator;
wherein said first activator, in the presence of said gRNA, positively regulates the gene of interest and/or a″) the second transcription unit for expressing the second activator;
preferably wherein the promoter of the gene of interest and or of a″) the second transcription unit is 7B-pMin having sequence comprising SEQ ID NO: 46 or variants thereof.

22. (canceled)

23. The engineered genetic circuit of claim 1, wherein the transcriptional activator that positively regulates the expression of at least one gene of interest is an inducible transcriptional activator and/or wherein:
a) the polynucleotide encoding the transcriptional activator that positively regulates the expression of at least one gene of interest has sequence comprising SEQ ID NO: 1 or 4 or variants thereof, preferably wherein the transcription unit for expressing said transcriptional activator has sequence comprising SEQ ID NO:6 or variants thereof and preferably the promoter operably linked to the polynucleotide encoding for the controller biomolecule has sequence comprising SEQ ID NO: 7 or 8, or variants thereof.

24. (canceled)

25. A biosensor comprising the genetic circuit of claim 2, said genetic circuit comprising:
a) a transcription unit, comprising a MRE promoter, having sequence SEQ ID NO: 45 or variants thereof, operably linked to a polynucleotide encoding a transcriptional activator VPR-dCas9 having sequence SEQ ID NO: 17 or SEQ ID NO: 24, which transcriptional activator induces expression of the gene of interest being a reporter gene comprising Direct Repeats of sequence SEQ ID NO: 3; wherein the controller biomolecule is under the control of a promoter which is negatively regulated by the transcriptional activator, preferably said promoter being CMV/2B having sequence SEQ ID NO: 48.

26. A biosensor comprising the genetic circuit of claim 12, said genetic circuit comprising:
a′) a first transcription unit, comprising a MRE promoter having sequence SEQ ID NO: 45, operably linked to a polynucleotide encoding a first transcriptional activator VPR-dCas9 having sequence SEQ ID NO: 17 or SEQ ID NO: 24,
and a″) a second transcription unit for expressing a second transcriptional activator being an inducible activator preferably rtTA;
said first transcriptional activator inducing the expression of said second transcriptional activator;
said second transcriptional activator inducing the expression of the gene of interest, being a reporter gene;
the controller biomolecule being a controller biomolecule having sequence SEQ ID NO: 18 or 23 or variants thereof, said controller biomolecule negatively regulating the first transcriptional activator,
preferably the biosensor further comprising a second controller biomolecule being CasRx, having sequence SEQ ID NO: 2;
wherein the second transcriptional activator negatively regulates said second controller biomolecule or wherein the first transcriptional activator negatively regulates said second controller biomolecule;
preferably wherein said second transcriptional activator comprises Direct Repeats of sequence SEQ ID NO: 3 recognized by the controller CasRx, more preferably said second transcriptional activator having sequence SEQ ID NO: 50.

27.-30. (canceled)

31. A cell line comprising the genetic circuits of claim 1, said cell line being a mammalian cell line preferably a stable cell line.

32.-40. (canceled)

41. Method of controlling expression of genes in a cell, comprising: introducing in the cell one or more plasmids bearing the transcription units of the genetic circuit of claim 1 and expressing said transcription units in the cell.
